(19) United States
(12) Patent Application Publication
(10) Pub. No.: US 2023/0141672 A1
(43) Pub. Date: May 11, 2023

(54) PLURALITY OF HOST MATERIALS AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: So-Young JUNG, Gyeonggi-do (KR); Jin-Ri HONG, Gyeonggi-do (KR); Su-Hyun LEE, Gyeonggi-do (KR); Sang-Hee CHO, Gyeonggi-do (KR); So-Mi PARK, Gyeonggi-do (KR); Seung-Hyun YOON, Gyeonggi-do (KR)

(21) Appl. No.: 17/952,499

(22) Filed: Sep. 26, 2022

(30) Foreign Application Priority Data
Oct. 22, 2021 (KR) 10-2021-0141870

(51) Int. Cl. H01L 51/00; C07D 307/91; C07D 345/00; C07D 405/10; C09K 11/02

(52) U.S. Cl. CPC H01L 51/006; C07D 307/91; C07D 345/00; H01L 51/0061; C07D 405/10; H01L 51/0067; H01L 51/0073; C09K 11/025; H01L 51/5016

(57) ABSTRACT

The present disclosure relates to a plurality of host materials comprising at least one first host compound and at least one second host compound, and an organic electroluminescent device comprising the same. By comprising a specific combination of compounds according to the present disclosure as a plurality of host materials, it is possible to provide an organic electroluminescent device having improved driving voltage, luminous efficiency, and/or lifetime properties.

### TECHNICAL FIELD

The present disclosure relates to a plurality of host materials and an organic electroluminescent device comprising the same.

### BACKGROUND ART

A small molecular green organic electroluminescent device (OLED) was first developed by Tang, et al., of Eastman Kodak in 1987 by using TPD/ALq3 bi-layer consisting of a light-emitting layer and a charge transport layer. Thereafter, the development of OLEDs was rapidly effected and OLEDs have been commercialized. At present, OLEDs primarily use phosphorescent materials having excellent luminous efficiency in panel implementation. However, an OLED having low driving voltage, high luminous efficiency and/or long lifetime characteristics is required for long time use and high resolution of a display.

In order to enhance luminous efficiency, driving voltage and/or lifetime, various materials or concepts for an organic layer of an OLED have been proposed. However, they were not satisfied in practical use.

Meanwhile, Korean Patent Application Laying-Open No. 2012-0062763 (published on Jun. 14, 2012) discloses a compound in which a diarylamino is bonded to a chrysene structure, and Korean Patent Application Laying-Open No. 2021-0056940 (published on May 20, 2021) discloses an organic electroluminescent device comprising a benzonaphthofuran or benzonaphthothiophene compound to which a nitrogen-containing heteroaryl is bonded, and a phenanthrene compound. However, the aforementioned references do not specifically disclose a specific combination of host materials claimed in the present disclosure. In addition, there has been a need to develop electroluminescent materials having improved performances, for example, improved driving voltage, luminous efficiency, power efficiency, and/or lifetime properties, compared to the previously disclosed specific compounds or a specific combination of compounds.

### DISCLOSURE OF INVENTION

### Technical Problem

The objective of the present disclosure is to provide a plurality of host materials capable of providing an organic electroluminescent device having low driving voltage, high luminous efficiency, and/or excellent lifetime properties. Another objective of the present disclosure is to provide an organic electroluminescent device having low driving voltage, high luminous efficiency, and/or excellent lifetime properties by comprising a plurality of host materials according to the present disclosure.

### Solution to Problem

As a result of intensive studies to solve the technical problems, the present inventors found that the above objective can be achieved by a plurality of host materials comprising at least one first host compound and at least one second host compound, wherein the first host compound is represented by the following formula 1, and the second host compound is represented by the following formula 2.

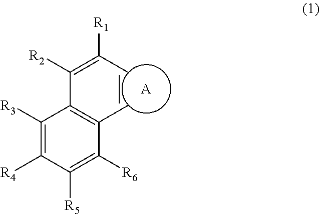

In formula 1, ring A represents

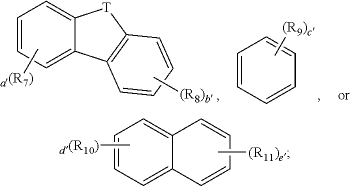

R₁ to R₁₁, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₁-N—(Ar₁)(Ar₂); with the proviso that at least one of R₁ to R₁₁ represents -L₁-N—(Ar₁)(Ar₂);

T represents O, S, or Se;

L₁, each independently, represents a single bond, a substituted or unsubstituted (C6-C30)arylene, or a substituted or unsubstituted (3- to 30-membered)heteroarylene; Ar₁ and Ar₂, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and

a′ to e′, each independently, represent an integer of 1 to 4, where if a′ to e′ are each an integer of 2 or more, each of R₇ to each of R₁ may be the same as or different from each other.

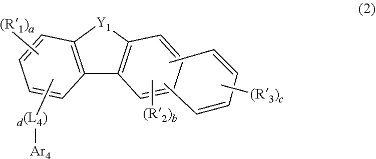

In formula 2,

Y₁ represents O or S;

R′₁ to R′₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;

L₄, each independently, represents a single bond, a substituted or unsubstituted (C6-C30)arylene, or a substituted or unsubstituted (3- to 30-membered)heteroarylene;

Ar₄ represents a substituted or unsubstituted (3- to 30-membered)heteroaryl containing a nitrogen(s);

a and d, each independently, represent an integer of 1 to 3, b represents an integer of 1 or 2, and c represents an integer of 1 to 4; where if a to d are each an integer of 2 or more, each of R′₁, each of R′₂, each of R′₃, and each of L₄ may be the same as or different from each other;

with the proviso that when the first host compound comprises a compound in which ring A represents

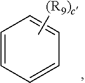

and the second host compound comprises a compound represented by the following formula 2′, at least one of R₃ to R₆, and R₉ is -L₁-N—(Ar₁)(Ar₂); and

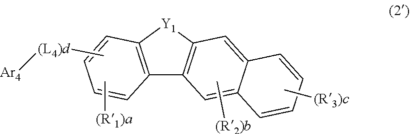

in formula 2′, Y₁, R′, to R′₃, L₄, Ar₄, and a to d are as defined in formula 2.

### Advantageous Effects of Invention

An organic electroluminescent device having low driving voltage, high luminous efficiency, and/or excellent lifetime properties compared to conventional organic electroluminescent devices is provided by comprising the plurality of host materials according to the present disclosure, and it is possible to produce a display system or a lighting system using the same.

### MODE FOR THE INVENTION

Hereinafter, the present disclosure will be described in detail. However, the following description is intended to explain the present disclosure and is not meant in any way to restrict the scope of the present disclosure.

The term “organic electroluminescent compound” in the present disclosure means a compound that may be used in an organic electroluminescent device, and may be comprised in any material layer constituting an organic electroluminescent device, as necessary.

The term “an organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

The term “a plurality of organic electroluminescent materials” in the present disclosure means an organic electroluminescent material comprising a combination of at least two compounds, which may be comprised in any organic layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials of the present disclosure may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. The at least two compounds may be comprised in the same layer or different layers, and may be mixture-evaporated or co-evaporated, or may be individually evaporated.

The term “a plurality of host materials” in the present disclosure means an organic electroluminescent material comprising a combination of at least two host materials. It may mean both a material before being comprised in an organic electroluminescent device (for example, before vapor deposition) and a material after being comprised in an organic electroluminescent device (for example, after vapor deposition). The plurality of host materials of the present disclosure may be comprised in any light-emitting layer constituting an organic electroluminescent device, at least two compounds comprised in the plurality of host materials of the present disclosure may be comprised together in one light-emitting layer or may respectively be comprised in different light-emitting layers. For example, the at least two host materials may be mixture-evaporated to form a layer, or separately co-evaporated at the same time to form a layer.

Herein, the term “(C1-C30)alkyl” is meant to be a linear or branched alkyl having 1 to 30 carbon atoms constituting the chain, in which the number of carbon atoms is preferably 1 to 20, and more preferably 1 to 10. The above alkyl may include methyl, ethyl. n-propyl, isopropyl, n-butyl, isobutyl, tert-butyl, sec-butyl, etc. The term “(C2-C30)alkenyl” in the present disclosure is meant to be a linear or branched alkenyl having 2 to 30 carbon atoms constituting the chain, in which the number of carbon atoms is preferably 2 to 20, and more preferably 2 to 10. The above alkenyl may include vinyl, 1-propenyl, 2-propenyl, 1-butenyl, 2-butenyl, 3-butenyl, 2-methylbut-2-enyl, etc. The term “(C2-C30)alkynyl” in the present disclosure is meant to be a linear or branched alkynyl having 2 to 30 carbon atoms constituting the chain, in which the number of carbon atoms is preferably 2 to 20, and more preferably 2 to 10. The above alkynyl may include ethynyl, 1-propynyl, 2-propynyl, 1-butynyl, 2-butynyl, 3-butynyl, 1-methylpent-2-ynyl, etc. The term “(C3-C30)cycloalkyl” is meant to be a mono- or polycyclic hydrocarbon having 3 to 30 ring backbone carbon atoms, in which the number of carbon atoms is preferably 3 to 20, and more preferably 3 to 7. The above cycloalkyl may include cyclopropyl, cyclobutyl, cyclopentyl, cyclohexyl, cyclopentylmethyl, cyclohexylmethyl, etc. The term “(3- to 7-membered)heterocycloalkyl” is meant to be a cycloalkyl having 3 to 7 ring backbone atoms, preferably 5 to 7 ring backbone atoms, and including at least one heteroatom selected from the group consisting of B, N, O, S, Si, and P, and preferably the group consisting of O, S, and N. The above heterocycloalkyl may include tetrahydrofuran, pyrrolidine, thiolan, tetrahydropyran, etc. The term “(C6-C30)aryl(ene)” is meant to be a monocyclic or fused ring radical derived from an aromatic hydrocarbon having 6 to 30 ring backbone carbon atoms, and may be partially saturated. The ring backbone carbon atoms is preferably 6 to 25, more preferably 6 to 18. The above aryl may comprise a spiro structure. The above aryl may include phenyl, biphenyl, terphenyl, naphthyl, binaphthyl, phenylnaphthyl, naphthylphenyl, phenylterphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, spirobifluorenyl, azulenyl, tetramethyldihydrophenanthrenyl, etc. Specifically, the above aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenyl, 3-biphenyl, 4-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xytyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-8-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

The term “(3- to 30-membered)heteroaryl(ene)” or “(3- to 50-membered)heteroaryl(ene)” is meant to be an aryl(ene) having 3 to 30 or 3 to 50 ring backbone atoms, and including at least one, preferably 1 to 4 heteroatoms selected from the group consisting of B, N, O, S, Si, and P. The above heteroaryl(ene) may be a monocyclic ring, or a fused ring condensed with at least one benzene ring; may be partially saturated; may be one formed by linking at least one heteroaryl or aryl group to a heteroaryl group via a single bond(s); and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, and pyridazinyl, and a fused ring-type heteroaryl such as benzofuranyl, benzothiophenyl, isobenzofuranyl, dibenzofuranyl, dibenzothiophenyl, dibenzoselenophenyl, naphthobenzofuranyl, naphthobenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzimidazolyl, benzothiazolyl, benzoisothiazolyl, benzoisoxazolyl, benzoxazolyl, isoindolyl, indolyl, benzoindolyl, indazolyl, benzothiadiazolyl, quinolyl, isoquinolyl, cinnolinyl, quinazolinyl, benzoquinazolinyl, quinoxalinyl, benzoquinoxalinyl, naphthyridinyl, carbazolyl, benzocarbazolyl, dibenzocarbazolyl, phenoxazinyl, phenothiazinyl, phenanthridinyl, benzodioxolyl, dihydroacridinyl, benzotriazolephenazinyl, imidazopyridyl, chromenoquinazolinyl, thiochromenoquinazolinyl, dimethylbenzoperimidinyl, indolocarbazolyl, indenocarbazolyl, etc. More specifically, the above heteroaryl may include 1-pyrrolyl, 2-pyrrolyl, 3-pyrrolyl, pyrazinyl, 2-pyridinyl, 2-pyrimidinyl, 4-pyrimidinyl, 5-pyrimidinyl, 6-pyrimidinyl, 1,2,3-triazin-4-yl, 1,2,4-triazin-3-yL, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridinyl, 3-imidazopyridinyl, 5-imidazopyridinyl, 6-imidazopyridinyl, 7-imidazopyridinyl, 8-imidazopyridinyl, 3-pyridinyl, 4-pyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-tert-butyl-1-indolyl, 4-tert-butyl-1-indolyl, 2-tert-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2,3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyridinyl, 6-benzofuro[3,2-d]pyrimidinyl, 7-benzofuro[3,2-d]pyridinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyridinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. Furthermore, “halogen” includes F, Cl, Br, and I.

In addition, “ortho (o-),” “meta (m-),” and “para (p-)” are prefixes, which represent the relative positions of substituents respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, “substituted” in the expression “substituted or unsubstituted” means that a hydrogen atom in a certain functional group is replaced with another atom or another functional group, i.e., a substituent, and also includes that the hydrogen atom is replaced with a group formed by a linkage of two or more substituents of the above substituents. For example, the “group formed by a linkage of two or more substituents” may be pyridine-triazine. That is, pyridine-triazine may be interpreted as a heteroaryl substituent, or as substituents in which two heteroaryl substituents are linked. Herein, the substituent(s) of the substituted alkyl, the substituted aryl, the substituted arylene, the substituted heteroaryl, the substituted heteroarylene, the substituted cycloalkyl, the substituted heterocycloalkyl, the substituted fused ring group of an aliphatic ring(s) and a aromatic ring(s), the substituted trialkylsilyl, the substituted dialkylarylsilyl, the substituted alkyldiarylsilyl, the substituted triarylsilyl, the substituted mono- or di-alkylamino, the substituted mono- or di-arylamino, and the substituted alkylarylamino, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of deuterium, a halogen(s), a cyano(s), a (C1-C30)alkyl(s), a (C6-C30)aryl(s), a (3- to 30-membered)heteroaryl(s) and a tri(C6-C30)arylsilyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl. According to one embodiment of the present disclosure, the substituent(s), each independently, are at least one selected from the group consisting of deuterium; a halogen; a cyano; a (C1-C10)alkyl; a (C6-C30)aryl unsubstituted or substituted with at least one of deuterium, a halogen(s), a cyano(s), a (C1-C10)alkyl(s), a (C6-C30)aryl(s), a (5- to 25-membered)heteroaryl(s) and a tri(C6-C18)arylsilyl(s); a (3- to 30-membered)heteroaryl unsubstituted or substituted with a (C6-C18)aryl(s); a tri(C6-C12)arylsilyl; and a (C6-C12)aryl(C1-C10)alkyl. According to another embodiment of the present disclosure, the substituent(s), each independently, are at least one selected from the group consisting of deuterium; a halogen; a cyano; a (C1-C6)alkyl; a (C6-C28)aryl unsubstituted or substituted with at least one of deuterium, a halogen(s), a cyano(s), a (C1-C6)alkyl(s), a (C6-C18)aryl(s), a (5- to 20-membered)heteroaryl(s) and a tri(C6-C12)arylsilyl(s); a (5- to 20-membered)heteroaryl unsubstituted or substituted with a (C6-C12)aryl(s); a tri(C6-C12)arylsilyl; and a (C6-C12)aryl(C1-C6)alkyl. Specifically, the substituent(s), each independently, may be at least one selected from the group consisting of deuterium, a fluoro, a cyano, a methyl, a tert-butyl, a phenyl, a biphenyl, a naphthylbiphenyl, a dibenzofuranylbiphenyl, a terphenyl, a naphthyl, a naphthyl substituted with deuterium, a dibenzofuranyinaphthyl, a dibenzothiophenylnaphthyl, a phenyinaphthyl, a naphthylphenyl, a phenanthrenyl, a triphenylenyl, a dimethylfluorenyl, a methylphenylfluorenyl, a phenyl substituted with deuterium, a pyridylphenyl, a dibenzofuranylphenyl, a dibenzothiophenylphenyl, a carbazolylphenyl, a triphenylsilylphenyl, a fluorophenyl, a benzonitrile, a dibenzofuranyl, a phenyldibenzofuranyl, a benzofuranylphenyl, a naphthyldibenzofuranyl, a phenanthrenyldibenzofuranyl, a dibenzothiophenyl, a carbazolyl, a phenylcarbazolyl, a naphthobenzofuranyl, a naphthobenzothiophenyl, a phenylpyridyl, a phenylbenzofuranyl, a triphenylsilyl and a phenylpropyl.

In the formulas of the present disclosure, a ring formed by a linkage of adjacent substituents means that at least two adjacent substituents are linked to each other to form a substituted or unsubstituted, mono- or polycyclic, (3- to 30-membered) alicyclic or aromatic ring, or the combination thereof. In addition, the formed ring may contain at least one heteroatom selected from B, N, O, S, Si, and P, preferably at least one heteroatom selected from N, O, and S. According to one embodiment of the present disclosure, the number of the ring backbone atoms is 5 to 20. According to another embodiment of the present disclosure, the number of the ring backbone atoms is 5 to 15.

In the formulas of the present disclosure, heteroaryl and heteroarylene may, each independently, contain at least one heteroatom selected from B, N, O, S, Si, and P. In addition, the heteroatom may be bonded to at least one selected from the group consisting of hydrogen, deuterium, a halogen, a cyano, a substituted or unsubstituted (C1-C30)alkyl, a substituted or unsubstituted (C6-C30)aryl, a substituted or unsubstituted (5- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, and a substituted or unsubstituted (C1-C30)alkyl(C6-C30)arylamino.

Hereinafter, the compound represented by formula 1 will be described in more detail.

In formula 1, ring A represents

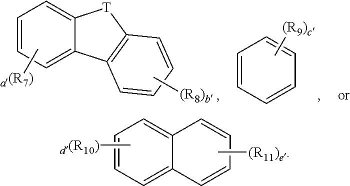

In formula 1, R₁ to R₁₁, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30)aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₁-N—(Ar₁)(Ar₂); with the proviso that at least one of R₁ to R₁₁ represents -L₁-N—(Ar₁)(Ar₂). According to one embodiment of the present disclosure. R₁ to R₁₁, each independently, represent hydrogen; deuterium; a (C6-C30)aryl unsubstituted or substituted with a (C6-C30)aryl(s); an unsubstituted (3- to 30-membered)heteroaryl; or -L₁-N—(Ar₁)(Ar₂); with the proviso that at least one of R₁ to R₁₁ represents -L₁-N—(Ar₁)(Ar₂). According to another embodiment of the present disclosure, R₁ to R₁₁, each independently, represent hydrogen; deuterium; a (C6-C25)aryl unsubstituted or substituted with a (C6-C18)aryl(s); an unsubstituted (3- to 20-membered)heteroaryl; or -L₁-N—(Ar₁)(Ar₂); with the proviso that at least one of R₁ to R₁₁ represents -L₁-N—(Ar₁)(Ar₂). For example, R₁ to R₁₁, each independently, may be hydrogen, deuterium, a phenyl, a terphenyl, a naphthyl, a phenyinaphthyl, a naphthylphenyl, a triphenylenyl, a phenanthrenyl, a pyridyl, a dibenzofuranyl, a quinolyl, or -L₁-N—(Ar₁)(Ar₂).

In formula 1, T represents O, S, or Se. For example, T may be Se.

In formula 1, L₁, each independently, represents a single bond, a substituted or unsubstituted (C6-C30)arylene, or a substituted or unsubstituted (3- to 30-membered)heteroarylene. According to one embodiment of the present disclosure. L₁, each independently, represents a single bond, an unsubstituted (C6-C18)arylene, or an unsubstituted (5- to 20-membered)heteroarylene. According to another embodiment of the present disclosure, L₁, each independently, represents a single bond, an unsubstituted (C6-C15)arylene, or an unsubstituted (5- to 12-membered)heteroarylene. For example, L₁, each independently, may be a single bond, a phenylene, a biphenylene, a naphthylene, a pyridylene, etc.

In formula 1, Ar₁ and Ar₂, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl. According to one embodiment of the present disclosure, Ar₁ and Ar₂, each independently, represent a (C6-C30)aryl unsubstituted or substituted with at least one of deuterium, a (C1-C30)alkyl(s), a (C6-C30)aryl(s), a (3- to 30-membered)heteroaryl(s), a tri(C6-C30)arylsilyl(s) and a (C6-C30)aryl(C1-C30)alkyl; or a (5- to 30-membered)heteroaryl unsubstituted or substituted with a (C6-C30)aryl(s). According to another embodiment of the present disclosure, Ar₁ and Ar₂, each independently, represent a (C6-C28)aryl unsubstituted or substituted with at least one of deuterium, a (C1-C10)alkyl(s), a (C6-C18)aryl(s), a (5- to 20-membered)heteroaryl(s), a tri(C6-C12)arylsilyl(s) and a (C6-C10)aryl(C1-C10)alkyl; or a (5- to 20-membered)heteroaryl unsubstituted or substituted with a (C6-C12)aryl(s). For example, Ar₁ and Ar₂, each independently, may be a phenyl unsubstituted or substituted with a tert-butyl(s), a naphthyl(s), a phenanthrenyl(s), a pyridyl(s), a dibenzofuranyl(s), a phenylpyridyl(s), a phenylbenzofuranyl(s), a triphenylsilyl(s) or a phenylpropyl(s); a biphenyl unsubstituted or substituted with deuterium or a terft-butyl(s); a terphenyl; a naphthyl unsubstituted or substituted with a phenyl(s); a phenanthrenyl; a chrysenyl; a dimethylfluorenyl; a diphenylfluorenyl; a spirobifluorenyl; a dibenzofuranyl unsubstituted or substituted with a phenyl(s); dibenzothiophenyl, etc.

In formula 1, a′ to e′, each independently, represent an integer of 1 to 4, where if a′ to e′ are each an integer of 2 or more, each of R₇ to each of R₁₁ may be the same as or different from each other.

Formula 1 may be represented by at least one of the following formulas 1-1 to 1-6:

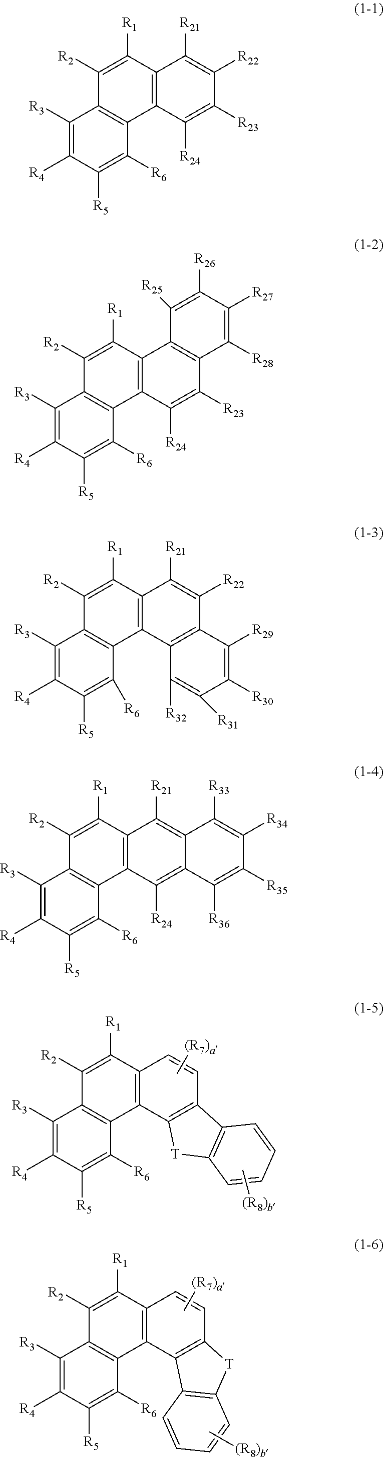

In formulas 1-1 to 1-6, R₂₁ to R₃₆, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₁-N—(Ar₁)(Ar₂). According to one embodiment of the present disclosure, R₂₁ to R₃₆, each independently, represent hydrogen, deuterium, a substituted or unsubstituted (C6-C18)aryl, a substituted or unsubstituted (3- to 20-membered)heteroaryl, or -L₁-N—(Ar₁)(Ar₂). According to another embodiment of the present disclosure, R₂₁ to R₃₆, each independently, represent hydrogen, deuterium, an unsubstituted (C6-C15)aryl, an unsubstituted (3- to 18-membered)heteroaryl, or -L₁-N—(Ar₁)(Ar₂). For example, R₂₁ to R₃₆, each independently, may be hydrogen, deuterium, a phenyl, a naphthyl, a pyridyl, a dibenzofuranyl, or -L₁-N—(Ar₁)(Ar₂).

In formulas 1-1 to 1-6, R₁ to R₈, L₁, Ar₁, Ar₂, T, a′ and b′ are as defined in formula 1.

In formula 2, Y₁ represents O or S.

In formula 2, R′, to R′₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino. According to one embodiment of the present disclosure, R′₁ to R′₃, each independently, represent hydrogen, deuterium, a substituted or unsubstituted (C6-C18)aryl, or a substituted or unsubstituted (5- to 20-membered)heteroaryl. According to another embodiment of the present disclosure, R′₁ to R′₃, each independently, represent hydrogen, deuterium, an unsubstituted (C6-C15)aryl, or an unsubstituted (5- to 20-membered)heteroaryl. For example, R′, to R′₃, each independently, may be hydrogen, deuterium, a phenyl, a biphenyl, a naphthyl, a naphthobenzofuranyl, etc.

In formula 2, L₄, each independently, represents a single bond, a substituted or unsubstituted (C6-C30)arylene, or a substituted or unsubstituted (3- to 30-membered)heteroarylene. According to one embodiment of the present disclosure, L₄, each independently, represents a single bond, a substituted or unsubstituted (C6-C18)arylene, or a substituted or unsubstituted (5- to 20-membered)heteroarylene. According to another embodiment of the present disclosure, L₄, each independently, represents a single bond, a (C6-C18)arylene unsubstituted or substituted deuterium or a (C6-C12)aryl(s); or an unsubstituted (5- to 20-membered)heteroarylene. For example, L₄, each independently, may be a single bond, a phenylene, a phenylene substituted with deuterium, a biphenylene, a naphthylene, a dibenzofuranylene, a dibenzothiophenylene, a naphthobenzofuranylene, etc.

In formula 2, Ar₄ represents a substituted or unsubstituted (3- to 30-membered)heteroaryl containing a nitrogen(s). According to one embodiment of the present disclosure, Ar₄ represents a (3- to 30-membered)heteroaryl containing a nitrogen(s) unsubstituted or substituted with at least one of a substituted or unsubstituted (C6-C30)aryl(s) and a substituted or unsubstituted (3- to 30-membered)heteroaryl(s). According to another embodiment of the present disclosure, Ar₄ represents a (5- to 20-membered)heteroaryl containing a nitrogen(s) unsubstituted or substituted with at least one of a substituted or unsubstituted (C6-C28)aryl(s) and a substituted or unsubstituted (5- to 30-membered)heteroaryl(s). For example, Ar₄ may be a substituted pyridyl, a substituted pyrimidinyl, a substituted triazinyl, a substituted quinoxalinyl, a substituted quinazolinyl, a substituted benzoquinoxalinyl, a substituted benzofuropyrimidinyl, etc., in which the substituent(s) of the substituted pyridyl, the substituted pyrimidinyl, the substituted triazinyl, the substituted quinoxalinyl, the substituted quinazolinyl, the substituted benzoquinoxalinyl, or the substituted benzofuropyrimidinyl, each independently, may be at least one selected from the group consisting of a phenyl, a biphenyl, a naphthylbiphenyl, a dibenzofuranylbiphenyl, a terphenyl, a triphenylbenzyl, a naphthyl, a naphthyl substituted with deuterium, a phenylnaphthyl, a dibenzofuranylnaphthyl, a dibenzothiophenylnaphthyl, a naphthylphenyl, a phenanthrenyl, a triphenylenyl, a dimethylfluorenyl, a methylphenylfluorenyl, a phenyl substituted with deuterium, a phenyl substituted with a cyano(s), a quaterphenyl, a dibenzofuranylphenyl, a dibenzothiophenylphenyl, a carbazolylphenyl, a triphenylsilylphenyl, a fluorophenyl, a benzonitrile, a dibenzofuranyl, a phenyldibenzofuranyl, a naphthyldibenzofuranyl, a phenanthrenyldibenzofuranyl, a dibenzothiophenyl, a phenylcarbazolyl, a naphthobenzofuranyl, and a naphthobenzothiophenyl.

In formula 2, a and d, each independently, represent an integer of 1 to 3, b represents an integer of 1 or 2, and c represents an integer of 1 to 4; where if a to d are each an integer of 2 or more, each of R′₁, each of R′₂, each of R′₃, and each of L₄ may be the same as or different from each other.

Formula 2 may be represented by at least one of the following formulas 2-1 to 2-12:

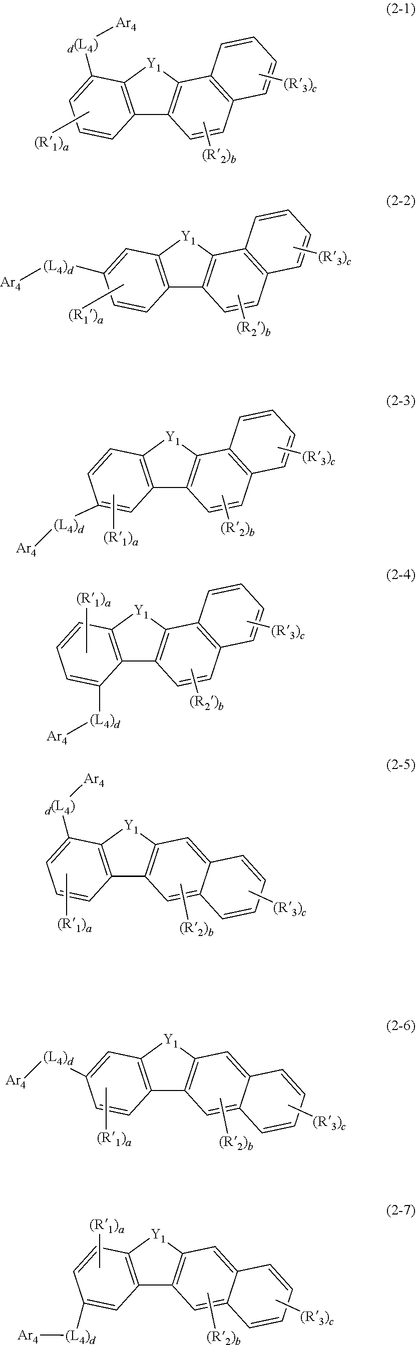

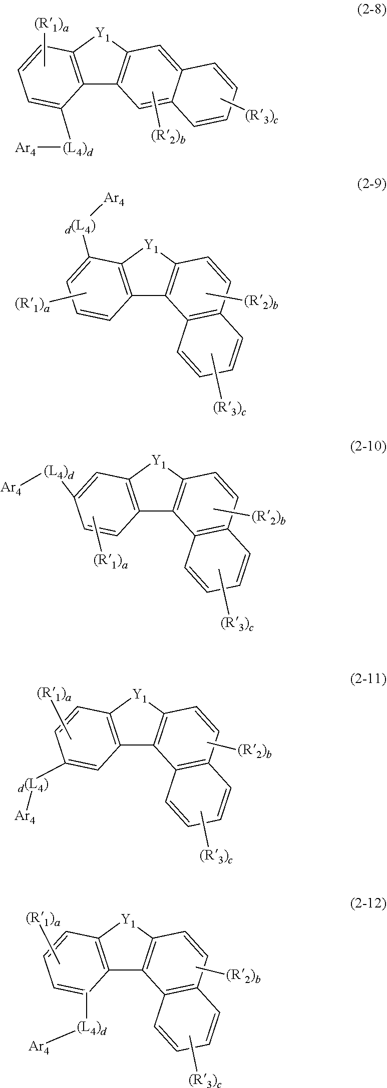

In formulas 2-1 to 2-12,

Y₁, R′₁ to R′₃, L₄, Ar₄, and a to d are as defined in formula 2.

Ar₄ in formula 2 may be represented by any one of the following formulas 2-1 to 2′-5:

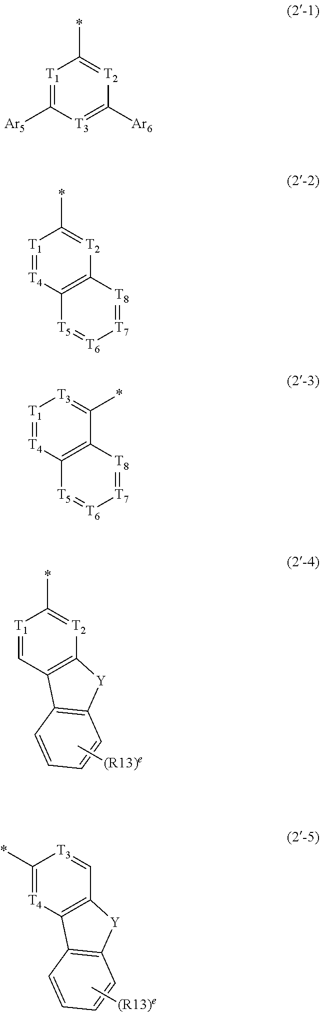

In formulas 2′-1 to 2′-5, T₁ to T₈, each independently, represent CR₁₂ or N, with the proviso that at least one of T₁ to T₃ in formula 2′-1 represents N, at least one of T₁, T₂, and T₄ to T₈ in formula 2′-2 represents N, at least one of T₁, and T₃ to T₈ in formula 2′-3 represents N, at least one of T₁ and T₂ in formula 2′-4 represents N, and at least one of T₃ and T₄ in formula 2′-5 represents N.

In formulas 2′-1 to 2′-5, Y represents O or S.

In formulas 2′-1 to 2′-5, R₁₂, Ar₅ and Ar₆, each independently, represent hydrogen, deuterium, a substituted or unsubstituted (C1-C30)alkyl, a substituted or unsubstituted (C6-C30)aryl, or a substituted or unsubstituted (3- to 30-membered)heteroaryl; or R₁₂ may be linked to an adjacent substituent(s) to form a ring(s). According to one embodiment of the present disclosure, R₁₂, Ar₅ and Ar₆, each independently, represent hydrogen; a (C6-C30)aryl unsubstituted or substituted with at least one of deuterium, a (C6-C30)aryl(s), a (3- to 30-membered)heteroaryl(s), a tri(C6-C30)arylsilyl(s), a halogen(s) and a cyano(s); or a (3- to 30-membered)heteroaryl unsubstituted or substituted with a (C6-C30)aryl(s); or R₁₂ may be linked to an adjacent substituent(s) to form a ring(s). According to another embodiment of the present disclosure, R₁₂, Ar₅ and Ar₆, each independently, represent hydrogen; a (C6-C25)aryl unsubstituted or substituted with at least one of deuterium, a (C6-C20)aryl(s), a (5- to 18-membered)heteroaryl(s), a tri(C6-C12)arylsilyl(s), a fluoro(s) and a cyano(s); or a (3- to 28-membered)heteroaryl unsubstituted or substituted with a (C6-C30)aryl(s); or R₁₂ may be linked to an adjacent substituent(s) to form a ring(s). For example, R₁₂, Ar₅ and Ar₆, each independently, may be hydrogen; a phenyl unsubstituted or substituted with at least one of deuterium, a naphthyl(s), a terphenyl(s), a dibenzofuranyl(s), a dibenzothiophenyl(s), a carbazolyl(s), a triphenylsilyl(s), a fluoro(s) and acyano(s); a biphenyl unsubstituted or substituted with a naphthyl(s) or a dibenzofuranyl(s); a terphenyl unsubstituted or substituted with a phenyl(s); a naphthyl unsubstituted or substituted with deuterium, a phenyl(s), a dibenzofuranyl(s) or a dibenzothiophenyl(s); a phenanthrenyl; a triphenylenyl; a dimethylfluorenyl; a methylphenylfluorenyl; a dibenzofuranyl unsubstituted or substituted with a phenyl(s), a naphthyl(s) or a phenanthrenyl(s); a dibenzothiophenyl; a phenylcarbazolyl; a naphthobenzofuranyl; a naphthobenzothiophenyl, etc.

In formulas 2-1 to 2′-5, R₁₃, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino. According to one embodiment of the present disclosure, R₁₃, each independently, represents hydrogen, or a substituted or unsubstituted (C6-C30)aryl. According to another embodiment of the present disclosure, R₁₃, each independently, represents hydrogen, or an unsubstituted (C6-C12)aryl. For example, R₁₃ may be hydrogen, a phenyl, etc.

In formulas 2′-1 to 2-5, * represents a bonding position to L; and e represents an integer of 1 to 4, where if e is an integer of 2 or more, each of R₁₃ may be the same as or different from each other.

The compound represented by formula 1 may be at least one selected from the following compounds, but is not limited thereto.

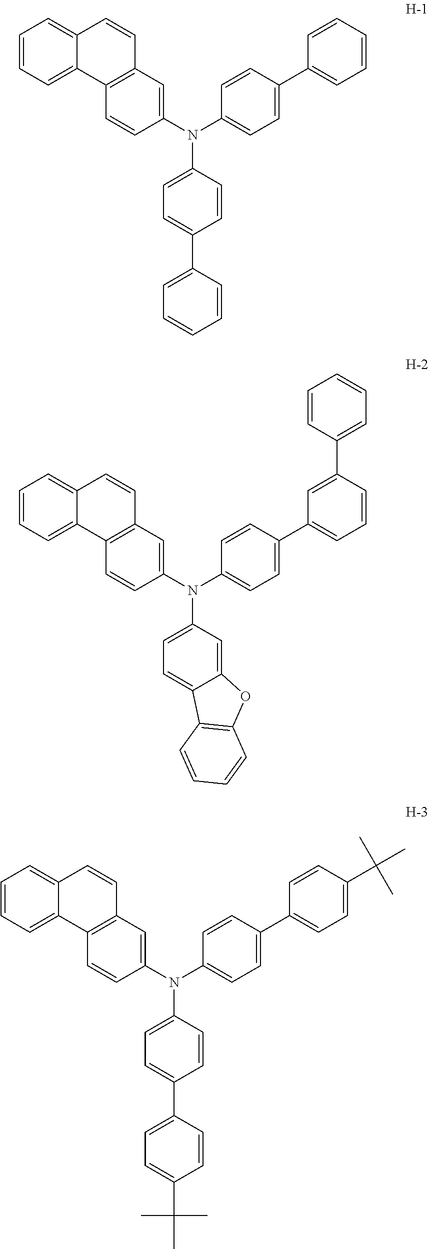 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

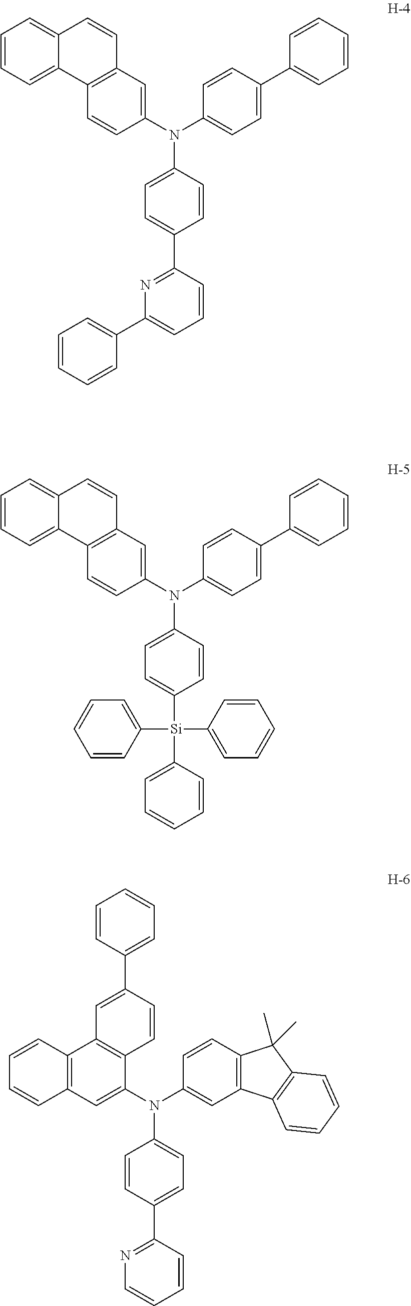 [SMILES: c1ccc(-c2ccc(N(c3ccc(-c4cccc(-c5ccccc5)n4)cc3)c3ccc4c(ccc5ccccc54)c3)cc2)cc1 ; c1ccc(-c2ccc(N(c3ccc([Si](c4ccccc4)(c4ccccc4)c4ccccc4)cc3)c3ccc4c(ccc5ccccc54)c3)cc2)cc1 ; CC1(C)c2ccccc2-c2cc(N(c3ccc(-c4ccccn4)cc3)c3cc4ccccc4c4cc(-c5ccccc5)ccc34)ccc21]

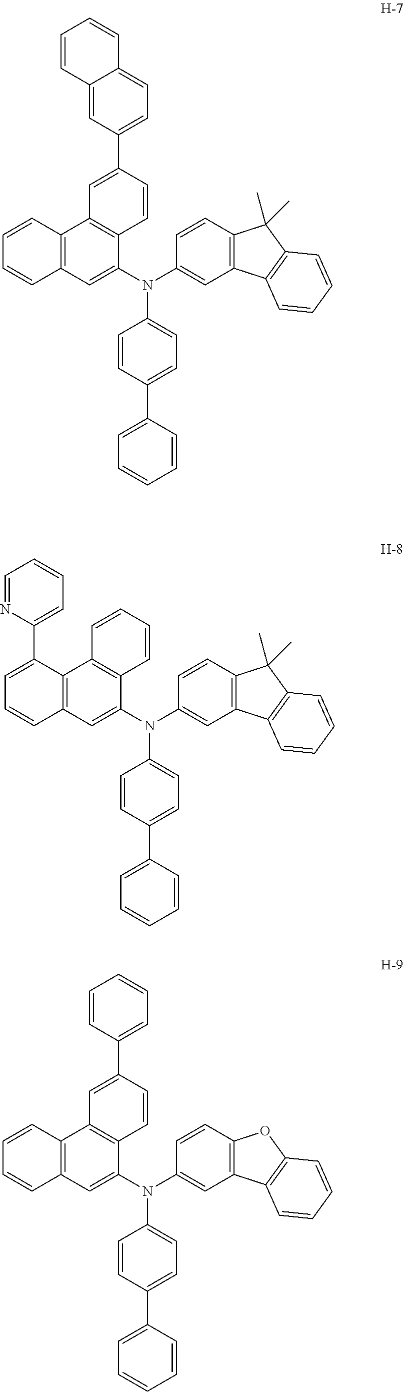 [SMILES: CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5ccccn5)c4c4ccccc34)ccc21 ; c1ccc(-c2ccc(N(c3ccc4oc5ccccc5c4c3)c3cc4ccccc4c4cc(-c5ccccc5)ccc34)cc2)cc1]

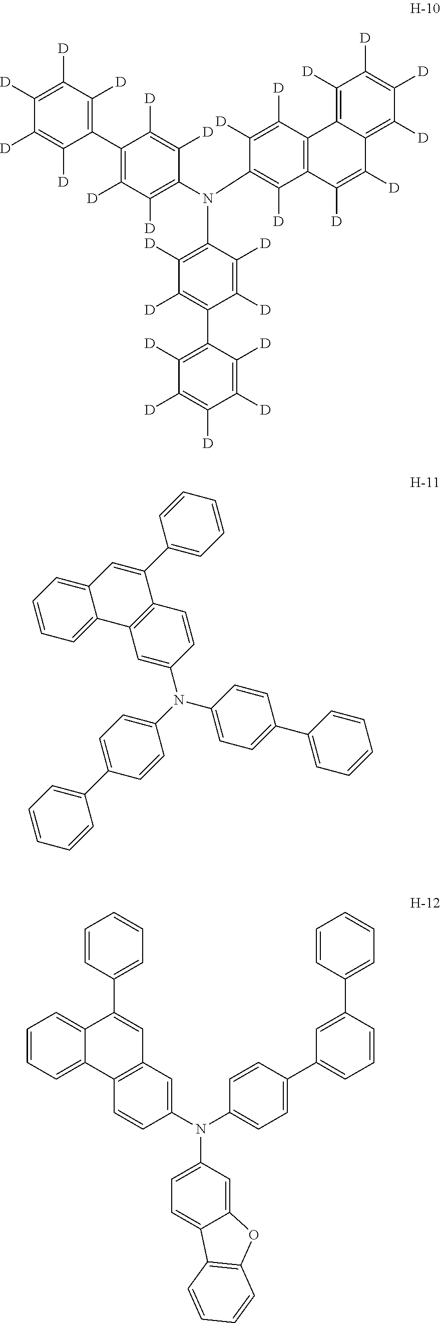 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c([2H])c([2H])c([2H])c([2H])c34)c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1 ; c1ccc(-c2cccc(-c3ccc(N(c4ccc5c(c4)cc(-c4ccccc4)c4ccccc45)c4ccc5c(c4)oc4ccccc45)cc3)c2)cc1]

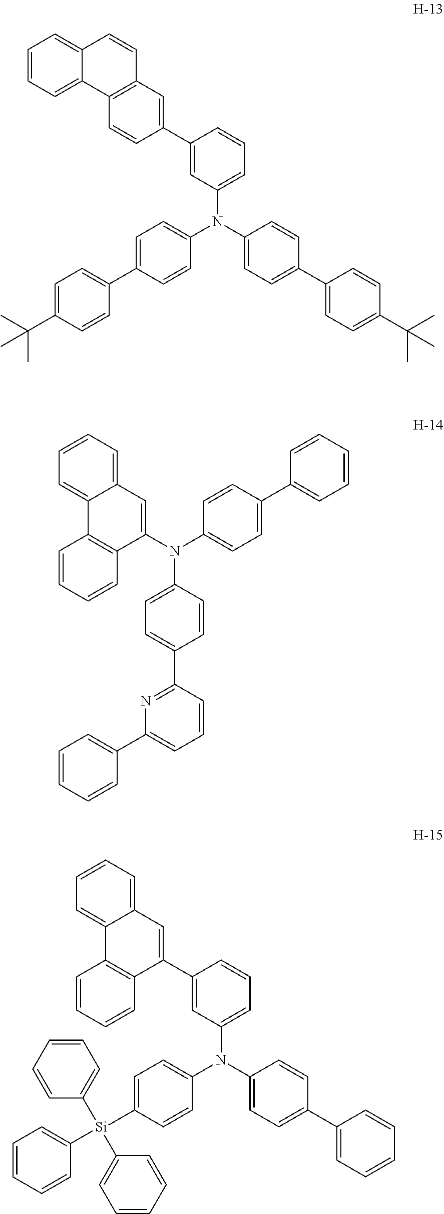 [SMILES: CC(C)(C)c1ccc(-c2ccc(N(c3ccc(-c4ccc(C(C)(C)C)cc4)cc3)c3cccc(-c4ccc5c(ccc6ccccc65)c4)c3)cc2)cc1 ; c1ccc(-c2ccc(N(c3ccc(-c4cccc(-c5ccccc5)n4)cc3)c3cc4ccccc4c4ccccc34)cc2)cc1 ; c1ccc(-c2ccc(N(c3ccc([Si](c4ccccc4)(c4ccccc4)c4ccccc4)cc3)c3cccc(-c4cc5ccccc5c5ccccc45)c3)cc2)cc1]

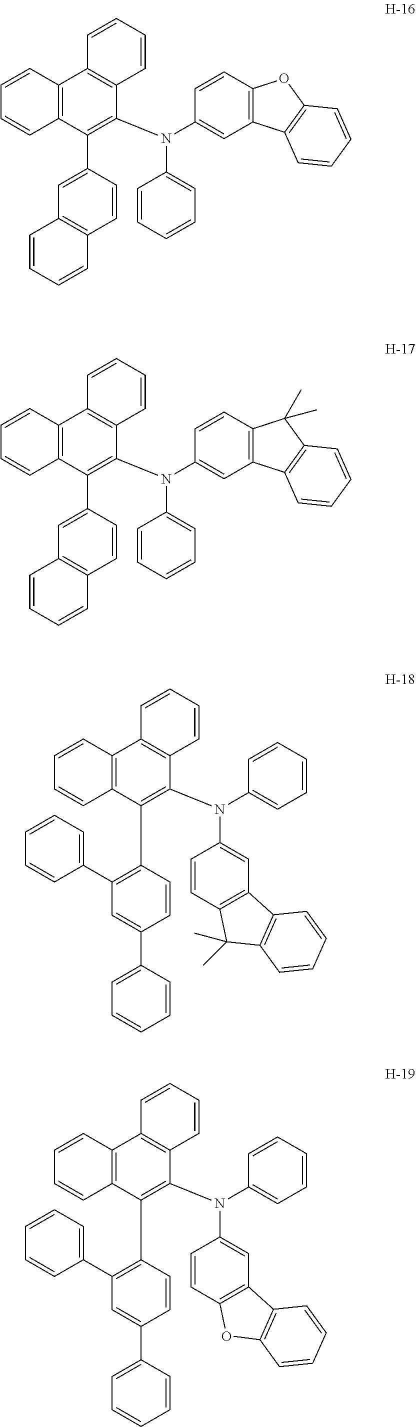 [SMILES: c1ccc(N(c2ccc3oc4ccccc4c3c2)c2c(-c3ccc4ccccc4c3)c3ccccc3c3ccccc23)cc1 ; CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21 ; c1ccc(-c2ccc(-c3c(N(c4ccccc4)c4ccc5oc6ccccc6c5c4)c4ccccc4c4ccccc34)c(-c3ccccc3)c2)cc1]

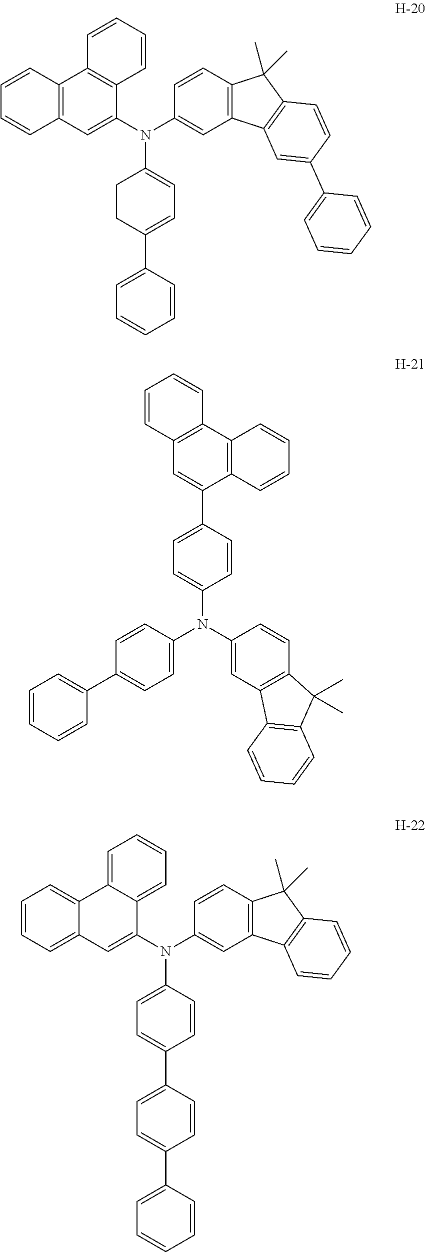 [SMILES: CC1(C)c2ccc(-c3ccccc3)cc2-c2cc(N(C3=CC=C(c4ccccc4)CC3)c3cc4ccccc4c4ccccc34)ccc21 ; CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4ccc(-c5ccccc5)cc4)cc3)c3cc4ccccc4c4ccccc34)ccc21]

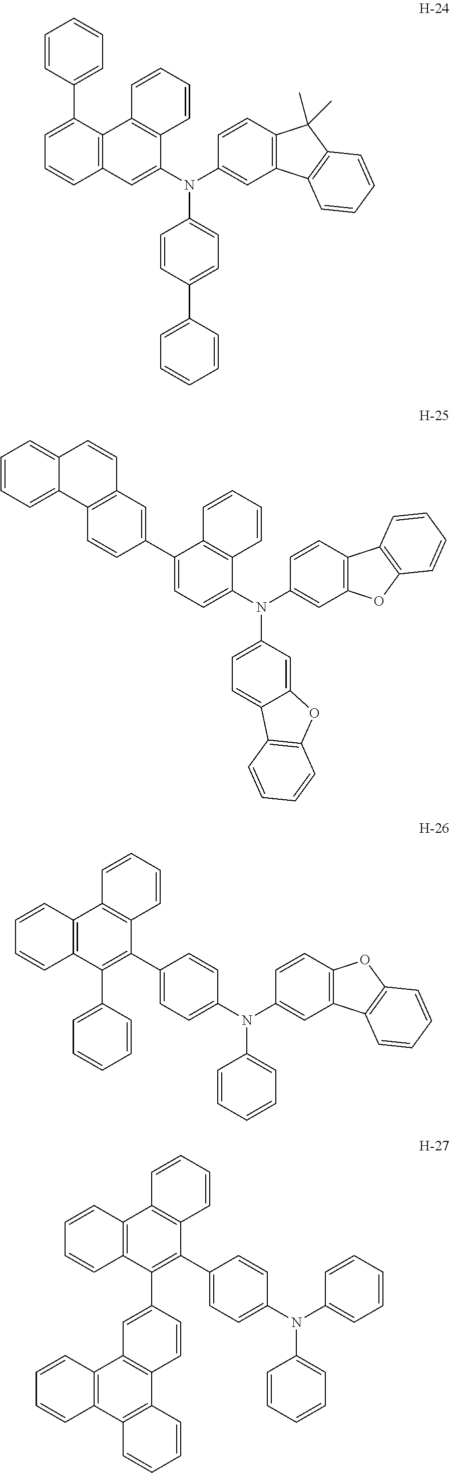 [SMILES: CC1(C)c2ccccc2-c2cc(N(c3ccc(-c4ccccc4)cc3)c3cc4cccc(-c5ccccc5)c4c4ccccc34)ccc21 ; c1ccc2c(c1)ccc1cc(-c3ccc(N(c4ccc5c(c4)oc4ccccc45)c4ccc5c(c4)oc4ccccc45)c4ccccc34)ccc12 ; c1ccc(-c2c(-c3ccc(N(c4ccccc4)c4ccc5oc6ccccc6c5c4)cc3)c3ccccc3c3ccccc23)cc1 ; c1ccc(N(c2ccccc2)c2ccc(-c3c(-c4ccc5c6ccccc6c6ccccc6c5c4)c4ccccc4c4ccccc34)cc2)cc1]

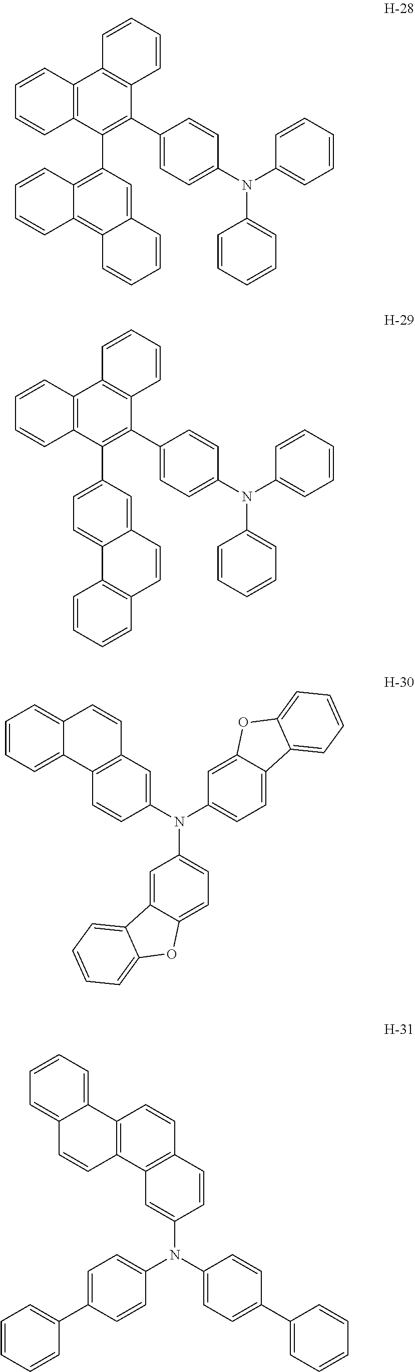

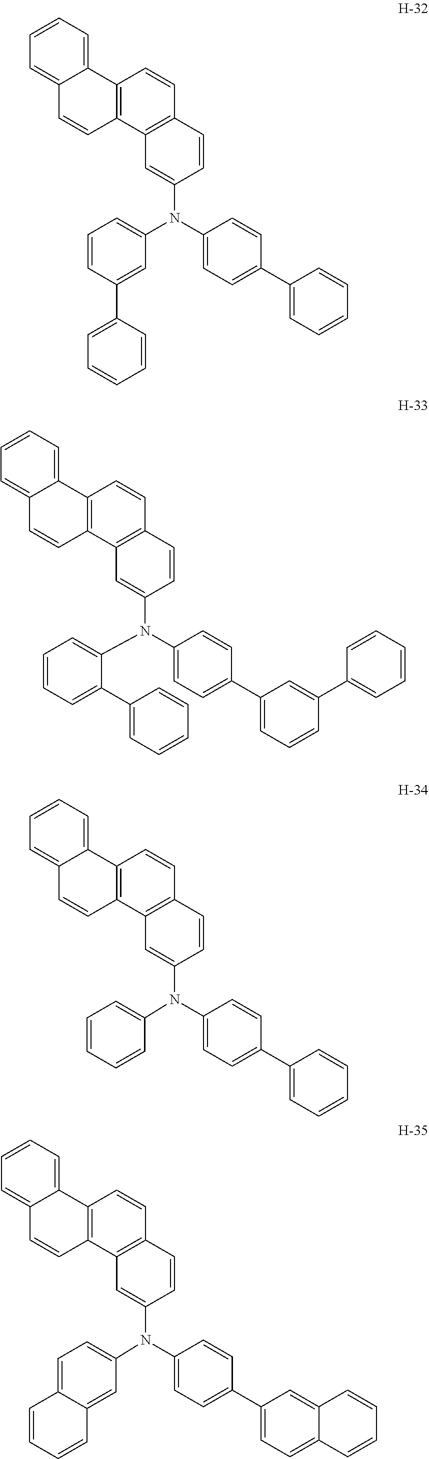 [SMILES: c1ccc(-c2ccc(N(c3cccc(-c4ccccc4)c3)c3ccc4ccc5c6ccccc6ccc5c4c3)cc2)cc1 ; c1ccc(-c2cccc(-c3ccc(N(c4ccc5ccc6c7ccccc7ccc6c5c4)c4ccccc4-c4ccccc4)cc3)c2)cc1 ; c1ccc(-c2ccc(N(c3ccccc3)c3ccc4ccc5c6ccccc6ccc5c4c3)cc2)cc1 ; c1ccc2cc(-c3ccc(N(c4ccc5ccccc5c4)c4ccc5ccc6c7ccccc7ccc6c5c4)cc3)ccc2c1]

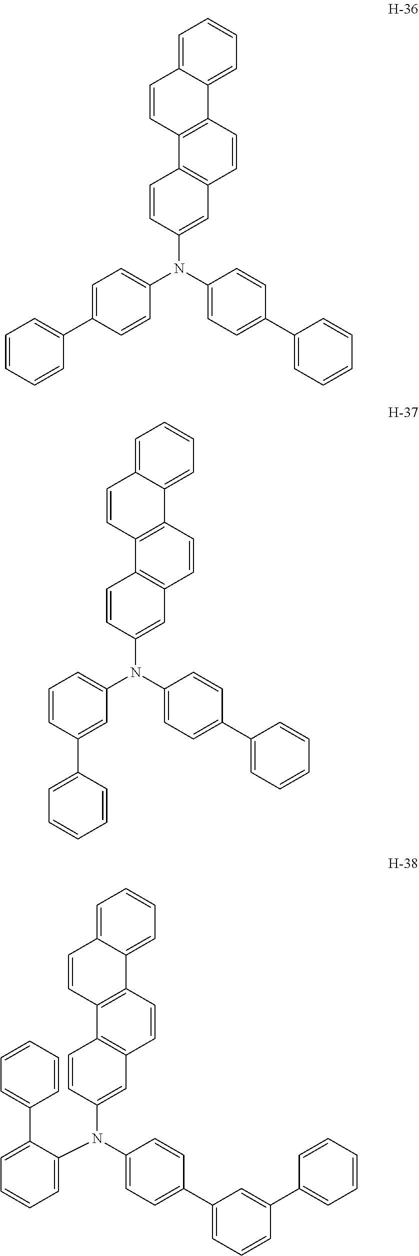 [SMILES: c1ccc(-c2ccc(N(c3ccc(-c4ccccc4)cc3)c3ccc4c(ccc5c6ccccc6ccc45)c3)cc2)cc1 ; c1ccc(-c2ccc(N(c3cccc(-c4ccccc4)c3)c3ccc4c(ccc5c6ccccc6ccc45)c3)cc2)cc1 ; c1ccc(-c2cccc(-c3ccc(N(c4ccc5c(ccc6c7ccccc7ccc56)c4)c4ccccc4-c4ccccc4)cc3)c2)cc1]

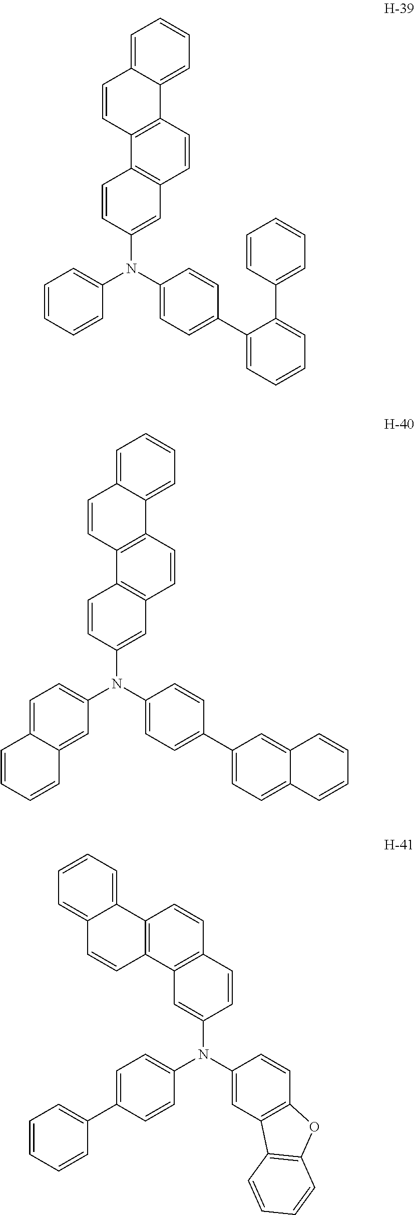 [SMILES: c1ccc(-c2ccccc2-c2ccc(N(c3ccccc3)c3ccc4c(ccc5c6ccccc6ccc45)c3)cc2)cc1 ; c1ccc2cc(-c3ccc(N(c4ccc5ccccc5c4)c4ccc5c(ccc6c7ccccc7ccc56)c4)cc3)ccc2c1 ; c1ccc(-c2ccc(N(c3ccc4ccc5c6ccccc6ccc5c4c3)c3ccc4oc5ccccc5c4c3)cc2)cc1]

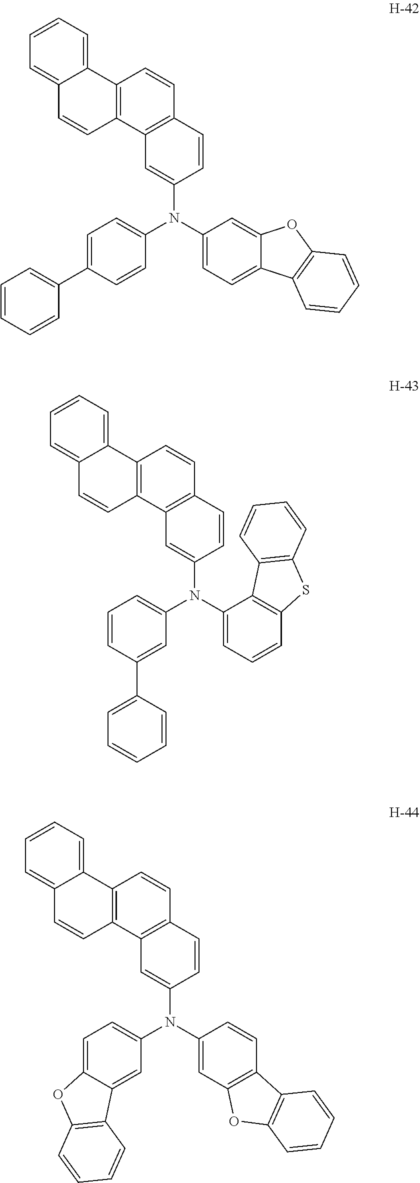 [SMILES: c1ccc(-c2ccc(N(c3ccc4c(c3)oc3ccccc34)c3ccc4ccc5c6ccccc6ccc5c4c3)cc2)cc1 ; c1ccc(-c2cccc(N(c3ccc4ccc5c6ccccc6ccc5c4c3)c3cccc4sc5ccccc5c34)c2)cc1 ; c1ccc2c(c1)ccc1c3cc(N(c4ccc5c(c4)oc4ccccc45)c4ccc5oc6ccccc6c5c4)ccc3ccc21]

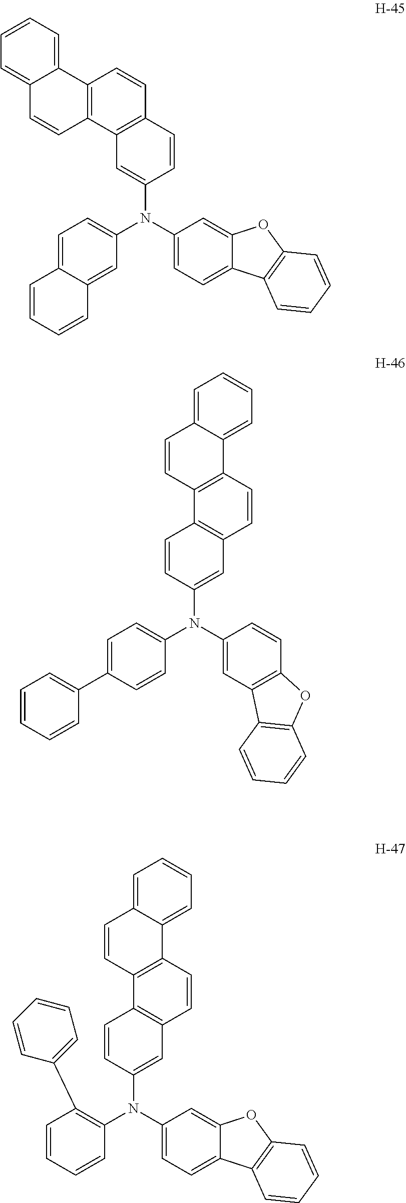 [SMILES: c1ccc2cc(N(c3ccc4c(c3)oc3ccccc34)c3ccc4ccc5c6ccccc6ccc5c4c3)ccc2c1 ; c1ccc(-c2ccc(N(c3ccc4c(ccc5c6ccccc6ccc45)c3)c3ccc4oc5ccccc5c4c3)cc2)cc1 ; c1ccc(-c2ccccc2N(c2ccc3c(ccc4c5ccccc5ccc34)c2)c2ccc3c(c2)oc2ccccc23)cc1]

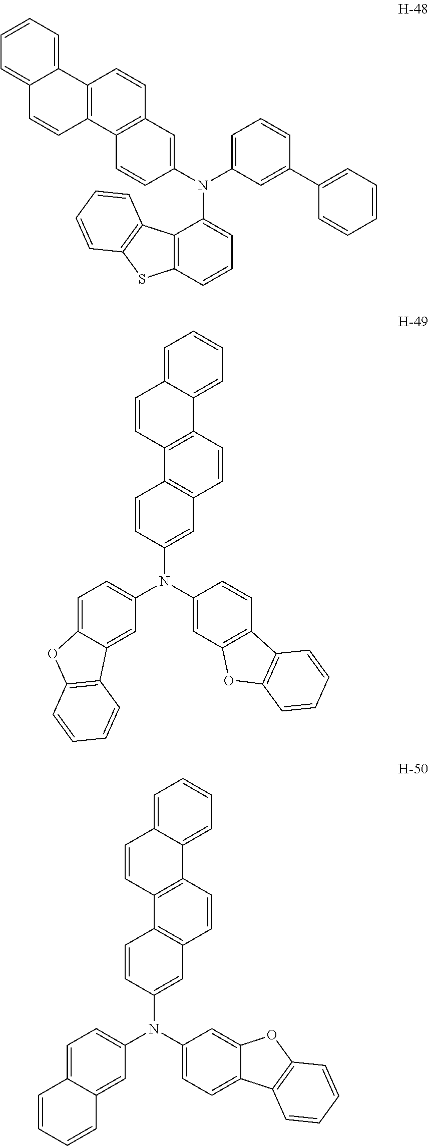 [SMILES: c1ccc(-c2cccc(N(c3ccc4c(ccc5c6ccccc6ccc45)c3)c3cccc4sc5ccccc5c34)c2)cc1 ; c1ccc2c(c1)ccc1c3ccc(N(c4ccc5c(c4)oc4ccccc45)c4ccc5oc6ccccc6c5c4)cc3ccc21 ; c1ccc2cc(N(c3ccc4c(ccc5c6ccccc6ccc45)c3)c3ccc4c(c3)oc3ccccc34)ccc2c1]

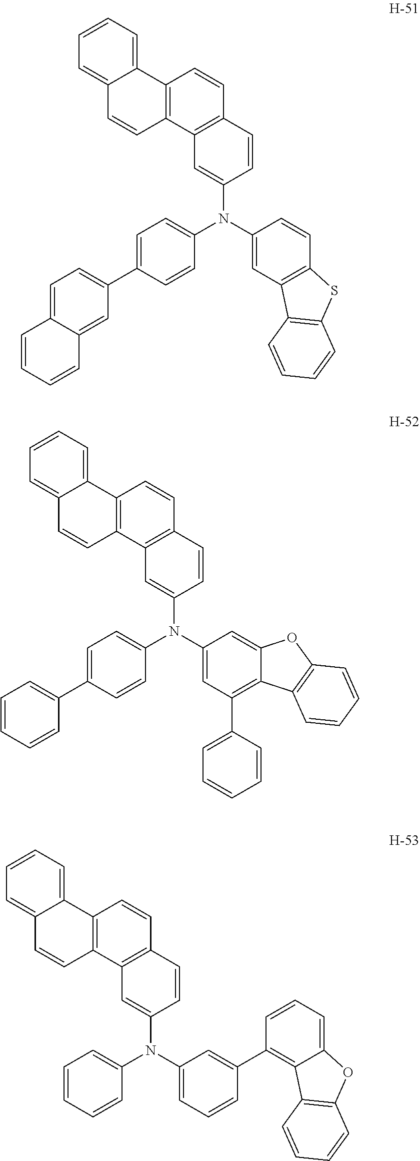 [SMILES: c1ccc2cc(-c3ccc(N(c4ccc5ccc6c7ccccc7ccc6c5c4)c4ccc5sc6ccccc6c5c4)cc3)ccc2c1 ; c1ccc(-c2ccc(N(c3cc(-c4ccccc4)c4c(c3)oc3ccccc34)c3ccc4ccc5c6ccccc6ccc5c4c3)cc2)cc1 ; c1ccc(N(c2cccc(-c3cccc4oc5ccccc5c34)c2)c2ccc3ccc4c5ccccc5ccc4c3c2)cc1]

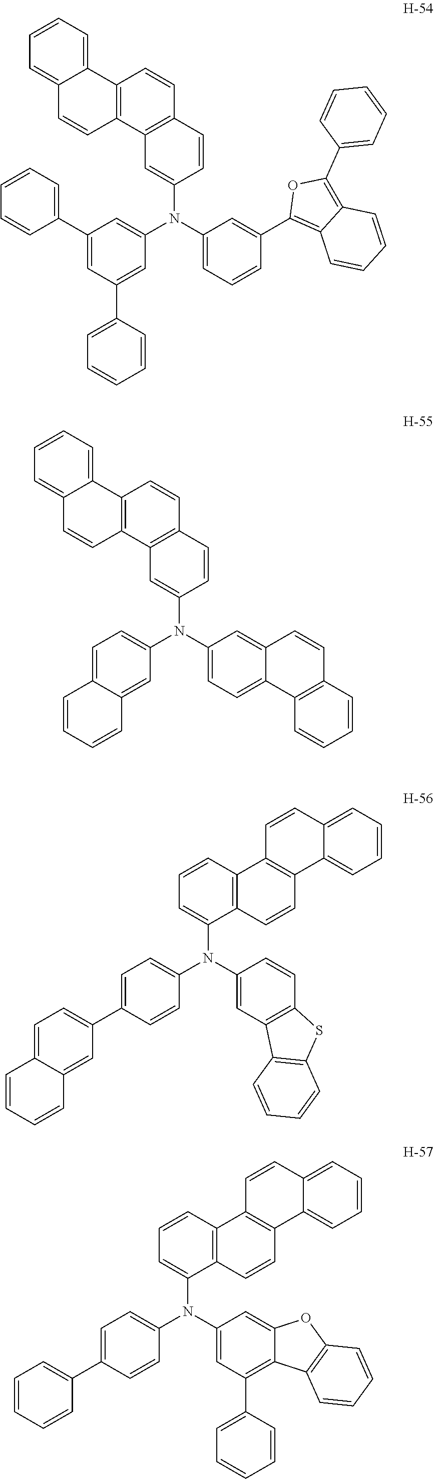 [SMILES: c1ccc(-c2cc(-c3ccccc3)cc(N(c3cccc(-c4oc(-c5ccccc5)c5ccccc45)c3)c3ccc4ccc5c6ccccc6ccc5c4c3)c2)cc1 ; c1ccc2cc(N(c3ccc4c(ccc5ccccc54)c3)c3ccc4ccc5c6ccccc6ccc5c4c3)ccc2c1 ; c1ccc2cc(-c3ccc(N(c4ccc5sc6ccccc6c5c4)c4cccc5c4ccc4c6ccccc6ccc54)cc3)ccc2c1 ; c1ccc(-c2ccc(N(c3cc(-c4ccccc4)c4c(c3)oc3ccccc34)c3cccc4c3ccc3c5ccccc5ccc43)cc2)cc1]

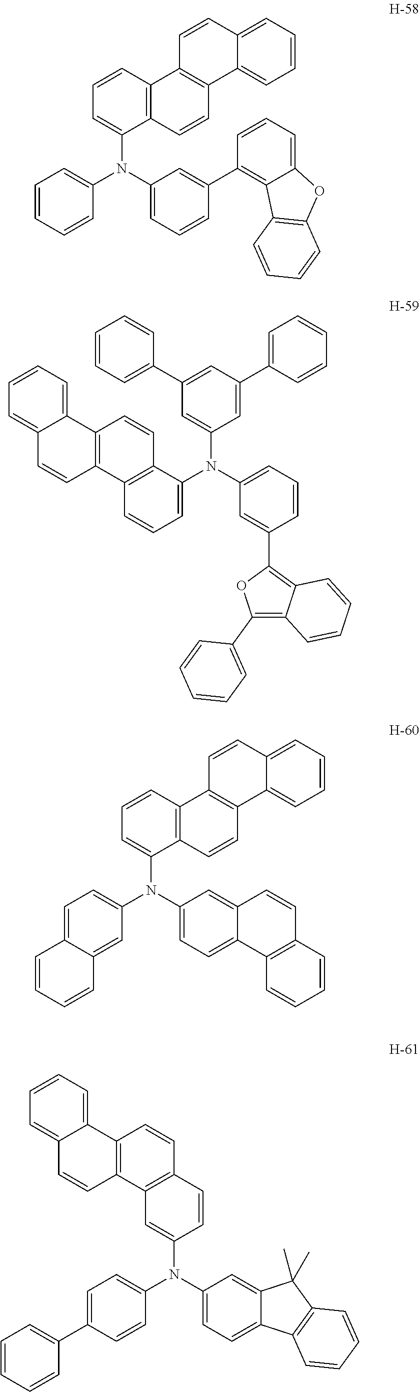

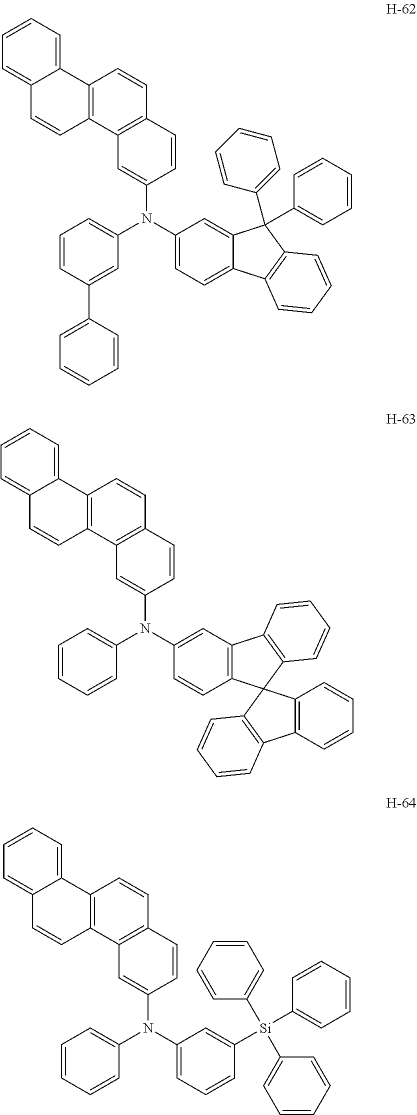 [SMILES: c1ccc(-c2cccc(N(c3ccc4c(c3)C(c3ccccc3)(c3ccccc3)c3ccccc3-4)c3ccc4ccc5c6ccccc6ccc5c4c3)c2)cc1 ; c1ccc(N(c2ccc3c(c2)-c2ccccc2C32c3ccccc3-c3ccccc32)c2ccc3ccc4c5ccccc5ccc4c3c2)cc1 ; c1ccc(N(c2cccc([Si](c3ccccc3)(c3ccccc3)c3ccccc3)c2)c2ccc3ccc4c5ccccc5ccc4c3c2)cc1]

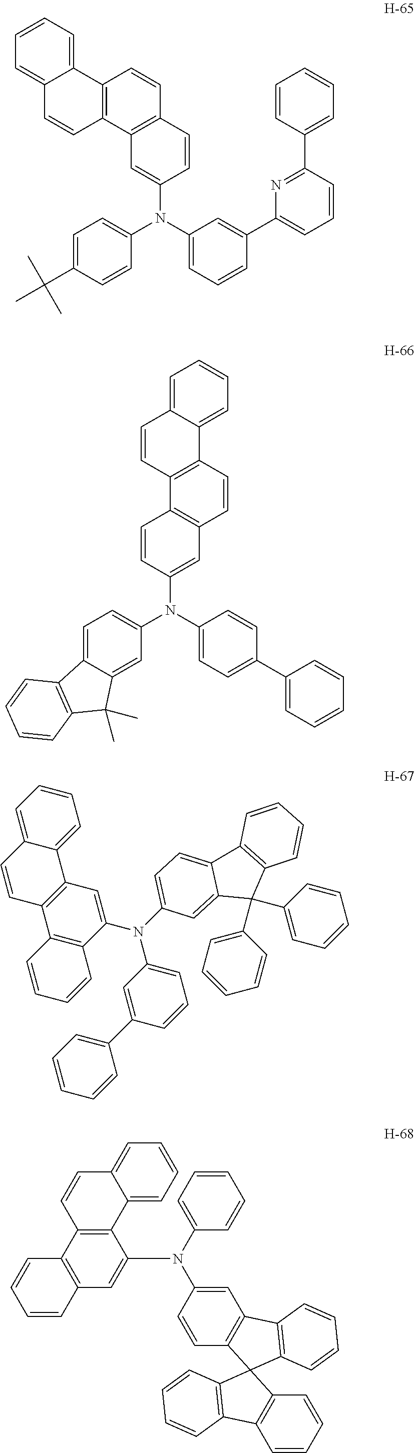 [SMILES: CC(C)(C)c1ccc(N(c2cccc(-c3cccc(-c4ccccc4)n3)c2)c2ccc3ccc4c5ccccc5ccc4c3c2)cc1 ; CC1(C)c2ccccc2-c2ccc(N(c3ccc(-c4ccccc4)cc3)c3ccc4c(ccc5c6ccccc6ccc45)c3)cc21 ; c1ccc(-c2cccc(N(c3ccc4c(c3)C(c3ccccc3)(c3ccccc3)c3ccccc3-4)c3cc4c5ccccc5ccc4c4ccccc34)c2)cc1 ; c1ccc(N(c2ccc3c(c2)-c2ccccc2C32c3ccccc3-c3ccccc32)c2cc3ccccc3c3ccc4ccccc4c23)cc1]

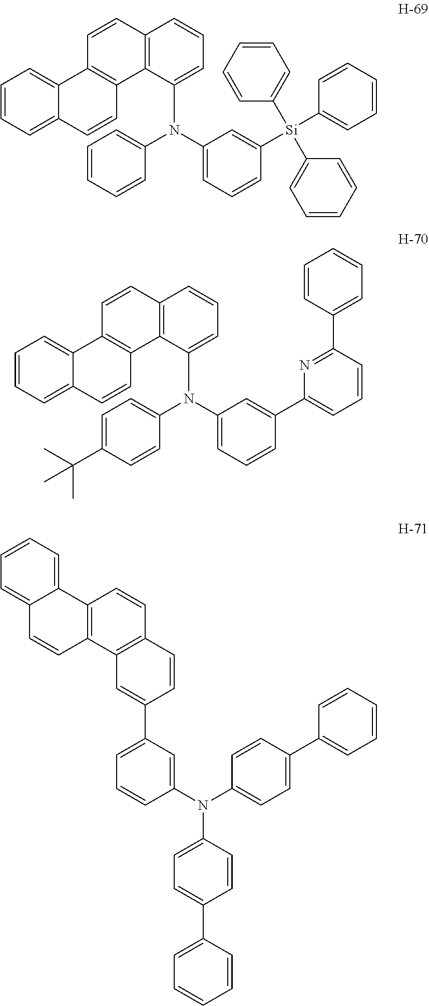

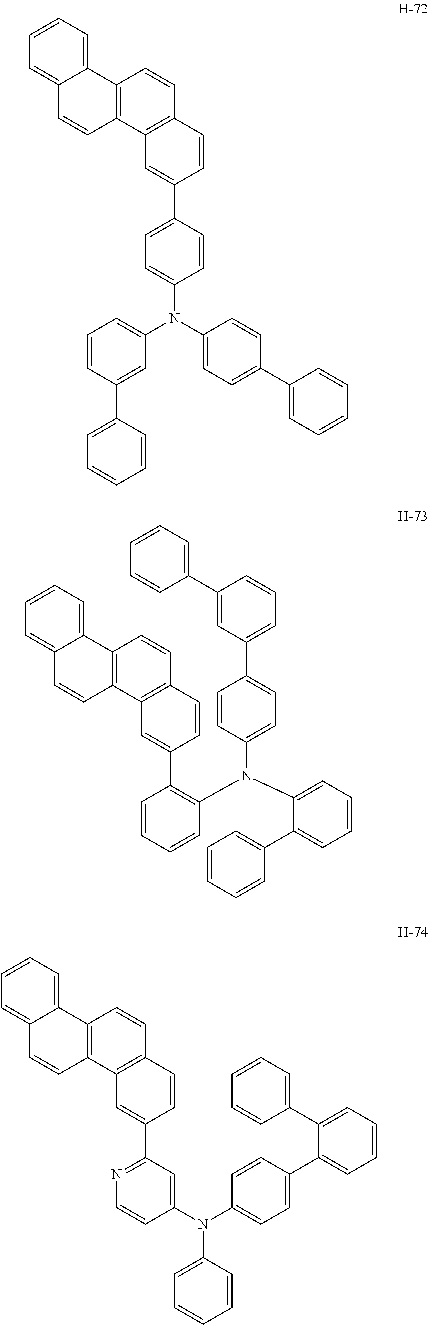 [SMILES: c1ccc(-c2ccc(N(c3ccc(-c4ccc5ccc6c7ccccc7ccc6c5c4)cc3)c3cccc(-c4ccccc4)c3)cc2)cc1 ; c1ccc(-c2cccc(-c3ccc(N(c4ccccc4-c4ccccc4)c4ccccc4-c4ccc5ccc6c7ccccc7ccc6c5c4)cc3)c2)cc1 ; c1ccc(-c2ccccc2-c2ccc(N(c3ccccc3)c3ccnc(-c4ccc5ccc6c7ccccc7ccc6c5c4)c3)cc2)cc1]

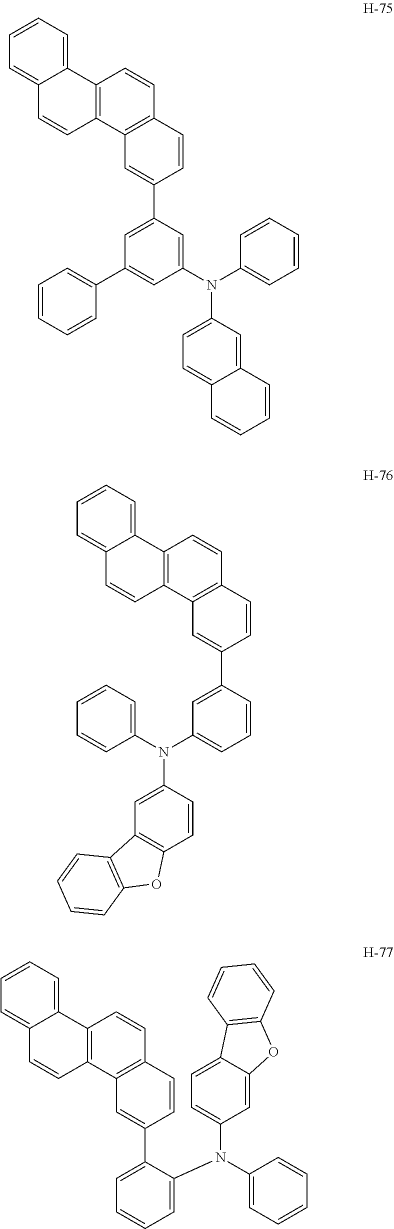 [SMILES: c1ccc(-c2cc(-c3ccc4ccc5c6ccccc6ccc5c4c3)cc(N(c3ccccc3)c3ccc4ccccc4c3)c2)cc1 ; c1ccc(N(c2cccc(-c3ccc4ccc5c6ccccc6ccc5c4c3)c2)c2ccc3oc4ccccc4c3c2)cc1 ; c1ccc(N(c2ccc3c(c2)oc2ccccc23)c2ccccc2-c2ccc3ccc4c5ccccc5ccc4c3c2)cc1]

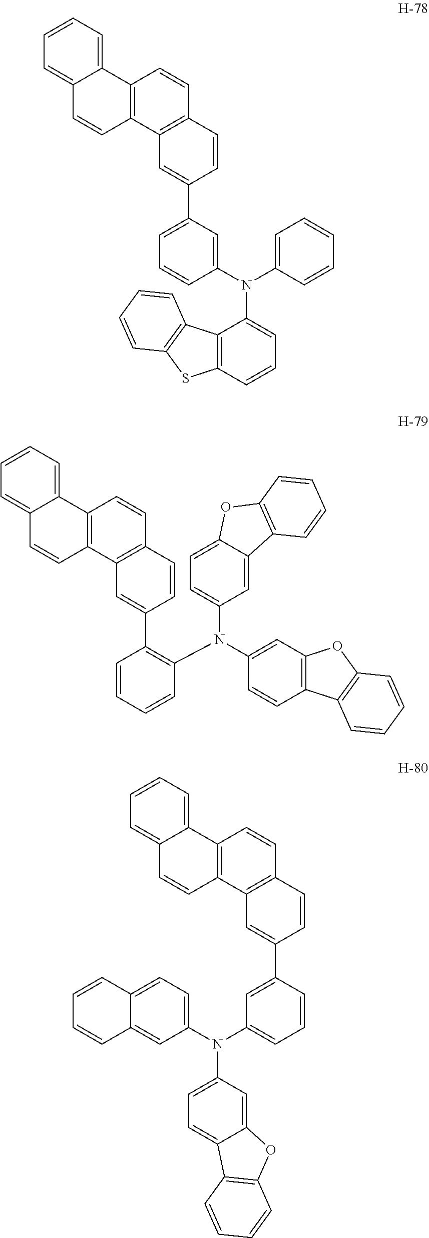 [SMILES: c1ccc(N(c2cccc(-c3ccc4ccc5c6ccccc6ccc5c4c3)c2)c2cccc3sc4ccccc4c23)cc1 ; c1ccc(N(c2ccc3c(c2)oc2ccccc23)c2ccc3oc4ccccc4c3c2)c(-c2ccc3ccc4c5ccccc5ccc4c3c2)c1 ; c1cc(-c2ccc3ccc4c5ccccc5ccc4c3c2)cc(N(c2ccc3ccccc3c2)c2ccc3c(c2)oc2ccccc23)c1]

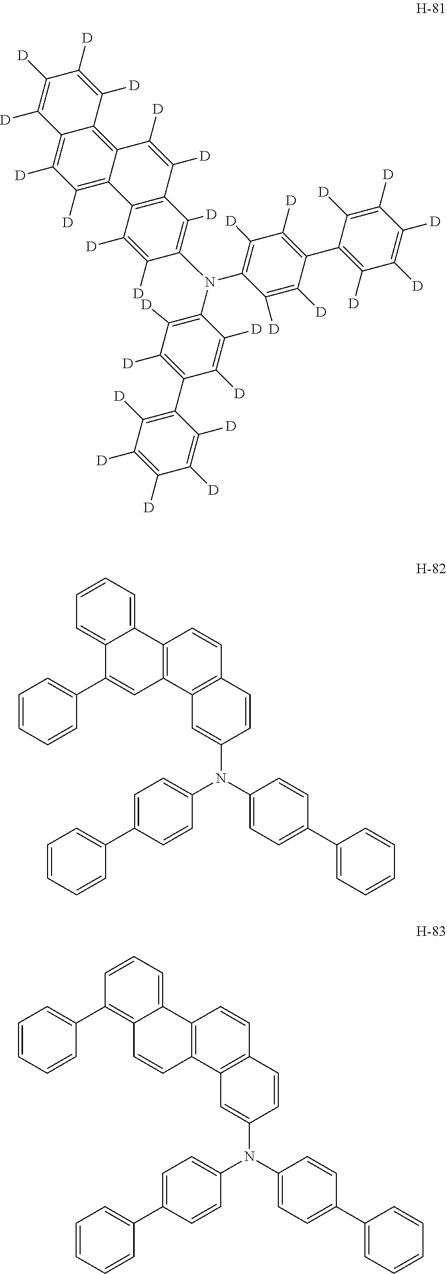 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c5c([2H])c([2H])c([2H])c([2H])c5c([2H])c([2H])c43)c([2H])c2[2H])c([2H])c1[2H] ; c1ccc(-c2ccc(N(c3ccc(-c4ccccc4)cc3)c3ccc4ccc5c6ccccc6c(-c6ccccc6)cc5c4c3)cc2)cc1 ; c1ccc(-c2ccc(N(c3ccc(-c4ccccc4)cc3)c3ccc4ccc5c6cccc(-c7ccccc7)c6ccc5c4c3)cc2)cc1]

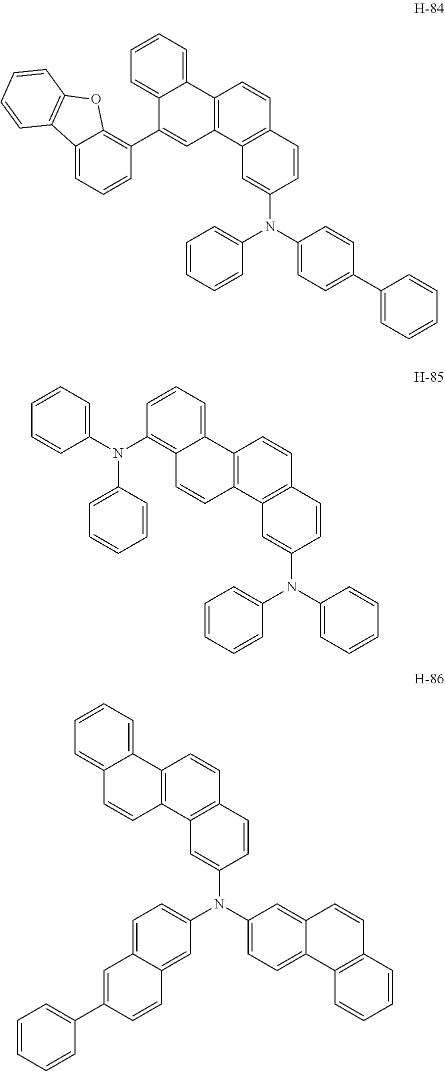 [SMILES: c1ccc(-c2ccc(N(c3ccccc3)c3ccc4ccc5c6ccccc6c(-c6cccc7c6oc6ccccc67)cc5c4c3)cc2)cc1 ; c1ccc(N(c2ccccc2)c2ccc3ccc4c5cccc(N(c6ccccc6)c6ccccc6)c5ccc4c3c2)cc1 ; c1ccc(-c2ccc3cc(N(c4ccc5c(ccc6ccccc65)c4)c4ccc5ccc6c7ccccc7ccc6c5c4)ccc3c2)cc1]

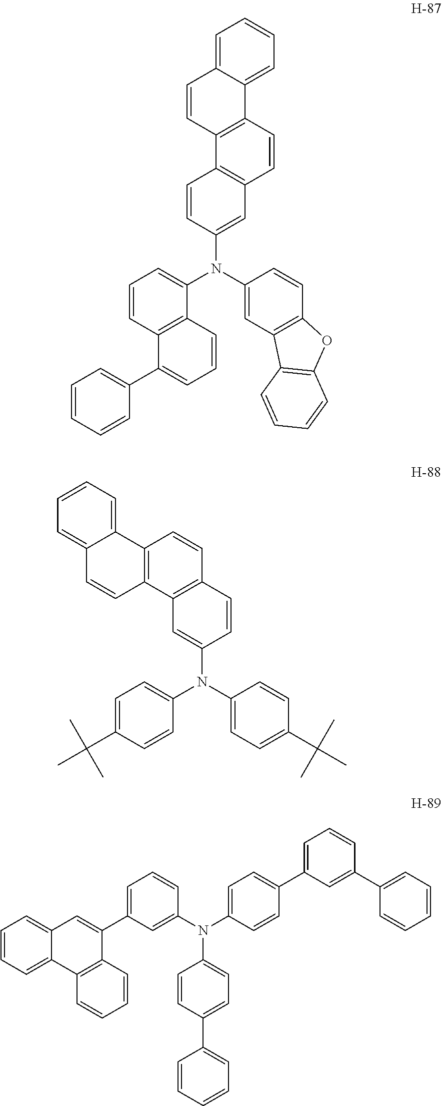 [SMILES: c1ccc(-c2cccc3c(N(c4ccc5c(ccc6c7ccccc7ccc56)c4)c4ccc5oc6ccccc6c5c4)cccc23)cc1 ; CC(C)(C)c1ccc(N(c2ccc(C(C)(C)C)cc2)c2ccc3ccc4c5ccccc5ccc4c3c2)cc1 ; c1ccc(-c2ccc(N(c3ccc(-c4cccc(-c5ccccc5)c4)cc3)c3cccc(-c4cc5ccccc5c5ccccc45)c3)cc2)cc1]

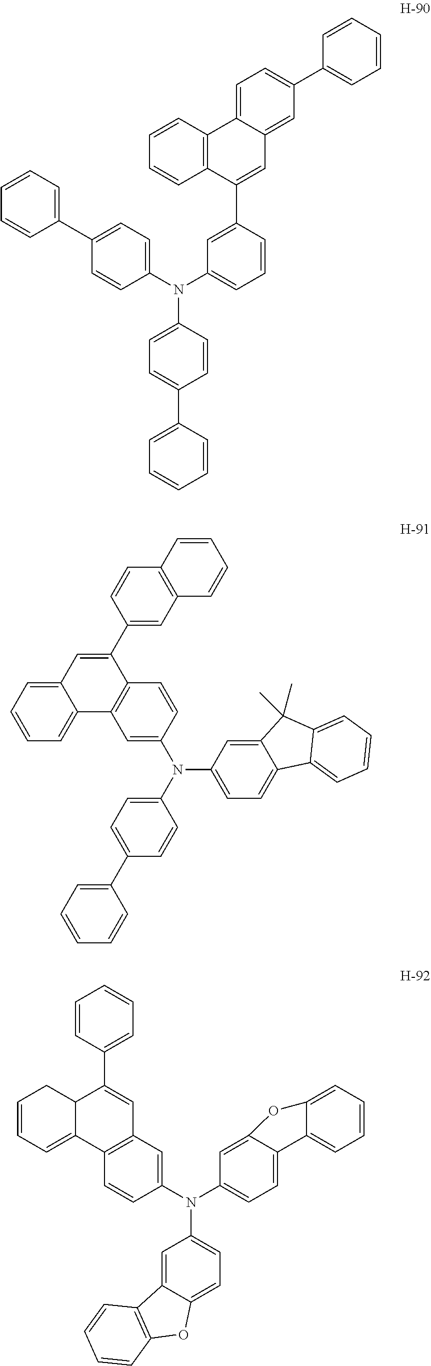

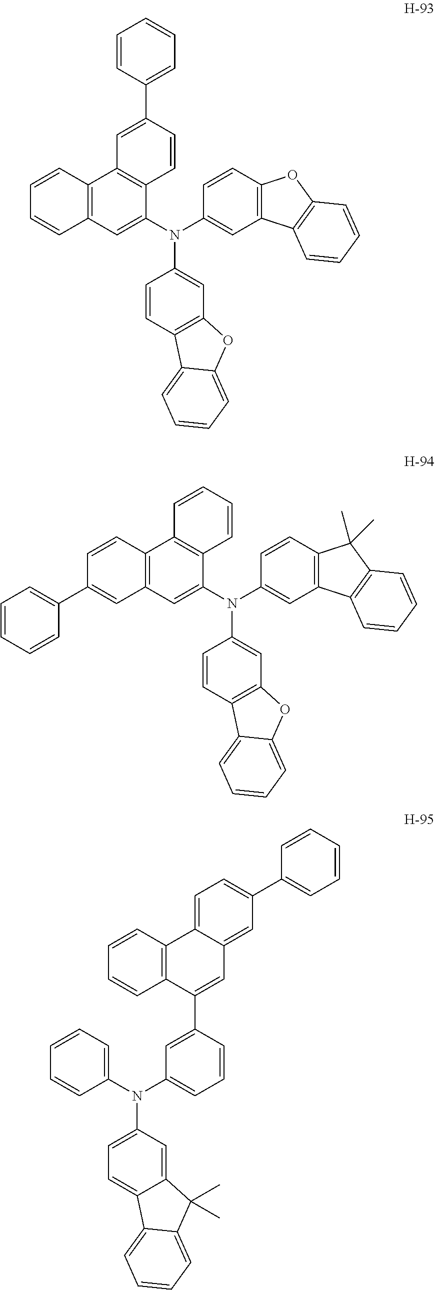 [SMILES: c1ccc(-c2ccc3c(N(c4ccc5c(c4)oc4ccccc45)c4ccc5oc6ccccc6c5c4)cc4ccccc4c3c2)cc1 ; CC1(C)c2ccccc2-c2cc(N(c3ccc4c(c3)oc3ccccc34)c3cc4cc(-c5ccccc5)ccc4c4ccccc34)ccc21 ; CC1(C)c2ccccc2-c2ccc(N(c3ccccc3)c3cccc(-c4cc5cc(-c6ccccc6)ccc5c5ccccc45)c3)cc21]

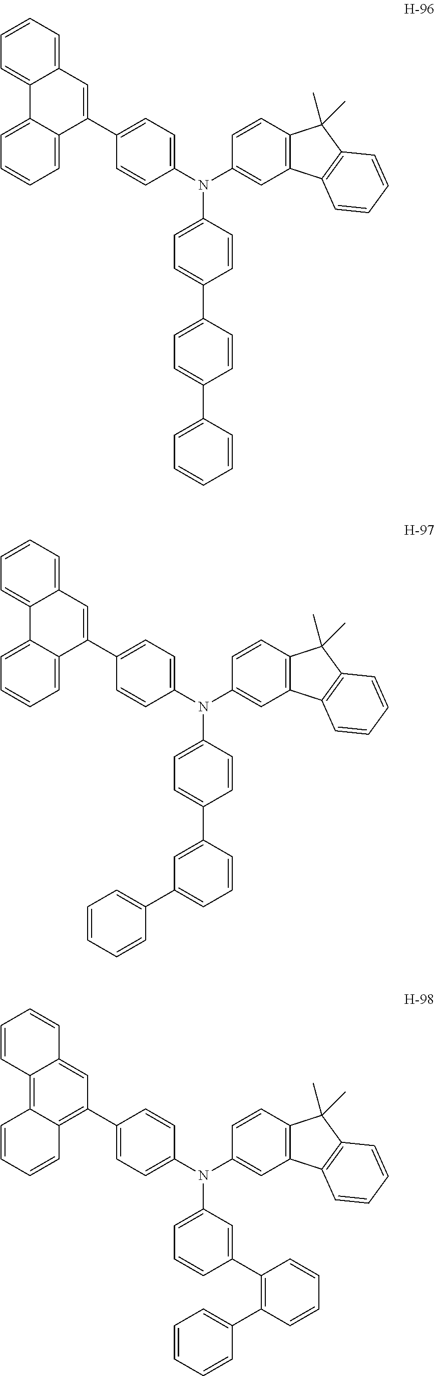 [SMILES: CC1(C)c2ccccc2-c2cc(N(c3ccc(-c4ccc(-c5ccccc5)cc4)cc3)c3ccc(-c4cc5ccccc5c5ccccc45)cc3)ccc21 ; CC1(C)c2ccccc2-c2cc(N(c3ccc(-c4cccc(-c5ccccc5)c4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3cccc(-c4ccccc4-c4ccccc4)c3)ccc21]

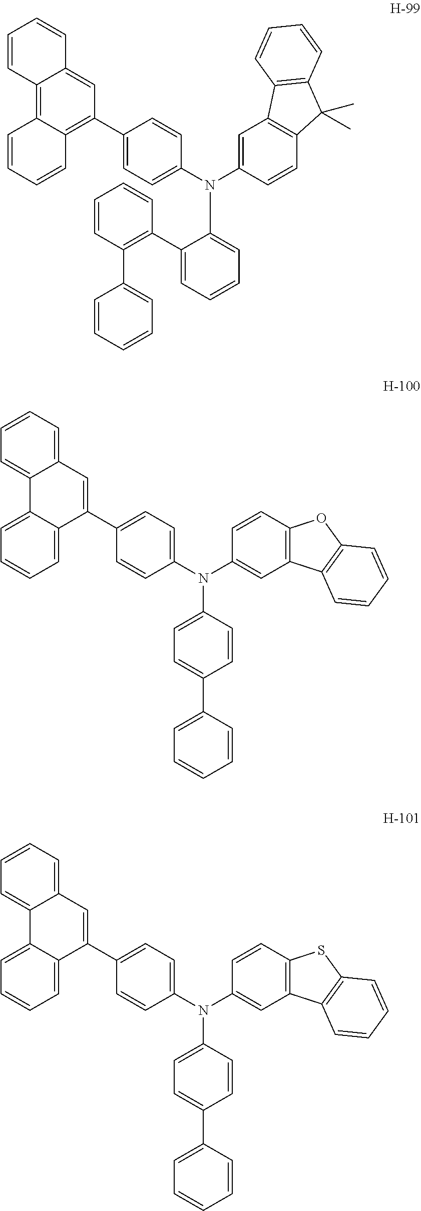 [SMILES: CC1(C)c2ccccc2-c2cc(N(c3ccc(-c4cc5ccccc5c5ccccc45)cc3)c3ccccc3-c3ccccc3-c3ccccc3)ccc21 ; c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4sc5ccccc5c4c3)cc2)cc1]

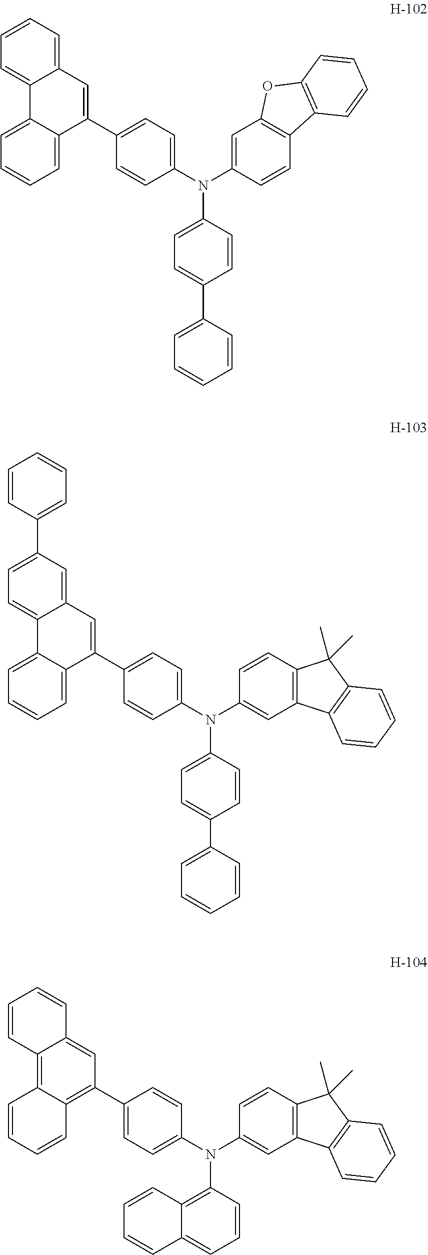 [SMILES: c1ccc(-c2ccc(N(c3ccc(-c4cc5ccccc5c5ccccc45)cc3)c3ccc4c(c3)oc3ccccc34)cc2)cc1 ; CC1(C)c2ccccc2-c2cc(N(c3ccc(-c4ccccc4)cc3)c3ccc(-c4cc5cc(-c6ccccc6)ccc5c5ccccc45)cc3)ccc21 ; CC1(C)c2ccccc2-c2cc(N(c3ccc(-c4cc5ccccc5c5ccccc45)cc3)c3cccc4ccccc34)ccc21]

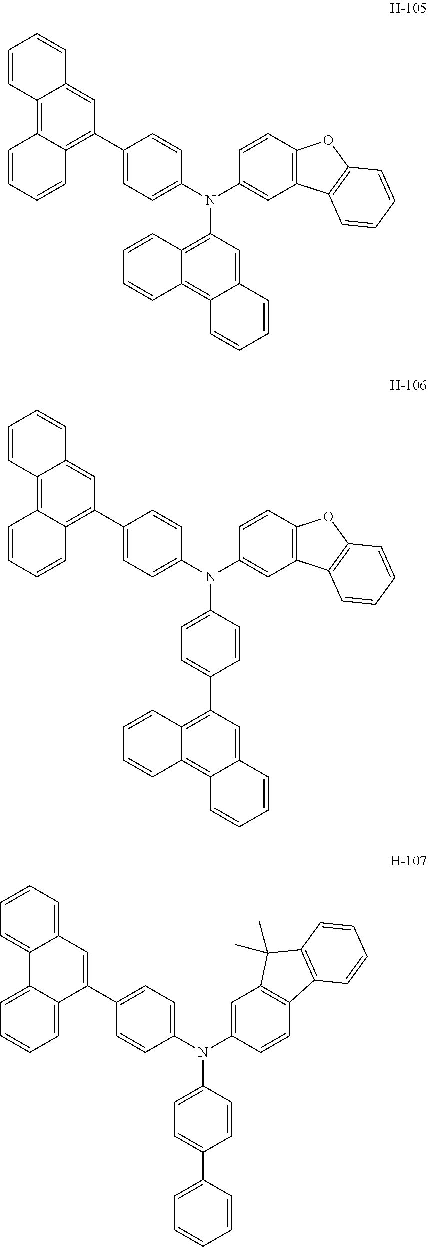 [SMILES: c1ccc2c(c1)cc(-c1ccc(N(c3ccc4oc5ccccc5c4c3)c3cc4ccccc4c4ccccc34)cc1)c1ccccc12 ; c1ccc2c(c1)cc(-c1ccc(N(c3ccc(-c4cc5ccccc5c5ccccc45)cc3)c3ccc4oc5ccccc5c4c3)cc1)c1ccccc12 ; CC1(C)c2ccccc2-c2ccc(N(c3ccc(-c4ccccc4)cc3)c3ccc(-c4cc5ccccc5c5ccccc45)cc3)cc21]

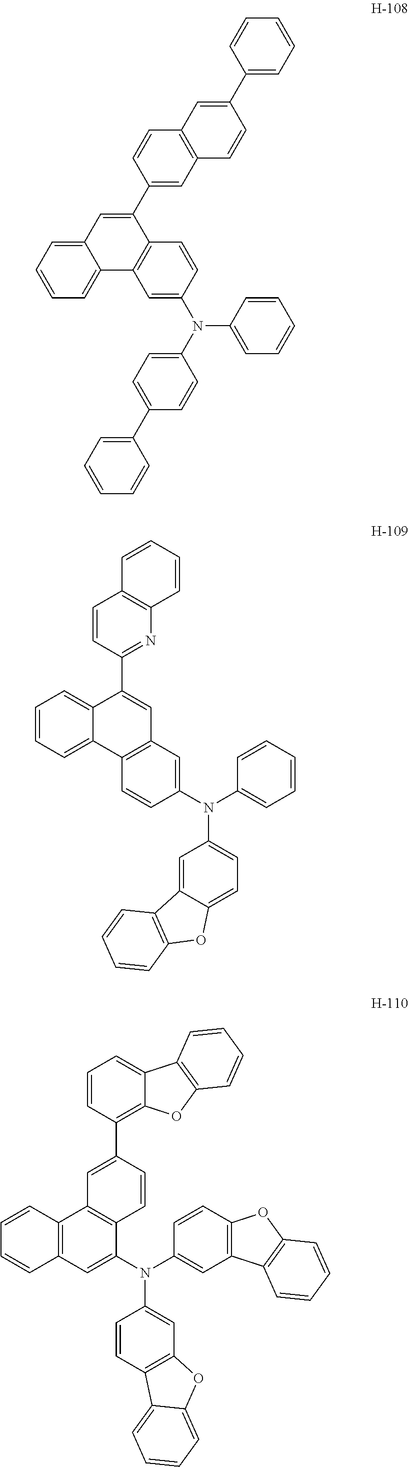 [SMILES: c1ccc(-c2ccc(N(c3ccccc3)c3ccc4c(-c5ccc6cc(-c7ccccc7)ccc6c5)cc5ccccc5c4c3)cc2)cc1 ; c1ccc(N(c2ccc3c(c2)cc(-c2ccc4ccccc4n2)c2ccccc23)c2ccc3oc4ccccc4c3c2)cc1 ; c1ccc2c(c1)cc(N(c1ccc3c(c1)oc1ccccc13)c1ccc3oc4ccccc4c3c1)c1ccc(-c3cccc4c3oc3ccccc34)cc12]

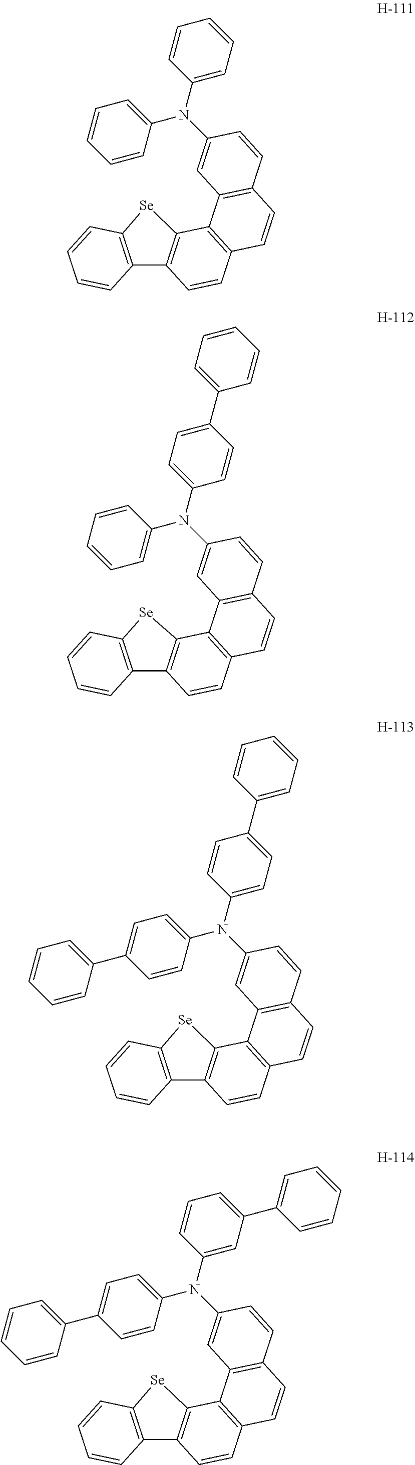

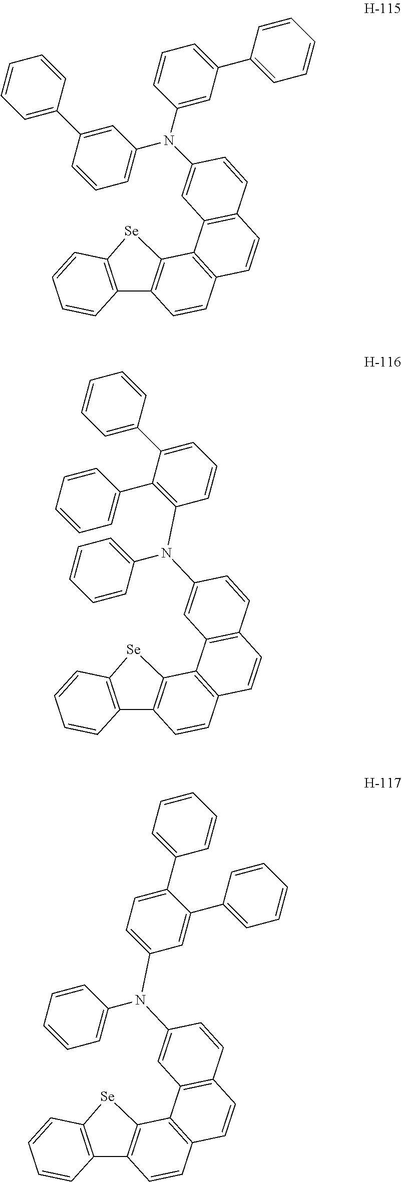 [SMILES: c1ccc(-c2cccc(N(c3cccc(-c4ccccc4)c3)c3ccc4ccc5ccc6c7ccccc7[se]c6c5c4c3)c2)cc1 ; c1ccc(-c2cccc(N(c3ccccc3)c3ccc4ccc5ccc6c7ccccc7[se]c6c5c4c3)c2-c2ccccc2)cc1 ; c1ccc(-c2ccc(N(c3ccccc3)c3ccc4ccc5ccc6c7ccccc7[se]c6c5c4c3)cc2-c2ccccc2)cc1]

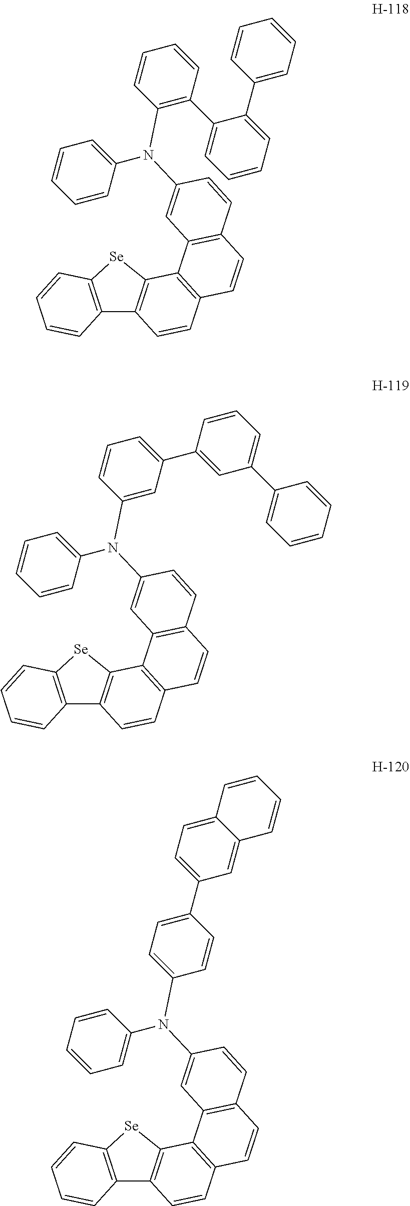 [SMILES: c1ccc(-c2ccccc2-c2ccccc2N(c2ccccc2)c2ccc3ccc4ccc5c6ccccc6[se]c5c4c3c2)cc1 ; c1ccc(-c2cccc(-c3cccc(N(c4ccccc4)c4ccc5ccc6ccc7c8ccccc8[se]c7c6c5c4)c3)c2)cc1 ; c1ccc(N(c2ccc(-c3ccc4ccccc4c3)cc2)c2ccc3ccc4ccc5c6ccccc6[se]c5c4c3c2)cc1]

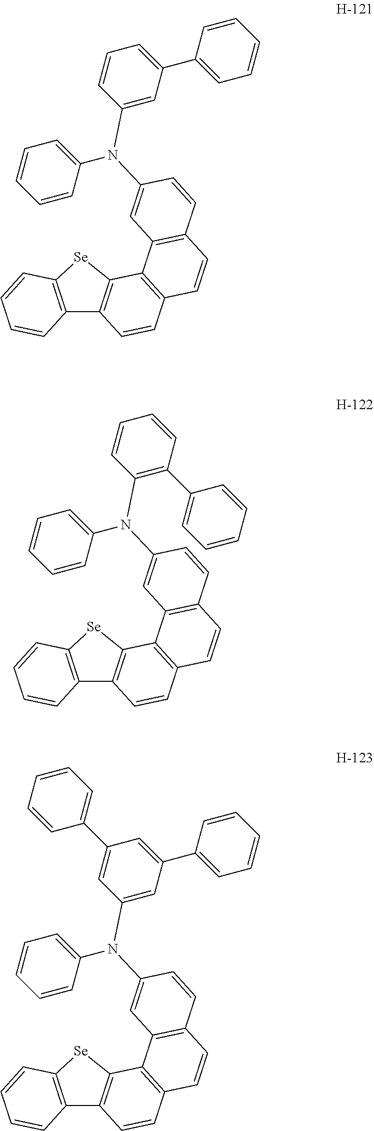 [SMILES: c1ccc(-c2cccc(N(c3ccccc3)c3ccc4ccc5ccc6c7ccccc7[se]c6c5c4c3)c2)cc1 ; c1ccc(-c2ccccc2N(c2ccccc2)c2ccc3ccc4ccc5c6ccccc6[se]c5c4c3c2)cc1 ; c1ccc(-c2cc(-c3ccccc3)cc(N(c3ccccc3)c3ccc4ccc5ccc6c7ccccc7[se]c6c5c4c3)c2)cc1]

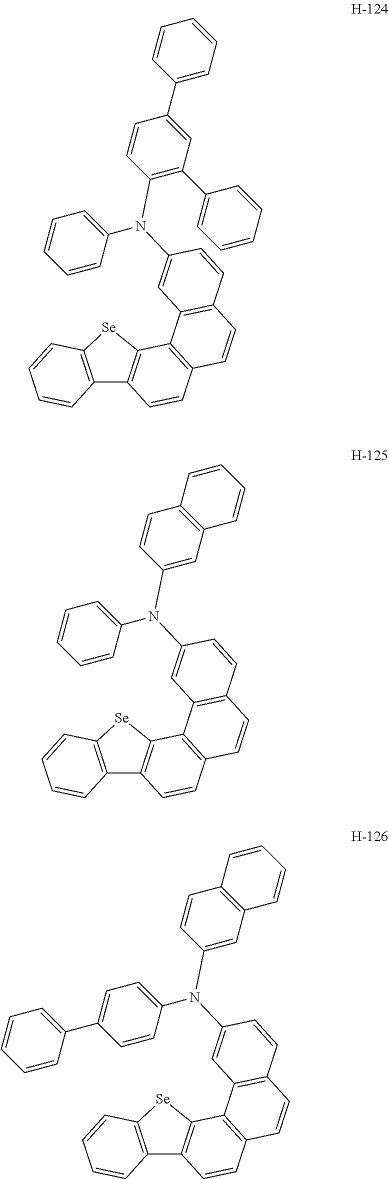 [SMILES: c1ccc(-c2ccc(N(c3ccccc3)c3ccc4ccc5ccc6c7ccccc7[se]c6c5c4c3)c(-c3ccccc3)c2)cc1 ; c1ccc(N(c2ccc3ccccc3c2)c2ccc3ccc4ccc5c6ccccc6[se]c5c4c3c2)cc1 ; c1ccc(-c2ccc(N(c3ccc4ccccc4c3)c3ccc4ccc5ccc6c7ccccc7[se]c6c5c4c3)cc2)cc1]

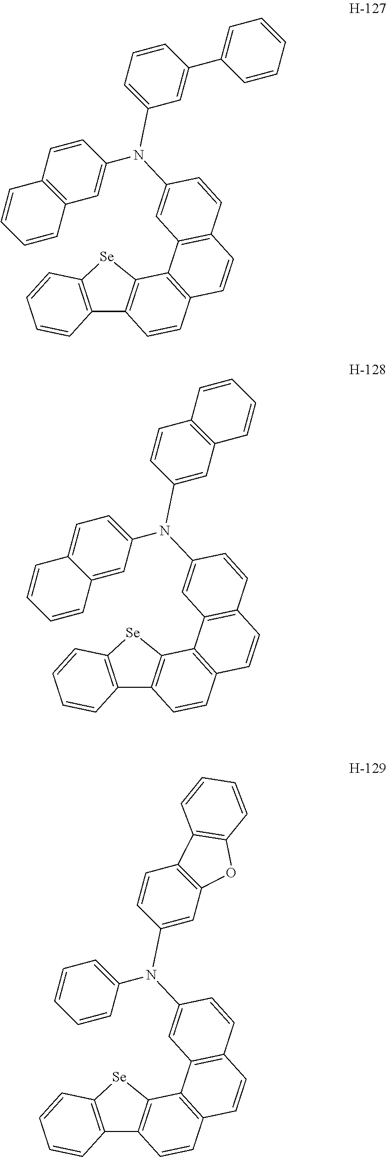 [SMILES: c1ccc(-c2cccc(N(c3ccc4ccccc4c3)c3ccc4ccc5ccc6c7ccccc7[se]c6c5c4c3)c2)cc1 ; c1ccc2cc(N(c3ccc4ccccc4c3)c3ccc4ccc5ccc6c7ccccc7[se]c6c5c4c3)ccc2c1 ; c1ccc(N(c2ccc3c(c2)oc2ccccc23)c2ccc3ccc4ccc5c6ccccc6[se]c5c4c3c2)cc1]

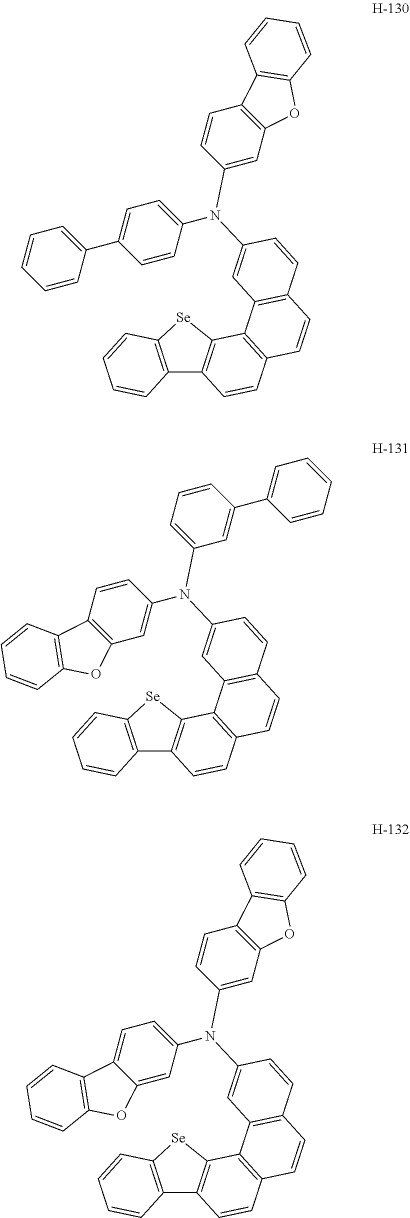 [SMILES: c1ccc(-c2ccc(N(c3ccc4c(c3)oc3ccccc34)c3ccc4ccc5ccc6c7ccccc7[se]c6c5c4c3)cc2)cc1 ; c1ccc(-c2cccc(N(c3ccc4c(c3)oc3ccccc34)c3ccc4ccc5ccc6c7ccccc7[se]c6c5c4c3)c2)cc1 ; c1ccc2c(c1)oc1cc(N(c3ccc4c(c3)oc3ccccc34)c3ccc4ccc5ccc6c7ccccc7[se]c6c5c4c3)ccc12]

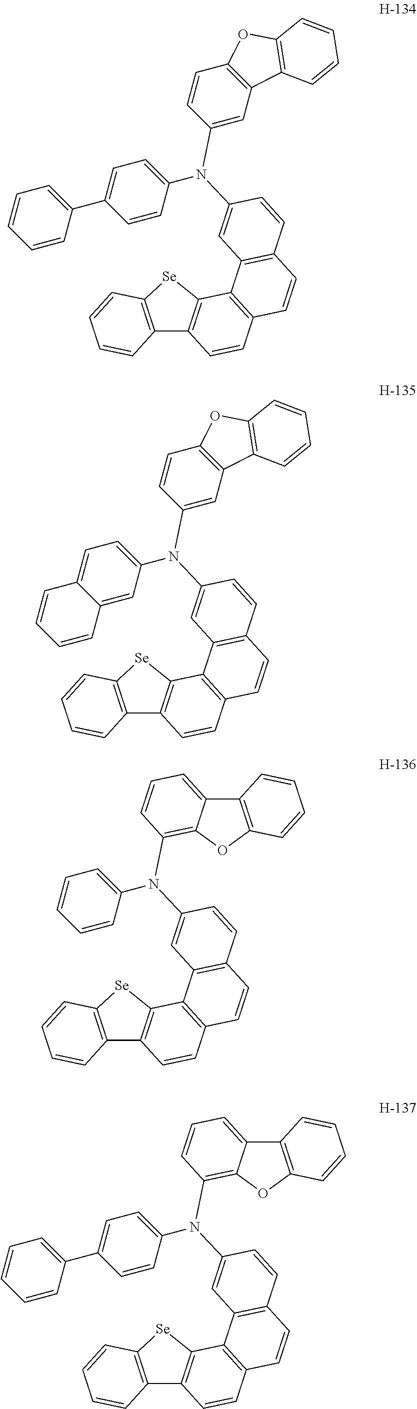

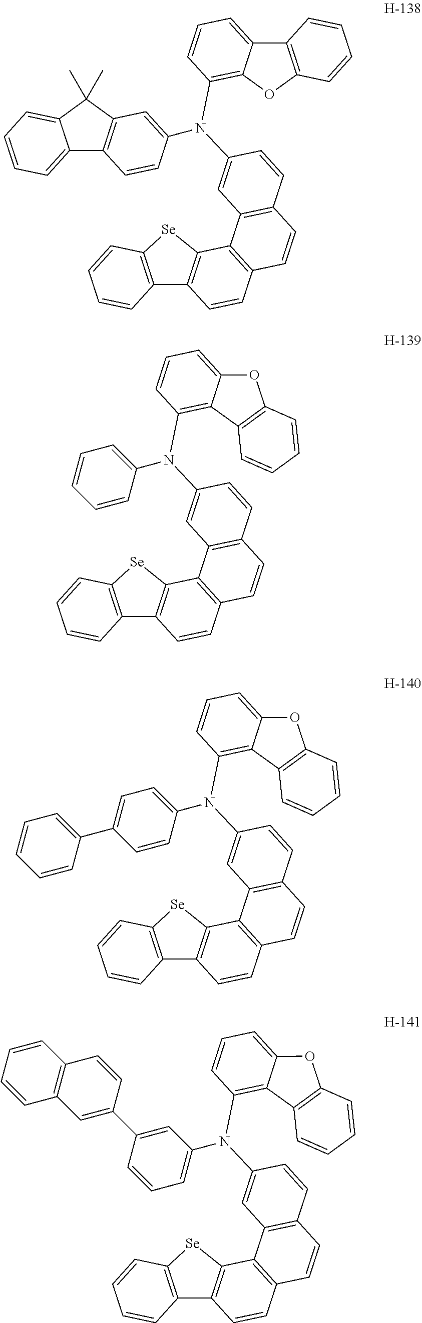

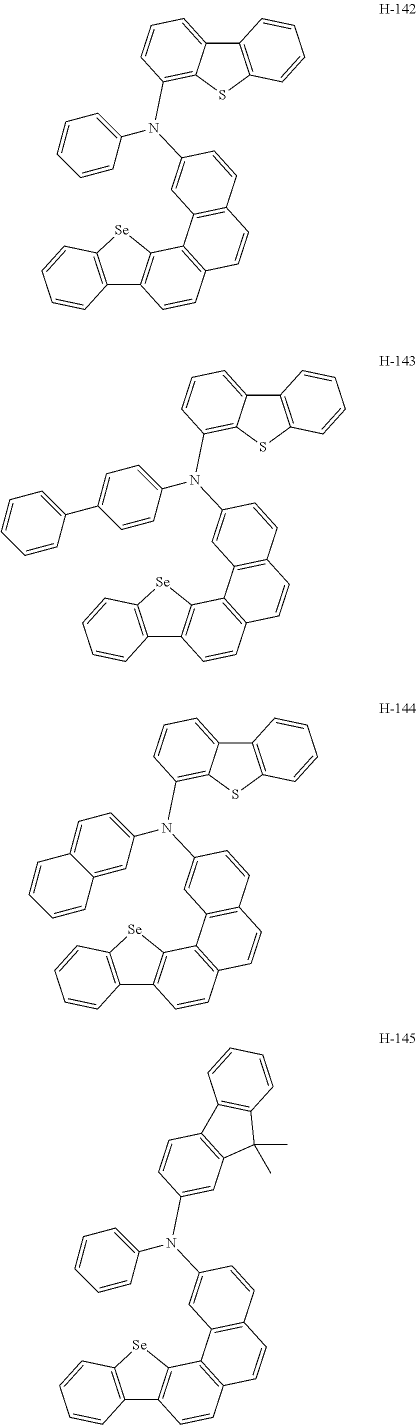 [SMILES: c1ccc(N(c2ccc3ccc4ccc5c6ccccc6[se]c5c4c3c2)c2cccc3c2sc2ccccc23)cc1 ; c1ccc(-c2ccc(N(c3ccc4ccc5ccc6c7ccccc7[se]c6c5c4c3)c3cccc4c3sc3ccccc34)cc2)cc1 ; c1ccc2cc(N(c3ccc4ccc5ccc6c7ccccc7[se]c6c5c4c3)c3cccc4c3sc3ccccc34)ccc2c1 ; CC1(C)c2ccccc2-c2ccc(N(c3ccccc3)c3ccc4ccc5ccc6c7ccccc7[se]c6c5c4c3)cc21]

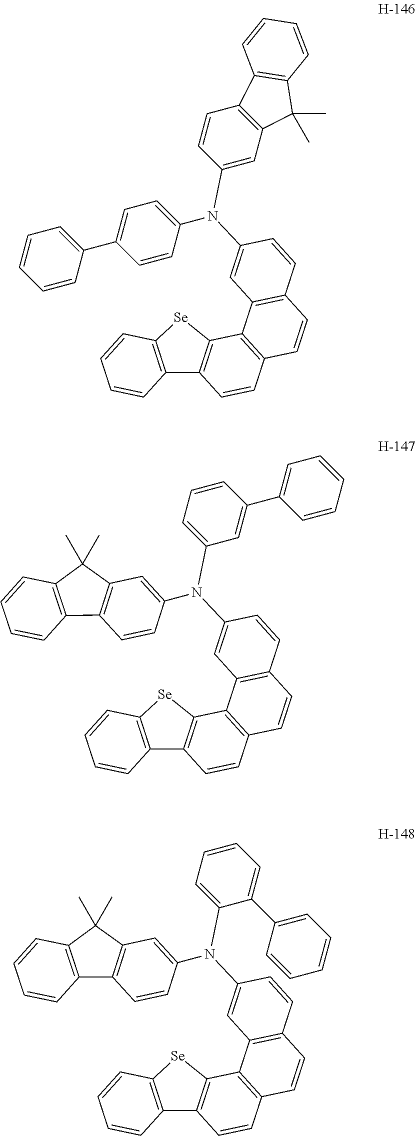 [SMILES: CC1(C)c2ccccc2-c2ccc(N(c3ccc(-c4ccccc4)cc3)c3ccc4ccc5ccc6c7ccccc7[se]c6c5c4c3)cc21 ; CC1(C)c2ccccc2-c2ccc(N(c3cccc(-c4ccccc4)c3)c3ccc4ccc5ccc6c7ccccc7[se]c6c5c4c3)cc21 ; CC1(C)c2ccccc2-c2ccc(N(c3ccc4ccc5ccc6c7ccccc7[se]c6c5c4c3)c3ccccc3-c3ccccc3)cc21]

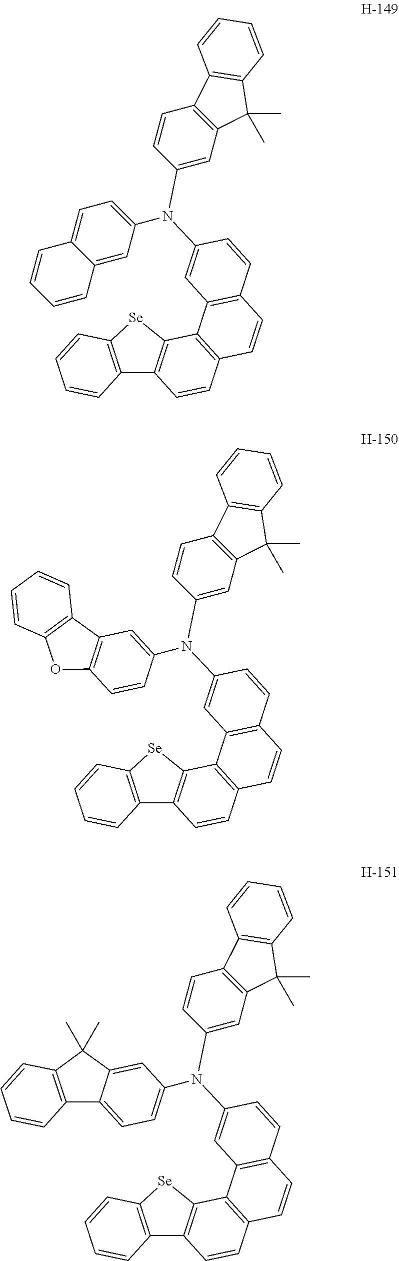 [SMILES: CC1(C)c2ccccc2-c2ccc(N(c3ccc4ccccc4c3)c3ccc4ccc5ccc6c7ccccc7[se]c6c5c4c3)cc21 ; CC1(C)c2ccccc2-c2ccc(N(c3ccc4oc5ccccc5c4c3)c3ccc4ccc5ccc6c7ccccc7[se]c6c5c4c3)cc21 ; CC1(C)c2ccccc2-c2ccc(N(c3ccc4c(c3)C(C)(C)c3ccccc3-4)c3ccc4ccc5ccc6c7ccccc7[se]c6c5c4c3)cc21]

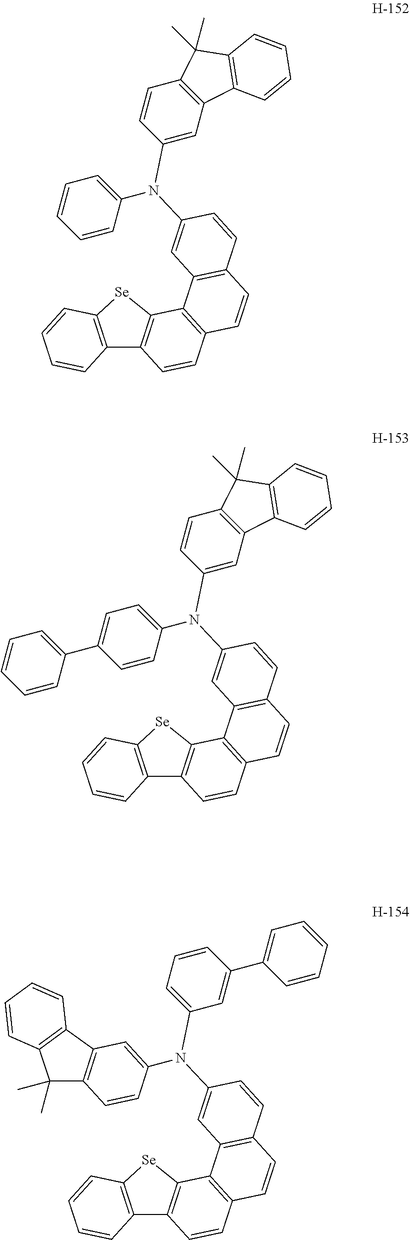 [SMILES: CC1(C)c2ccccc2-c2cc(N(c3ccccc3)c3ccc4ccc5ccc6c7ccccc7[se]c6c5c4c3)ccc21 ; CC1(C)c2ccccc2-c2cc(N(c3ccc(-c4ccccc4)cc3)c3ccc4ccc5ccc6c7ccccc7[se]c6c5c4c3)ccc21 ; CC1(C)c2ccccc2-c2cc(N(c3cccc(-c4ccccc4)c3)c3ccc4ccc5ccc6c7ccccc7[se]c6c5c4c3)ccc21]

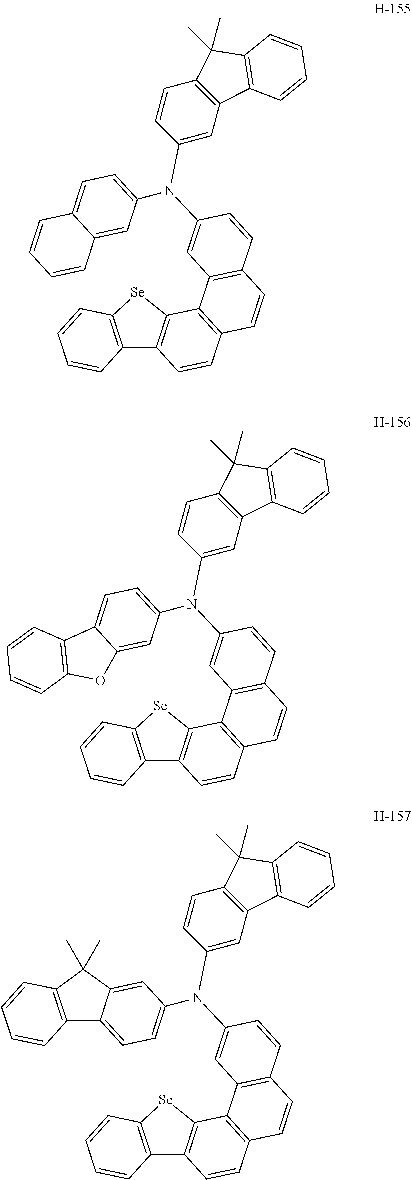 [SMILES: CC1(C)c2ccccc2-c2cc(N(c3ccc4ccccc4c3)c3ccc4ccc5ccc6c7ccccc7[se]c6c5c4c3)ccc21 ; CC1(C)c2ccccc2-c2cc(N(c3ccc4c(c3)oc3ccccc34)c3ccc4ccc5ccc6c7ccccc7[se]c6c5c4c3)ccc21 ; CC1(C)c2ccccc2-c2cc(N(c3ccc4c(c3)C(C)(C)c3ccccc3-4)c3ccc4ccc5ccc6c7ccccc7[se]c6c5c4c3)ccc21]

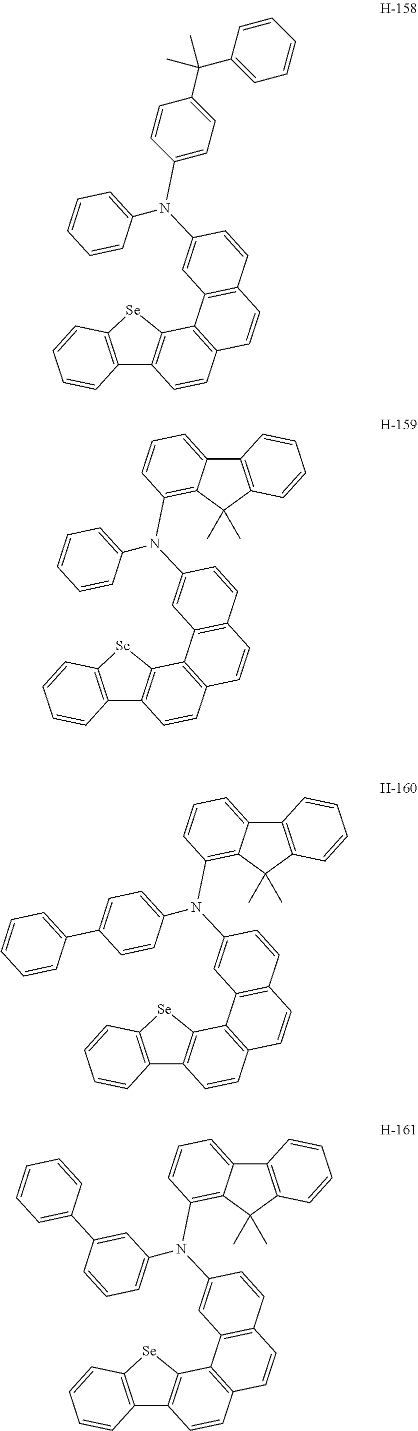

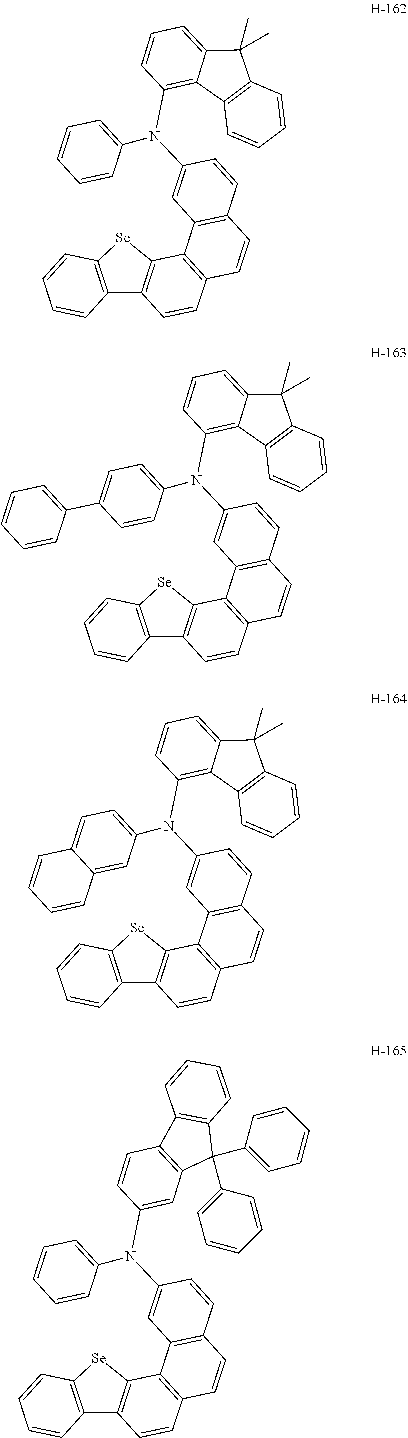 [SMILES: CC1(C)c2ccccc2-c2c(N(c3ccccc3)c3ccc4ccc5ccc6c7ccccc7[se]c6c5c4c3)cccc21 ; CC1(C)c2ccccc2-c2c(N(c3ccc(-c4ccccc4)cc3)c3ccc4ccc5ccc6c7ccccc7[se]c6c5c4c3)cccc21 ; CC1(C)c2ccccc2-c2c(N(c3ccc4ccccc4c3)c3ccc4ccc5ccc6c7ccccc7[se]c6c5c4c3)cccc21 ; c1ccc(N(c2ccc3c(c2)C(c2ccccc2)(c2ccccc2)c2ccccc2-3)c2ccc3ccc4ccc5c6ccccc6[se]c5c4c3c2)cc1]

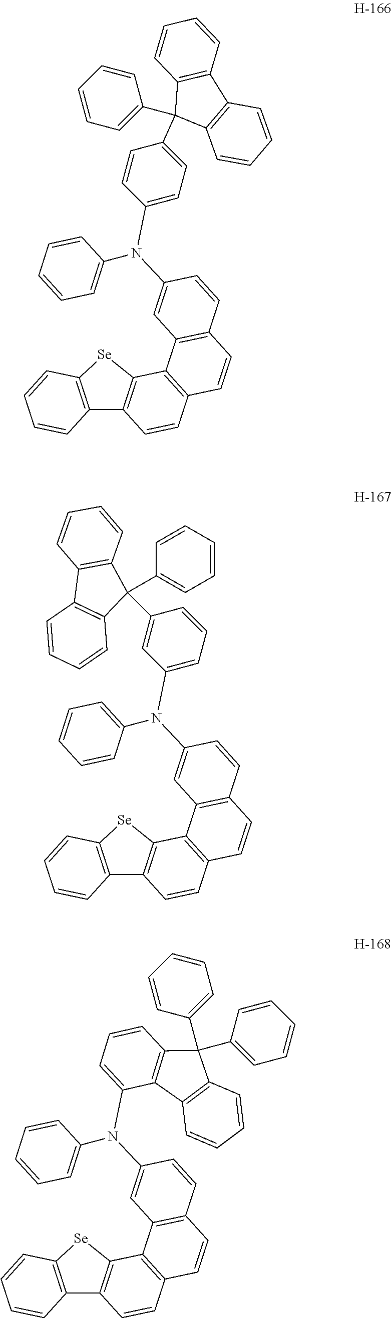 [SMILES: c1ccc(N(c2ccc(C3(c4ccccc4)c4ccccc4-c4ccccc43)cc2)c2ccc3ccc4ccc5c6ccccc6[se]c5c4c3c2)cc1 ; c1ccc(N(c2cccc(C3(c4ccccc4)c4ccccc4-c4ccccc43)c2)c2ccc3ccc4ccc5c6ccccc6[se]c5c4c3c2)cc1 ; c1ccc(N(c2ccc3ccc4ccc5c6ccccc6[se]c5c4c3c2)c2cccc3c2-c2ccccc2C3(c2ccccc2)c2ccccc2)cc1]

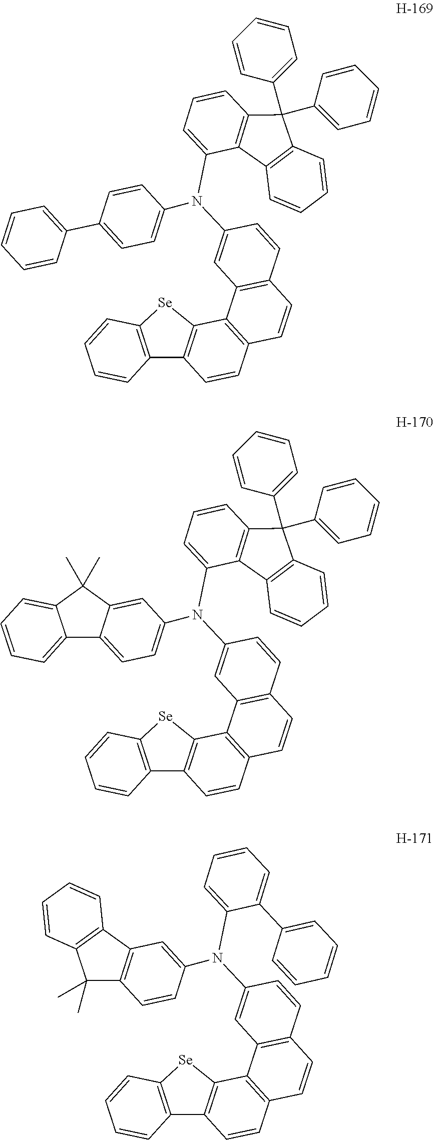 [SMILES: c1ccc(-c2ccc(N(c3ccc4ccc5ccc6c7ccccc7[se]c6c5c4c3)c3cccc4c3-c3ccccc3C4(c3ccccc3)c3ccccc3)cc2)cc1 ; CC1(C)c2ccccc2-c2ccc(N(c3ccc4ccc5ccc6c7ccccc7[se]c6c5c4c3)c3cccc4c3-c3ccccc3C4(c3ccccc3)c3ccccc3)cc21 ; CC1(C)c2ccccc2-c2cc(N(c3ccc4ccc5ccc6c7ccccc7[se]c6c5c4c3)c3ccccc3-c3ccccc3)ccc21]

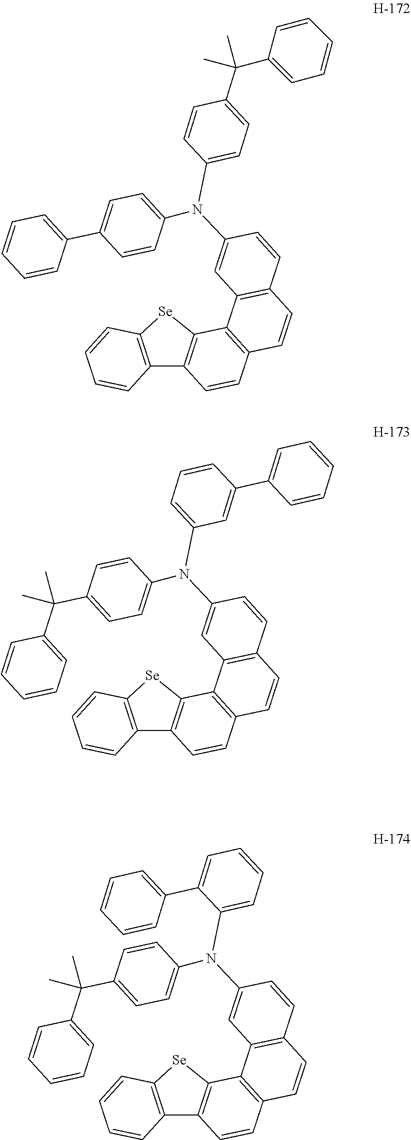 [SMILES: CC(C)(c1ccccc1)c1ccc(N(c2ccc(-c3ccccc3)cc2)c2ccc3ccc4ccc5c6ccccc6[se]c5c4c3c2)cc1 ; CC(C)(c1ccccc1)c1ccc(N(c2cccc(-c3ccccc3)c2)c2ccc3ccc4ccc5c6ccccc6[se]c5c4c3c2)cc1 ; CC(C)(c1ccccc1)c1ccc(N(c2ccc3ccc4ccc5c6ccccc6[se]c5c4c3c2)c2ccccc2-c2ccccc2)cc1]

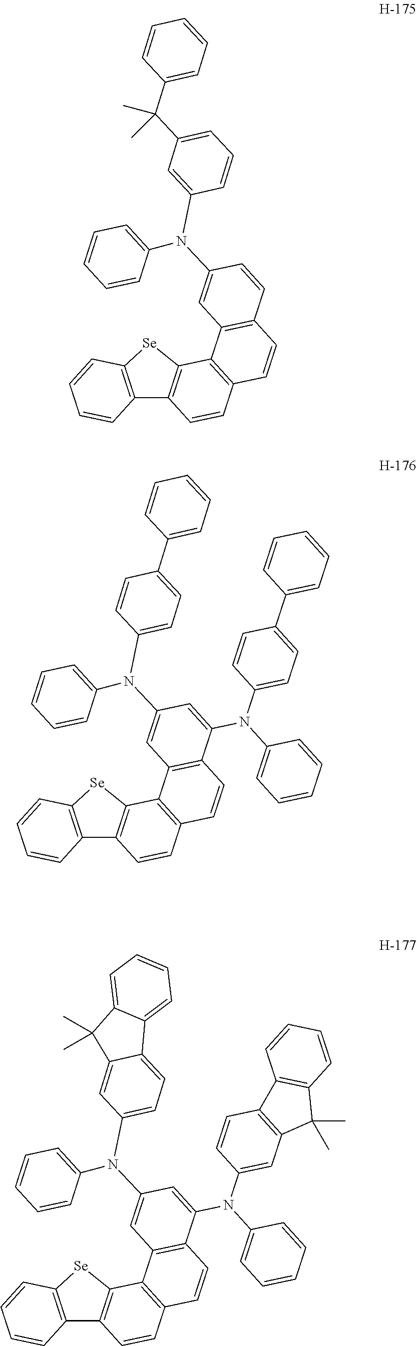 [SMILES: CC(C)(c1ccccc1)c1cccc(N(c2ccccc2)c2ccc3ccc4ccc5c6ccccc6[se]c5c4c3c2)c1 ; c1ccc(-c2ccc(N(c3ccccc3)c3cc(N(c4ccccc4)c4ccc(-c5ccccc5)cc4)c4ccc5ccc6c7ccccc7[se]c6c5c4c3)cc2)cc1 ; CC1(C)c2ccccc2-c2ccc(N(c3ccccc3)c3cc(N(c4ccccc4)c4ccc5c(c4)C(C)(C)c4ccccc4-5)c4ccc5ccc6c7ccccc7[se]c6c5c4c3)cc21]

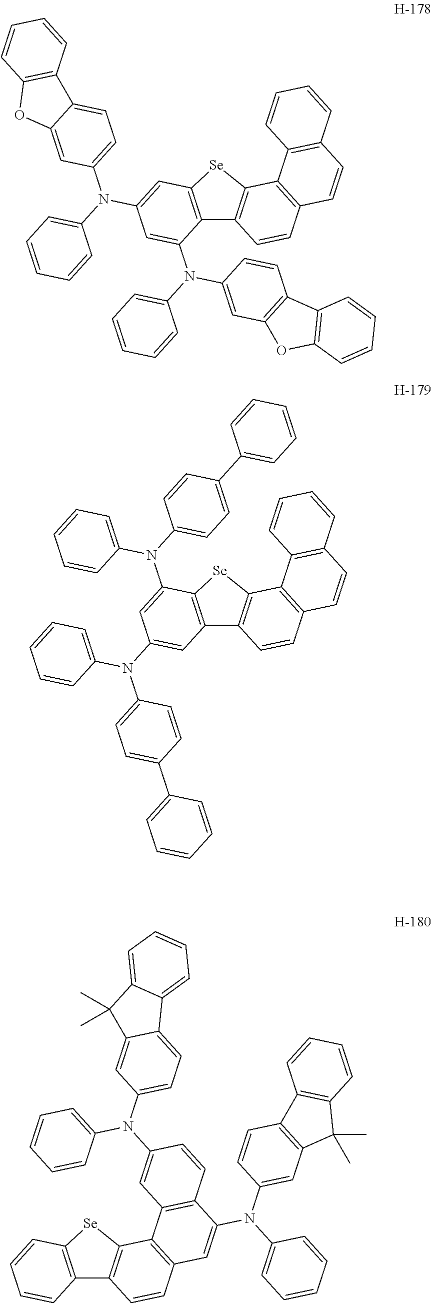 [SMILES: c1ccc(N(c2ccc3c(c2)oc2ccccc23)c2cc(N(c3ccccc3)c3ccc4c(c3)oc3ccccc34)c3c(c2)[se]c2c3ccc3ccc4ccccc4c32)cc1 ; c1ccc(-c2ccc(N(c3ccccc3)c3cc(N(c4ccccc4)c4ccc(-c5ccccc5)cc4)c4[se]c5c(ccc6ccc7ccccc7c65)c4c3)cc2)cc1 ; CC1(C)c2ccccc2-c2ccc(N(c3ccccc3)c3ccc4c(N(c5ccccc5)c5ccc6c(c5)C(C)(C)c5ccccc5-6)cc5ccc6c7ccccc7[se]c6c5c4c3)cc21]

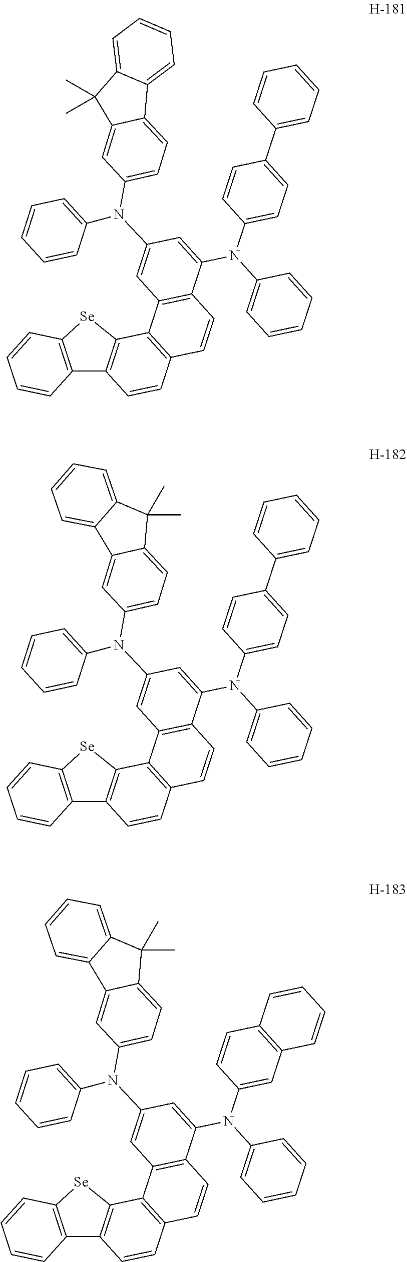 [SMILES: CC1(C)c2ccccc2-c2ccc(N(c3ccccc3)c3cc(N(c4ccccc4)c4ccc(-c5ccccc5)cc4)c4ccc5ccc6c7ccccc7[se]c6c5c4c3)cc21 ; CC1(C)c2ccccc2-c2cc(N(c3ccccc3)c3cc(N(c4ccccc4)c4ccc(-c5ccccc5)cc4)c4ccc5ccc6c7ccccc7[se]c6c5c4c3)ccc21 ; CC1(C)c2ccccc2-c2cc(N(c3ccccc3)c3cc(N(c4ccccc4)c4ccc5ccccc5c4)c4ccc5ccc6c7ccccc7[se]c6c5c4c3)ccc21]

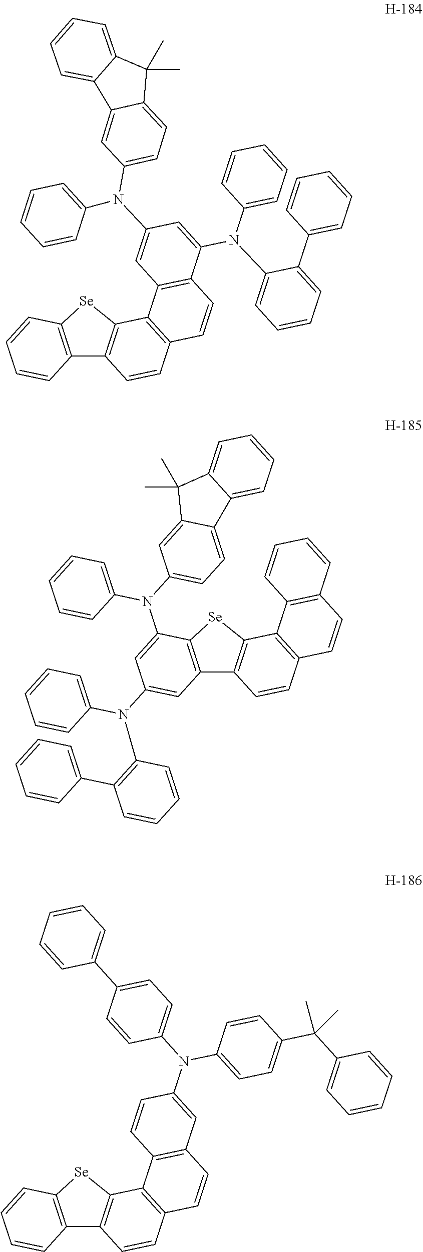 [SMILES: CC1(C)c2ccccc2-c2cc(N(c3ccccc3)c3cc(N(c4ccccc4)c4ccccc4-c4ccccc4)c4ccc5ccc6c7ccccc7[se]c6c5c4c3)ccc21 ; CC1(C)c2ccccc2-c2ccc(N(c3ccccc3)c3cc(N(c4ccccc4)c4ccccc4-c4ccccc4)cc4c3[se]c3c4ccc4ccc5ccccc5c43)cc21 ; CC(C)(c1ccccc1)c1ccc(N(c2ccc(-c3ccccc3)cc2)c2ccc3c(ccc4ccc5c6ccccc6[se]c5c43)c2)cc1]

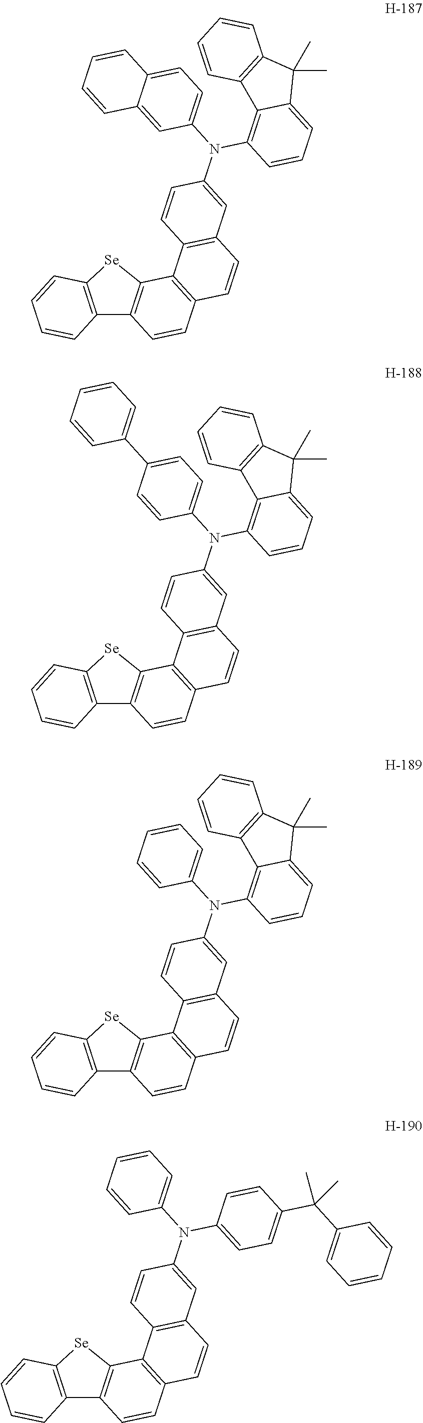 [SMILES: CC1(C)c2ccccc2-c2c(N(c3ccc4ccccc4c3)c3ccc4c(ccc5ccc6c7ccccc7[se]c6c54)c3)cccc21 ; CC1(C)c2ccccc2-c2c(N(c3ccc(-c4ccccc4)cc3)c3ccc4c(ccc5ccc6c7ccccc7[se]c6c54)c3)cccc21 ; CC1(C)c2ccccc2-c2c(N(c3ccccc3)c3ccc4c(ccc5ccc6c7ccccc7[se]c6c54)c3)cccc21 ; CC(C)(c1ccccc1)c1ccc(N(c2ccccc2)c2ccc3c(ccc4ccc5c6ccccc6[se]c5c43)c2)cc1]

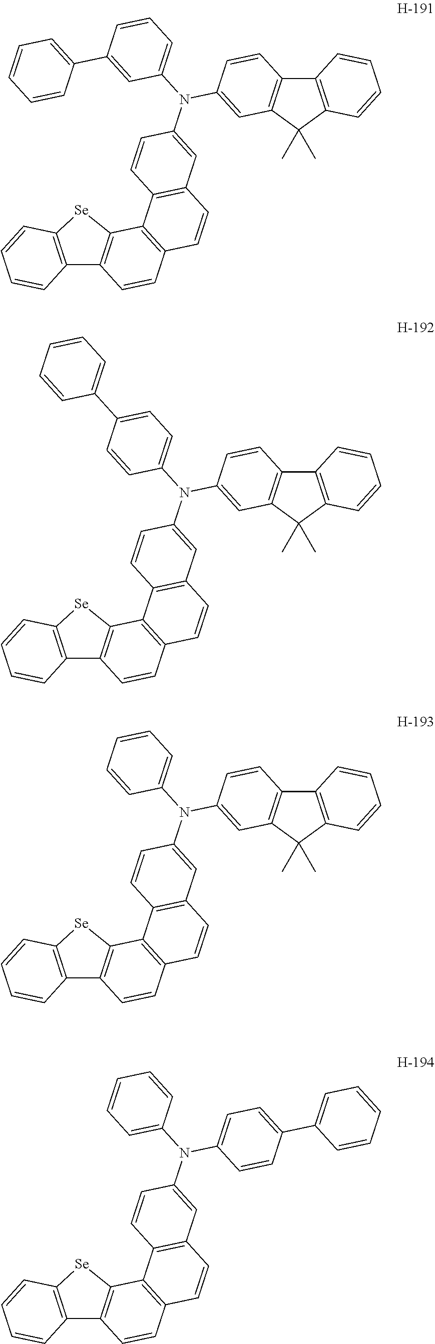 [SMILES: CC1(C)c2ccccc2-c2ccc(N(c3cccc(-c4ccccc4)c3)c3ccc4c(ccc5ccc6c7ccccc7[se]c6c54)c3)cc21 ; CC1(C)c2ccccc2-c2ccc(N(c3ccc(-c4ccccc4)cc3)c3ccc4c(ccc5ccc6c7ccccc7[se]c6c54)c3)cc21 ; CC1(C)c2ccccc2-c2ccc(N(c3ccccc3)c3ccc4c(ccc5ccc6c7ccccc7[se]c6c54)c3)cc21 ; c1ccc(-c2ccc(N(c3ccccc3)c3ccc4c(ccc5ccc6c7ccccc7[se]c6c54)c3)cc2)cc1]

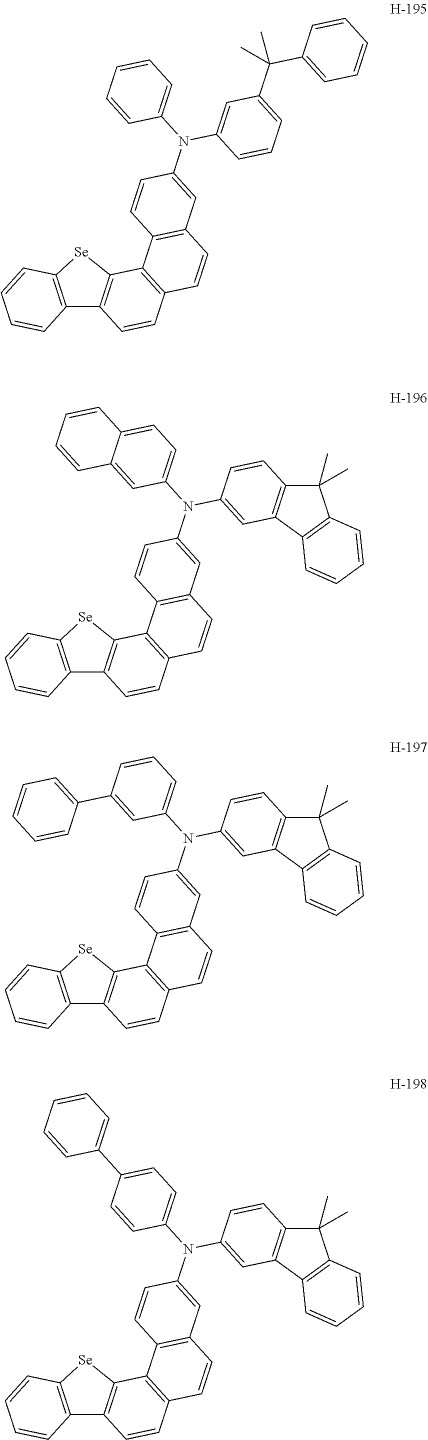 [SMILES: CC(C)(c1ccccc1)c1cccc(N(c2ccccc2)c2ccc3c(ccc4ccc5c6ccccc6[se]c5c43)c2)c1 ; CC1(C)c2ccccc2-c2cc(N(c3ccc4ccccc4c3)c3ccc4c(ccc5ccc6c7ccccc7[se]c6c54)c3)ccc21 ; CC1(C)c2ccccc2-c2cc(N(c3cccc(-c4ccccc4)c3)c3ccc4c(ccc5ccc6c7ccccc7[se]c6c54)c3)ccc21 ; CC1(C)c2ccccc2-c2cc(N(c3ccc(-c4ccccc4)cc3)c3ccc4c(ccc5ccc6c7ccccc7[se]c6c54)c3)ccc21]

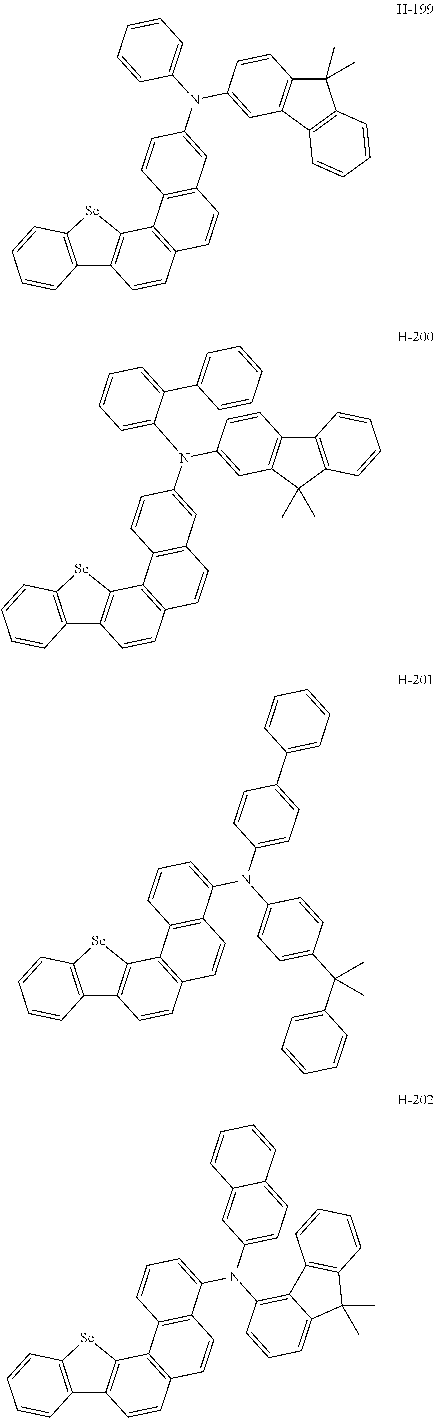 [SMILES: CC1(C)c2ccccc2-c2cc(N(c3ccccc3)c3ccc4c(ccc5ccc6c7ccccc7[se]c6c54)c3)ccc21 ; CC1(C)c2ccccc2-c2ccc(N(c3ccc4c(ccc5ccc6c7ccccc7[se]c6c54)c3)c3ccccc3-c3ccccc3)cc21 ; CC(C)(c1ccccc1)c1ccc(N(c2ccc(-c3ccccc3)cc2)c2cccc3c2ccc2ccc4c5ccccc5[se]c4c23)cc1 ; CC1(C)c2ccccc2-c2c(N(c3ccc4ccccc4c3)c3cccc4c3ccc3ccc5c6ccccc6[se]c5c34)cccc21]

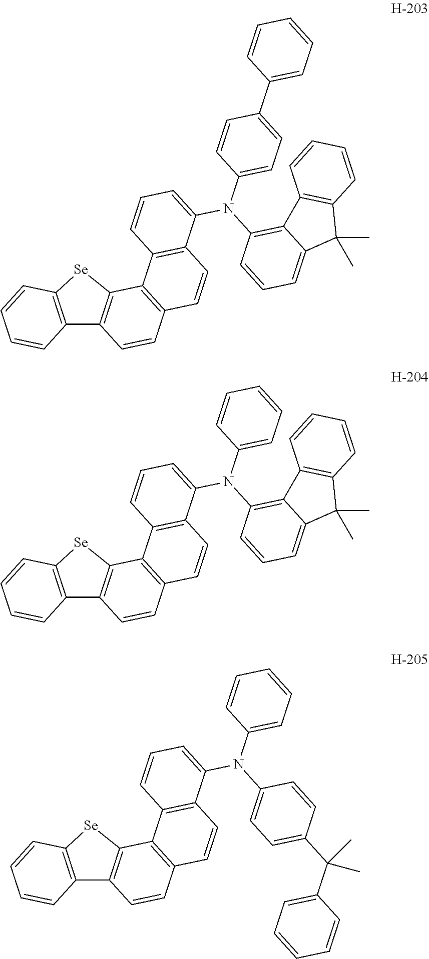 [SMILES: CC1(C)c2ccccc2-c2c(N(c3ccc(-c4ccccc4)cc3)c3cccc4c3ccc3ccc5c6ccccc6[se]c5c34)cccc21 ; CC1(C)c2ccccc2-c2c(N(c3ccccc3)c3cccc4c3ccc3ccc5c6ccccc6[se]c5c34)cccc21 ; CC(C)(c1ccccc1)c1ccc(N(c2ccccc2)c2cccc3c2ccc2ccc4c5ccccc5[se]c4c23)cc1]

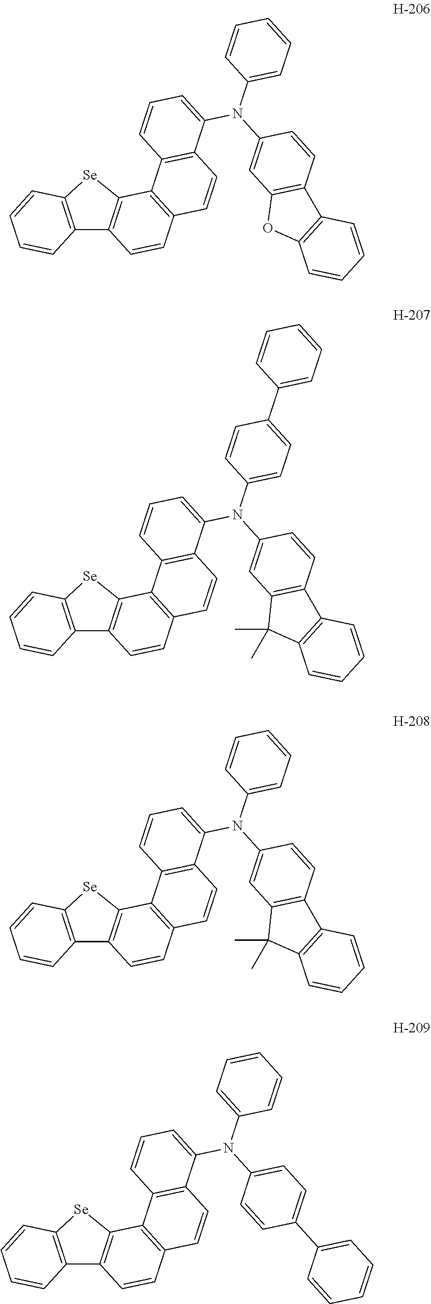 [SMILES: c1ccc(N(c2ccc3c(c2)oc2ccccc23)c2cccc3c2ccc2ccc4c5ccccc5[se]c4c23)cc1 ; CC1(C)c2ccccc2-c2ccc(N(c3ccc(-c4ccccc4)cc3)c3cccc4c3ccc3ccc5c6ccccc6[se]c5c34)cc21 ; CC1(C)c2ccccc2-c2ccc(N(c3ccccc3)c3cccc4c3ccc3ccc5c6ccccc6[se]c5c34)cc21 ; c1ccc(-c2ccc(N(c3ccccc3)c3cccc4c3ccc3ccc5c6ccccc6[se]c5c34)cc2)cc1]

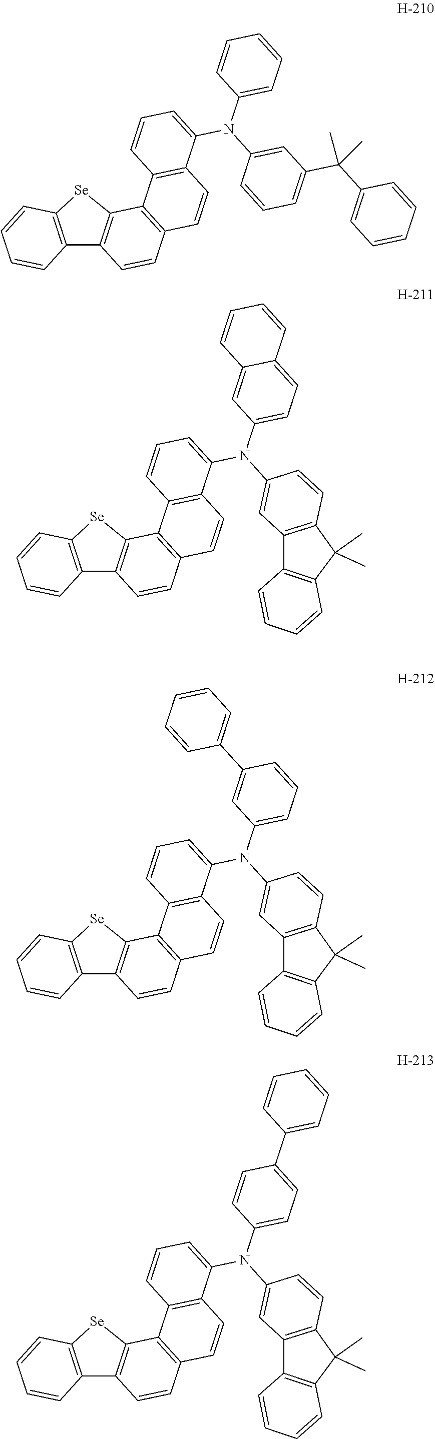

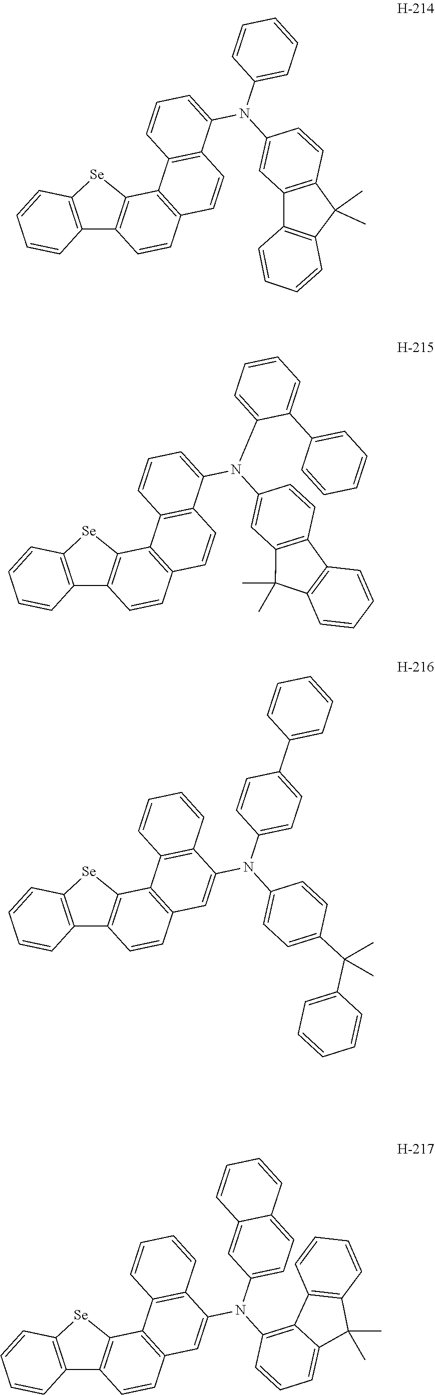 [SMILES: CC1(C)c2ccccc2-c2cc(N(c3ccccc3)c3cccc4c3ccc3ccc5c6ccccc6[se]c5c34)ccc21 ; CC1(C)c2ccccc2-c2ccc(N(c3ccccc3-c3ccccc3)c3cccc4c3ccc3ccc5c6ccccc6[se]c5c34)cc21 ; CC(C)(c1ccccc1)c1ccc(N(c2ccc(-c3ccccc3)cc2)c2cc3ccc4c5ccccc5[se]c4c3c3ccccc23)cc1 ; CC1(C)c2ccccc2-c2c(N(c3ccc4ccccc4c3)c3cc4ccc5c6ccccc6[se]c5c4c4ccccc34)cccc21]

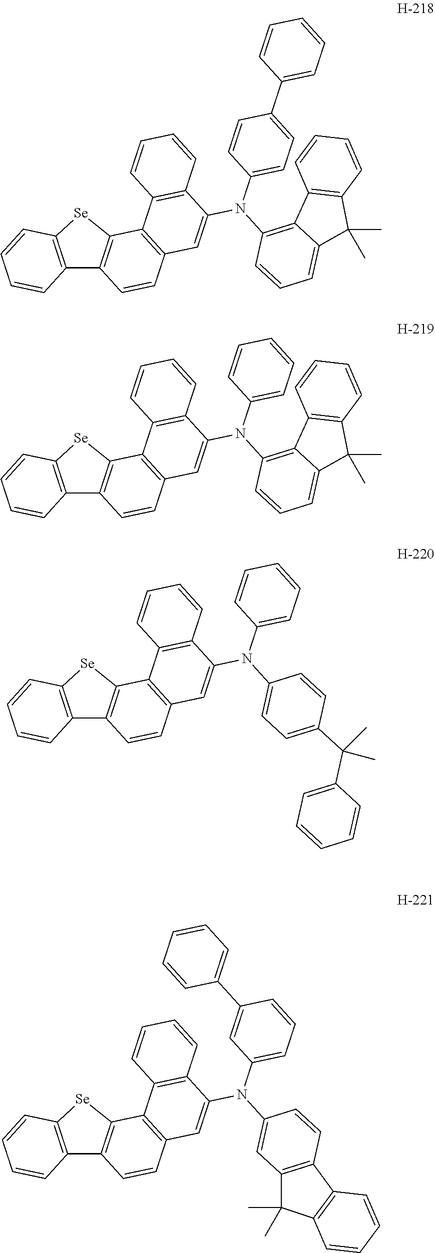 [SMILES: CC1(C)c2ccccc2-c2c(N(c3ccc(-c4ccccc4)cc3)c3cc4ccc5c6ccccc6[se]c5c4c4ccccc34)cccc21 ; CC1(C)c2ccccc2-c2c(N(c3ccccc3)c3cc4ccc5c6ccccc6[se]c5c4c4ccccc34)cccc21 ; CC(C)(c1ccccc1)c1ccc(N(c2ccccc2)c2cc3ccc4c5ccccc5[se]c4c3c3ccccc23)cc1 ; CC1(C)c2ccccc2-c2ccc(N(c3cccc(-c4ccccc4)c3)c3cc4ccc5c6ccccc6[se]c5c4c4ccccc34)cc21]

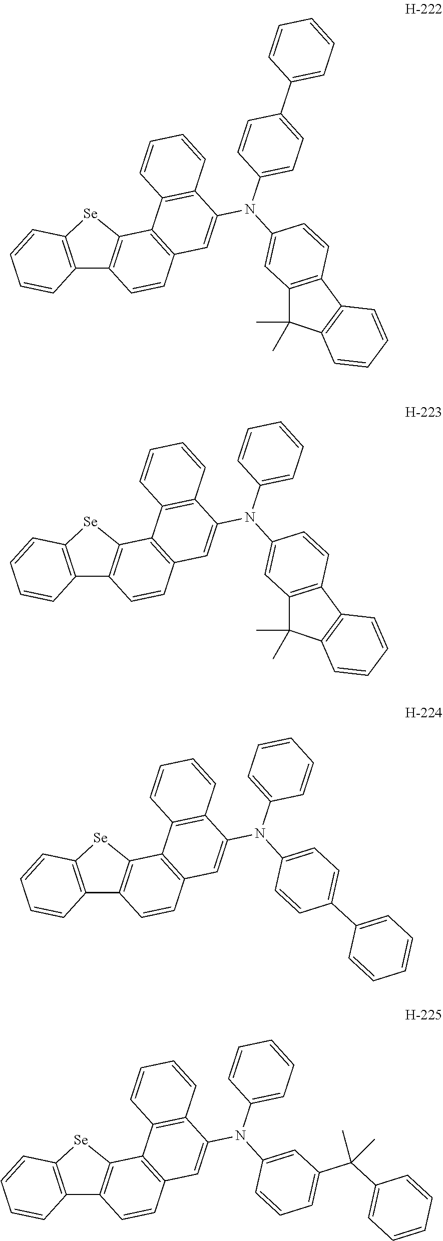 [SMILES: CC1(C)c2ccccc2-c2ccc(N(c3ccc(-c4ccccc4)cc3)c3cc4ccc5c6ccccc6[se]c5c4c4ccccc34)cc21 ; CC1(C)c2ccccc2-c2ccc(N(c3ccccc3)c3cc4ccc5c6ccccc6[se]c5c4c4ccccc34)cc21 ; c1ccc(-c2ccc(N(c3ccccc3)c3cc4ccc5c6ccccc6[se]c5c4c4ccccc34)cc2)cc1 ; CC(C)(c1ccccc1)c1cccc(N(c2ccccc2)c2cc3ccc4c5ccccc5[se]c4c3c3ccccc23)c1]

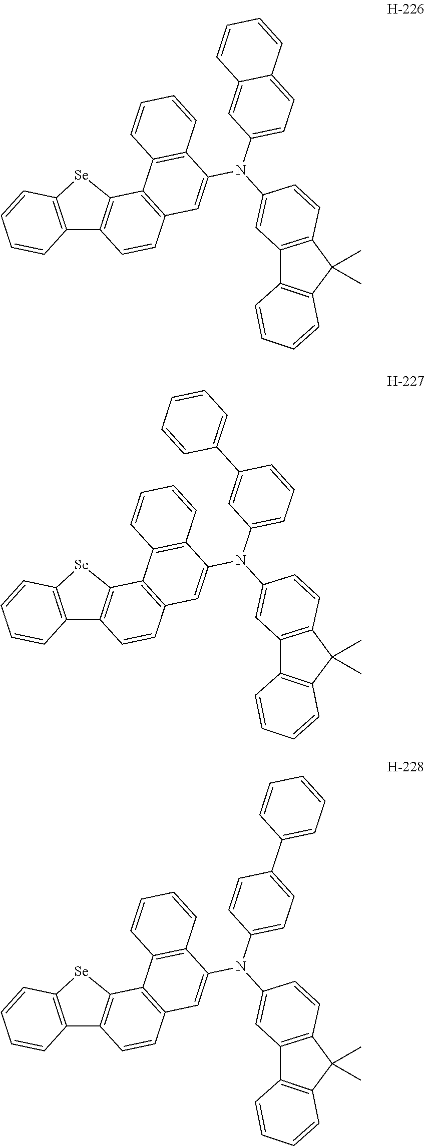

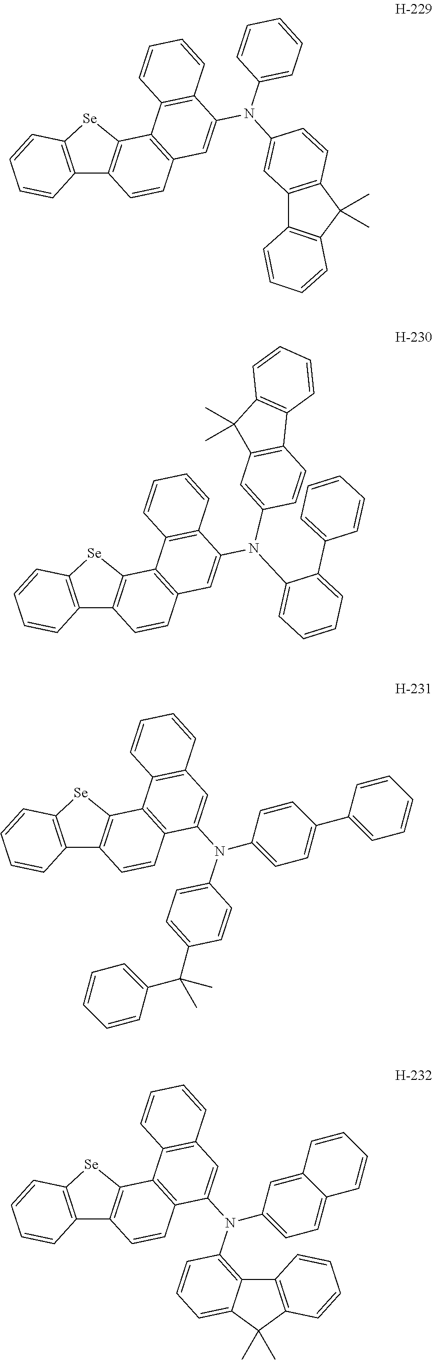 [SMILES: CC1(C)c2ccccc2-c2cc(N(c3ccccc3)c3cc4ccc5c6ccccc6[se]c5c4c4ccccc34)ccc21 ; CC1(C)c2ccccc2-c2ccc(N(c3ccccc3-c3ccccc3)c3cc4ccc5c6ccccc6[se]c5c4c4ccccc34)cc21 ; CC(C)(c1ccccc1)c1ccc(N(c2ccc(-c3ccccc3)cc2)c2cc3ccccc3c3c2ccc2c4ccccc4[se]c23)cc1 ; CC1(C)c2ccccc2-c2c(N(c3ccc4ccccc4c3)c3cc4ccccc4c4c3ccc3c5ccccc5[se]c34)cccc21]

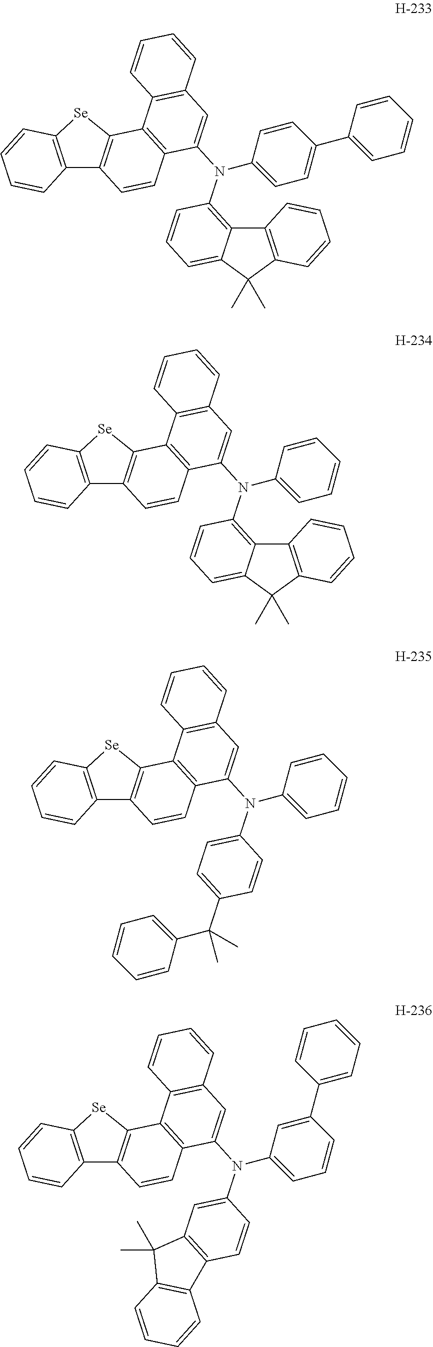 [SMILES: CC1(C)c2ccccc2-c2c(N(c3ccc(-c4ccccc4)cc3)c3cc4ccccc4c4c3ccc3c5ccccc5[se]c34)cccc21 ; CC1(C)c2ccccc2-c2c(N(c3ccccc3)c3cc4ccccc4c4c3ccc3c5ccccc5[se]c34)cccc21 ; CC(C)(c1ccccc1)c1ccc(N(c2ccccc2)c2cc3ccccc3c3c2ccc2c4ccccc4[se]c23)cc1 ; CC1(C)c2ccccc2-c2ccc(N(c3cccc(-c4ccccc4)c3)c3cc4ccccc4c4c3ccc3c5ccccc5[se]c34)cc21]

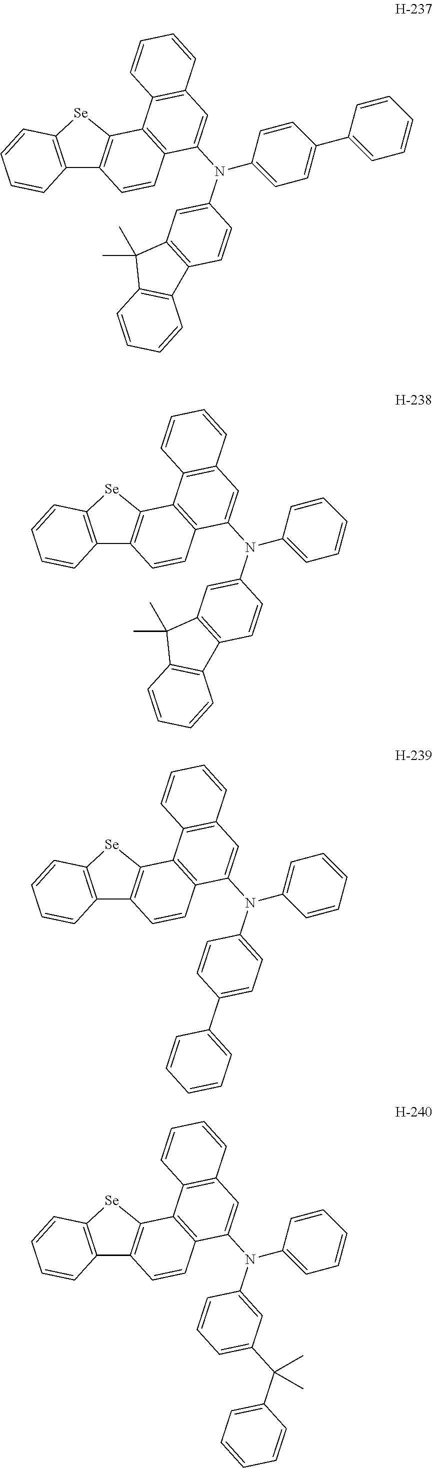

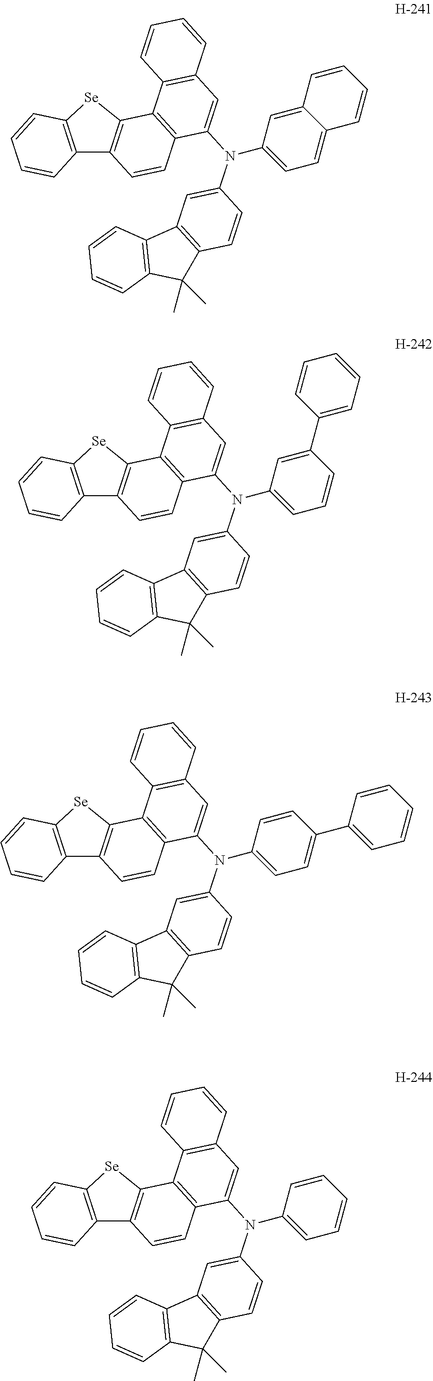 [SMILES: CC1(C)c2ccccc2-c2cc(N(c3ccc4ccccc4c3)c3cc4ccccc4c4c3ccc3c5ccccc5[se]c34)ccc21 ; CC1(C)c2ccccc2-c2cc(N(c3cccc(-c4ccccc4)c3)c3cc4ccccc4c4c3ccc3c5ccccc5[se]c34)ccc21 ; CC1(C)c2ccccc2-c2cc(N(c3ccc(-c4ccccc4)cc3)c3cc4ccccc4c4c3ccc3c5ccccc5[se]c34)ccc21 ; CC1(C)c2ccccc2-c2cc(N(c3ccccc3)c3cc4ccccc4c4c3ccc3c5ccccc5[se]c34)ccc21]

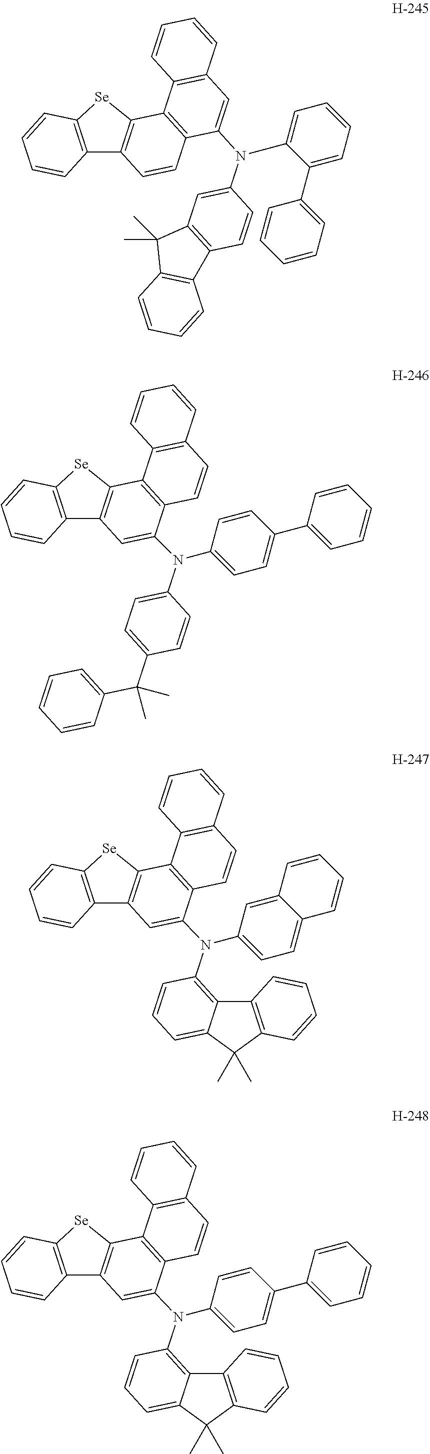 [SMILES: CC1(C)c2ccccc2-c2ccc(N(c3ccccc3-c3ccccc3)c3cc4ccccc4c4c3ccc3c5ccccc5[se]c34)cc21 ; CC(C)(c1ccccc1)c1ccc(N(c2ccc(-c3ccccc3)cc2)c2cc3c4ccccc4[se]c3c3c2ccc2ccccc23)cc1 ; CC1(C)c2ccccc2-c2c(N(c3ccc4ccccc4c3)c3cc4c5ccccc5[se]c4c4c3ccc3ccccc34)cccc21 ; CC1(C)c2ccccc2-c2c(N(c3ccc(-c4ccccc4)cc3)c3cc4c5ccccc5[se]c4c4c3ccc3ccccc34)cccc21]

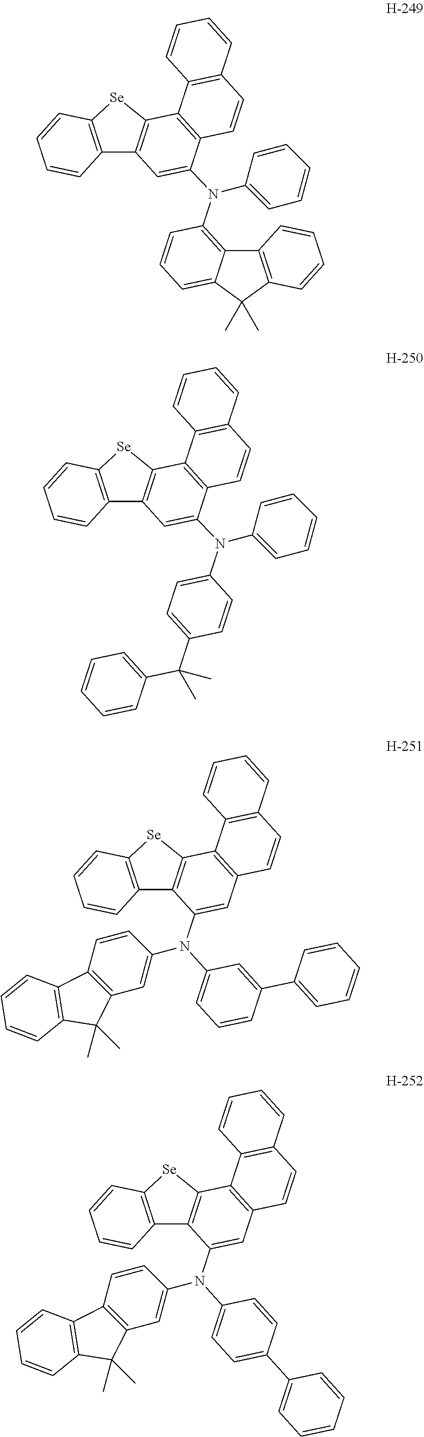

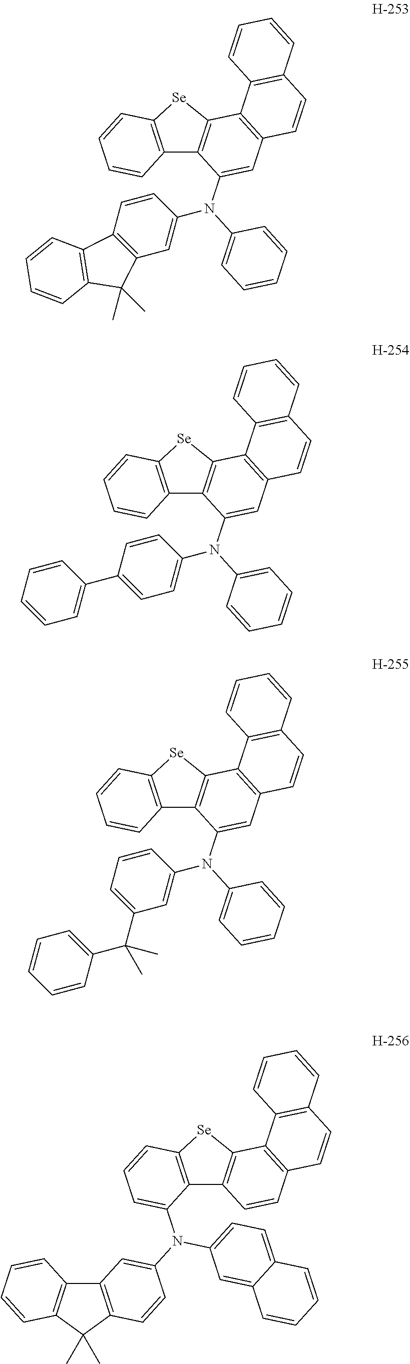 [SMILES: CC1(C)c2ccccc2-c2ccc(N(c3ccccc3)c3cc4ccc5ccccc5c4c4[se]c5ccccc5c34)cc21 ; c1ccc(-c2ccc(N(c3ccccc3)c3cc4ccc5ccccc5c4c4[se]c5ccccc5c34)cc2)cc1 ; CC(C)(c1ccccc1)c1cccc(N(c2ccccc2)c2cc3ccc4ccccc4c3c3[se]c4ccccc4c23)c1 ; CC1(C)c2ccccc2-c2cc(N(c3ccc4ccccc4c3)c3cccc4[se]c5c(ccc6ccc7ccccc7c65)c34)ccc21]

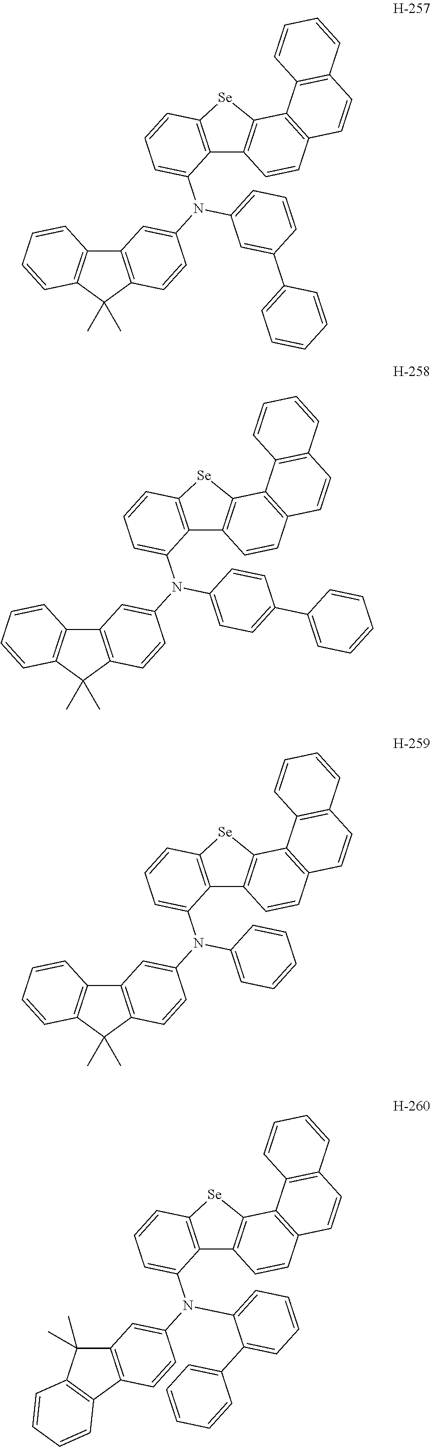 [SMILES: CC1(C)c2ccccc2-c2cc(N(c3cccc(-c4ccccc4)c3)c3cccc4[se]c5c(ccc6ccc7ccccc7c65)c34)ccc21 ; CC1(C)c2ccccc2-c2cc(N(c3ccc(-c4ccccc4)cc3)c3cccc4[se]c5c(ccc6ccc7ccccc7c65)c34)ccc21 ; CC1(C)c2ccccc2-c2cc(N(c3ccccc3)c3cccc4[se]c5c(ccc6ccc7ccccc7c65)c34)ccc21 ; CC1(C)c2ccccc2-c2ccc(N(c3ccccc3-c3ccccc3)c3cccc4[se]c5c(ccc6ccc7ccccc7c65)c34)cc21]

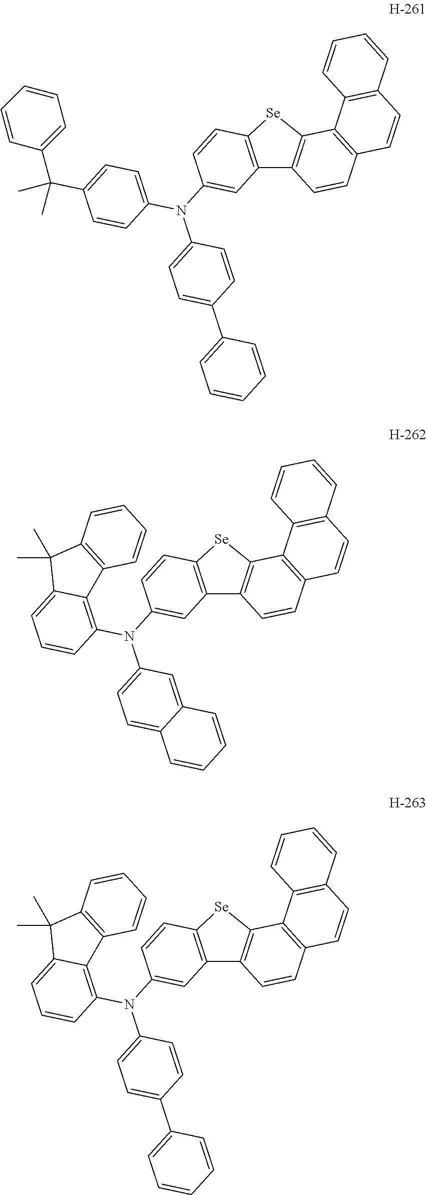 [SMILES: CC(C)(c1ccccc1)c1ccc(N(c2ccc(-c3ccccc3)cc2)c2ccc3[se]c4c(ccc5ccc6ccccc6c54)c3c2)cc1 ; CC1(C)c2ccccc2-c2c(N(c3ccc4ccccc4c3)c3ccc4[se]c5c(ccc6ccc7ccccc7c65)c4c3)cccc21 ; CC1(C)c2ccccc2-c2c(N(c3ccc(-c4ccccc4)cc3)c3ccc4[se]c5c(ccc6ccc7ccccc7c65)c4c3)cccc21]

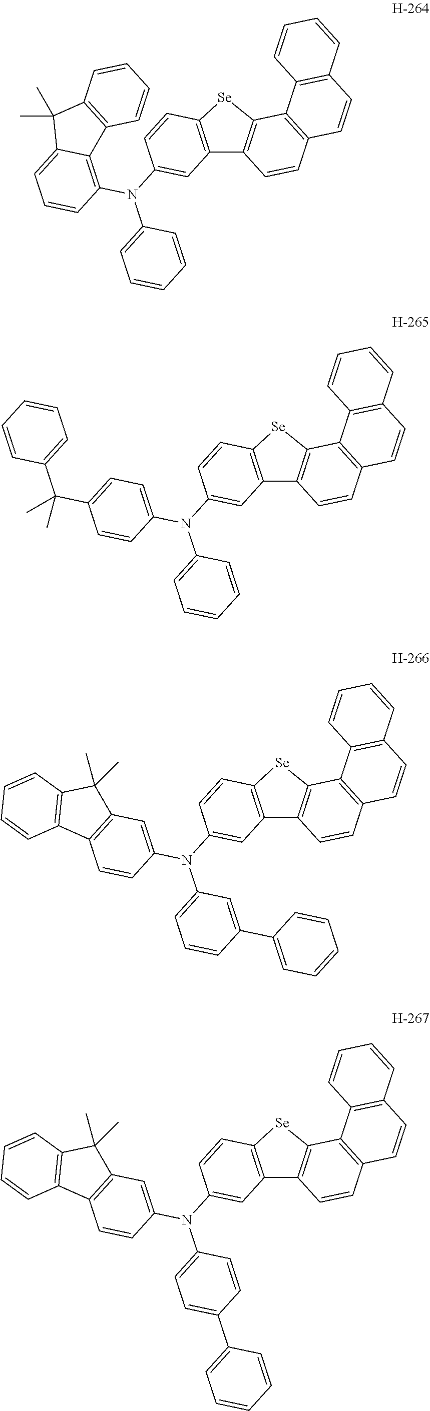 [SMILES: CC1(C)c2ccccc2-c2c(N(c3ccccc3)c3ccc4[se]c5c(ccc6ccc7ccccc7c65)c4c3)cccc21 ; CC(C)(c1ccccc1)c1ccc(N(c2ccccc2)c2ccc3[se]c4c(ccc5ccc6ccccc6c54)c3c2)cc1 ; CC1(C)c2ccccc2-c2ccc(N(c3cccc(-c4ccccc4)c3)c3ccc4[se]c5c(ccc6ccc7ccccc7c65)c4c3)cc21 ; CC1(C)c2ccccc2-c2ccc(N(c3ccc(-c4ccccc4)cc3)c3ccc4[se]c5c(ccc6ccc7ccccc7c65)c4c3)cc21]

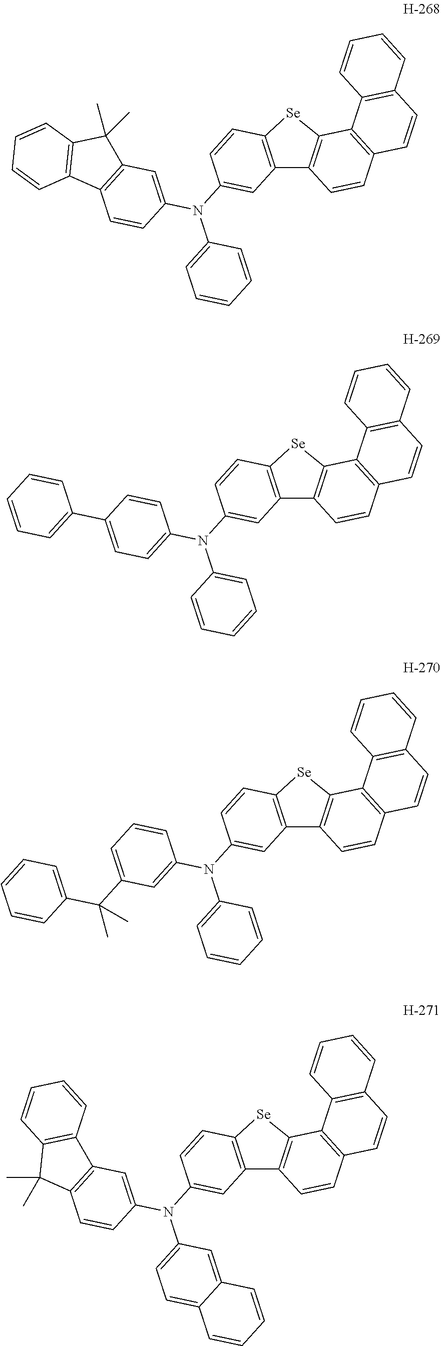 [SMILES: CC1(C)c2ccccc2-c2ccc(N(c3ccccc3)c3ccc4[se]c5c(ccc6ccc7ccccc7c65)c4c3)cc21 ; c1ccc(-c2ccc(N(c3ccccc3)c3ccc4[se]c5c(ccc6ccc7ccccc7c65)c4c3)cc2)cc1 ; CC(C)(c1ccccc1)c1cccc(N(c2ccccc2)c2ccc3[se]c4c(ccc5ccc6ccccc6c54)c3c2)c1 ; CC1(C)c2ccccc2-c2cc(N(c3ccc4ccccc4c3)c3ccc4[se]c5c(ccc6ccc7ccccc7c65)c4c3)ccc21]

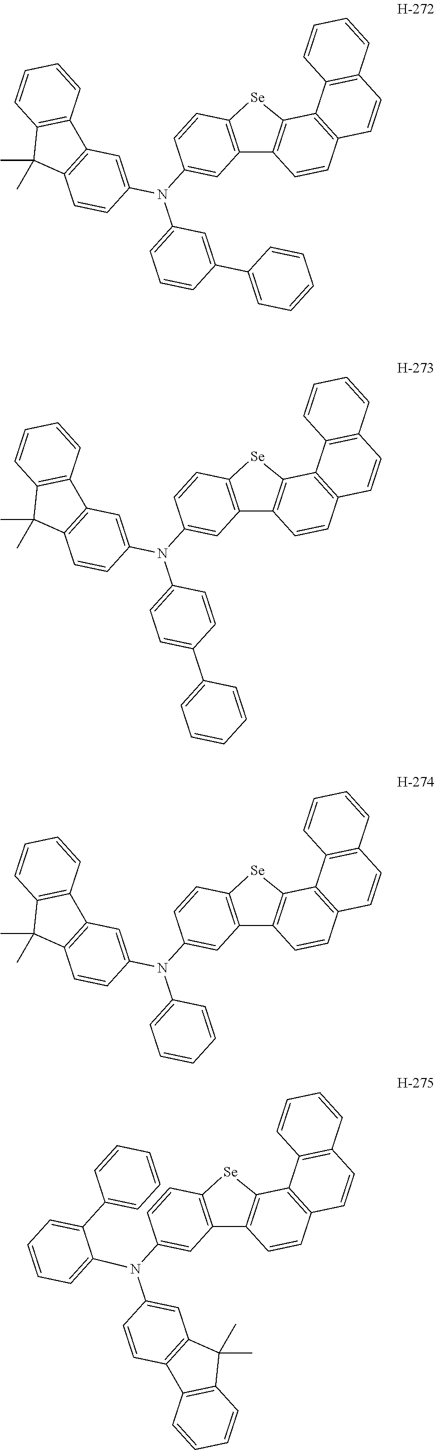 [SMILES: CC1(C)c2ccccc2-c2cc(N(c3cccc(-c4ccccc4)c3)c3ccc4[se]c5c(ccc6ccc7ccccc7c65)c4c3)ccc21 ; CC1(C)c2ccccc2-c2cc(N(c3ccc(-c4ccccc4)cc3)c3ccc4[se]c5c(ccc6ccc7ccccc7c65)c4c3)ccc21 ; CC1(C)c2ccccc2-c2cc(N(c3ccccc3)c3ccc4[se]c5c(ccc6ccc7ccccc7c65)c4c3)ccc21 ; CC1(C)c2ccccc2-c2ccc(N(c3ccc4[se]c5c(ccc6ccc7ccccc7c65)c4c3)c3ccccc3-c3ccccc3)cc21]

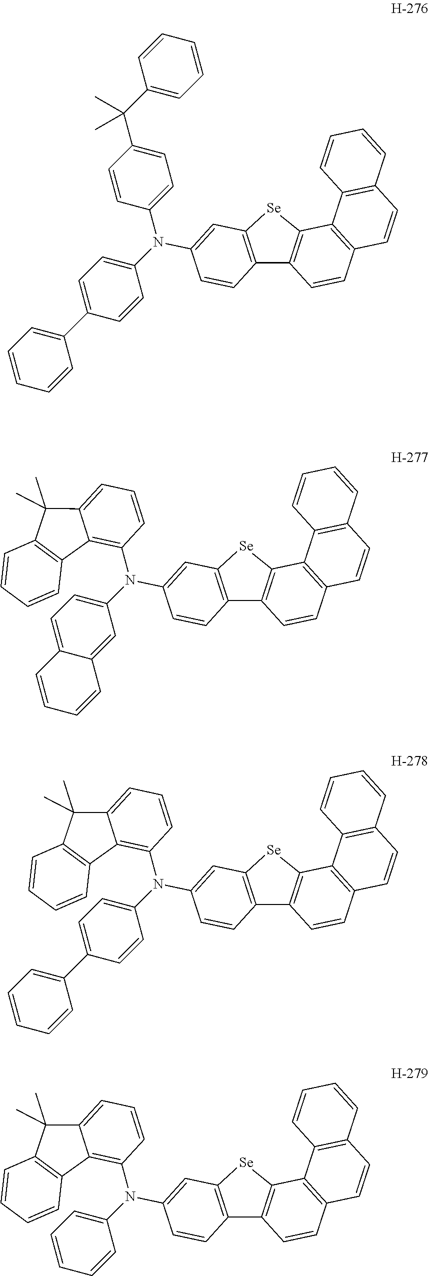 [SMILES: CC(C)(c1ccccc1)c1ccc(N(c2ccc(-c3ccccc3)cc2)c2ccc3c(c2)[se]c2c3ccc3ccc4ccccc4c32)cc1 ; CC1(C)c2ccccc2-c2c(N(c3ccc4ccccc4c3)c3ccc4c(c3)[se]c3c4ccc4ccc5ccccc5c43)cccc21 ; CC1(C)c2ccccc2-c2c(N(c3ccc(-c4ccccc4)cc3)c3ccc4c(c3)[se]c3c4ccc4ccc5ccccc5c43)cccc21 ; CC1(C)c2ccccc2-c2c(N(c3ccccc3)c3ccc4c(c3)[se]c3c4ccc4ccc5ccccc5c43)cccc21]

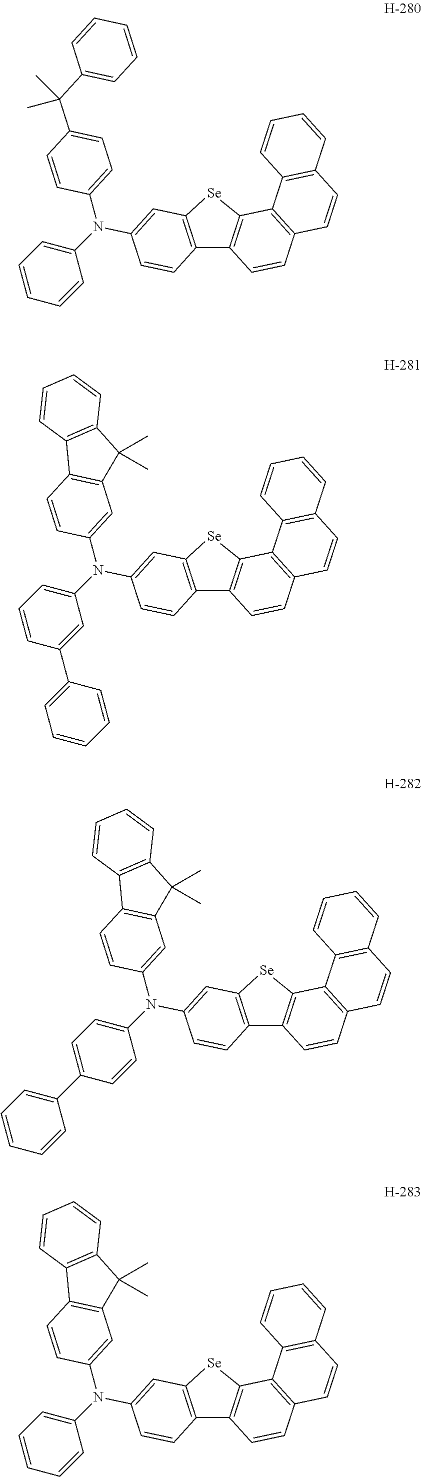 [SMILES: CC(C)(c1ccccc1)c1ccc(N(c2ccccc2)c2ccc3c(c2)[se]c2c3ccc3ccc4ccccc4c32)cc1 ; CC1(C)c2ccccc2-c2ccc(N(c3cccc(-c4ccccc4)c3)c3ccc4c(c3)[se]c3c4ccc4ccc5ccccc5c43)cc21 ; CC1(C)c2ccccc2-c2ccc(N(c3ccc(-c4ccccc4)cc3)c3ccc4c(c3)[se]c3c4ccc4ccc5ccccc5c43)cc21 ; CC1(C)c2ccccc2-c2ccc(N(c3ccccc3)c3ccc4c(c3)[se]c3c4ccc4ccc5ccccc5c43)cc21]

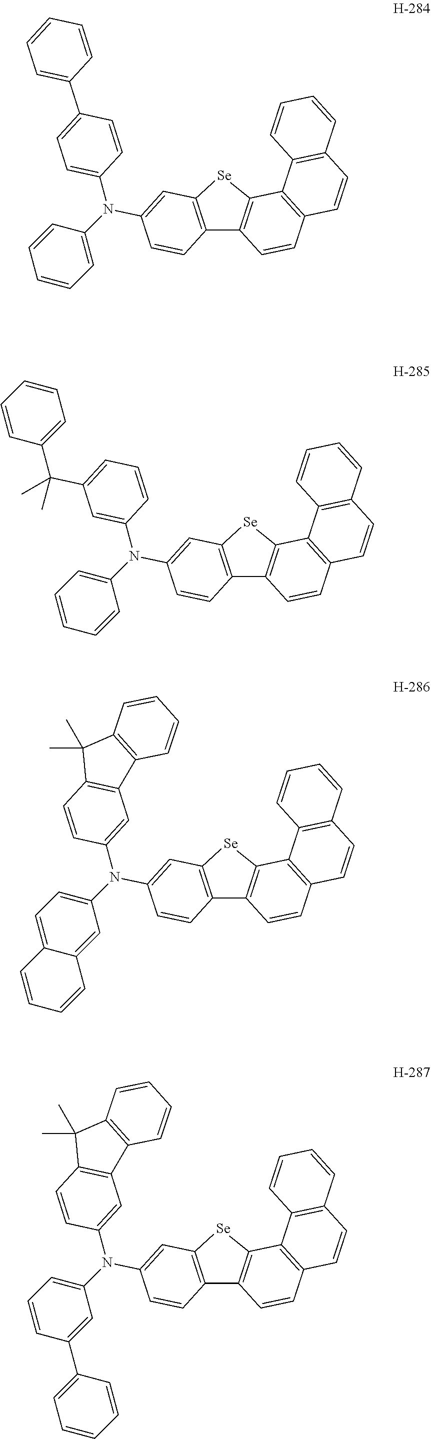 [SMILES: c1ccc(-c2ccc(N(c3ccccc3)c3ccc4c(c3)[se]c3c4ccc4ccc5ccccc5c43)cc2)cc1 ; CC(C)(c1ccccc1)c1cccc(N(c2ccccc2)c2ccc3c(c2)[se]c2c3ccc3ccc4ccccc4c32)c1 ; CC1(C)c2ccccc2-c2cc(N(c3ccc4ccccc4c3)c3ccc4c(c3)[se]c3c4ccc4ccc5ccccc5c43)ccc21 ; CC1(C)c2ccccc2-c2cc(N(c3cccc(-c4ccccc4)c3)c3ccc4c(c3)[se]c3c4ccc4ccc5ccccc5c43)ccc21]

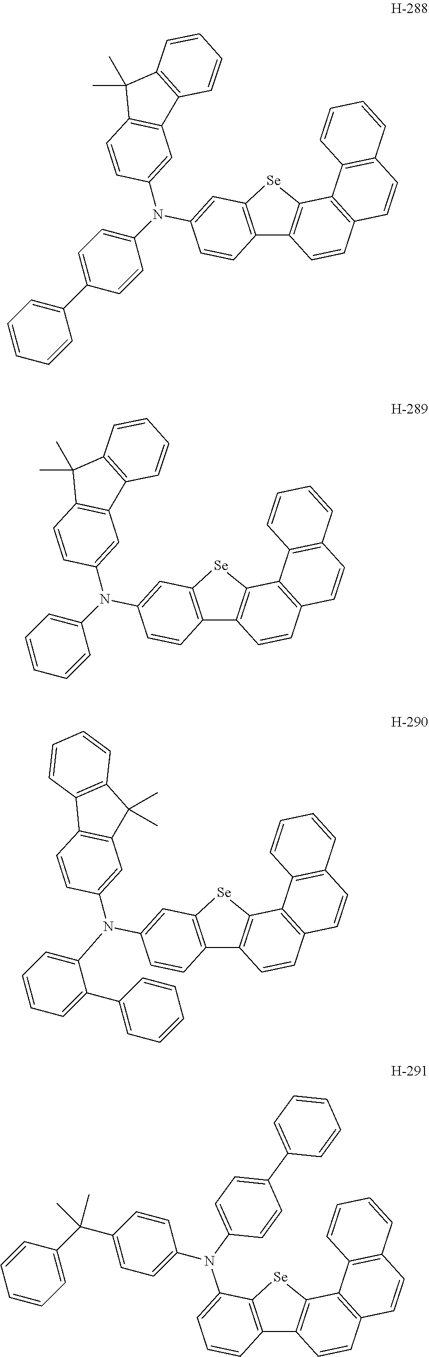 [SMILES: CC1(C)c2ccccc2-c2cc(N(c3ccc(-c4ccccc4)cc3)c3ccc4c(c3)[se]c3c4ccc4ccc5ccccc5c43)ccc21 ; CC1(C)c2ccccc2-c2cc(N(c3ccccc3)c3ccc4c(c3)[se]c3c4ccc4ccc5ccccc5c43)ccc21 ; CC1(C)c2ccccc2-c2ccc(N(c3ccc4c(c3)[se]c3c4ccc4ccc5ccccc5c43)c3ccccc3-c3ccccc3)cc21 ; CC(C)(c1ccccc1)c1ccc(N(c2ccc(-c3ccccc3)cc2)c2cccc3c2[se]c2c3ccc3ccc4ccccc4c32)cc1]

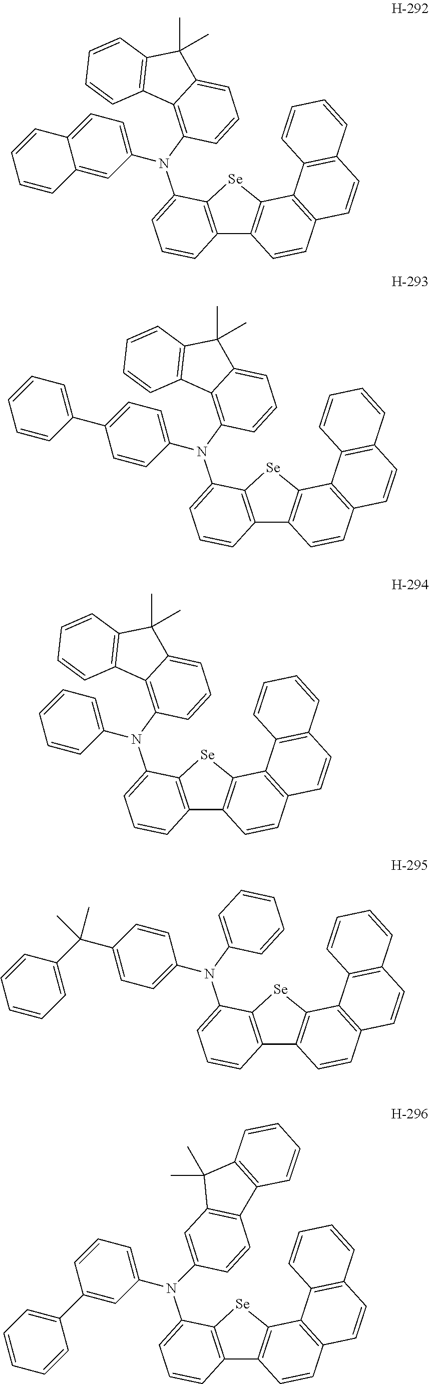 [SMILES: CC1(C)c2ccccc2-c2c(N(c3ccc4ccccc4c3)c3cccc4c3[se]c3c4ccc4ccc5ccccc5c43)cccc21 ; CC1(C)c2ccccc2-c2c(N(c3ccc(-c4ccccc4)cc3)c3cccc4c3[se]c3c4ccc4ccc5ccccc5c43)cccc21 ; CC1(C)c2ccccc2-c2c(N(c3ccccc3)c3cccc4c3[se]c3c4ccc4ccc5ccccc5c43)cccc21 ; CC(C)(c1ccccc1)c1ccc(N(c2ccccc2)c2cccc3c2[se]c2c3ccc3ccc4ccccc4c32)cc1 ; CC1(C)c2ccccc2-c2ccc(N(c3cccc(-c4ccccc4)c3)c3cccc4c3[se]c3c4ccc4ccc5ccccc5c43)cc21]

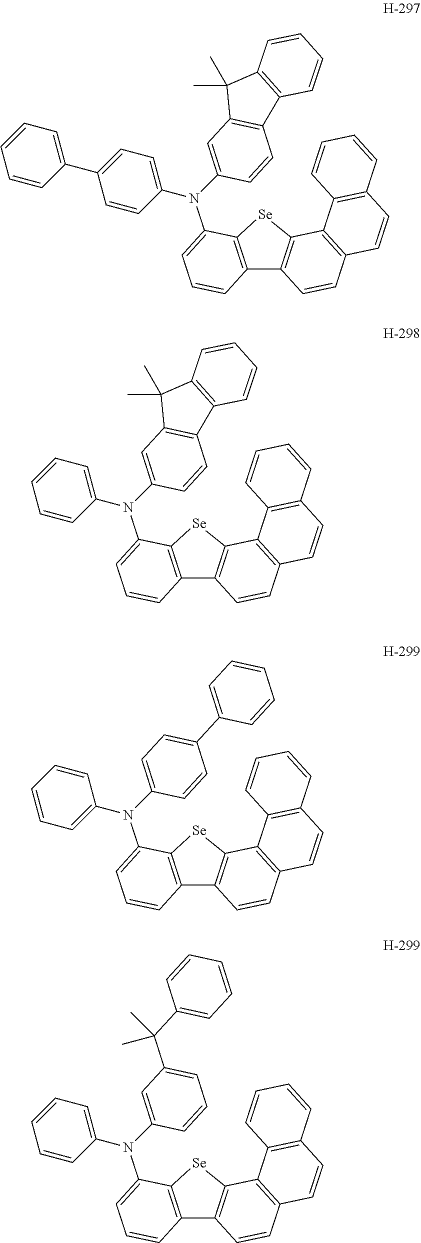 [SMILES: CC1(C)c2ccccc2-c2ccc(N(c3ccc(-c4ccccc4)cc3)c3cccc4c3[se]c3c4ccc4ccc5ccccc5c43)cc21 ; CC1(C)c2ccccc2-c2ccc(N(c3ccccc3)c3cccc4c3[se]c3c4ccc4ccc5ccccc5c43)cc21 ; c1ccc(-c2ccc(N(c3ccccc3)c3cccc4c3[se]c3c4ccc4ccc5ccccc5c43)cc2)cc1 ; CC(C)(c1ccccc1)c1cccc(N(c2ccccc2)c2cccc3c2[se]c2c3ccc3ccc4ccccc4c32)c1]

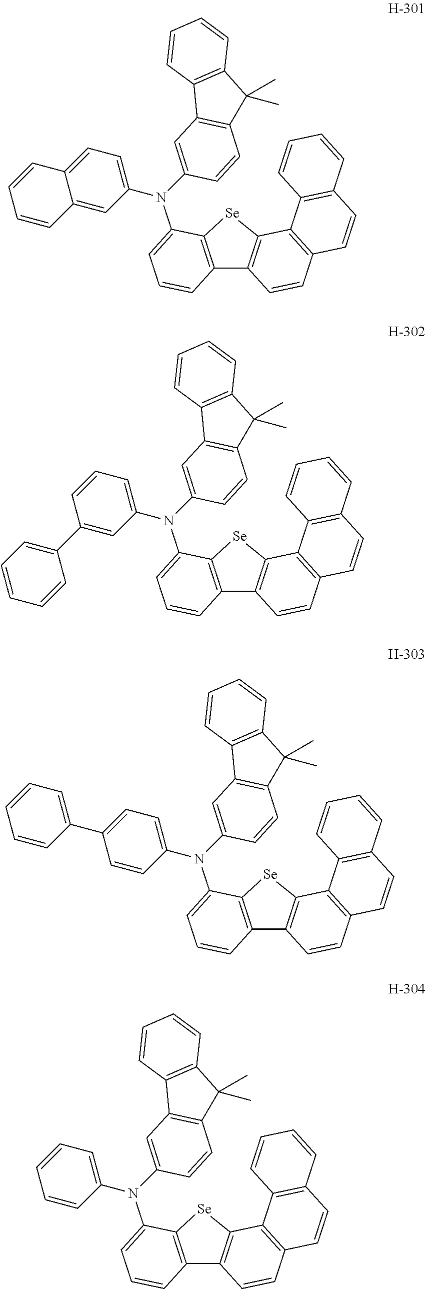 [SMILES: CC1(C)c2ccccc2-c2cc(N(c3ccc4ccccc4c3)c3cccc4c3[se]c3c4ccc4ccc5ccccc5c43)ccc21 ; CC1(C)c2ccccc2-c2cc(N(c3cccc(-c4ccccc4)c3)c3cccc4c3[se]c3c4ccc4ccc5ccccc5c43)ccc21 ; CC1(C)c2ccccc2-c2cc(N(c3ccc(-c4ccccc4)cc3)c3cccc4c3[se]c3c4ccc4ccc5ccccc5c43)ccc21 ; CC1(C)c2ccccc2-c2cc(N(c3ccccc3)c3cccc4c3[se]c3c4ccc4ccc5ccccc5c43)ccc21]

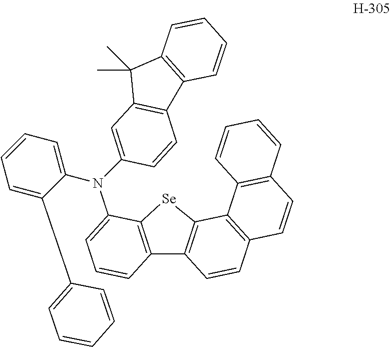 [SMILES: CC1(C)c2ccccc2-c2ccc(N(c3ccccc3-c3ccccc3)c3cccc4c3[se]c3c4ccc4ccc5ccccc5c43)cc21]

The compound represented by formula 2 may be at least one selected from the following compounds, but is not limited thereto.

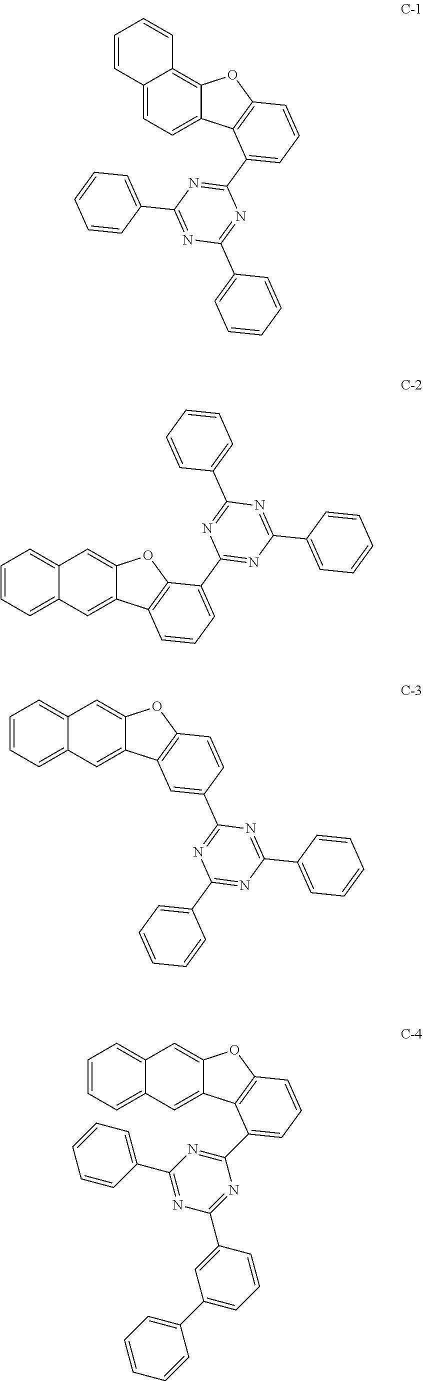 [SMILES: c1ccc(-c2nc(-c3ccccc3)nc(-c3cccc4oc5c6ccccc6ccc5c34)n2)cc1 ; c1ccc(-c2nc(-c3ccccc3)nc(-c3cccc4c3oc3cc5ccccc5cc34)n2)cc1 ; c1ccc(-c2nc(-c3ccccc3)nc(-c3ccc4oc5cc6ccccc6cc5c4c3)n2)cc1 ; c1ccc(-c2cccc(-c3nc(-c4ccccc4)nc(-c4cccc5oc6cc7ccccc7cc6c45)n3)c2)cc1]

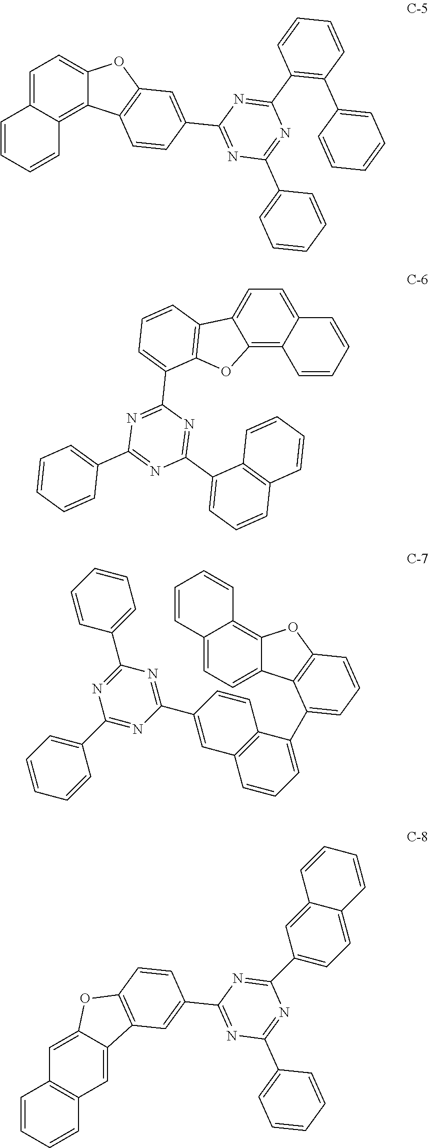

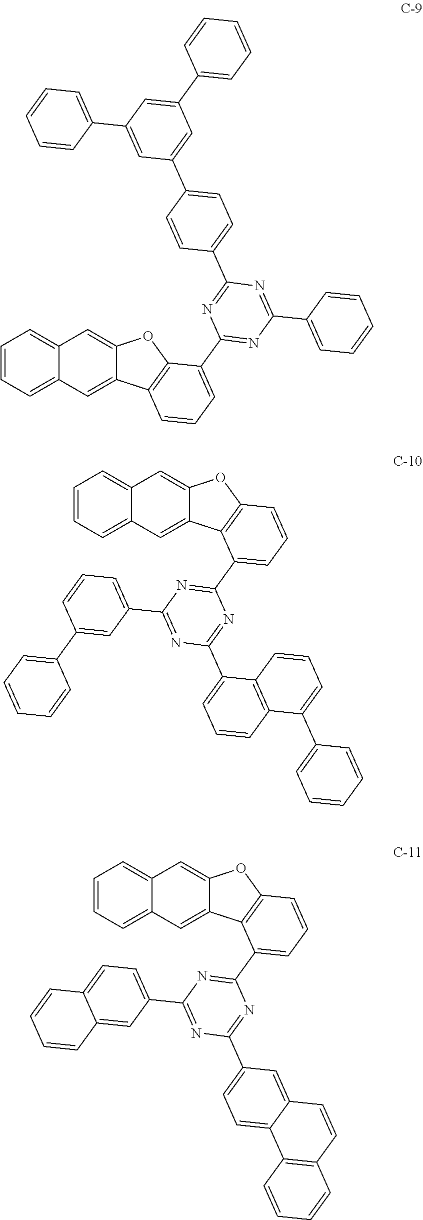 [SMILES: c1ccc(-c2cc(-c3ccccc3)cc(-c3ccc(-c4nc(-c5ccccc5)nc(-c5cccc6c5oc5cc7ccccc7cc56)n4)cc3)c2)cc1 ; c1ccc(-c2cccc(-c3nc(-c4cccc5c(-c6ccccc6)cccc45)nc(-c4cccc5oc6cc7ccccc7cc6c45)n3)c2)cc1 ; c1ccc2cc(-c3nc(-c4ccc5c(ccc6ccccc65)c4)nc(-c4cccc5oc6cc7ccccc7cc6c45)n3)ccc2c1]

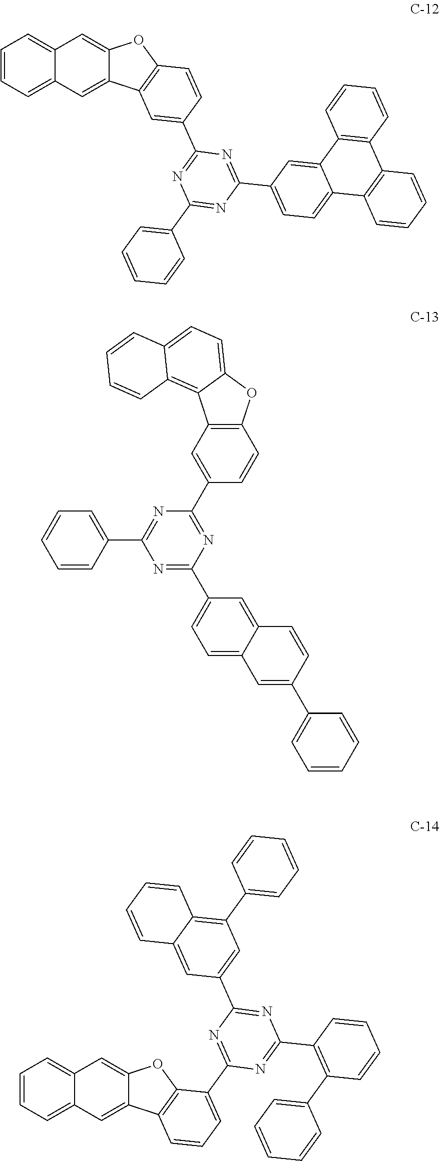 [SMILES: c1ccc(-c2nc(-c3ccc4oc5cc6ccccc6cc5c4c3)nc(-c3ccc4c5ccccc5c5ccccc5c4c3)n2)cc1 ; c1ccc(-c2ccc3cc(-c4nc(-c5ccccc5)nc(-c5ccc6oc7ccc8ccccc8c7c6c5)n4)ccc3c2)cc1 ; c1ccc(-c2ccccc2-c2nc(-c3cc(-c4ccccc4)c4ccccc4c3)nc(-c3cccc4c3oc3cc5ccccc5cc34)n2)cc1]

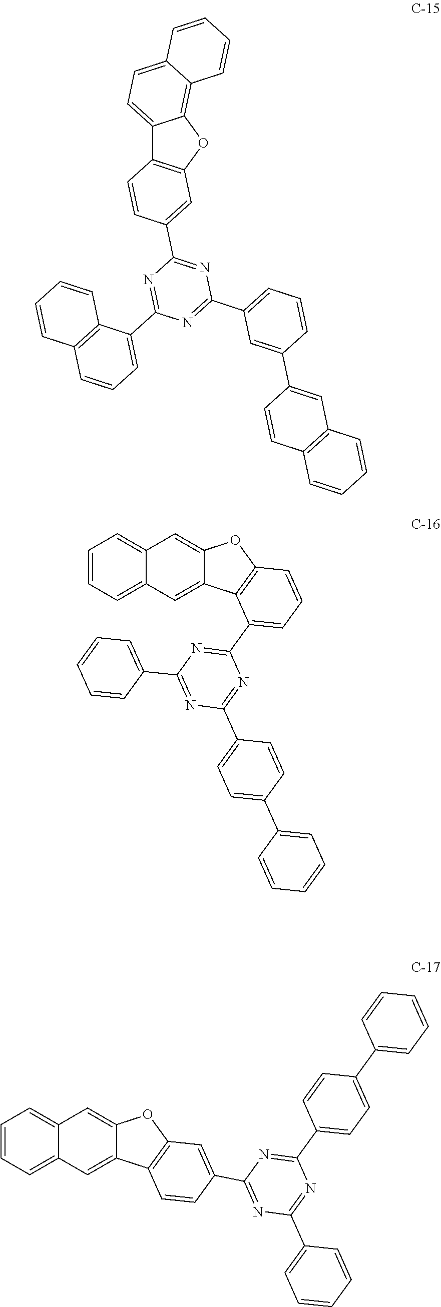 [SMILES: c1cc(-c2ccc3ccccc3c2)cc(-c2nc(-c3ccc4c(c3)oc3c5ccccc5ccc43)nc(-c3cccc4ccccc34)n2)c1 ; c1ccc(-c2ccc(-c3nc(-c4ccccc4)nc(-c4cccc5oc6cc7ccccc7cc6c45)n3)cc2)cc1 ; c1ccc(-c2ccc(-c3nc(-c4ccccc4)nc(-c4ccc5c(c4)oc4cc6ccccc6cc45)n3)cc2)cc1]

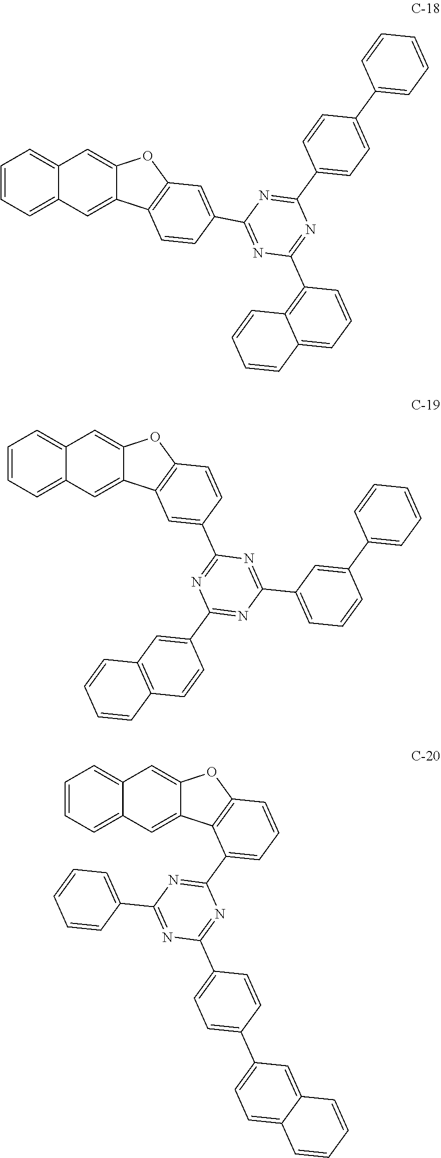 [SMILES: c1ccc(-c2ccc(-c3nc(-c4ccc5c(c4)oc4cc6ccccc6cc45)nc(-c4cccc5ccccc45)n3)cc2)cc1 ; c1ccc(-c2cccc(-c3nc(-c4ccc5ccccc5c4)nc(-c4ccc5oc6cc7ccccc7cc6c5c4)n3)c2)cc1 ; c1ccc(-c2nc(-c3ccc(-c4ccc5ccccc5c4)cc3)nc(-c3cccc4oc5cc6ccccc6cc5c34)n2)cc1]

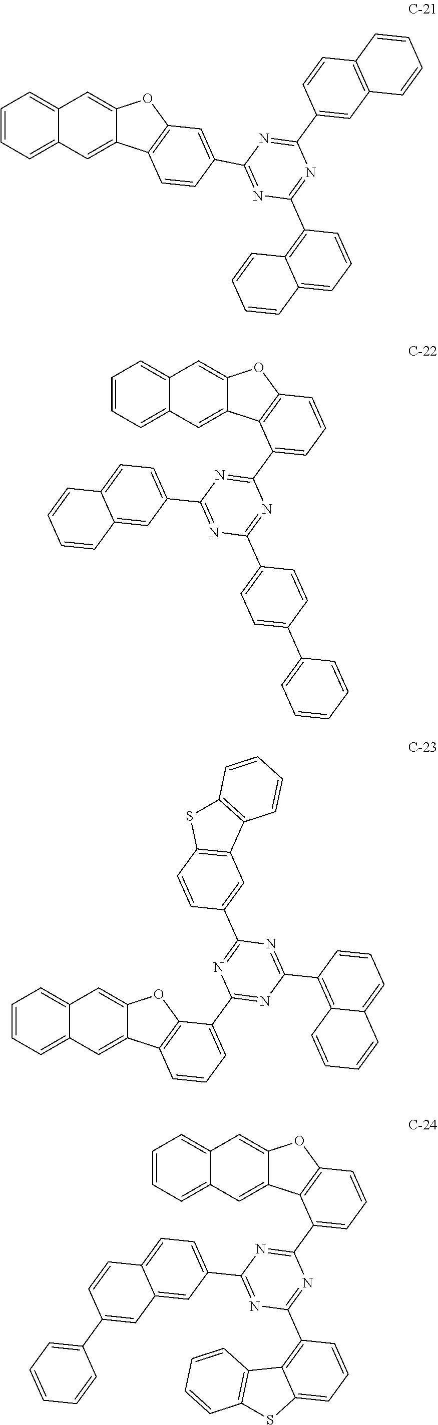 [SMILES: c1ccc2cc(-c3nc(-c4ccc5c(c4)oc4cc6ccccc6cc45)nc(-c4cccc5ccccc45)n3)ccc2c1 ; c1ccc(-c2ccc(-c3nc(-c4ccc5ccccc5c4)nc(-c4cccc5oc6cc7ccccc7cc6c45)n3)cc2)cc1 ; c1ccc2cc3c(cc2c1)oc1c(-c2nc(-c4ccc5sc6ccccc6c5c4)nc(-c4cccc5ccccc45)n2)cccc13 ; c1ccc(-c2ccc3ccc(-c4nc(-c5cccc6oc7cc8ccccc8cc7c56)nc(-c5cccc6sc7ccccc7c56)n4)cc3c2)cc1]

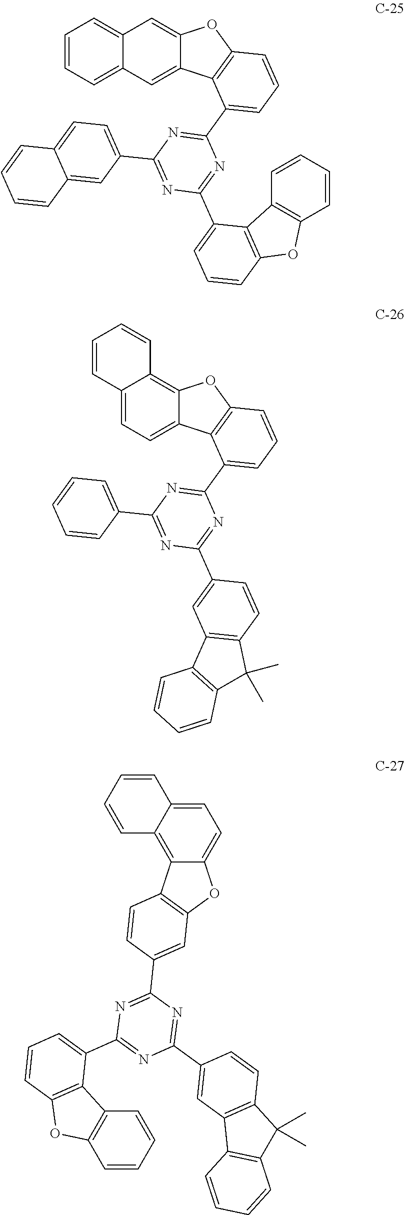 [SMILES: c1ccc2cc(-c3nc(-c4cccc5oc6ccccc6c45)nc(-c4cccc5oc6cc7ccccc7cc6c45)n3)ccc2c1 ; CC1(C)c2ccccc2-c2cc(-c3nc(-c4ccccc4)nc(-c4cccc5oc6c7ccccc7ccc6c45)n3)ccc21 ; CC1(C)c2ccccc2-c2cc(-c3nc(-c4ccc5c(c4)oc4ccc6ccccc6c45)nc(-c4cccc5oc6ccccc6c45)n3)ccc21]

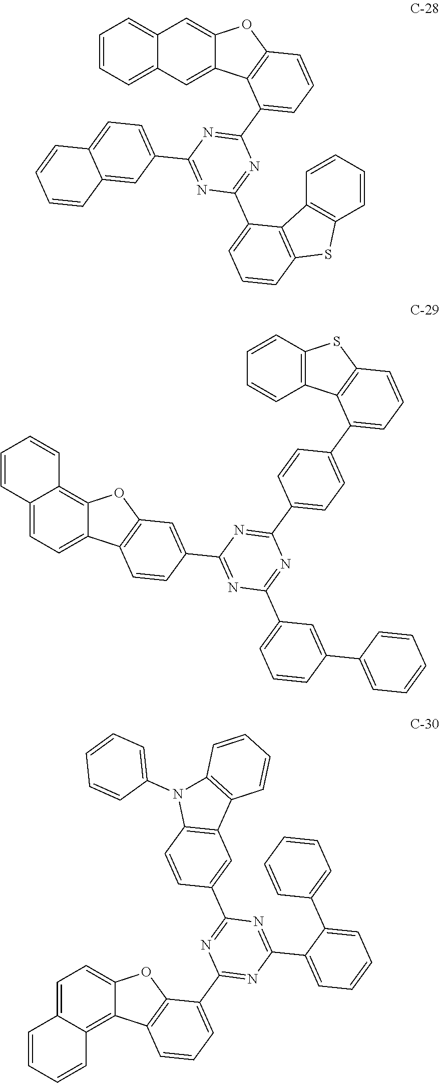 [SMILES: c1ccc2cc(-c3nc(-c4cccc5oc6cc7ccccc7cc6c45)nc(-c4cccc5sc6ccccc6c45)n3)ccc2c1 ; c1ccc(-c2cccc(-c3nc(-c4ccc(-c5cccc6sc7ccccc7c56)cc4)nc(-c4ccc5c(c4)oc4c6ccccc6ccc54)n3)c2)cc1 ; c1ccc(-c2ccccc2-c2nc(-c3ccc4c(c3)c3ccccc3n4-c3ccccc3)nc(-c3cccc4c3oc3ccc5ccccc5c34)n2)cc1]

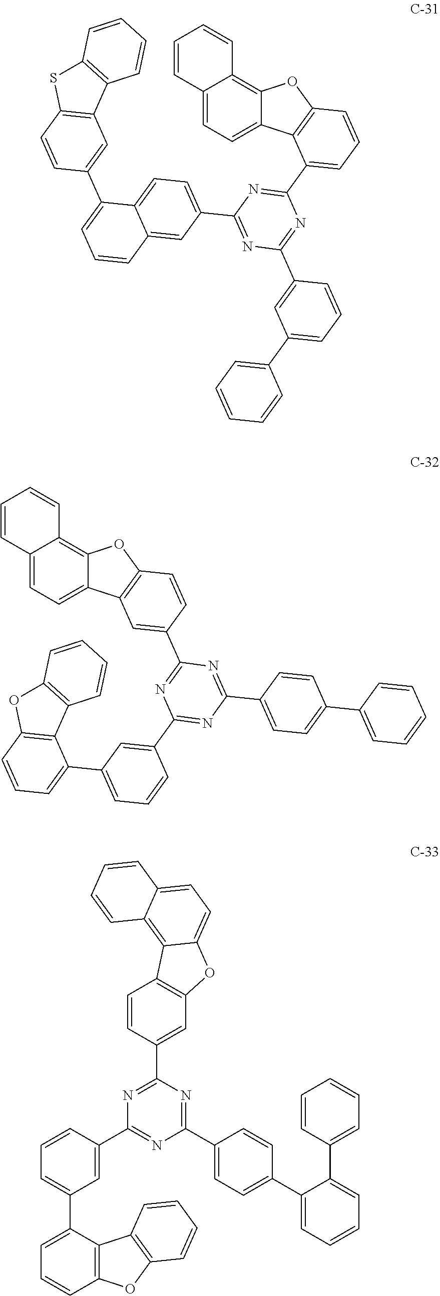 [SMILES: c1ccc(-c2cccc(-c3nc(-c4ccc5c(-c6ccc7sc8ccccc8c7c6)cccc5c4)nc(-c4cccc5oc6c7ccccc7ccc6c45)n3)c2)cc1 ; c1ccc(-c2ccc(-c3nc(-c4cccc(-c5cccc6oc7ccccc7c56)c4)nc(-c4ccc5oc6c7ccccc7ccc6c5c4)n3)cc2)cc1 ; c1ccc(-c2ccccc2-c2ccc(-c3nc(-c4cccc(-c5cccc6oc7ccccc7c56)c4)nc(-c4ccc5c(c4)oc4ccc6ccccc6c45)n3)cc2)cc1]

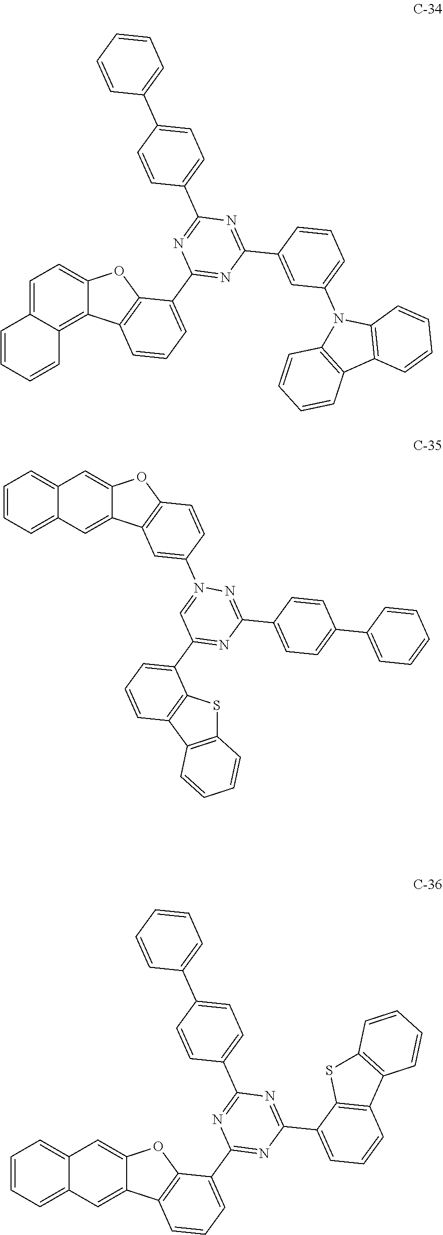

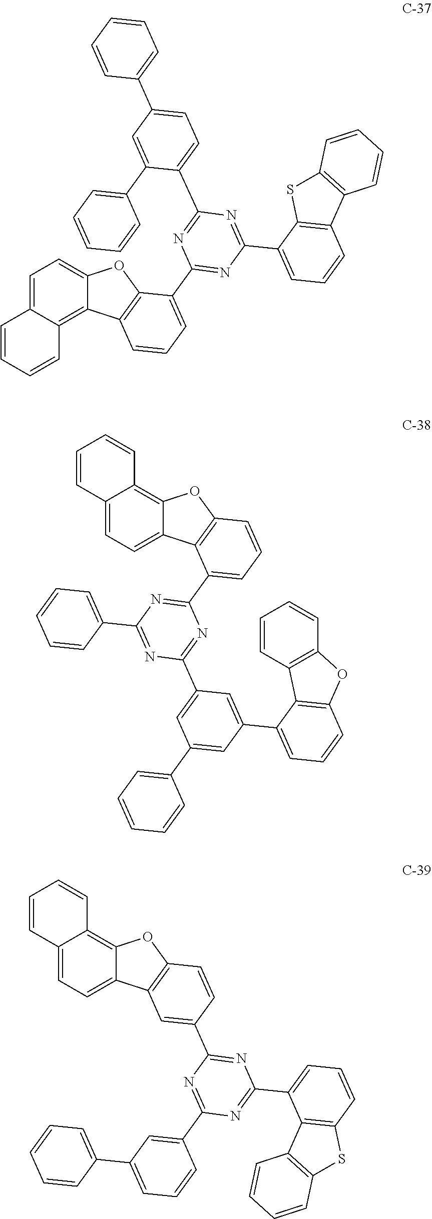 [SMILES: c1ccc(-c2ccc(-c3nc(-c4cccc5c4oc4ccc6ccccc6c45)nc(-c4cccc5c4sc4ccccc45)n3)c(-c3ccccc3)c2)cc1 ; c1ccc(-c2cc(-c3nc(-c4ccccc4)nc(-c4cccc5oc6c7ccccc7ccc6c45)n3)cc(-c3cccc4oc5ccccc5c34)c2)cc1 ; c1ccc(-c2cccc(-c3nc(-c4ccc5oc6c7ccccc7ccc6c5c4)nc(-c4cccc5sc6ccccc6c45)n3)c2)cc1]

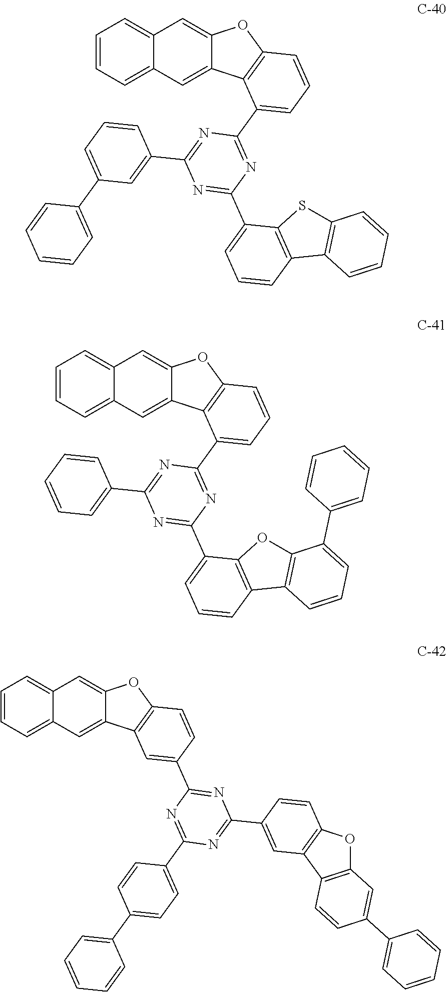 [SMILES: c1ccc(-c2cccc(-c3nc(-c4cccc5c4sc4ccccc45)nc(-c4cccc5oc6cc7ccccc7cc6c45)n3)c2)cc1 ; c1ccc(-c2nc(-c3cccc4c3oc3c(-c5ccccc5)cccc34)nc(-c3cccc4oc5cc6ccccc6cc5c34)n2)cc1 ; c1ccc(-c2ccc(-c3nc(-c4ccc5oc6cc(-c7ccccc7)ccc6c5c4)nc(-c4ccc5oc6cc7ccccc7cc6c5c4)n3)cc2)cc1]

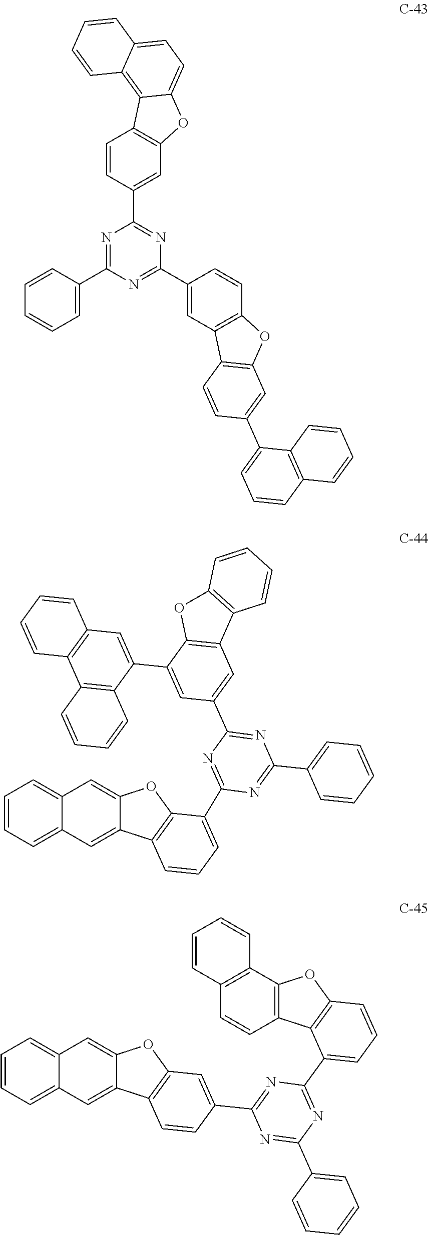 [SMILES: c1ccc(-c2nc(-c3ccc4c(c3)oc3ccc5ccccc5c34)nc(-c3ccc4oc5cc(-c6cccc7ccccc67)ccc5c4c3)n2)cc1 ; c1ccc(-c2nc(-c3cc(-c4cc5ccccc5c5ccccc45)c4oc5ccccc5c4c3)nc(-c3cccc4c3oc3cc5ccccc5cc34)n2)cc1 ; c1ccc(-c2nc(-c3ccc4c(c3)oc3cc5ccccc5cc34)nc(-c3cccc4oc5c6ccccc6ccc5c34)n2)cc1]

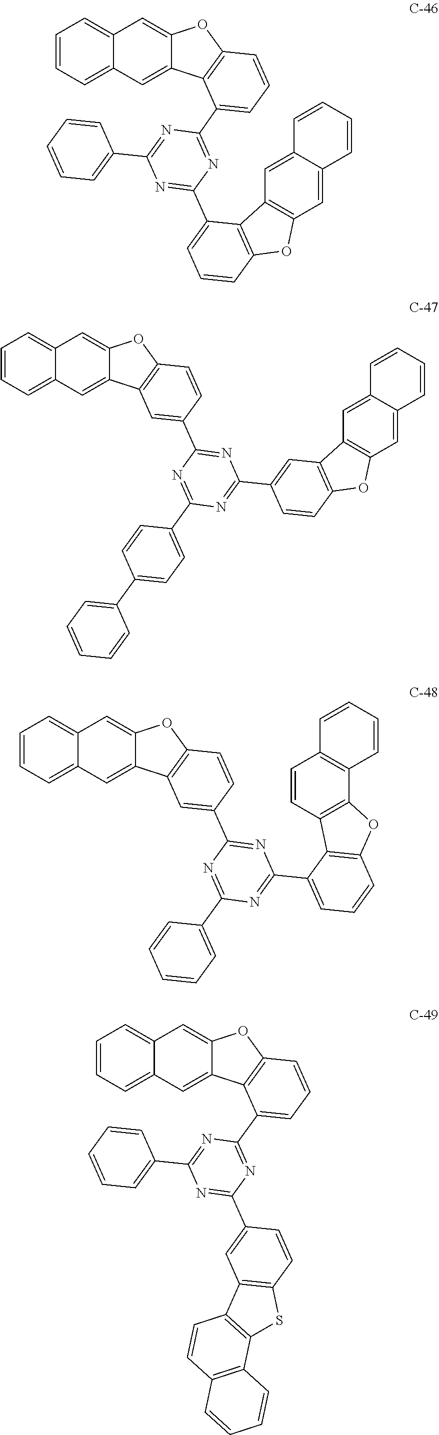

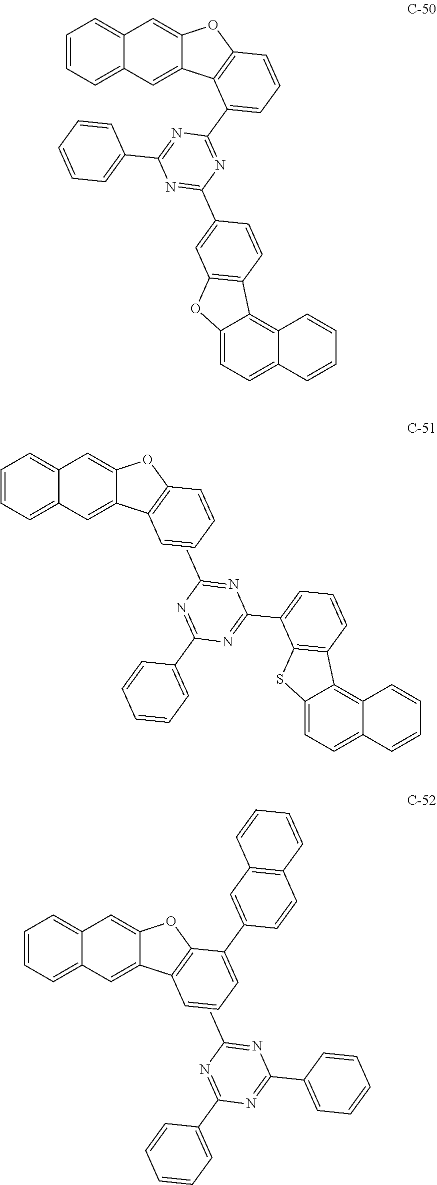

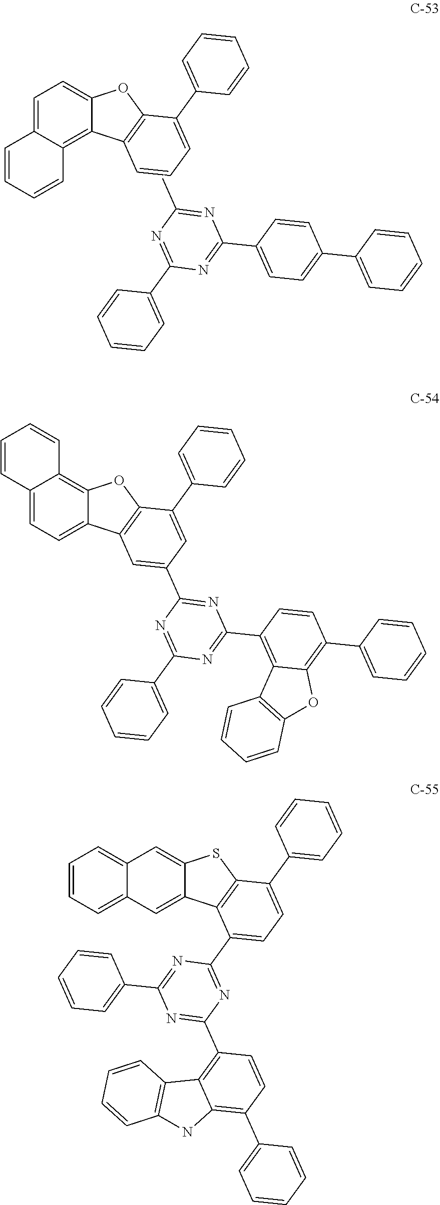

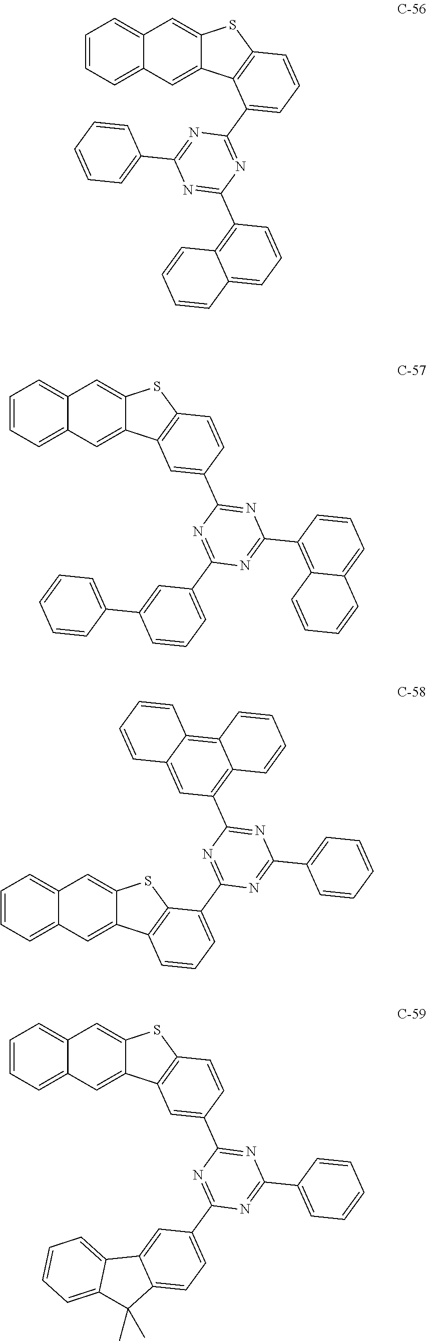 [SMILES: c1ccc(-c2nc(-c3cccc4ccccc34)nc(-c3cccc4sc5cc6ccccc6cc5c34)n2)cc1 ; c1ccc(-c2cccc(-c3nc(-c4ccc5sc6cc7ccccc7cc6c5c4)nc(-c4cccc5ccccc45)n3)c2)cc1 ; c1ccc(-c2nc(-c3cc4ccccc4c4ccccc34)nc(-c3cccc4c3sc3cc5ccccc5cc34)n2)cc1 ; CC1(C)c2ccccc2-c2cc(-c3nc(-c4ccccc4)nc(-c4ccc5sc6cc7ccccc7cc6c5c4)n3)ccc21]

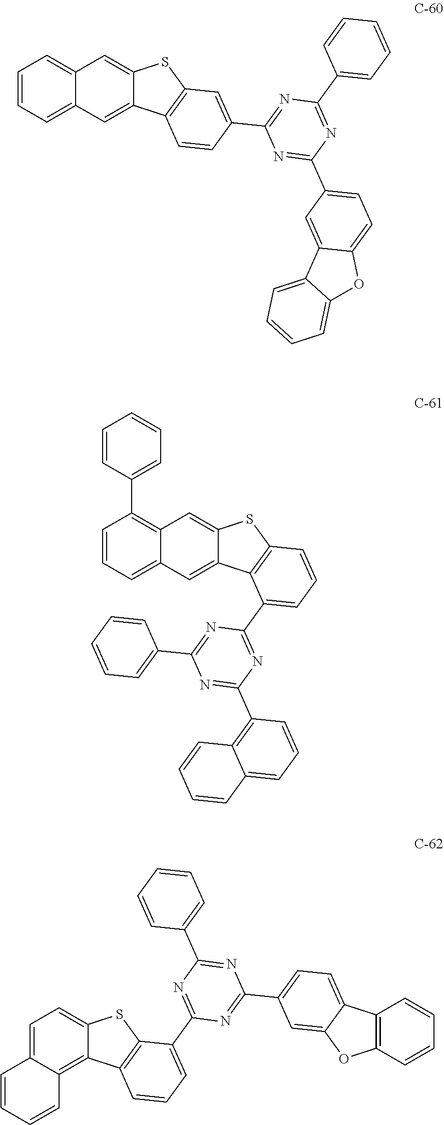 [SMILES: c1ccc(-c2nc(-c3ccc4c(c3)sc3cc5ccccc5cc34)nc(-c3ccc4oc5ccccc5c4c3)n2)cc1 ; c1ccc(-c2nc(-c3cccc4ccccc34)nc(-c3cccc4sc5cc6c(-c7ccccc7)cccc6cc5c34)n2)cc1 ; c1ccc(-c2nc(-c3ccc4c(c3)oc3ccccc34)nc(-c3cccc4c3sc3ccc5ccccc5c34)n2)cc1]

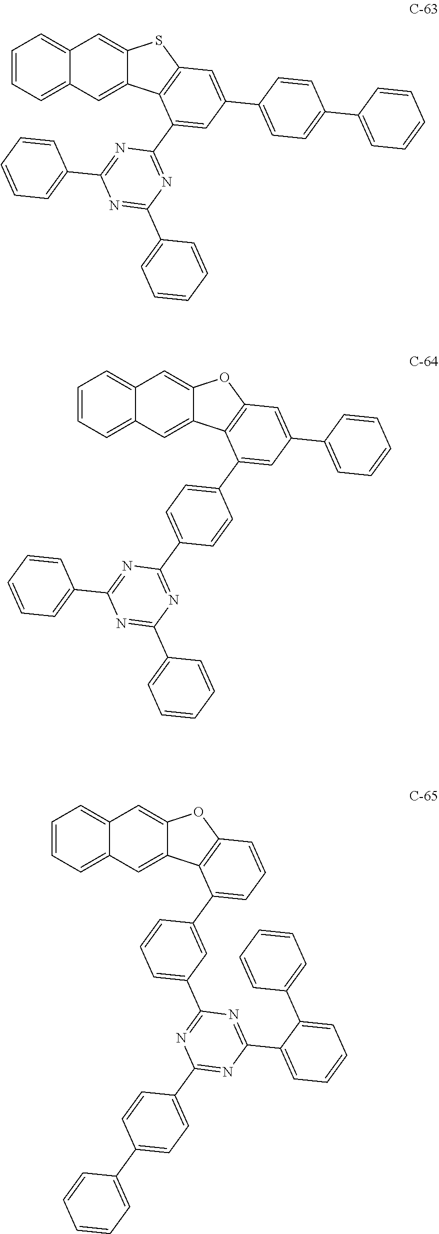 [SMILES: c1ccc(-c2ccc(-c3cc(-c4nc(-c5ccccc5)nc(-c5ccccc5)n4)c4c(c3)sc3cc5ccccc5cc34)cc2)cc1 ; c1ccc(-c2cc(-c3ccc(-c4nc(-c5ccccc5)nc(-c5ccccc5)n4)cc3)c3c(c2)oc2cc4ccccc4cc23)cc1 ; c1ccc(-c2ccc(-c3nc(-c4cccc(-c5cccc6oc7cc8ccccc8cc7c56)c4)nc(-c4ccccc4-c4ccccc4)n3)cc2)cc1]

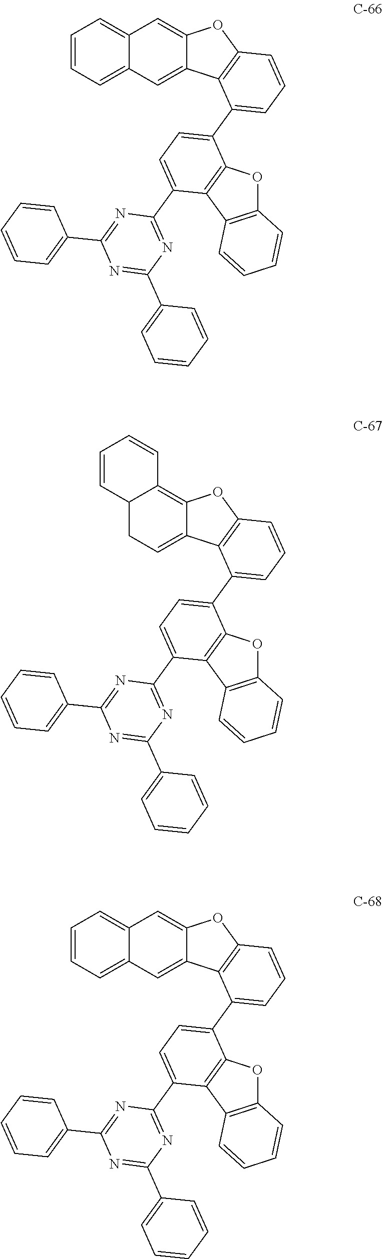 [SMILES: c1ccc(-c2nc(-c3ccccc3)nc(-c3ccc(-c4cccc5oc6cc7ccccc7cc6c45)c4oc5ccccc5c34)n2)cc1 ; C1=CC2=c3oc4cccc(-c5ccc(-c6nc(-c7ccccc7)nc(-c7ccccc7)n6)c6c5oc5ccccc56)c4c3=CCC2C=C1 ; c1ccc(-c2nc(-c3ccccc3)nc(-c3ccc(-c4cccc5oc6cc7ccccc7cc6c45)c4oc5ccccc5c34)n2)cc1]

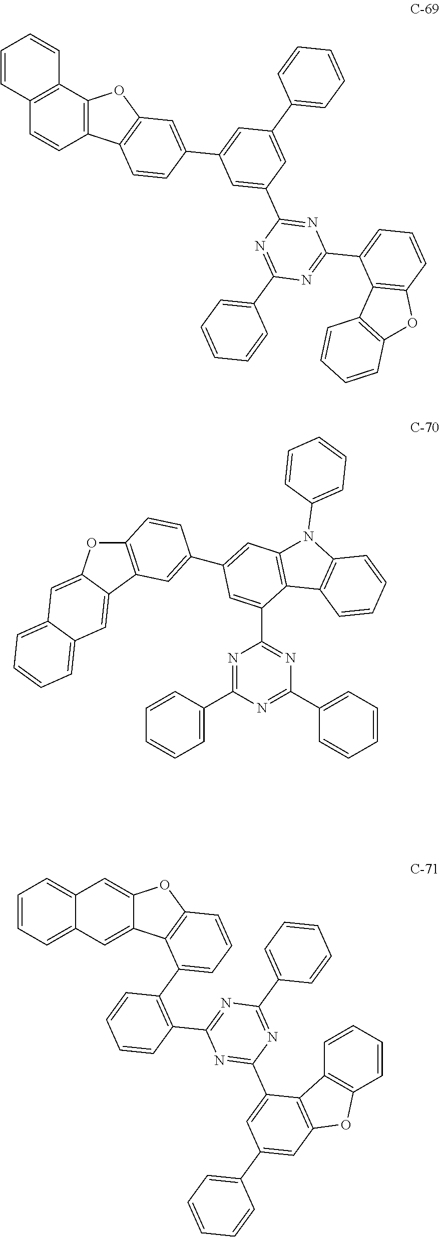 [SMILES: c1ccc(-c2cc(-c3ccc4c(c3)oc3c5ccccc5ccc43)cc(-c3nc(-c4ccccc4)nc(-c4cccc5oc6ccccc6c45)n3)c2)cc1 ; c1ccc(-c2nc(-c3ccccc3)nc(-c3cc(-c4ccc5oc6cc7ccccc7cc6c5c4)cc4c3c3ccccc3n4-c3ccccc3)n2)cc1 ; c1ccc(-c2cc(-c3nc(-c4ccccc4)nc(-c4ccccc4-c4cccc5oc6cc7ccccc7cc6c45)n3)c3c(c2)oc2ccccc23)cc1]

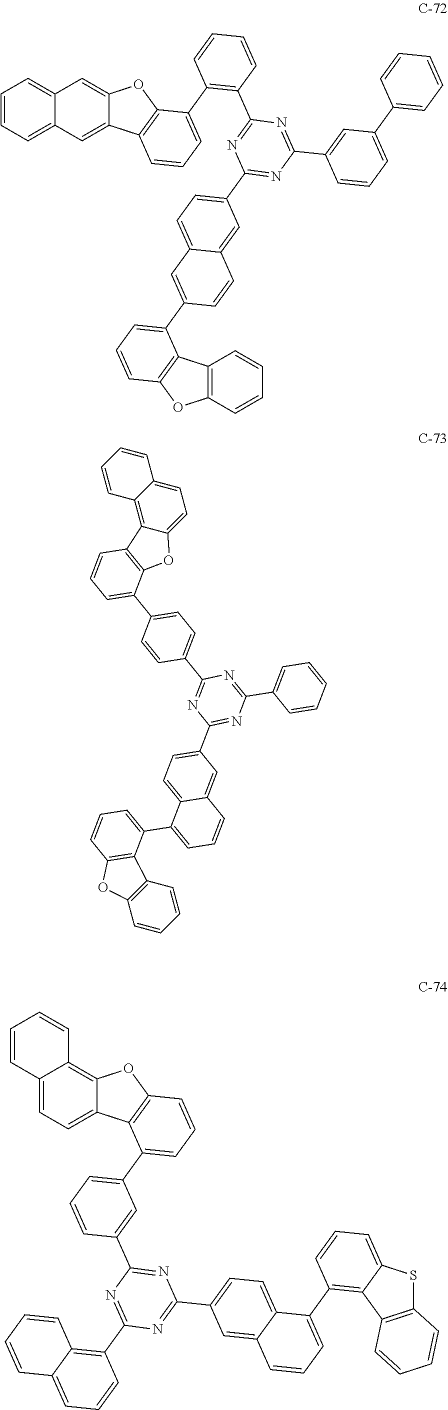 [SMILES: c1ccc(-c2cccc(-c3nc(-c4ccc5cc(-c6cccc7oc8ccccc8c67)ccc5c4)nc(-c4ccccc4-c4cccc5c4oc4cc6ccccc6cc45)n3)c2)cc1 ; c1ccc(-c2nc(-c3ccc(-c4cccc5c4oc4ccc6ccccc6c45)cc3)nc(-c3ccc4c(-c5cccc6oc7ccccc7c56)cccc4c3)n2)cc1 ; c1cc(-c2nc(-c3ccc4c(-c5cccc6sc7ccccc7c56)cccc4c3)nc(-c3cccc4ccccc34)n2)cc(-c2cccc3oc4c5ccccc5ccc4c23)c1]

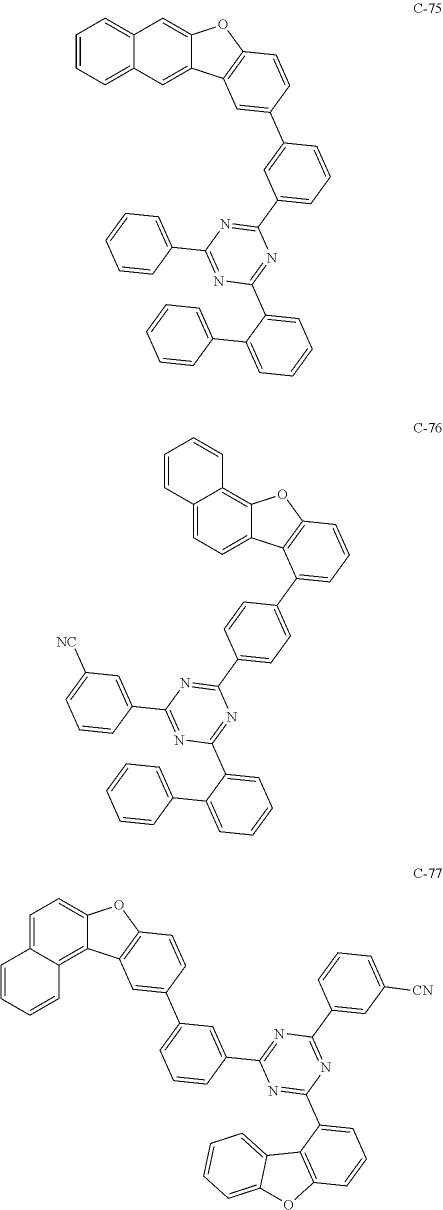 [SMILES: c1ccc(-c2nc(-c3cccc(-c4ccc5oc6cc7ccccc7cc6c5c4)c3)nc(-c3ccccc3-c3ccccc3)n2)cc1 ; N#Cc1cccc(-c2nc(-c3ccc(-c4cccc5oc6c7ccccc7ccc6c45)cc3)nc(-c3ccccc3-c3ccccc3)n2)c1 ; N#Cc1cccc(-c2nc(-c3cccc(-c4ccc5oc6ccc7ccccc7c6c5c4)c3)nc(-c3cccc4oc5ccccc5c34)n2)c1]

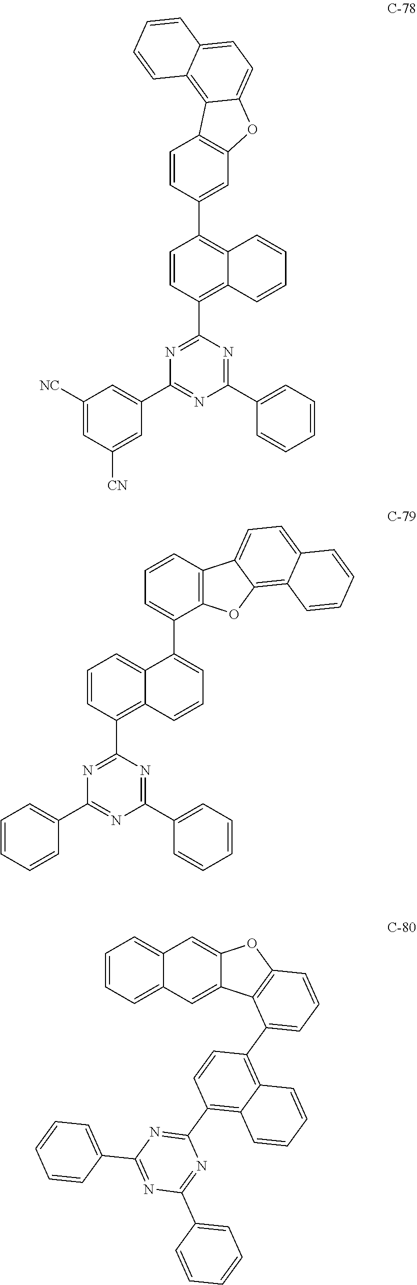 [SMILES: N#Cc1cc(C#N)cc(-c2nc(-c3ccccc3)nc(-c3ccc(-c4ccc5c(c4)oc4ccc6ccccc6c45)c4ccccc34)n2)c1 ; c1ccc(-c2nc(-c3ccccc3)nc(-c3cccc4c(-c5cccc6c5oc5c7ccccc7ccc65)cccc34)n2)cc1 ; c1ccc(-c2nc(-c3ccccc3)nc(-c3ccc(-c4cccc5oc6cc7ccccc7cc6c45)c4ccccc34)n2)cc1]

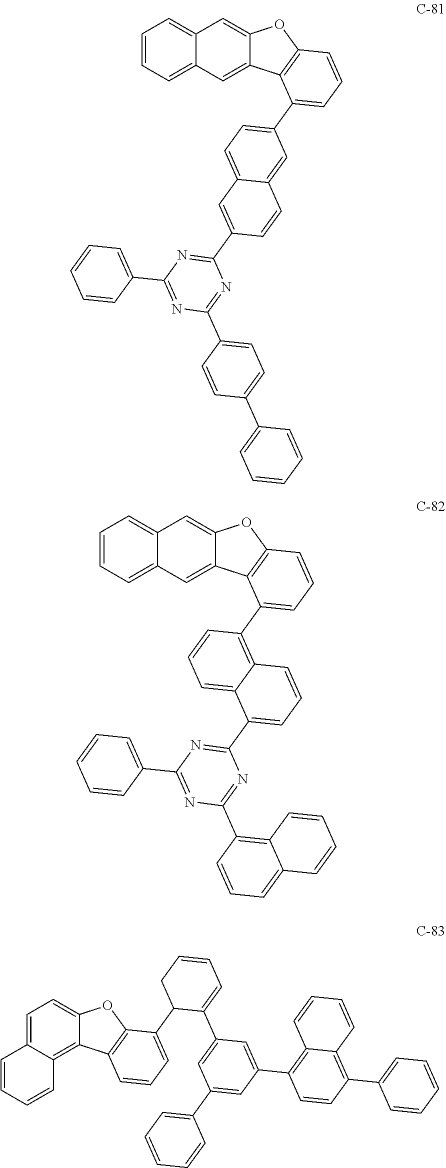

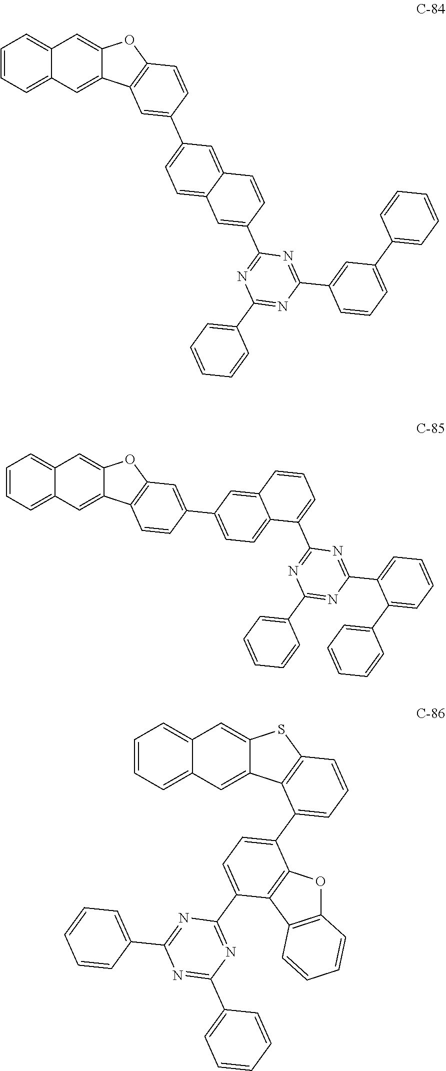 [SMILES: c1ccc(-c2cccc(-c3nc(-c4ccccc4)nc(-c4ccc5cc(-c6ccc7oc8cc9ccccc9cc8c7c6)ccc5c4)n3)c2)cc1 ; c1ccc(-c2nc(-c3ccccc3-c3ccccc3)nc(-c3cccc4cc(-c5ccc6c(c5)oc5cc7ccccc7cc56)ccc34)n2)cc1 ; c1ccc(-c2nc(-c3ccccc3)nc(-c3ccc(-c4cccc5sc6cc7ccccc7cc6c45)c4oc5ccccc5c34)n2)cc1]

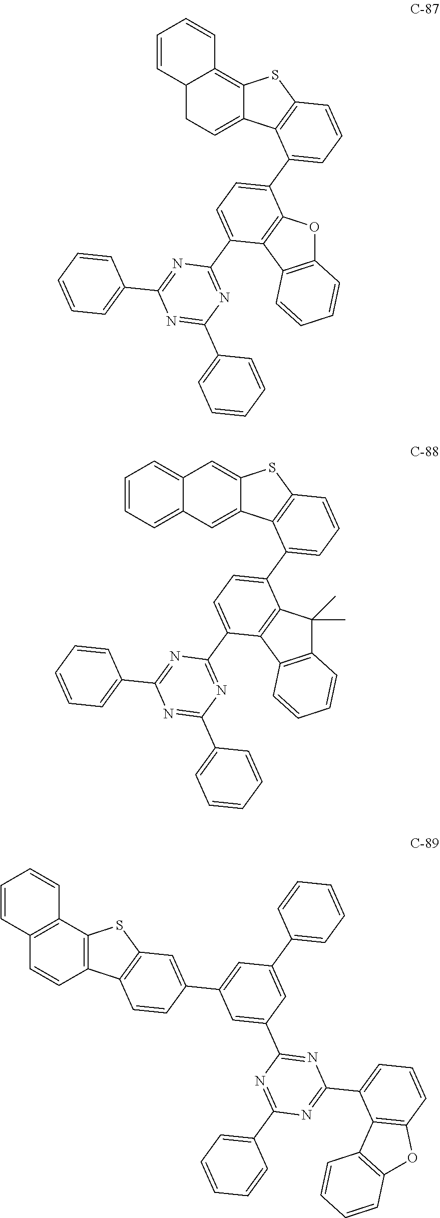 [SMILES: C1=CC2=c3sc4cccc(-c5ccc(-c6nc(-c7ccccc7)nc(-c7ccccc7)n6)c6c5oc5ccccc56)c4c3=CCC2C=C1 ; CC1(C)c2ccccc2-c2c(-c3nc(-c4ccccc4)nc(-c4ccccc4)n3)ccc(-c3cccc4sc5cc6ccccc6cc5c34)c21 ; c1ccc(-c2cc(-c3ccc4c(c3)sc3c5ccccc5ccc43)cc(-c3nc(-c4ccccc4)nc(-c4cccc5oc6ccccc6c45)n3)c2)cc1]

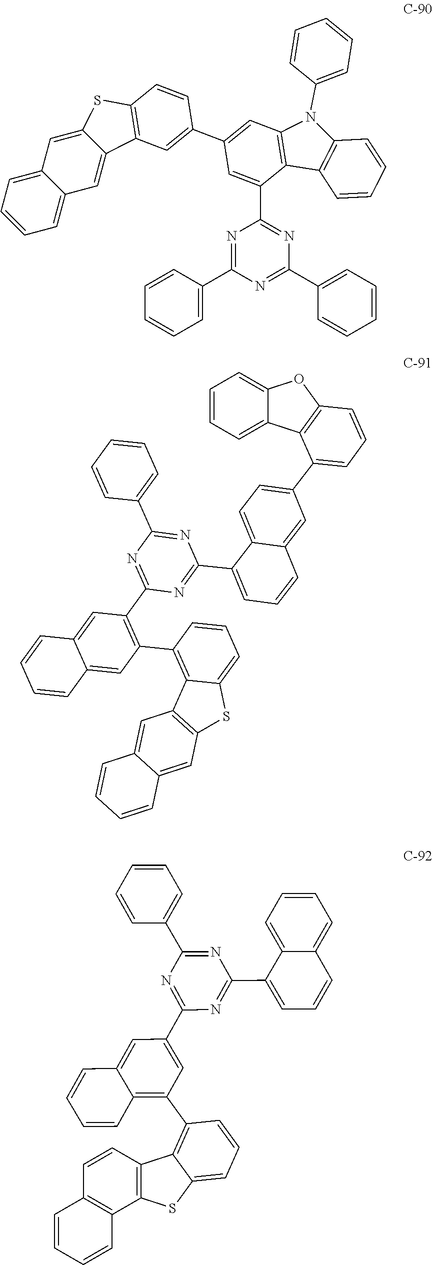 [SMILES: c1ccc(-c2nc(-c3ccccc3)nc(-c3cc(-c4ccc5sc6cc7ccccc7cc6c5c4)cc4c3c3ccccc3n4-c3ccccc3)n2)cc1 ; c1ccc(-c2nc(-c3cc4ccccc4cc3-c3cccc4sc5cc6ccccc6cc5c34)nc(-c3cccc4cc(-c5cccc6oc7ccccc7c56)ccc34)n2)cc1 ; c1ccc(-c2nc(-c3cc(-c4cccc5sc6c7ccccc7ccc6c45)c4ccccc4c3)nc(-c3cccc4ccccc34)n2)cc1]

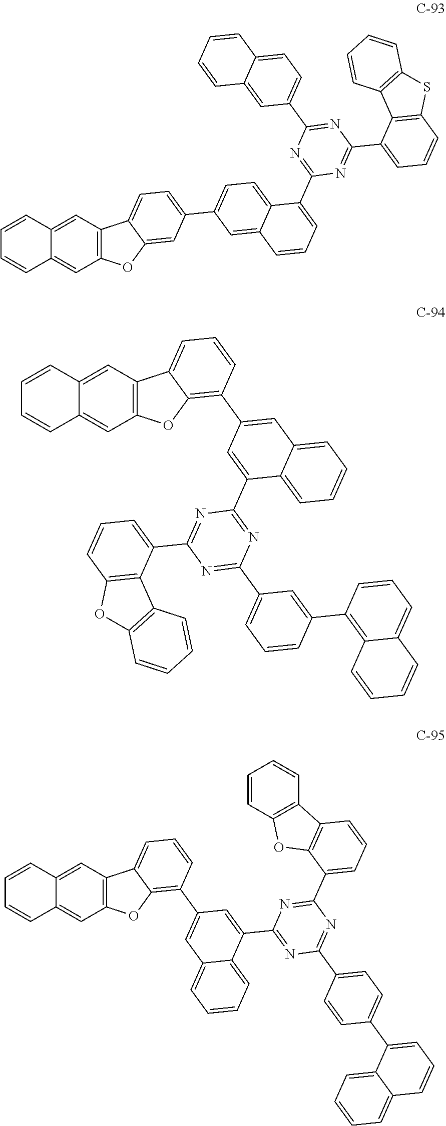 [SMILES: c1ccc2cc(-c3nc(-c4cccc5cc(-c6ccc7c(c6)oc6cc8ccccc8cc67)ccc45)nc(-c4cccc5sc6ccccc6c45)n3)ccc2c1 ; c1cc(-c2nc(-c3cc(-c4cccc5c4oc4cc6ccccc6cc45)cc4ccccc34)nc(-c3cccc4oc5ccccc5c34)n2)cc(-c2cccc3ccccc23)c1 ; c1ccc2cc3c(cc2c1)oc1c(-c2cc(-c4nc(-c5ccc(-c6cccc7ccccc67)cc5)nc(-c5cccc6c5oc5ccccc56)n4)c4ccccc4c2)cccc13]

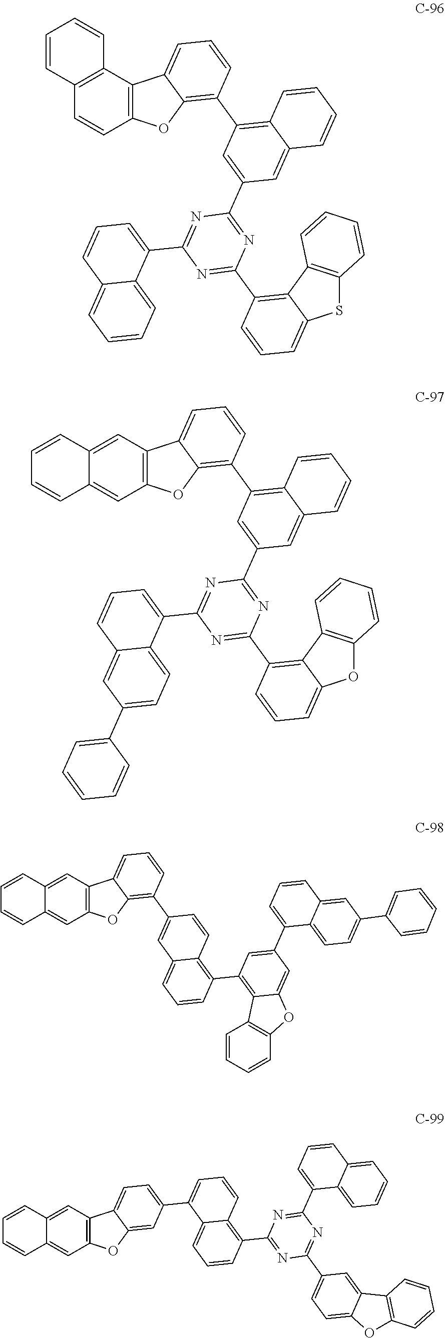 [SMILES: c1ccc2c(-c3nc(-c4cc(-c5cccc6c5oc5ccc7ccccc7c56)c5ccccc5c4)nc(-c4cccc5sc6ccccc6c45)n3)cccc2c1 ; c1ccc(-c2ccc3c(-c4nc(-c5cc(-c6cccc7c6oc6cc8ccccc8cc67)c6ccccc6c5)nc(-c5cccc6oc7ccccc7c56)n4)cccc3c2)cc1 ; c1ccc(-c2ccc3c(-c4cc(-c5cccc6cc(-c7cccc8c7oc7cc9ccccc9cc78)ccc56)c5c(c4)oc4ccccc45)cccc3c2)cc1 ; c1ccc2cc3c(cc2c1)oc1cc(-c2cccc4c(-c5nc(-c6ccc7oc8ccccc8c7c6)nc(-c6cccc7ccccc67)n5)cccc24)ccc13]

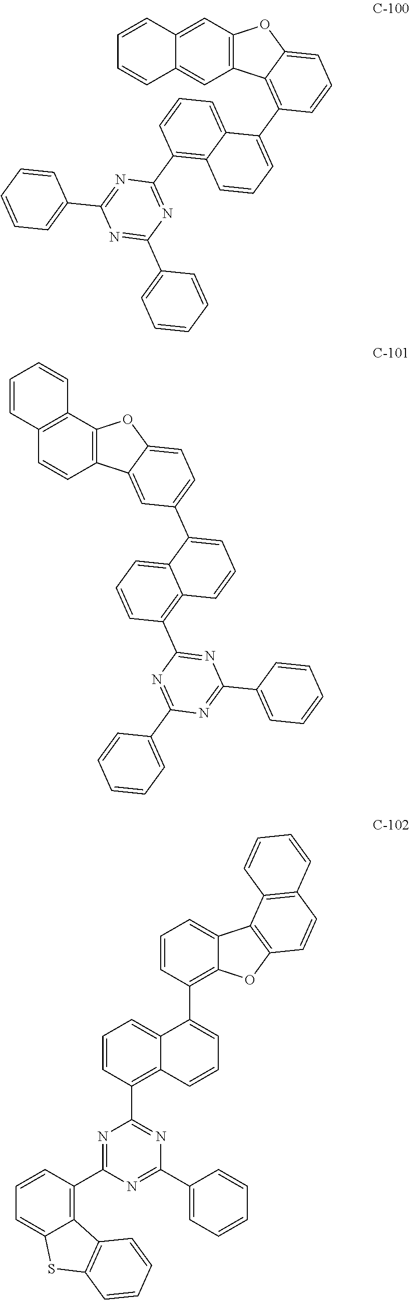 [SMILES: c1ccc(-c2nc(-c3ccccc3)nc(-c3cccc4c(-c5cccc6oc7cc8ccccc8cc7c56)cccc34)n2)cc1 ; c1ccc(-c2nc(-c3ccccc3)nc(-c3cccc4c(-c5ccc6oc7c8ccccc8ccc7c6c5)cccc34)n2)cc1 ; c1ccc(-c2nc(-c3cccc4c(-c5cccc6c5oc5ccc7ccccc7c56)cccc34)nc(-c3cccc4sc5ccccc5c34)n2)cc1]

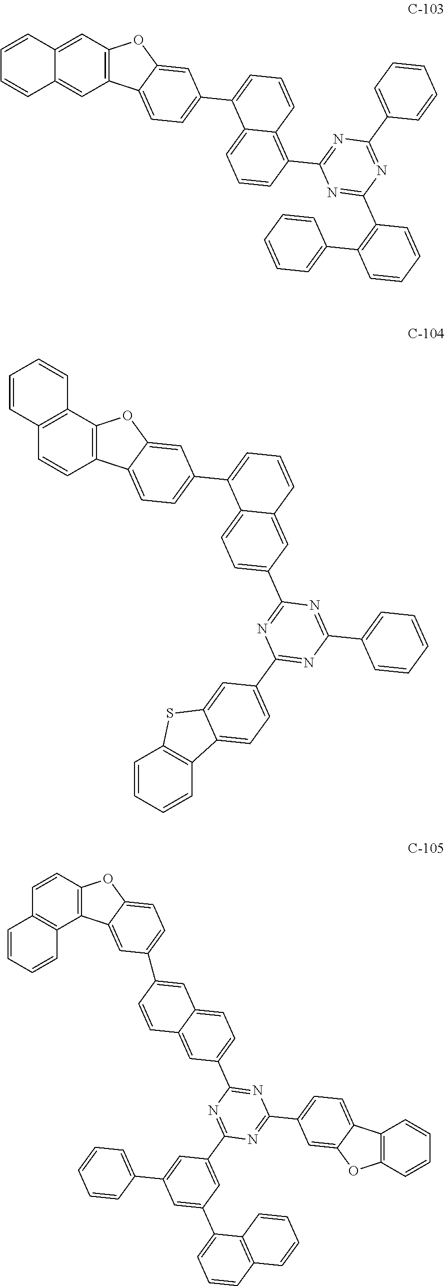 [SMILES: c1ccc(-c2nc(-c3ccccc3-c3ccccc3)nc(-c3cccc4c(-c5ccc6c(c5)oc5cc7ccccc7cc56)cccc34)n2)cc1 ; c1ccc(-c2nc(-c3ccc4c(-c5ccc6c(c5)oc5c7ccccc7ccc65)cccc4c3)nc(-c3ccc4c(c3)sc3ccccc34)n2)cc1 ; c1ccc(-c2cc(-c3nc(-c4ccc5cc(-c6ccc7oc8ccc9ccccc9c8c7c6)ccc5c4)nc(-c4ccc5c(c4)oc4ccccc45)n3)cc(-c3cccc4ccccc34)c2)cc1]

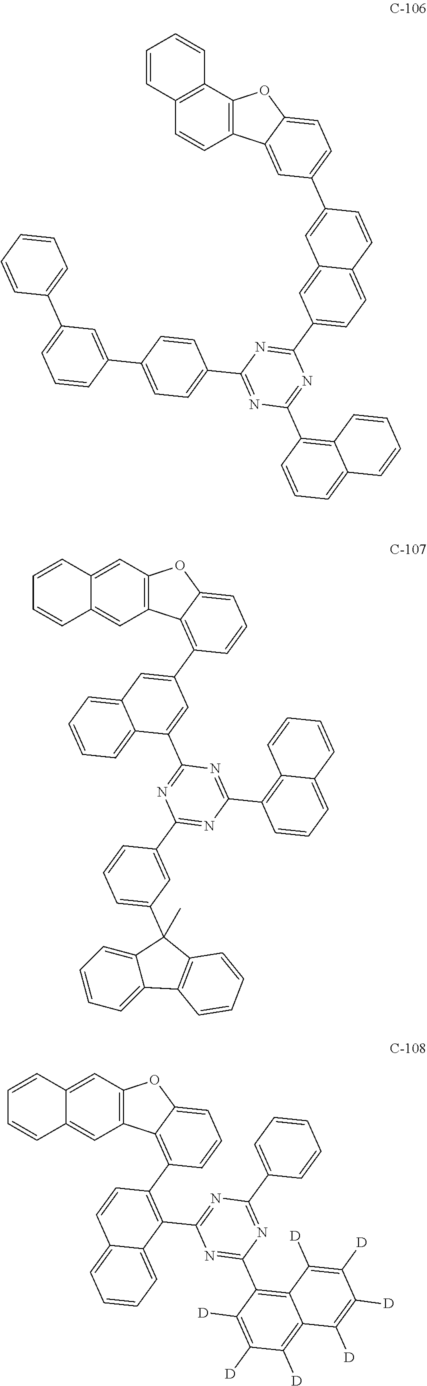 [SMILES: c1ccc(-c2cccc(-c3ccc(-c4nc(-c5ccc6ccc(-c7ccc8oc9c%10ccccc%10ccc9c8c7)cc6c5)nc(-c5cccc6ccccc56)n4)cc3)c2)cc1 ; CC1(c2cccc(-c3nc(-c4cccc5ccccc45)nc(-c4cc(-c5cccc6oc7cc8ccccc8cc7c56)cc5ccccc45)n3)c2)c2ccccc2-c2ccccc21 ; [2H]c1c([2H])c([2H])c2c(-c3nc(-c4ccccc4)nc(-c4c(-c5cccc6oc7cc8ccccc8cc7c56)ccc5ccccc45)n3)c([2H])c([2H])c([2H])c2c1[2H]]

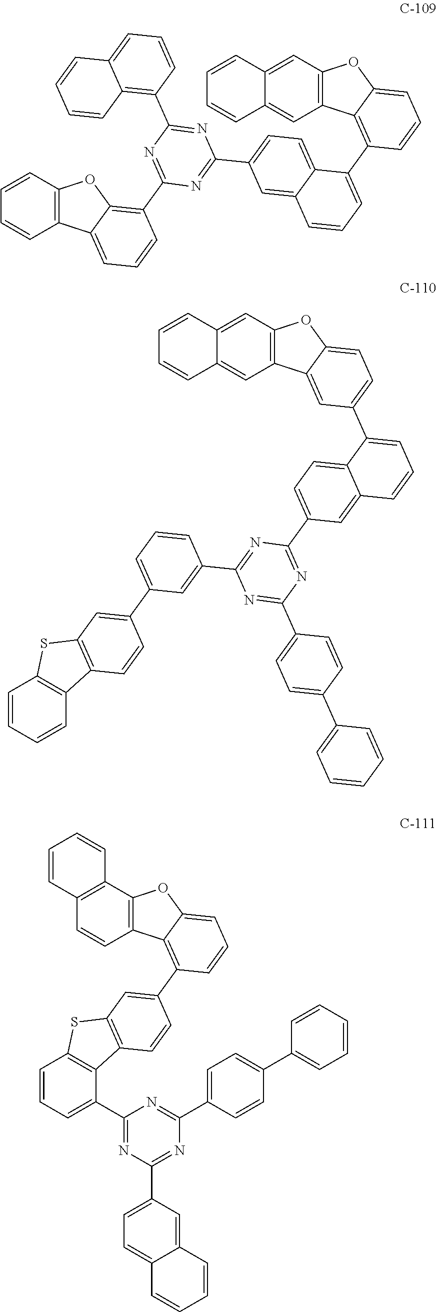 [SMILES: c1ccc2cc3c(cc2c1)oc1cccc(-c2cccc4cc(-c5nc(-c6cccc7ccccc67)nc(-c6cccc7c6oc6ccccc67)n5)ccc24)c13 ; c1ccc(-c2ccc(-c3nc(-c4cccc(-c5ccc6c(c5)sc5ccccc56)c4)nc(-c4ccc5c(-c6ccc7oc8cc9ccccc9cc8c7c6)cccc5c4)n3)cc2)cc1 ; c1ccc(-c2ccc(-c3nc(-c4ccc5ccccc5c4)nc(-c4cccc5sc6cc(-c7cccc8oc9c%10ccccc%10ccc9c78)ccc6c45)n3)cc2)cc1]

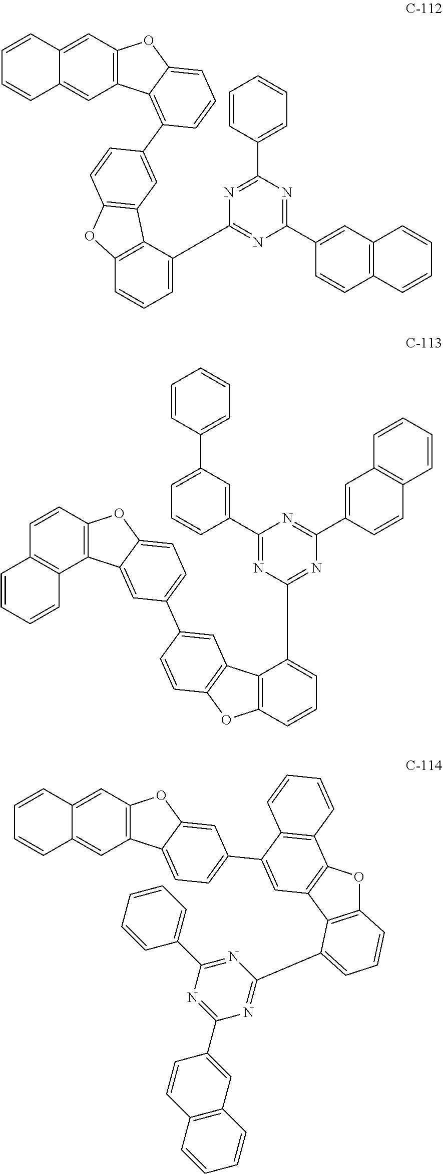 [SMILES: c1ccc(-c2nc(-c3ccc4ccccc4c3)nc(-c3cccc4oc5ccc(-c6cccc7oc8cc9ccccc9cc8c67)cc5c34)n2)cc1 ; c1ccc(-c2cccc(-c3nc(-c4ccc5ccccc5c4)nc(-c4cccc5oc6ccc(-c7ccc8oc9ccc%10ccccc%10c9c8c7)cc6c45)n3)c2)cc1 ; c1ccc(-c2nc(-c3ccc4ccccc4c3)nc(-c3cccc4oc5c6ccccc6c(-c6ccc7c(c6)oc6cc8ccccc8cc67)cc5c34)n2)cc1]

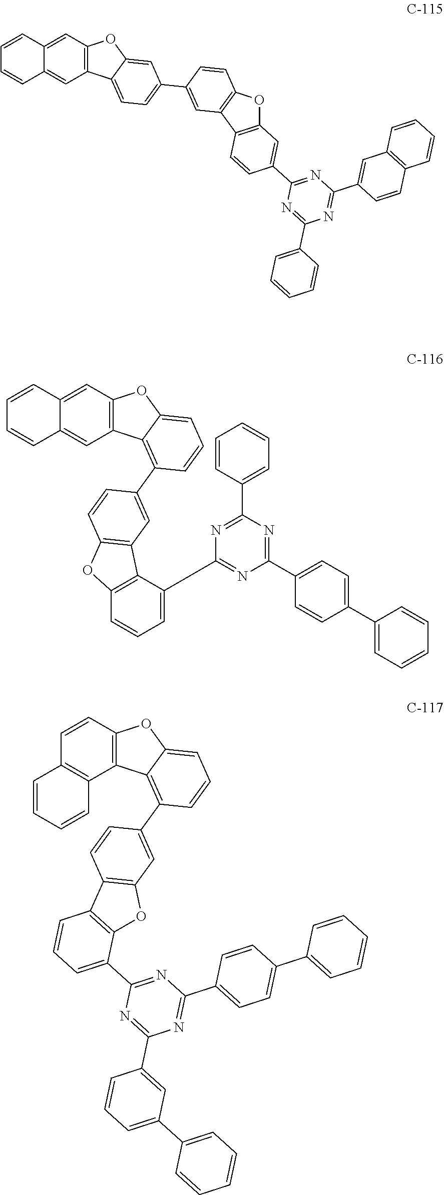 [SMILES: c1ccc(-c2nc(-c3ccc4ccccc4c3)nc(-c3ccc4c(c3)oc3ccc(-c5ccc6c(c5)oc5cc7ccccc7cc56)cc34)n2)cc1 ; c1ccc(-c2ccc(-c3nc(-c4ccccc4)nc(-c4cccc5oc6ccc(-c7cccc8oc9cc%10ccccc%10cc9c78)cc6c45)n3)cc2)cc1 ; c1ccc(-c2ccc(-c3nc(-c4cccc(-c5ccccc5)c4)nc(-c4cccc5c4oc4cc(-c6cccc7oc8ccc9ccccc9c8c67)ccc45)n3)cc2)cc1]

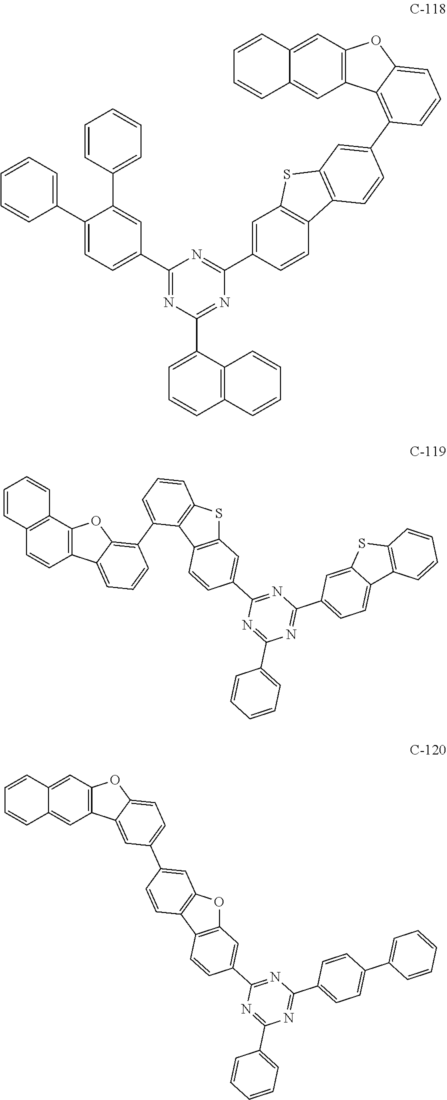 [SMILES: c1ccc(-c2ccc(-c3nc(-c4ccc5c(c4)sc4cc(-c6cccc7oc8cc9ccccc9cc8c67)ccc45)nc(-c4cccc5ccccc45)n3)cc2-c2ccccc2)cc1 ; c1ccc(-c2nc(-c3ccc4c(c3)sc3ccccc34)nc(-c3ccc4c(c3)sc3cccc(-c5cccc6c5oc5c7ccccc7ccc65)c34)n2)cc1 ; c1ccc(-c2ccc(-c3nc(-c4ccccc4)nc(-c4ccc5c(c4)oc4cc(-c6ccc7oc8cc9ccccc9cc8c7c6)ccc45)n3)cc2)cc1]

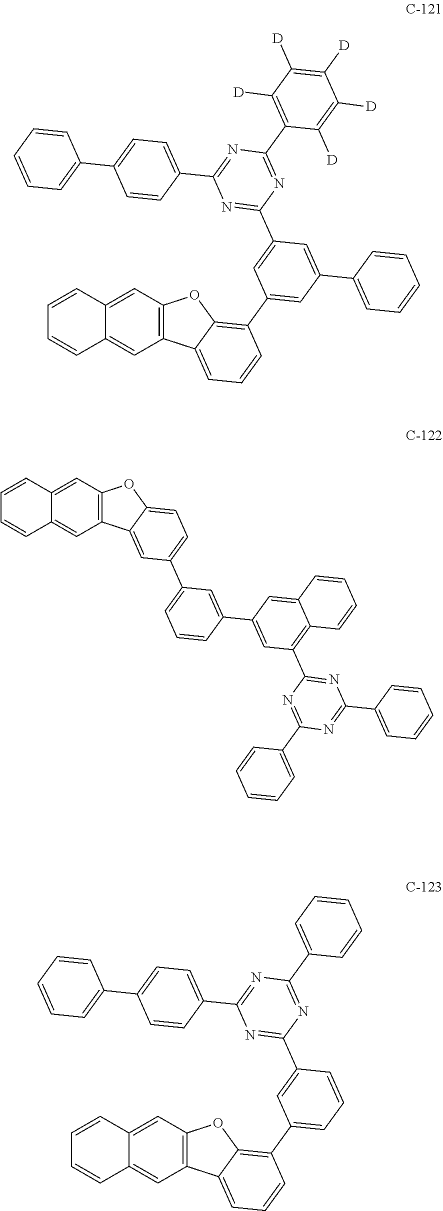

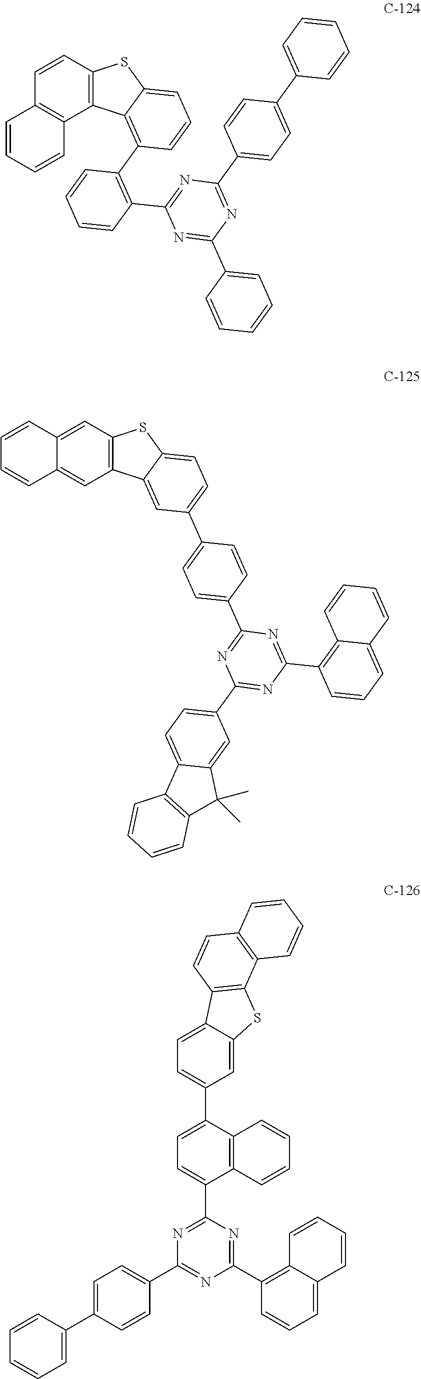

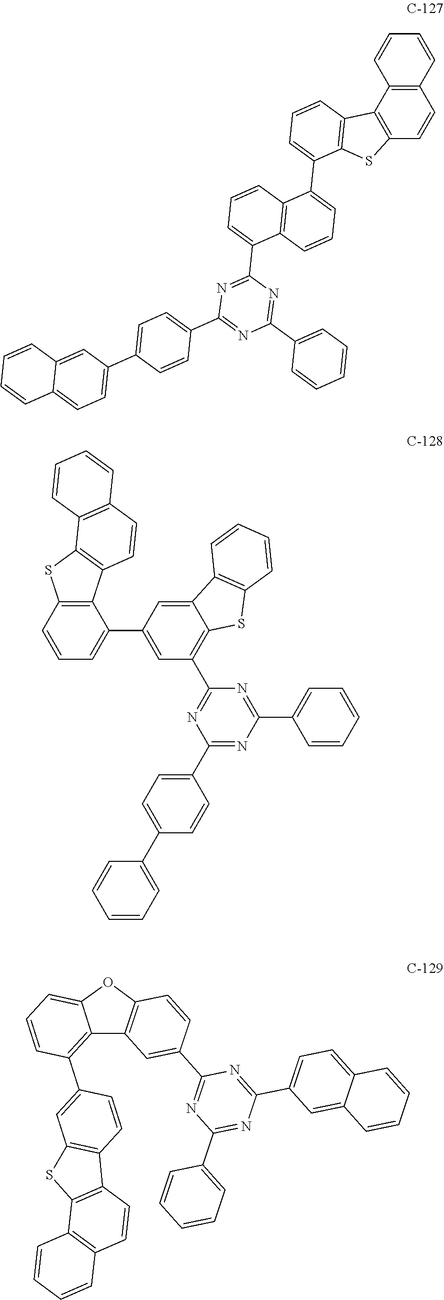

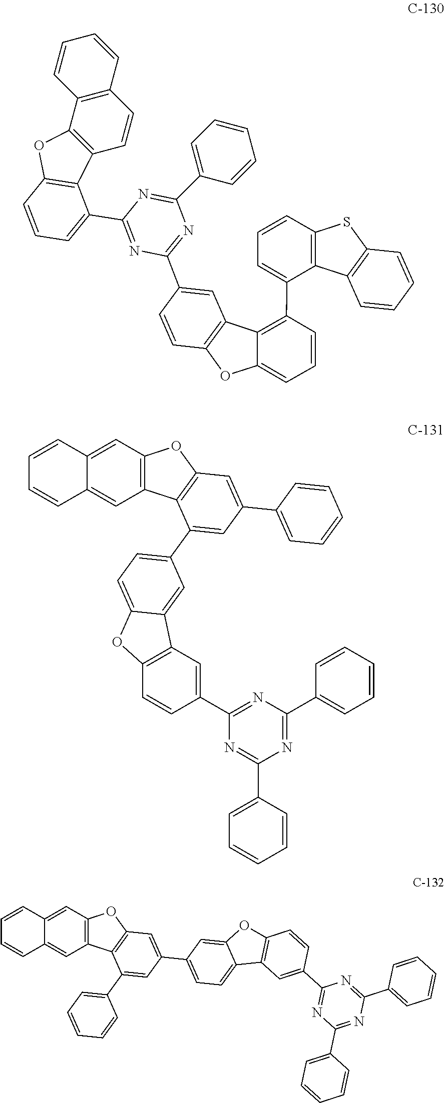 [SMILES: c1ccc(-c2nc(-c3ccc4oc5cccc(-c6cccc7sc8ccccc8c67)c5c4c3)nc(-c3cccc4oc5c6ccccc6ccc5c34)n2)cc1 ; c1ccc(-c2cc(-c3ccc4oc5ccc(-c6nc(-c7ccccc7)nc(-c7ccccc7)n6)cc5c4c3)c3c(c2)oc2cc4ccccc4cc23)cc1 ; c1ccc(-c2nc(-c3ccccc3)nc(-c3ccc4oc5cc(-c6cc(-c7ccccc7)c7c(c6)oc6cc8ccccc8cc67)ccc5c4c3)n2)cc1]

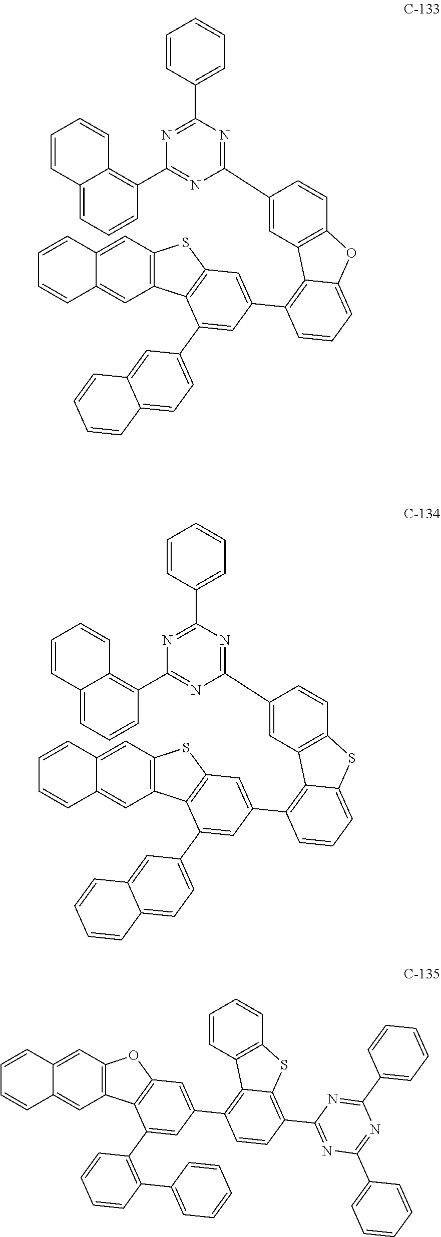 [SMILES: c1ccc(-c2nc(-c3ccc4oc5cccc(-c6cc(-c7ccc8ccccc8c7)c7c(c6)sc6cc8ccccc8cc67)c5c4c3)nc(-c3cccc4ccccc34)n2)cc1 ; c1ccc(-c2nc(-c3ccc4sc5cccc(-c6cc(-c7ccc8ccccc8c7)c7c(c6)sc6cc8ccccc8cc67)c5c4c3)nc(-c3cccc4ccccc34)n2)cc1 ; c1ccc(-c2nc(-c3ccccc3)nc(-c3ccc(-c4cc(-c5ccccc5-c5ccccc5)c5c(c4)oc4cc6ccccc6cc45)c4c3sc3ccccc34)n2)cc1]

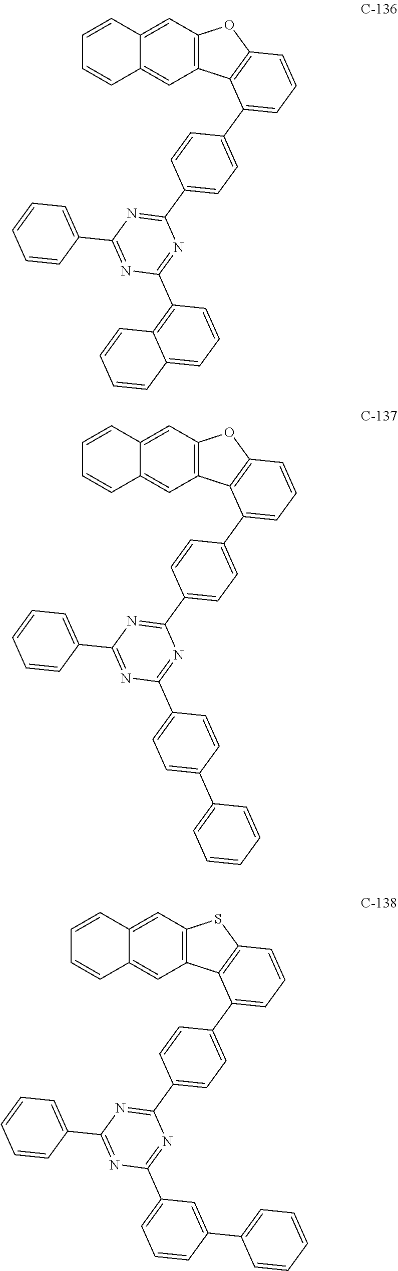

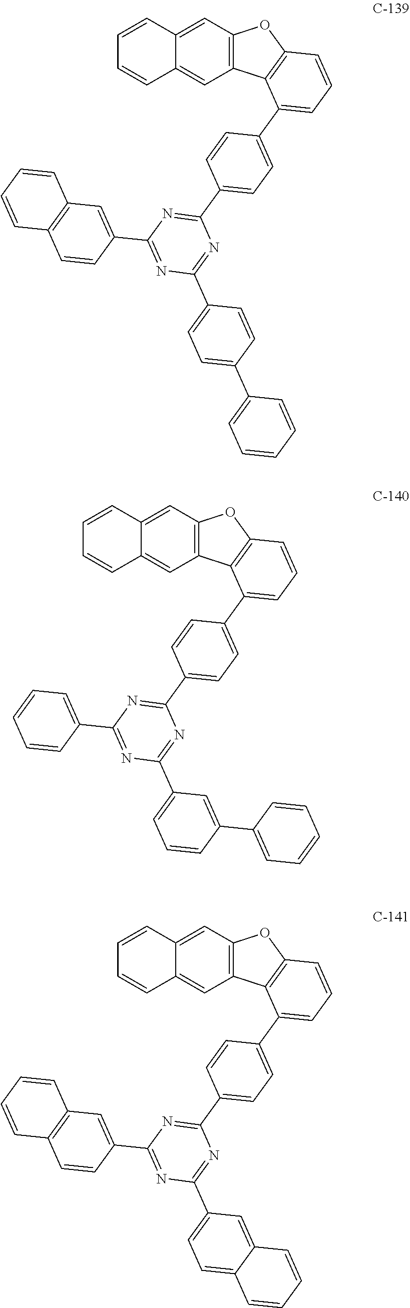 [SMILES: c1ccc(-c2ccc(-c3nc(-c4ccc(-c5cccc6oc7cc8ccccc8cc7c56)cc4)nc(-c4ccc5ccccc5c4)n3)cc2)cc1 ; c1ccc(-c2cccc(-c3nc(-c4ccccc4)nc(-c4ccc(-c5cccc6oc7cc8ccccc8cc7c56)cc4)n3)c2)cc1 ; c1ccc2cc(-c3nc(-c4ccc(-c5cccc6oc7cc8ccccc8cc7c56)cc4)nc(-c4ccc5ccccc5c4)n3)ccc2c1]

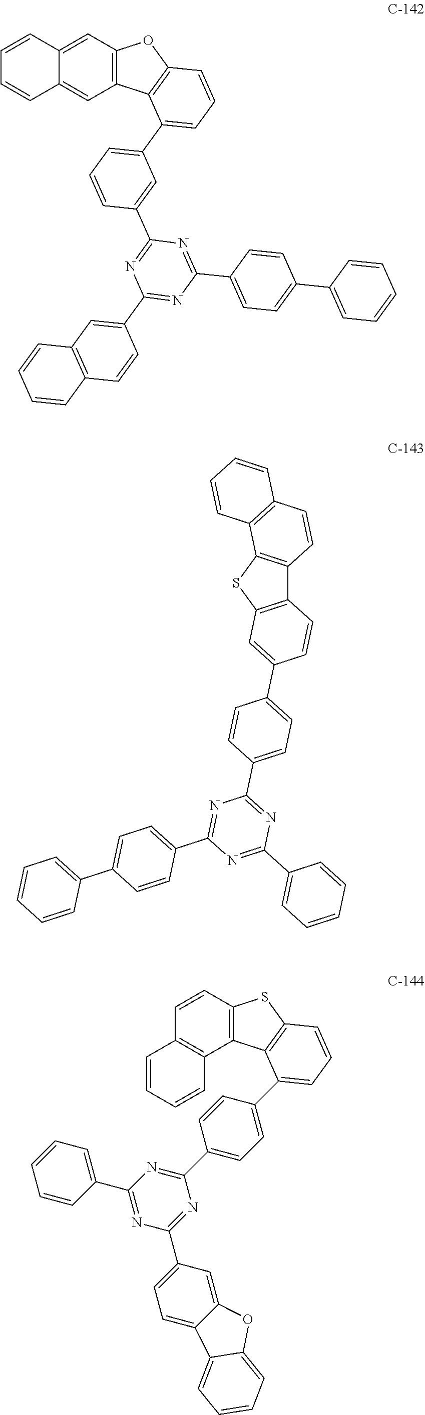 [SMILES: c1ccc(-c2ccc(-c3nc(-c4cccc(-c5cccc6oc7cc8ccccc8cc7c56)c4)nc(-c4ccc5ccccc5c4)n3)cc2)cc1 ; c1ccc(-c2ccc(-c3nc(-c4ccccc4)nc(-c4ccc(-c5ccc6c(c5)sc5c7ccccc7ccc65)cc4)n3)cc2)cc1 ; c1ccc(-c2nc(-c3ccc(-c4cccc5sc6ccc7ccccc7c6c45)cc3)nc(-c3ccc4c(c3)oc3ccccc34)n2)cc1]

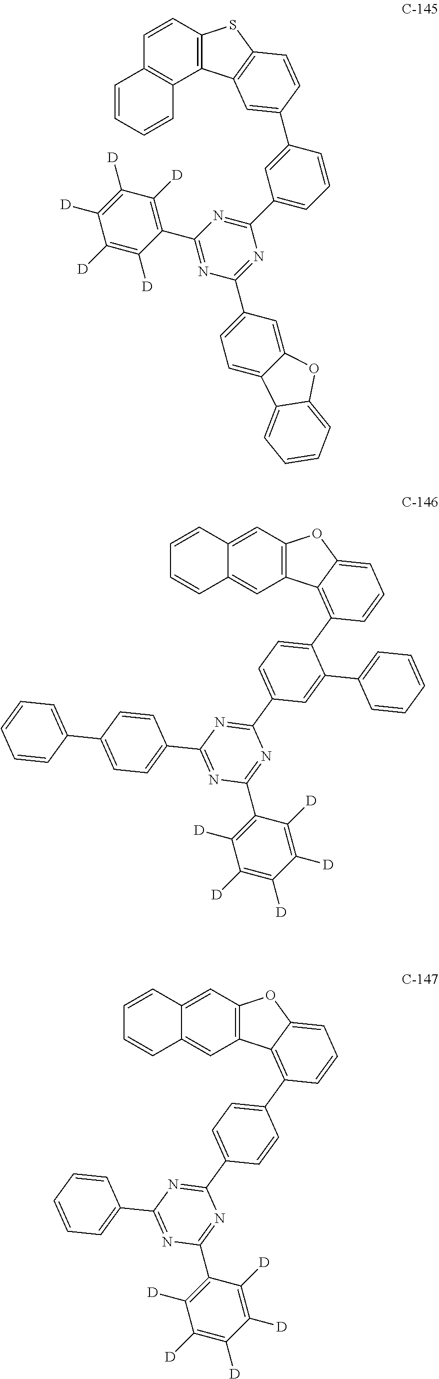 [SMILES: [2H]c1c([2H])c([2H])c(-c2nc(-c3cccc(-c4ccc5sc6ccc7ccccc7c6c5c4)c3)nc(-c3ccc4c(c3)oc3ccccc34)n2)c([2H])c1[2H] ; [2H]c1c([2H])c([2H])c(-c2nc(-c3ccc(-c4ccccc4)cc3)nc(-c3ccc(-c4cccc5oc6cc7ccccc7cc6c45)c(-c4ccccc4)c3)n2)c([2H])c1[2H] ; [2H]c1c([2H])c([2H])c(-c2nc(-c3ccccc3)nc(-c3ccc(-c4cccc5oc6cc7ccccc7cc6c45)cc3)n2)c([2H])c1[2H]]

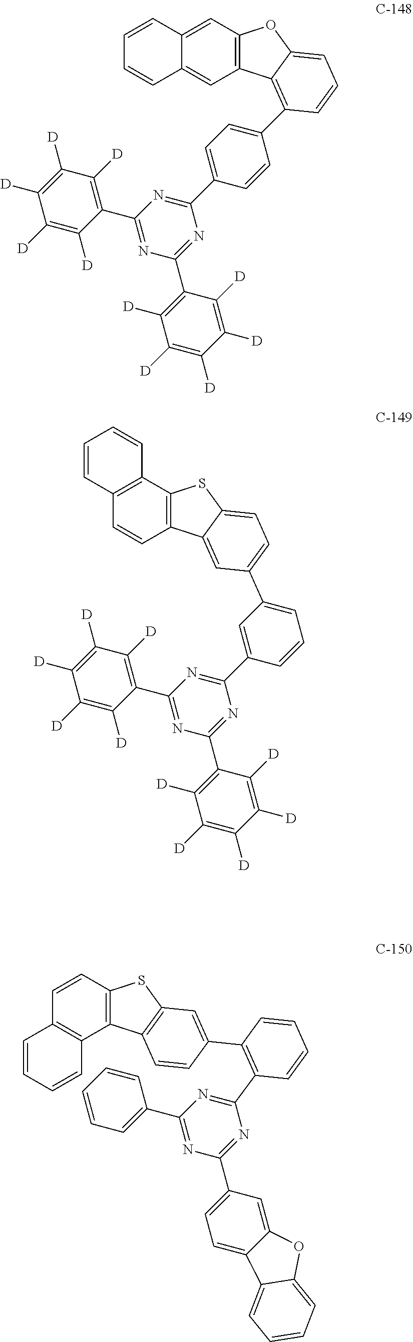 [SMILES: [2H]c1c([2H])c([2H])c(-c2nc(-c3ccc(-c4cccc5oc6cc7ccccc7cc6c45)cc3)nc(-c3c([2H])c([2H])c([2H])c([2H])c3[2H])n2)c([2H])c1[2H] ; [2H]c1c([2H])c([2H])c(-c2nc(-c3cccc(-c4ccc5sc6c7ccccc7ccc6c5c4)c3)nc(-c3c([2H])c([2H])c([2H])c([2H])c3[2H])n2)c([2H])c1[2H] ; c1ccc(-c2nc(-c3ccc4c(c3)oc3ccccc34)nc(-c3ccccc3-c3ccc4c(c3)sc3ccc5ccccc5c34)n2)cc1]

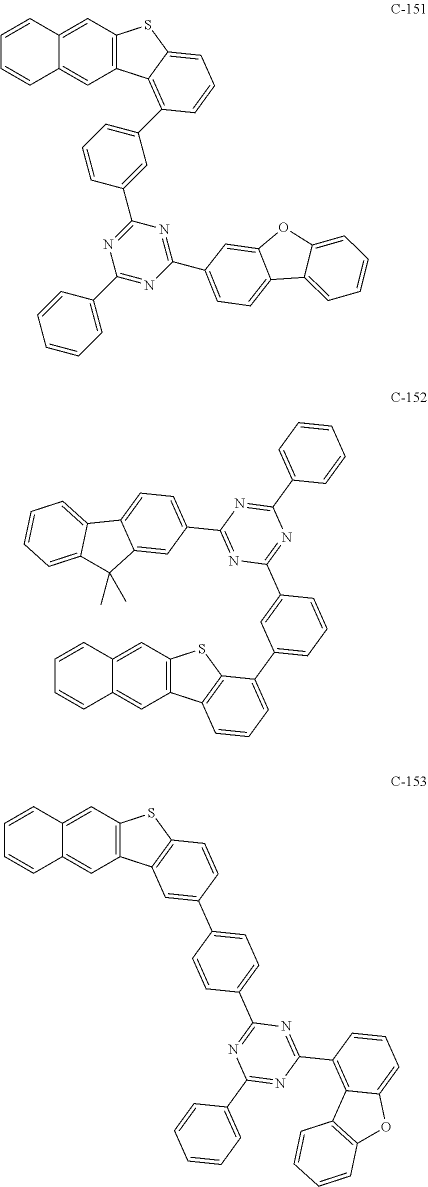 [SMILES: c1ccc(-c2nc(-c3cccc(-c4cccc5sc6cc7ccccc7cc6c45)c3)nc(-c3ccc4c(c3)oc3ccccc34)n2)cc1 ; CC1(C)c2ccccc2-c2ccc(-c3nc(-c4ccccc4)nc(-c4cccc(-c5cccc6c5sc5cc7ccccc7cc56)c4)n3)cc21 ; c1ccc(-c2nc(-c3ccc(-c4ccc5sc6cc7ccccc7cc6c5c4)cc3)nc(-c3cccc4oc5ccccc5c34)n2)cc1]

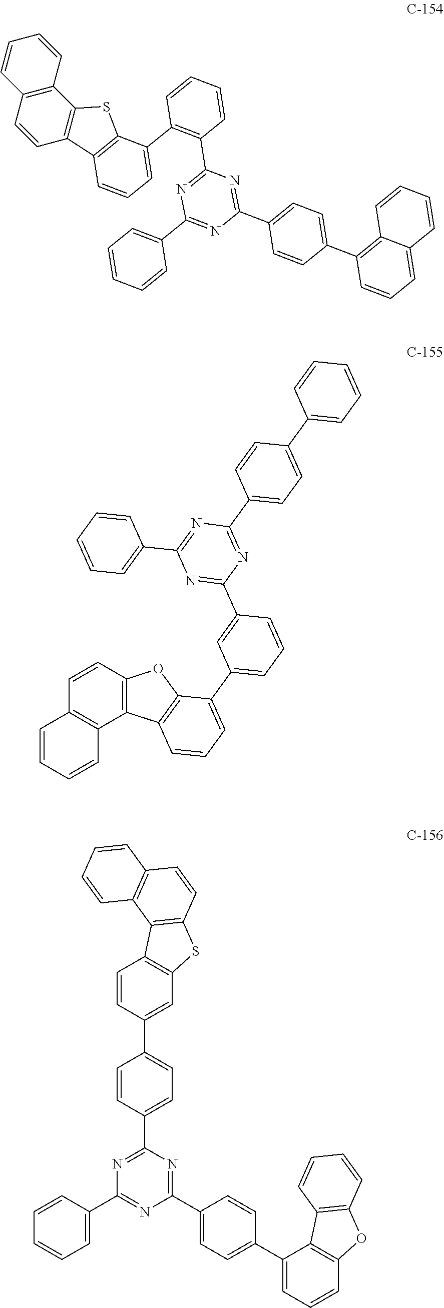 [SMILES: c1ccc(-c2nc(-c3ccc(-c4cccc5ccccc45)cc3)nc(-c3ccccc3-c3cccc4c3sc3c5ccccc5ccc43)n2)cc1 ; c1ccc(-c2ccc(-c3nc(-c4ccccc4)nc(-c4cccc(-c5cccc6c5oc5ccc7ccccc7c56)c4)n3)cc2)cc1 ; c1ccc(-c2nc(-c3ccc(-c4ccc5c(c4)sc4ccc6ccccc6c45)cc3)nc(-c3ccc(-c4cccc5oc6ccccc6c45)cc3)n2)cc1]

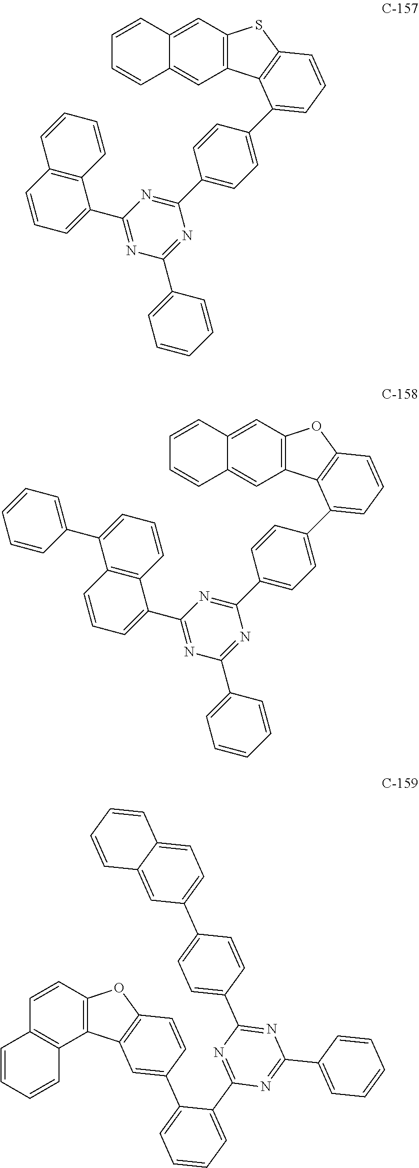 [SMILES: c1ccc(-c2nc(-c3ccc(-c4cccc5sc6cc7ccccc7cc6c45)cc3)nc(-c3cccc4ccccc34)n2)cc1 ; c1ccc(-c2nc(-c3ccc(-c4cccc5oc6cc7ccccc7cc6c45)cc3)nc(-c3cccc4c(-c5ccccc5)cccc34)n2)cc1 ; c1ccc(-c2nc(-c3ccc(-c4ccc5ccccc5c4)cc3)nc(-c3ccccc3-c3ccc4oc5ccc6ccccc6c5c4c3)n2)cc1]

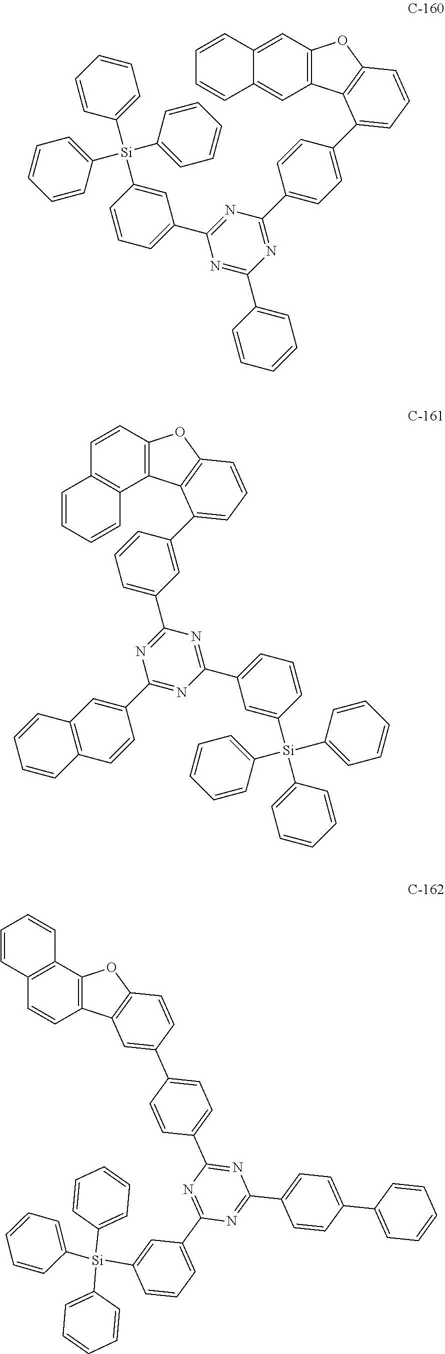 [SMILES: c1ccc(-c2nc(-c3ccc(-c4cccc5oc6cc7ccccc7cc6c45)cc3)nc(-c3cccc([Si](c4ccccc4)(c4ccccc4)c4ccccc4)c3)n2)cc1 ; c1ccc([Si](c2ccccc2)(c2ccccc2)c2cccc(-c3nc(-c4cccc(-c5cccc6oc7ccc8ccccc8c7c56)c4)nc(-c4ccc5ccccc5c4)n3)c2)cc1 ; c1ccc(-c2ccc(-c3nc(-c4ccc(-c5ccc6oc7c8ccccc8ccc7c6c5)cc4)nc(-c4cccc([Si](c5ccccc5)(c5ccccc5)c5ccccc5)c4)n3)cc2)cc1]

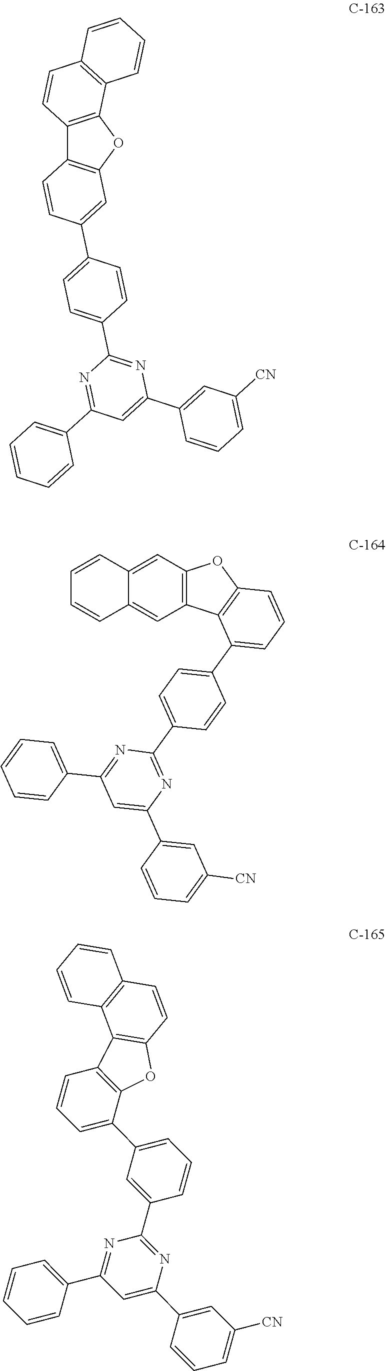 [SMILES: N#Cc1cccc(-c2cc(-c3ccccc3)nc(-c3ccc(-c4ccc5c(c4)oc4c6ccccc6ccc54)cc3)n2)c1 ; N#Cc1cccc(-c2cc(-c3ccccc3)nc(-c3ccc(-c4cccc5oc6cc7ccccc7cc6c45)cc3)n2)c1 ; N#Cc1cccc(-c2cc(-c3ccccc3)nc(-c3cccc(-c4cccc5c4oc4ccc6ccccc6c45)c3)n2)c1]

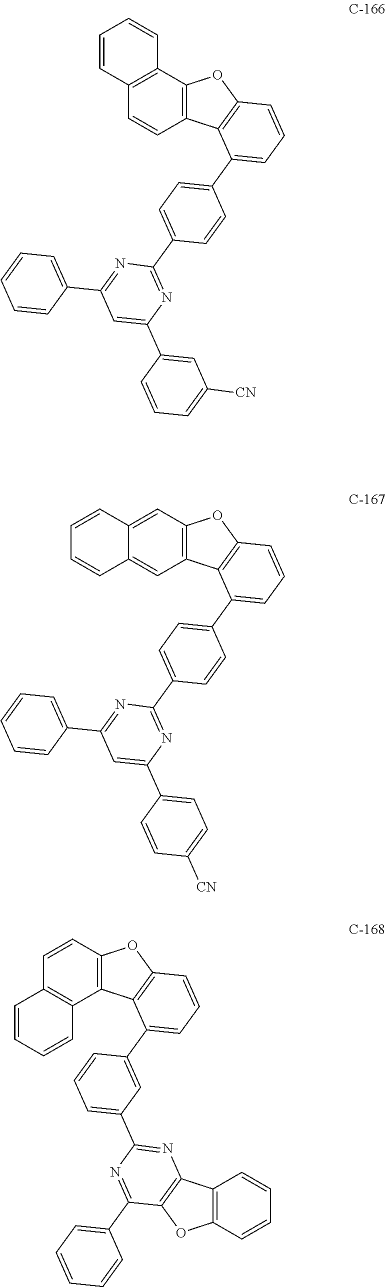 [SMILES: N#Cc1cccc(-c2cc(-c3ccccc3)nc(-c3ccc(-c4cccc5oc6c7ccccc7ccc6c45)cc3)n2)c1 ; N#Cc1ccc(-c2cc(-c3ccccc3)nc(-c3ccc(-c4cccc5oc6cc7ccccc7cc6c45)cc3)n2)cc1 ; c1ccc(-c2nc(-c3cccc(-c4cccc5oc6ccc7ccccc7c6c45)c3)nc3c2oc2ccccc23)cc1]

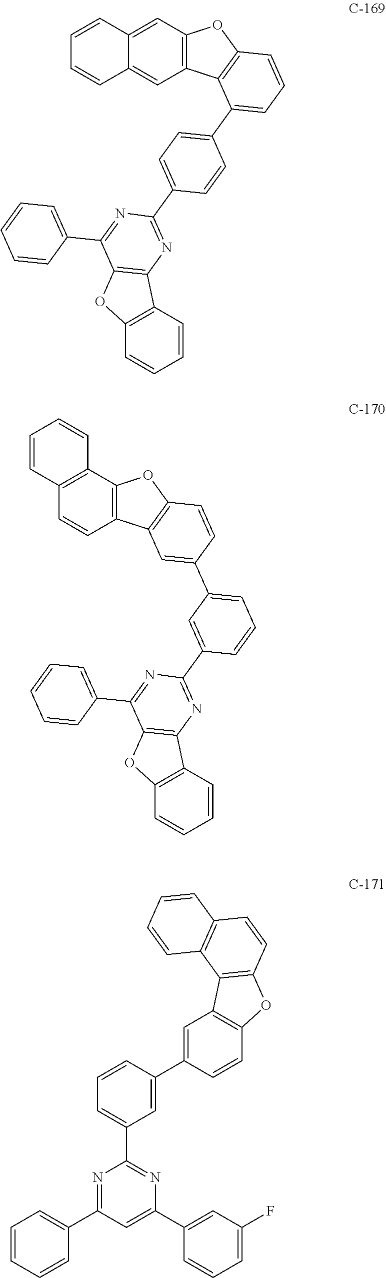 [SMILES: c1ccc(-c2nc(-c3ccc(-c4cccc5oc6cc7ccccc7cc6c45)cc3)nc3c2oc2ccccc23)cc1 ; c1ccc(-c2nc(-c3cccc(-c4ccc5oc6c7ccccc7ccc6c5c4)c3)nc3c2oc2ccccc23)cc1 ; Fc1cccc(-c2cc(-c3ccccc3)nc(-c3cccc(-c4ccc5oc6ccc7ccccc7c6c5c4)c3)n2)c1]

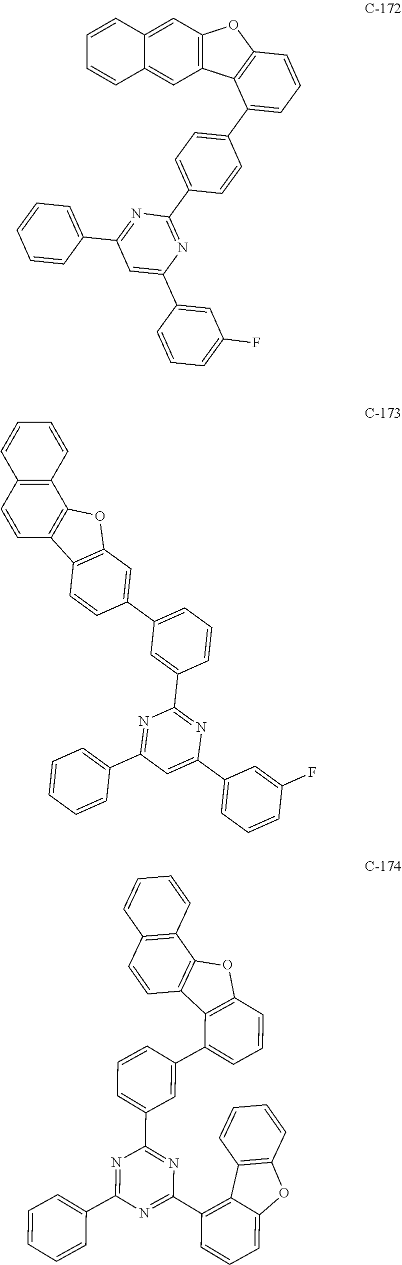 [SMILES: Fc1cccc(-c2cc(-c3ccccc3)nc(-c3ccc(-c4cccc5oc6cc7ccccc7cc6c45)cc3)n2)c1 ; Fc1cccc(-c2cc(-c3ccccc3)nc(-c3cccc(-c4ccc5c(c4)oc4c6ccccc6ccc54)c3)n2)c1 ; c1ccc(-c2nc(-c3cccc(-c4cccc5oc6c7ccccc7ccc6c45)c3)nc(-c3cccc4oc5ccccc5c34)n2)cc1]

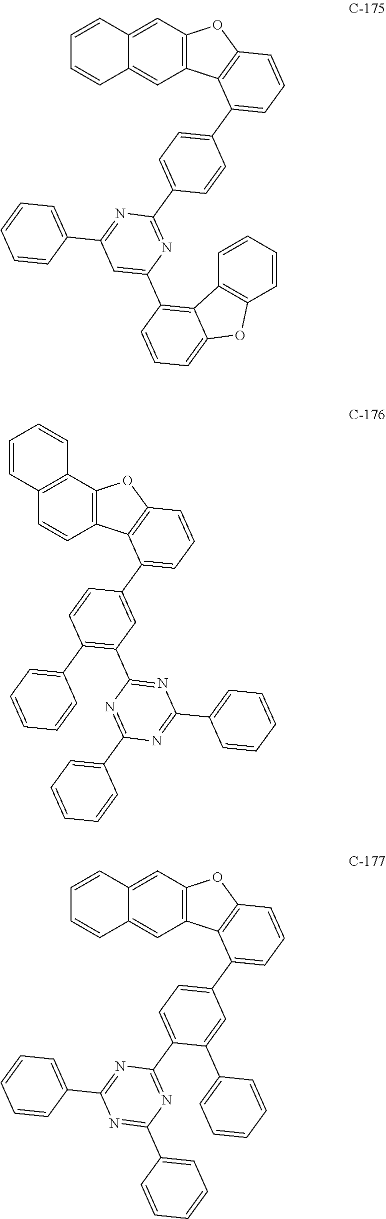 [SMILES: c1ccc(-c2cc(-c3cccc4oc5ccccc5c34)nc(-c3ccc(-c4cccc5oc6cc7ccccc7cc6c45)cc3)n2)cc1 ; c1ccc(-c2nc(-c3ccccc3)nc(-c3cc(-c4cccc5oc6c7ccccc7ccc6c45)ccc3-c3ccccc3)n2)cc1 ; c1ccc(-c2nc(-c3ccccc3)nc(-c3ccc(-c4cccc5oc6cc7ccccc7cc6c45)cc3-c3ccccc3)n2)cc1]

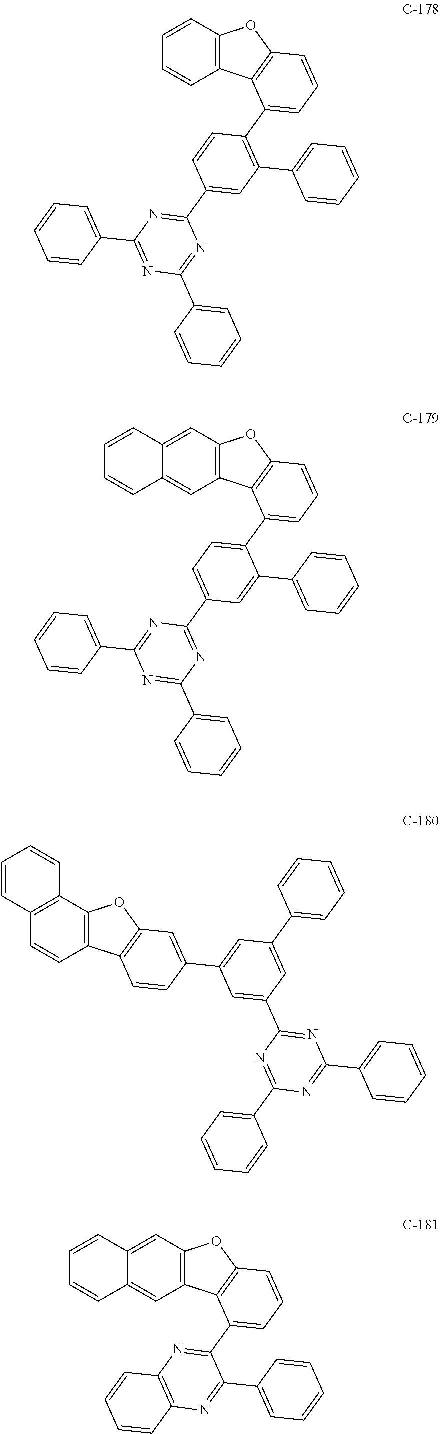 [SMILES: c1ccc(-c2nc(-c3ccccc3)nc(-c3ccc(-c4cccc5oc6ccccc6c45)c(-c4ccccc4)c3)n2)cc1 ; c1ccc(-c2nc(-c3ccccc3)nc(-c3ccc(-c4cccc5oc6cc7ccccc7cc6c45)c(-c4ccccc4)c3)n2)cc1 ; c1ccc(-c2cc(-c3ccc4c(c3)oc3c5ccccc5ccc43)cc(-c3nc(-c4ccccc4)nc(-c4ccccc4)n3)c2)cc1 ; c1ccc(-c2nc3ccccc3nc2-c2cccc3oc4cc5ccccc5cc4c23)cc1]

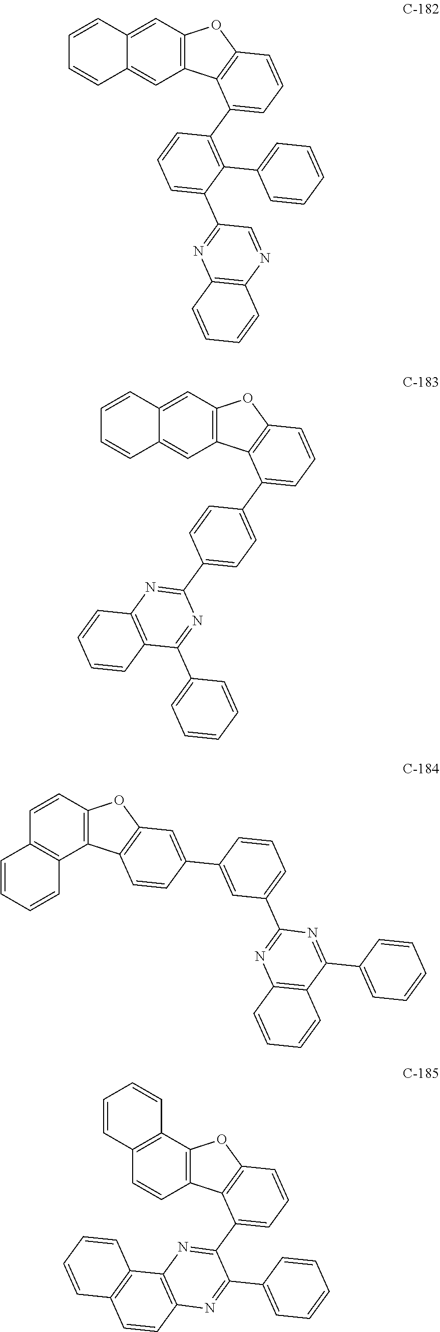 [SMILES: c1ccc(-c2c(-c3cnc4ccccc4n3)cccc2-c2cccc3oc4cc5ccccc5cc4c23)cc1 ; c1ccc(-c2nc(-c3ccc(-c4cccc5oc6cc7ccccc7cc6c45)cc3)nc3ccccc23)cc1 ; c1ccc(-c2nc(-c3cccc(-c4ccc5c(c4)oc4ccc6ccccc6c45)c3)nc3ccccc23)cc1 ; c1ccc(-c2nc3ccc4ccccc4c3nc2-c2cccc3oc4c5ccccc5ccc4c23)cc1]

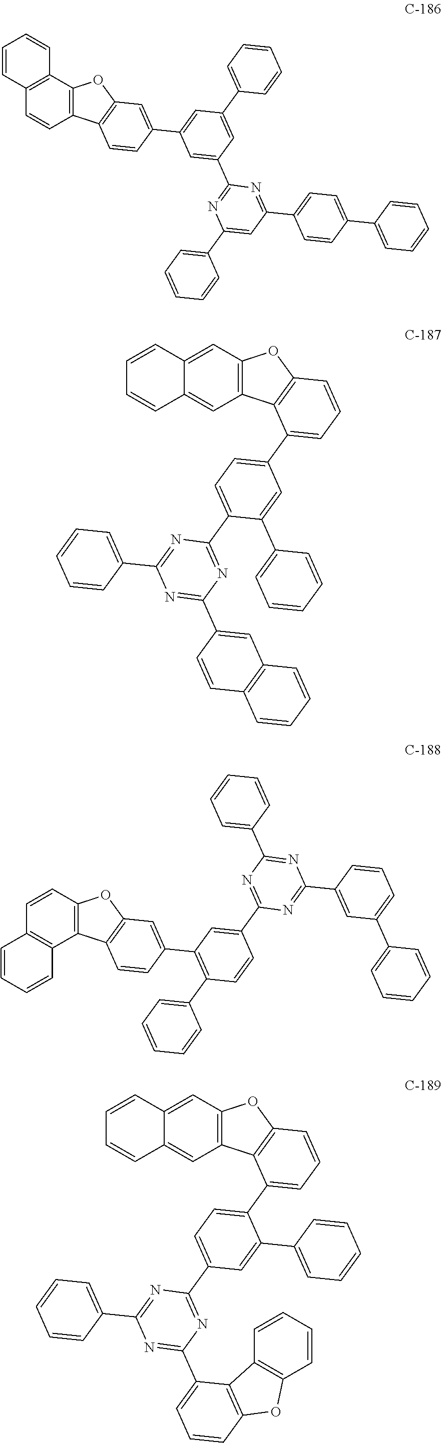

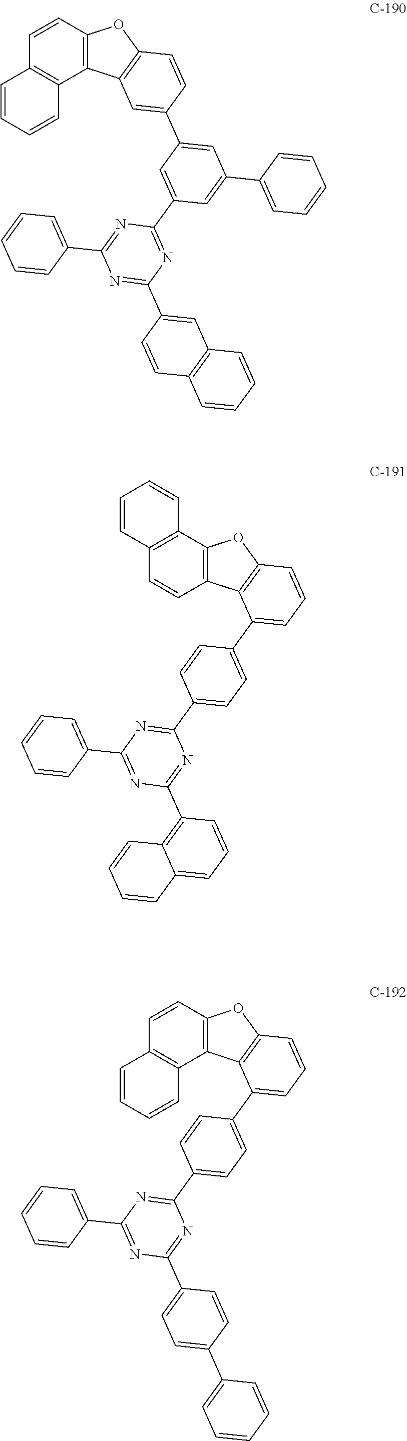 [SMILES: c1ccc(-c2cc(-c3ccc4oc5ccc6ccccc6c5c4c3)cc(-c3nc(-c4ccccc4)nc(-c4ccc5ccccc5c4)n3)c2)cc1 ; c1ccc(-c2nc(-c3ccc(-c4cccc5oc6c7ccccc7ccc6c45)cc3)nc(-c3cccc4ccccc34)n2)cc1 ; c1ccc(-c2ccc(-c3nc(-c4ccccc4)nc(-c4ccc(-c5cccc6oc7ccc8ccccc8c7c56)cc4)n3)cc2)cc1]

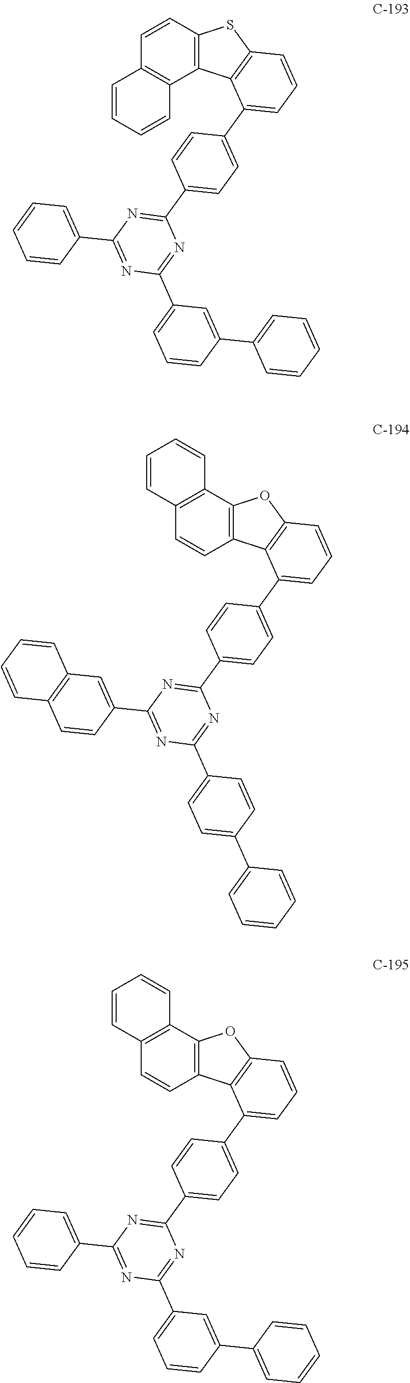 [SMILES: c1ccc(-c2cccc(-c3nc(-c4ccccc4)nc(-c4ccc(-c5cccc6sc7ccc8ccccc8c7c56)cc4)n3)c2)cc1 ; c1ccc(-c2ccc(-c3nc(-c4ccc(-c5cccc6oc7c8ccccc8ccc7c56)cc4)nc(-c4ccc5ccccc5c4)n3)cc2)cc1 ; c1ccc(-c2cccc(-c3nc(-c4ccccc4)nc(-c4ccc(-c5cccc6oc7c8ccccc8ccc7c56)cc4)n3)c2)cc1]

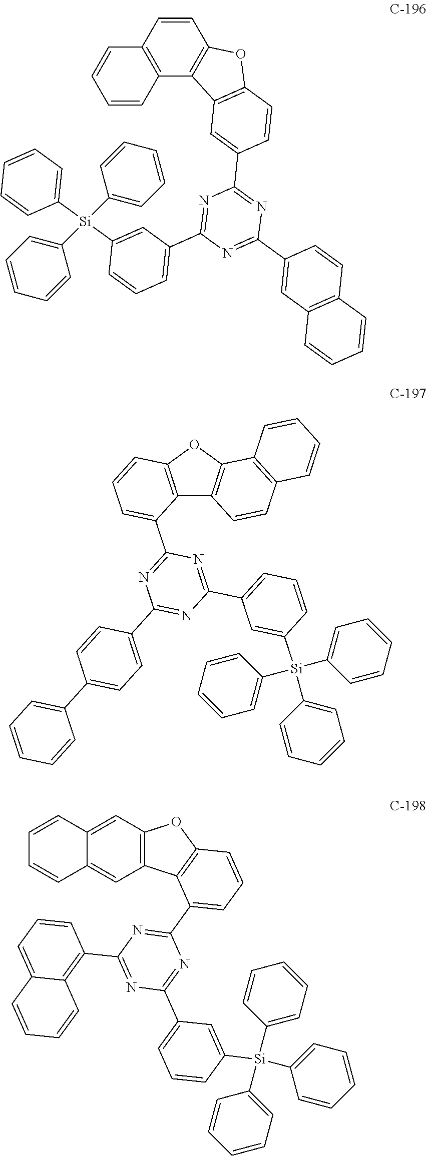 [SMILES: c1ccc([Si](c2ccccc2)(c2ccccc2)c2cccc(-c3nc(-c4ccc5ccccc5c4)nc(-c4ccc5oc6ccc7ccccc7c6c5c4)n3)c2)cc1 ; c1ccc(-c2ccc(-c3nc(-c4cccc([Si](c5ccccc5)(c5ccccc5)c5ccccc5)c4)nc(-c4cccc5oc6c7ccccc7ccc6c45)n3)cc2)cc1 ; c1ccc([Si](c2ccccc2)(c2ccccc2)c2cccc(-c3nc(-c4cccc5ccccc45)nc(-c4cccc5oc6cc7ccccc7cc6c45)n3)c2)cc1]

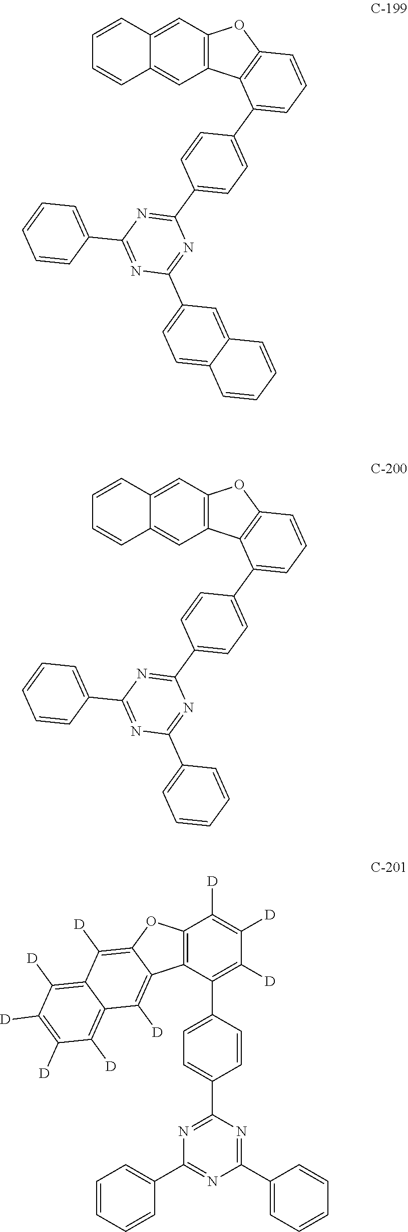 [SMILES: c1ccc(-c2nc(-c3ccc(-c4cccc5oc6cc7ccccc7cc6c45)cc3)nc(-c3ccc4ccccc4c3)n2)cc1 ; c1ccc(-c2nc(-c3ccccc3)nc(-c3ccc(-c4cccc5oc6cc7ccccc7cc6c45)cc3)n2)cc1 ; [2H]c1c([2H])c(-c2ccc(-c3nc(-c4ccccc4)nc(-c4ccccc4)n3)cc2)c2c(oc3c([2H])c4c([2H])c([2H])c([2H])c([2H])c4c([2H])c32)c1[2H]]

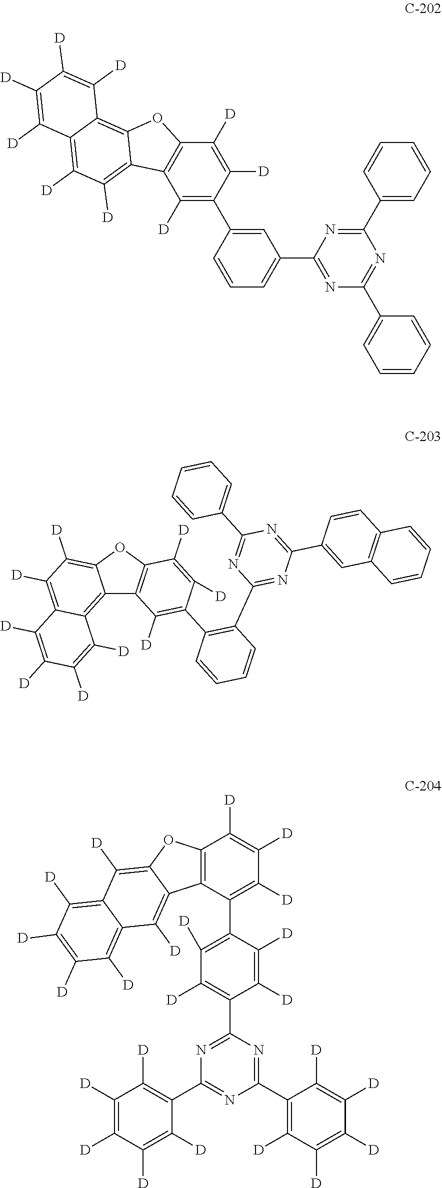 [SMILES: [2H]c1c(-c2cccc(-c3nc(-c4ccccc4)nc(-c4ccccc4)n3)c2)c([2H])c2c(oc3c4c([2H])c([2H])c([2H])c([2H])c4c([2H])c([2H])c32)c1[2H] ; [2H]c1c(-c2ccccc2-c2nc(-c3ccccc3)nc(-c3ccc4ccccc4c3)n2)c([2H])c2c(oc3c([2H])c([2H])c4c([2H])c([2H])c([2H])c([2H])c4c32)c1[2H] ; [2H]c1c([2H])c([2H])c(-c2nc(-c3c([2H])c([2H])c([2H])c([2H])c3[2H])nc(-c3c([2H])c([2H])c(-c4c([2H])c([2H])c([2H])c5oc6c([2H])c7c([2H])c([2H])c([2H])c([2H])c7c([2H])c6c45)c([2H])c3[2H])n2)c([2H])c1[2H]]

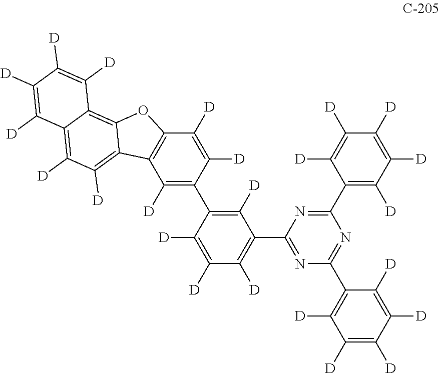 [SMILES: [2H]c1c([2H])c([2H])c(-c2nc(-c3c([2H])c([2H])c([2H])c([2H])c3[2H])nc(-c3c([2H])c([2H])c([2H])c(-c4c([2H])c([2H])c5oc6c7c([2H])c([2H])c([2H])c([2H])c7c([2H])c([2H])c6c5c4[2H])c3[2H])n2)c([2H])c1[2H]]

The combination of at least one of compounds H-1 to H-305 and at least one of compounds C-1 to C-205 may be used in an organic electroluminescent device.

The compound represented by formula 1 according to the present disclosure may be produced by a synthetic method known to one skilled in the art, and for example, by referring to the following reaction scheme 1, but is not limited thereto.

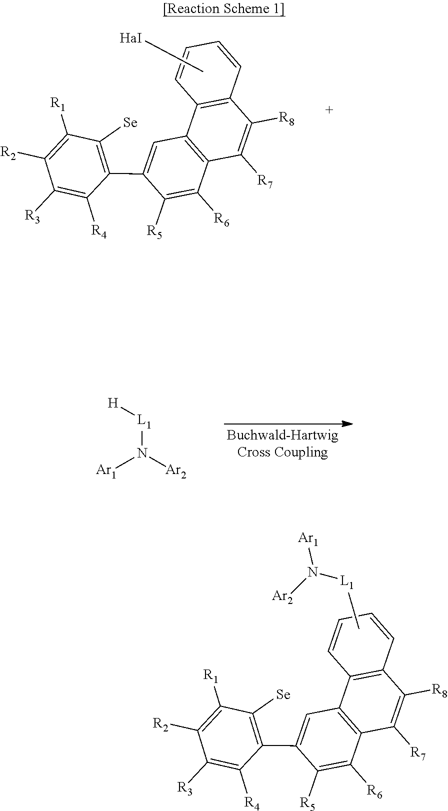

In reaction scheme 1, R₁ to R₈, L₁, Ar₁, and Ar₂ are as defined in formula 1, and Hal represents a halogen.

Although illustrative synthesis examples of the compounds represented by formula 1 of the present disclosure are described above, one skilled in the art will be able to readily understand that all of them are based on a Buchwald-Hartwig cross-coupling reaction, an N-arylation reaction, a H-mont-mediated etherification reaction, a Miyaura borylation reaction, a Suzuki cross-coupling reaction, an Intramolecular acid-induced cyclization reaction, a Pd(II)-catalyzed oxidative cyclization reaction, a Grignard reaction, a Heck reaction, a Cyclic Dehydration reaction, an SN₁ substitution reaction, an SN₂ substitution reaction, and a Phosphine-mediated reductive cyclization reaction, etc., and the reactions above proceed even when substituents which are defined in formula 1 above, but are not specified in the specific synthesis examples, are bonded.

The compound represented by formula 2 according to the present disclosure may be produced by a synthetic method known to one skilled in the art.

The organic electroluminescent device according to the present disclosure may comprise an anode, a cathode, and at least one organic layer between the anode and cathode, in which the organic layer may comprise a plurality of organic electroluminescent materials, including the compound represented by formula 1 as the first organic electroluminescent material, and the compound represented by formula 2 as the second organic electroluminescent material. According to one embodiment of the present disclosure, the organic electroluminescent device according to the present disclosure may comprise an anode, a cathode, and at least one light-emitting layer between the anode and cathode, in which the light-emitting layer may comprise the compound represented by formula 1 and the compound represented by formula 2.

The light-emitting layer includes a host and a dopant, in which the host includes a plurality of host materials, and the compound represented by formula 1 may be included as the first host compound of the plurality of host materials, and the compound represented by formula 2 may be included as the second host compound of the plurality of host materials. The weight ratio of the first host compound and the second host compound is about 1:99 to about 99:1, preferably about 10:90 to about 90:10, more preferably about 30:70 to about 70:30, even more preferably about 40:60 to about 60:40, and still more preferably about 50:50.

In the present disclosure, the light-emitting layer is a layer from which light is emitted, and may be a single layer or a mufti-layer of which two or more layers are stacked. All of the first host material and the second host material may be included in one layer, or the first host material and the second host material may be included in respective different light-emitting layers. According to one embodiment of the present disclosure, the doping concentration of the dopant compound with respect to the host compound in the light-emitting layer may be less than 20 wt %.

The organic electroluminescent device of the present disclosure may further comprise at least one layer selected from a hole injection layer, a hole transport layer, a hole auxiliary layer, a light-emitting auxiliary layer, an electron transport layer, an electron injection layer, an interlayer, an electron buffer layer, a hole blocking layer, and an electron blocking layer. According to one embodiment of the present disclosure, the organic electroluminescent device of the present disclosure may further comprise an amine-based compound as at least one of a hole injection material, a hole transport material, a hole auxiliary material, a light-emitting material, a light-emitting auxiliary material, and an electron blocking material, besides the plurality of host materials of the present disclosure. In addition, according to another embodiment, the organic electroluminescent device of the present disclosure may further comprise an azine-based compound as at least one of an electron transport material, an electron injection material, an electron buffer material, and a hole blocking material, besides the plurality of host materials of the present disclosure.

The plurality of host materials according to the present disclosure may be used as a light-emitting material for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. The plurality of host materials according to the present disclosure may also be used in an organic electroluminescent device comprising a quantum dot (QD).

A hole injection layer, a hole transport layer, an electron blocking layer, or a combination thereof can be used between the anode and the light-emitting layer. The hole injection layer may be multi-layers in order to lower the hole injection barrier (or hole injection voltage) from the anode to the hole transport layer or the electron blocking layer, wherein each of the multi-layers may use two compounds simultaneously. In addition, the hole injection layer may be doped with a p-dopant. Further, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. The hole transport layer or the electron blocking layer may also be multi-layers, wherein each of the multi-layers may use a plurality of compounds.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. The hole blocking layer or the electron transport layer may also be multi-layers, wherein each of the multi-layers may use a plurality of compounds. In addition, the electron injection layer may be doped with an n-dopant.

The dopant comprised in the organic electroluminescent device of the present disclosure may be at least one phosphorescent or fluorescent dopant, and is preferably a phosphorescent dopant. The phosphorescent dopant material applied to the organic electroluminescent device of the present disclosure is not particularly limited, but may be a complex compounds of a metal atom selected from iridium (Ir), osmium (Os), copper (Cu) and platinum (Pt), and preferably ortho-metallated complex compounds of a metal atom selected from iridium (Ir), osmium (Os), copper (Cu), and platinum (Pt), and more preferably ortho-metallated iridium complex compounds.

The dopant comprised in the organic electroluminescent device of the present disclosure may comprise a compound represented by the following formula 101, but is not limited thereto.

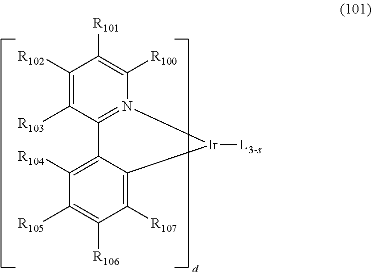

In formula 101,

L is selected from the following structures 1 to 3:

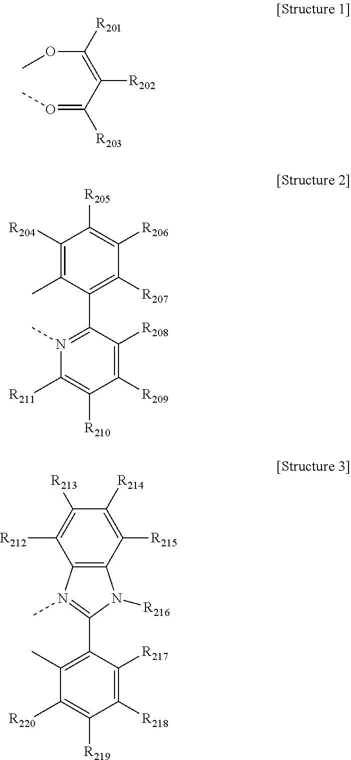

R₁₀₀ to R₁₀₃, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a cyano, a substituted or unsubstituted (3- to 30-membered)heteroaryl, or a substituted or unsubstituted (C1-C30)alkoxy; or may be linked to an adjacent substituent(s) to form a ring(s), e.g., a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;

R₁₀₄ to R₁₀₇, each independently, represent hydrogen, deuterium, a halogen, a (C1-C30)alkyl unsubstituted or substituted with deuterium and/or a halogen(s), a substituted or unsubstituted (C3-C30)cycloalkyl, a substituted or unsubstituted (C6-C30)aryl, a substituted or unsubstituted (3- to 30-membered)heteroaryl, a cyano, or a substituted or unsubstituted (C1-C30)alkoxy; or may be linked to an adjacent substituent(s) to form a ring(s), e.g., a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;

R₂₀₁ to R₂₂₀, each independently, represent hydrogen, deuterium, a halogen, a (C1-C30)alkyl unsubstituted or substituted with deuterium and/or a halogen(s), a substituted or unsubstituted (C3-C30)cycloalkyl, or a substituted or unsubstituted (C6-C30)aryl; or may be linked to an adjacent substituent(s) to form a substituted or unsubstituted ring(s); and

s represents an integer of 1 to 3.

The specific examples of the dopant compound are as follows, but are not limited thereto.

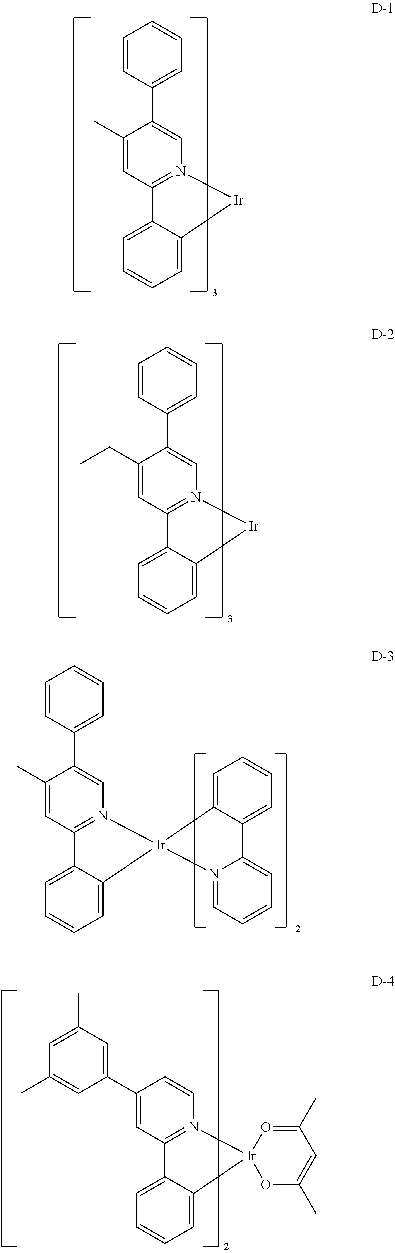

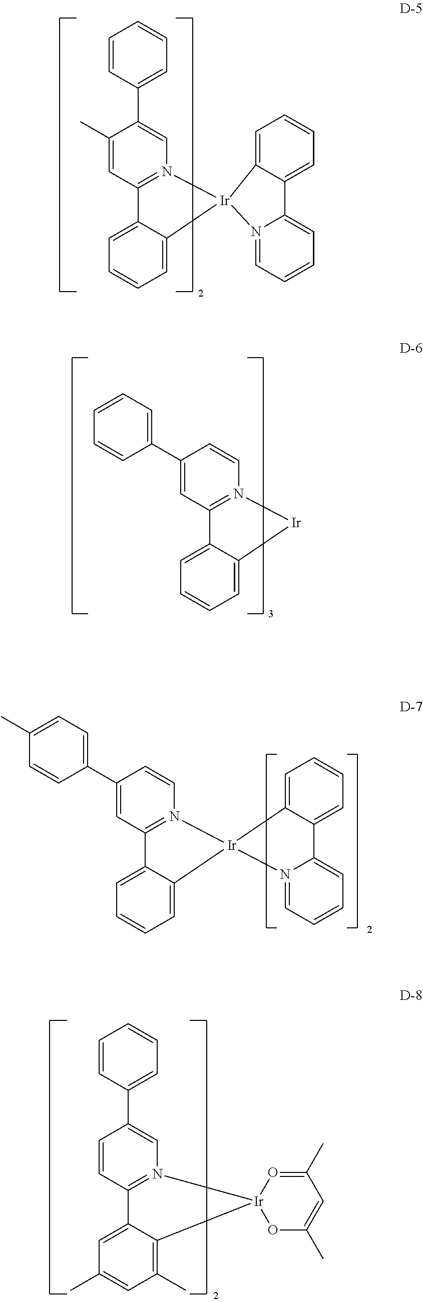

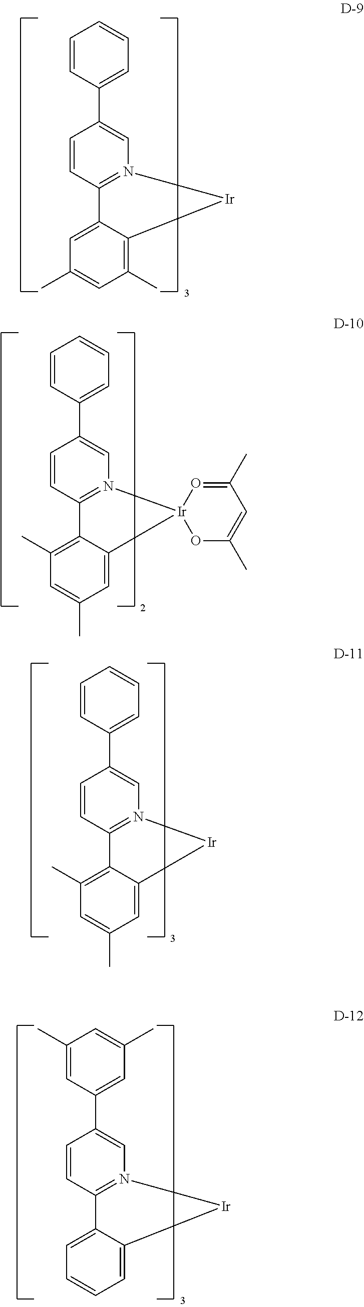

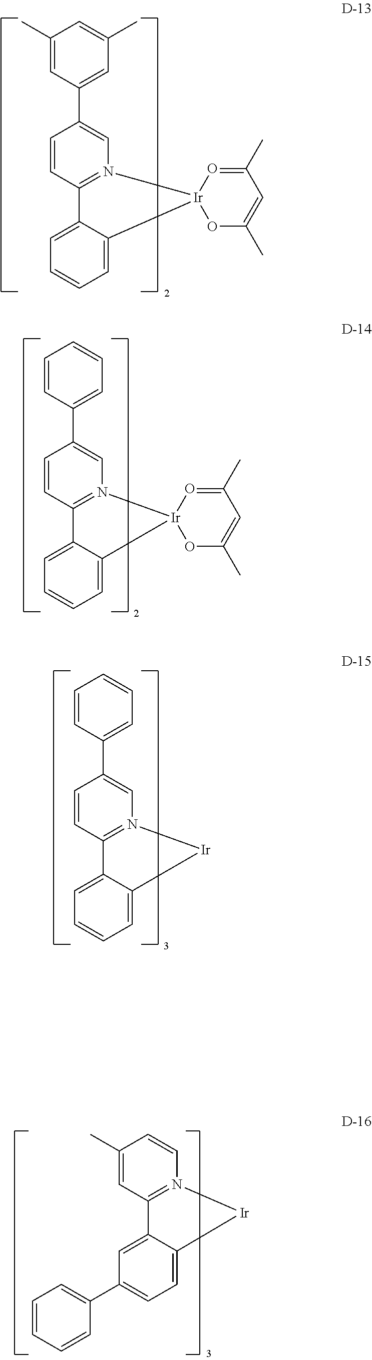

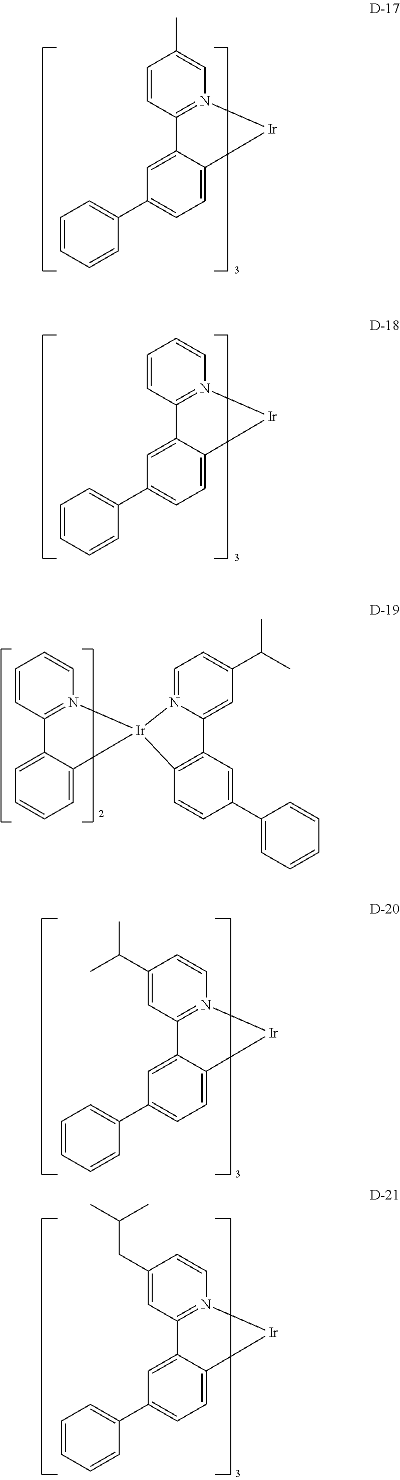

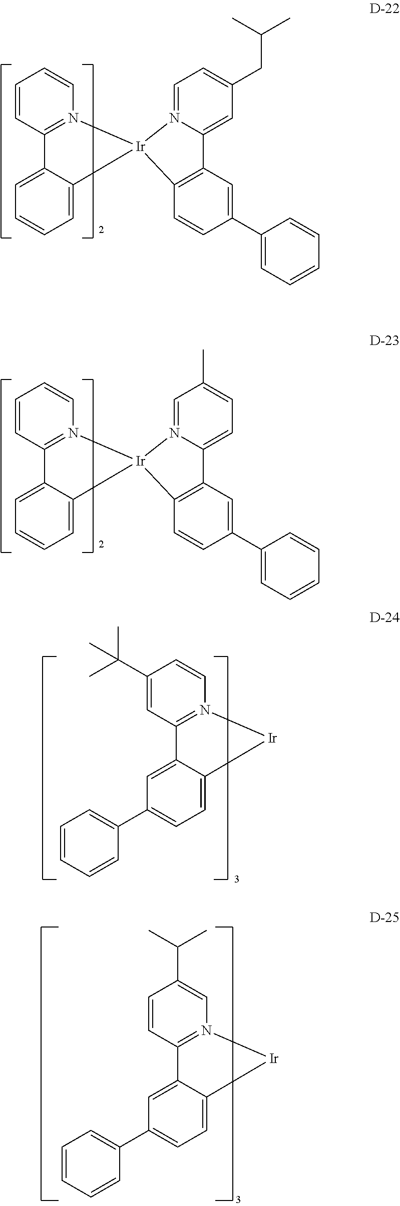

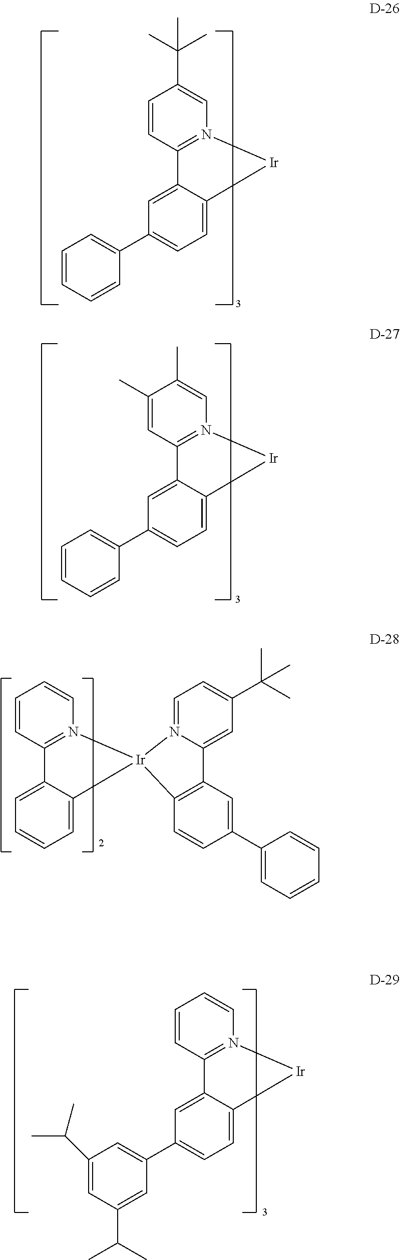

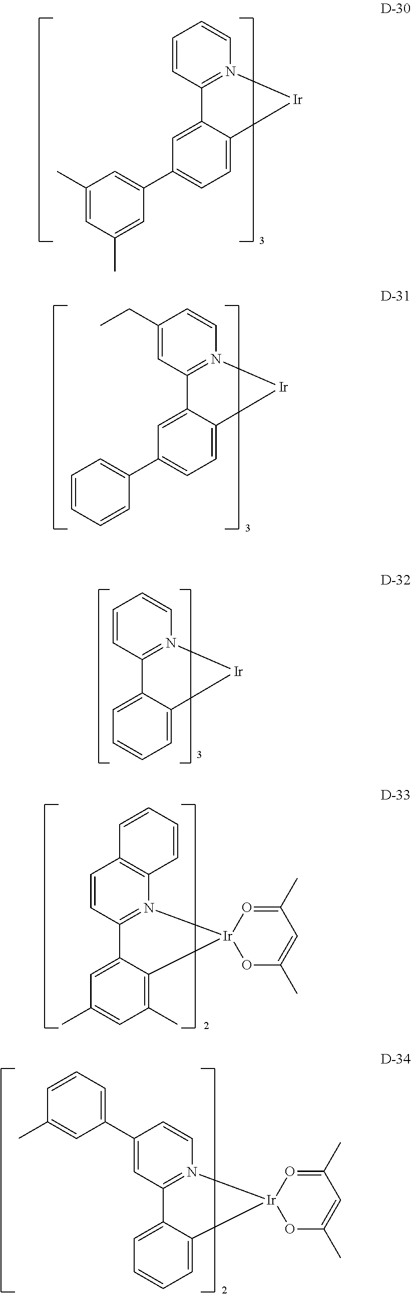

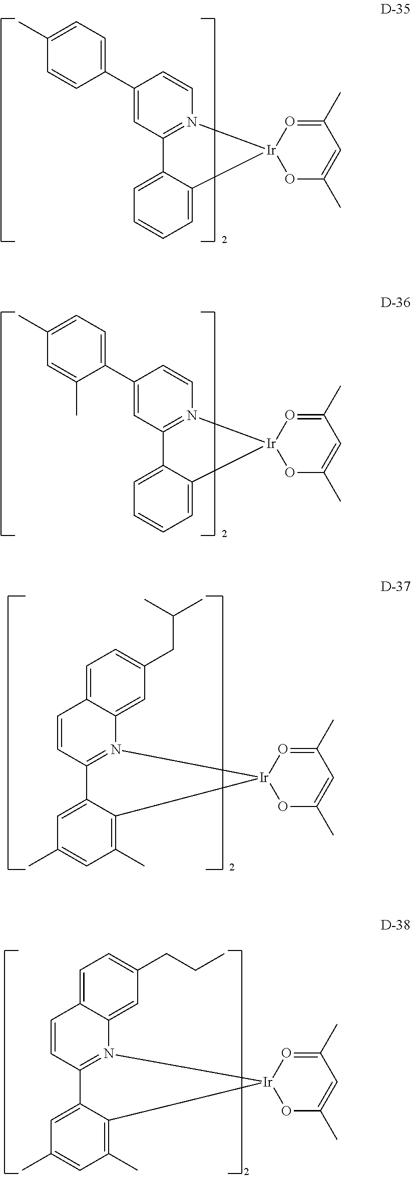

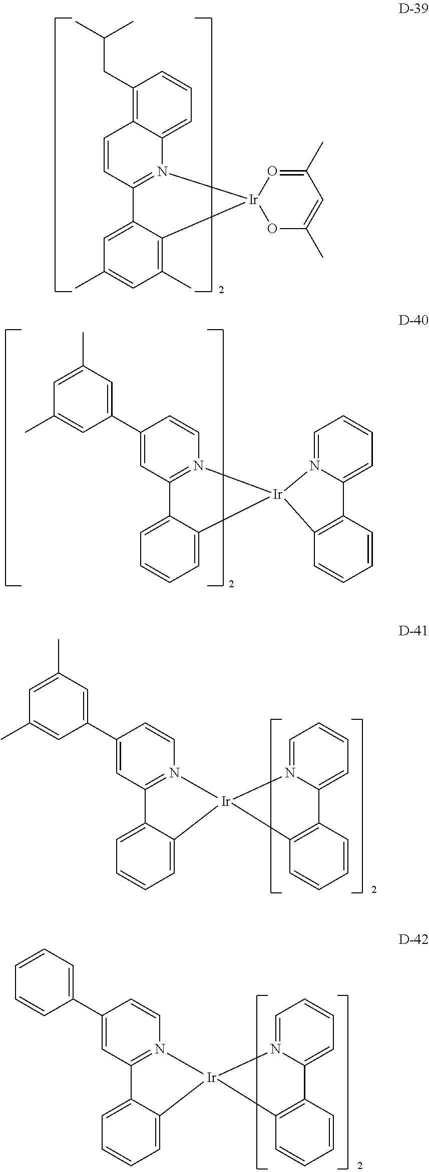

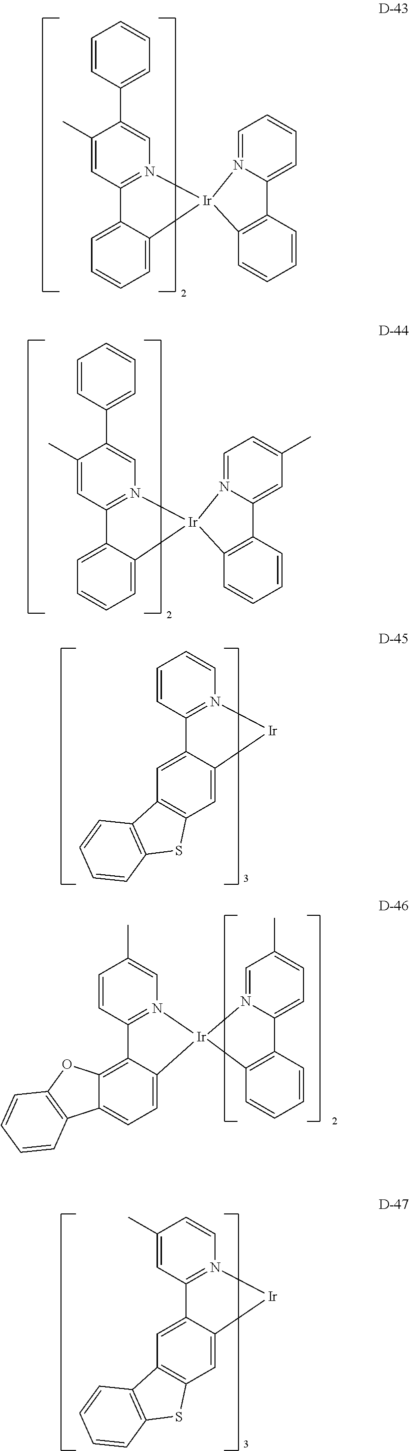

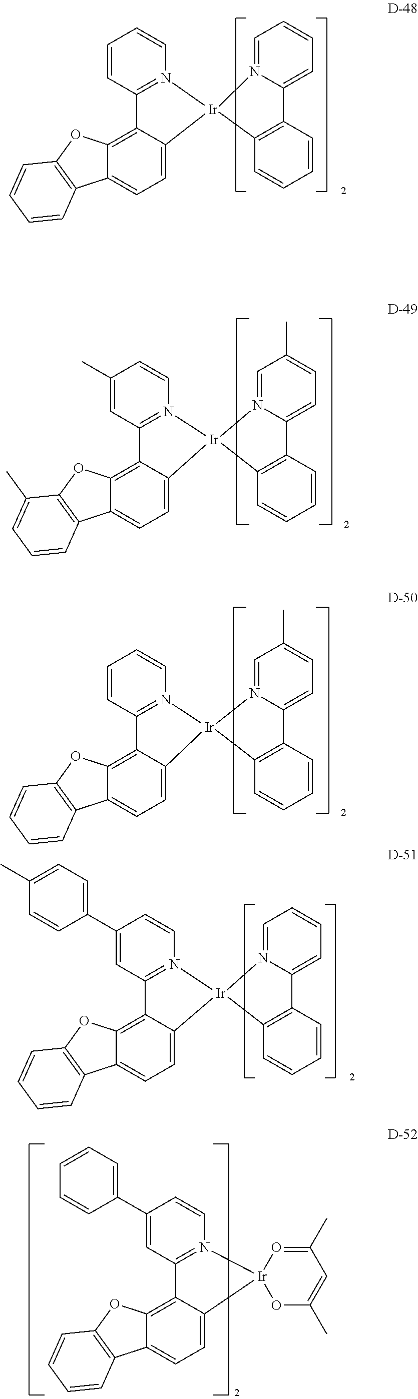

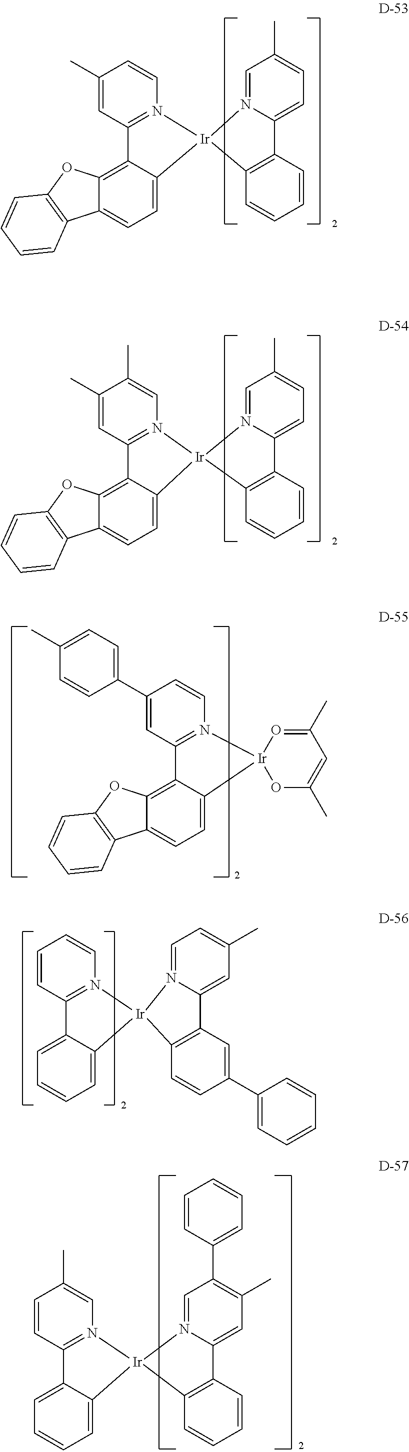

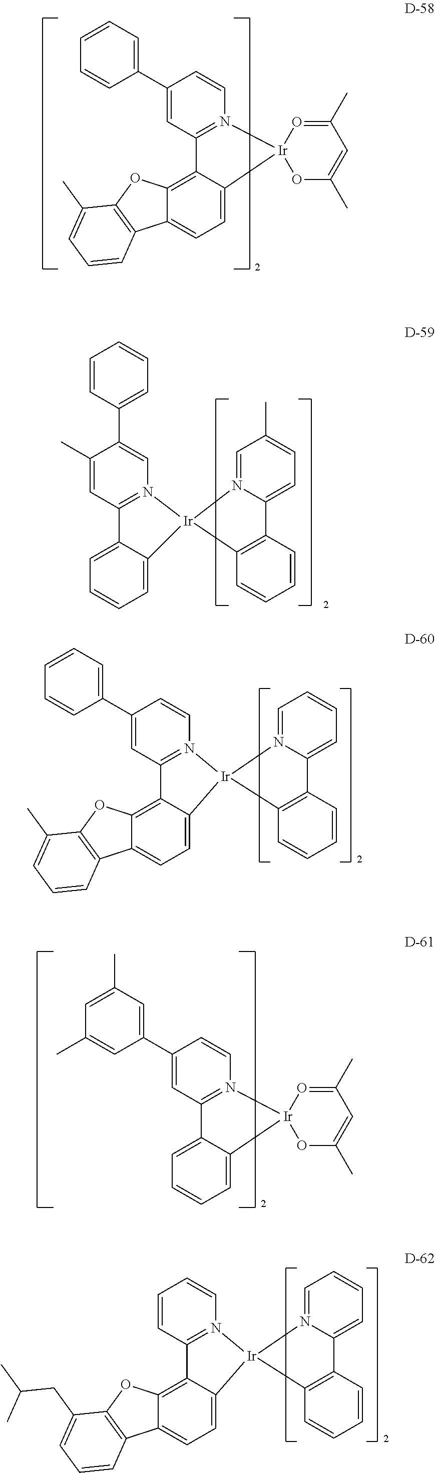

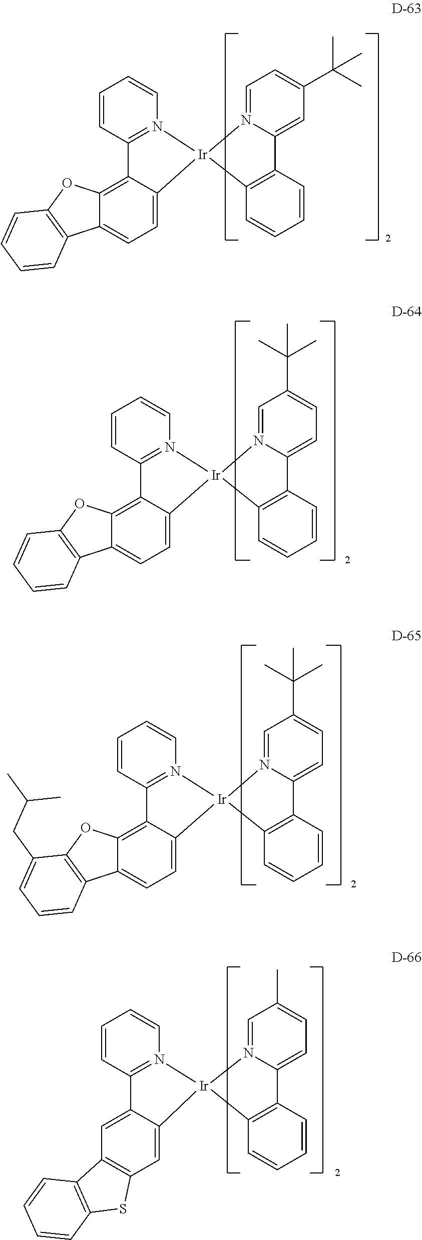

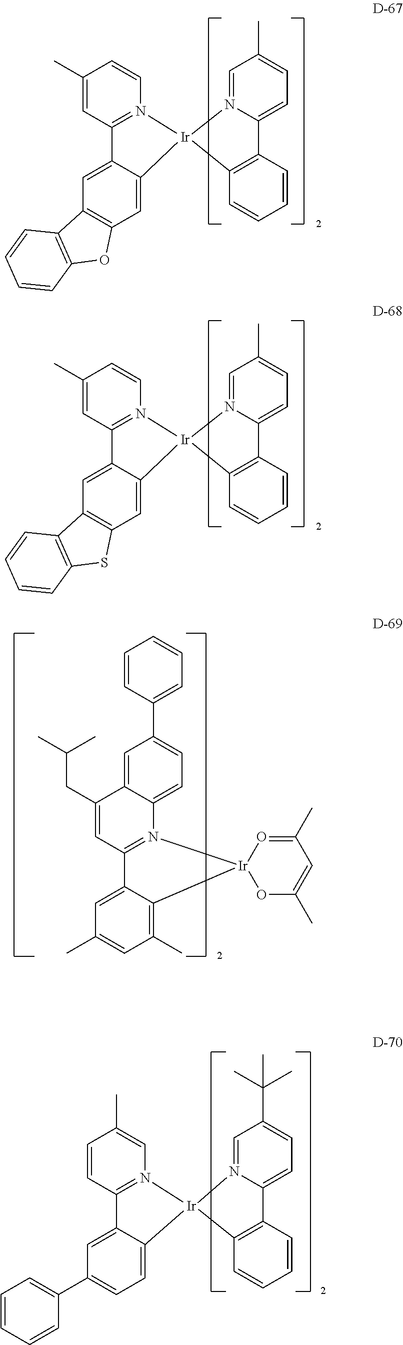

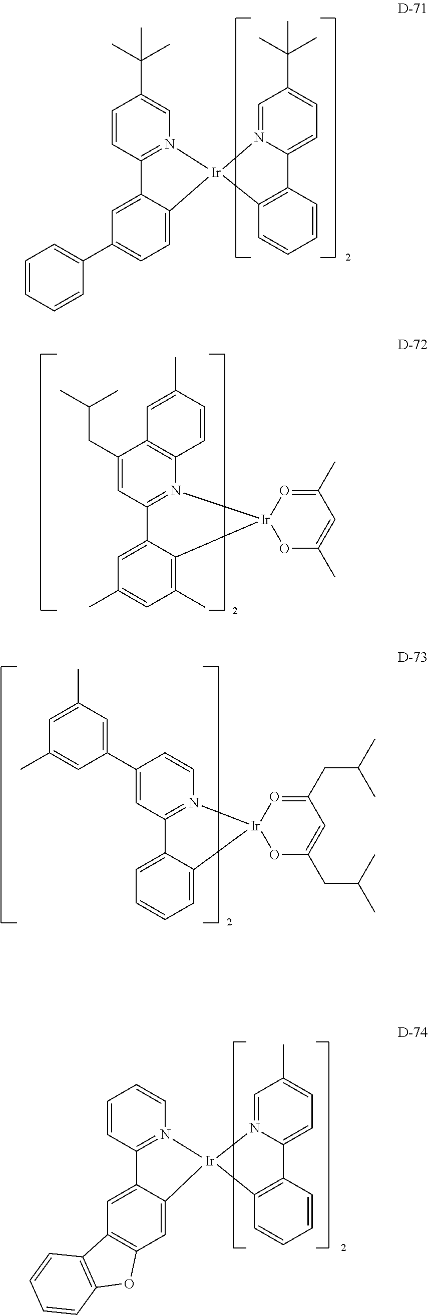

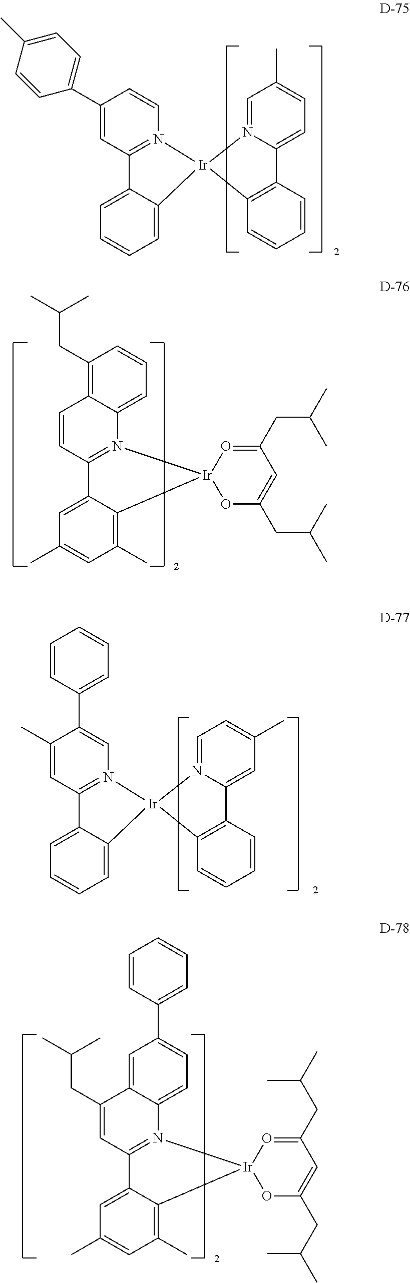

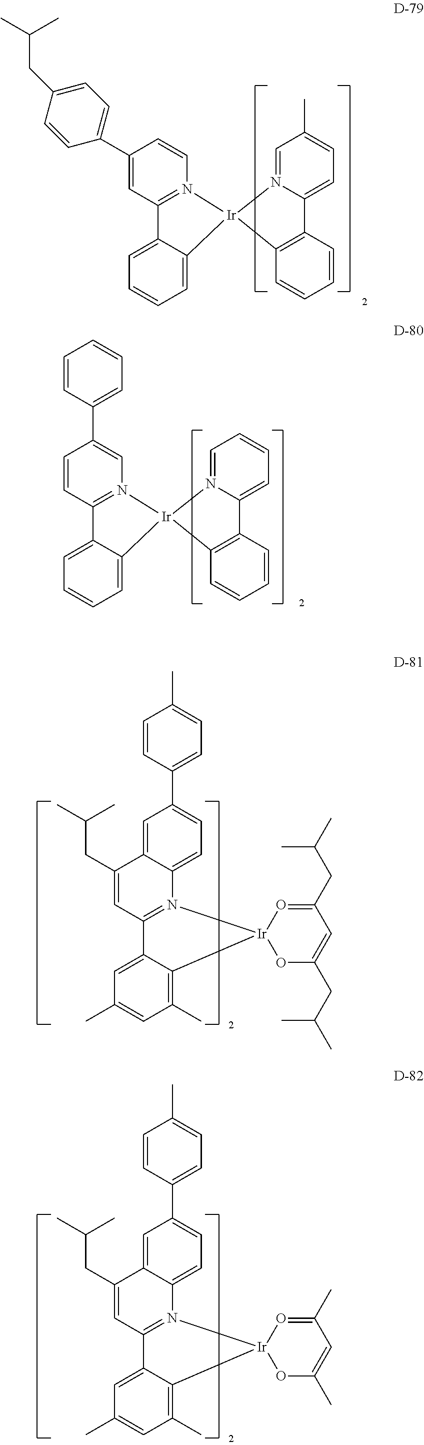

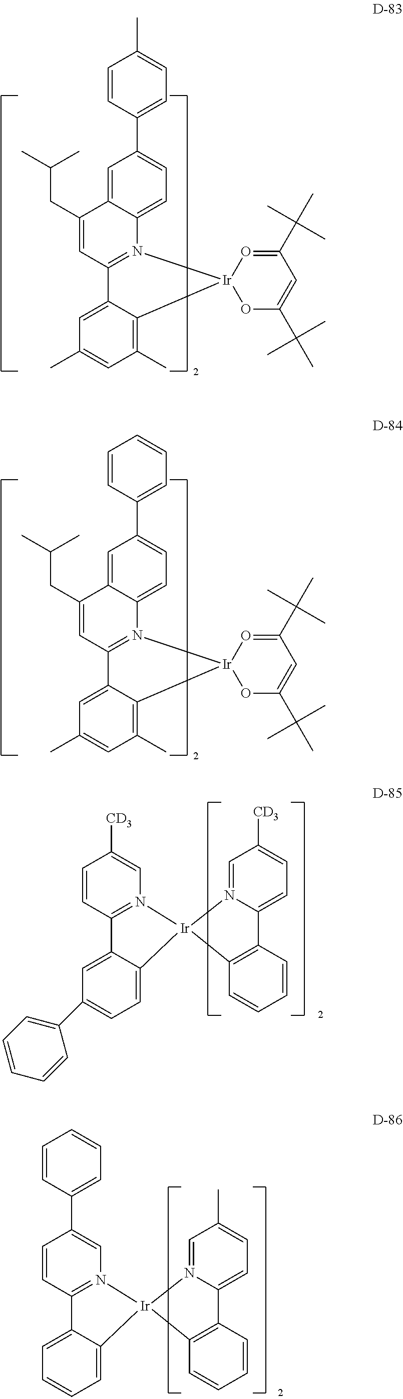

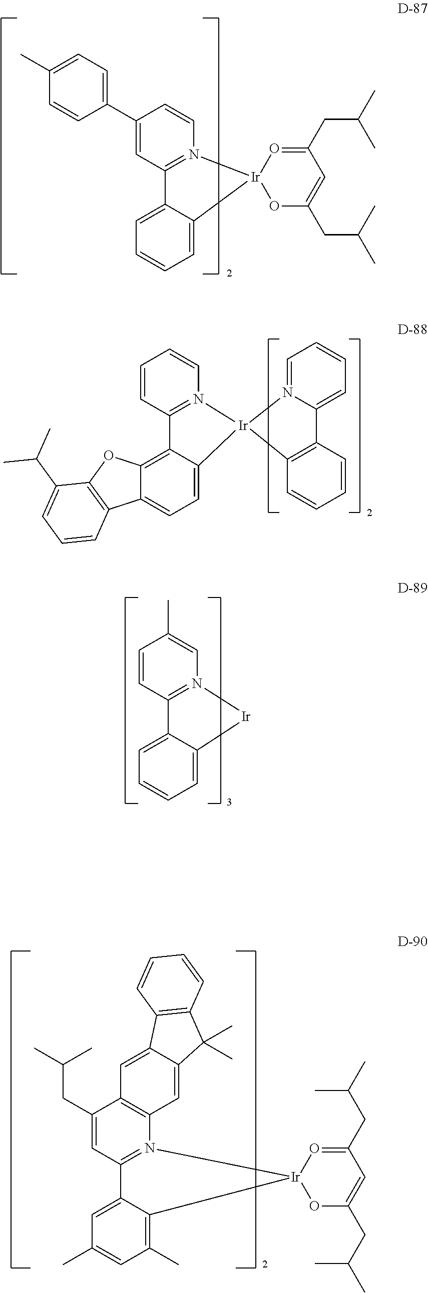

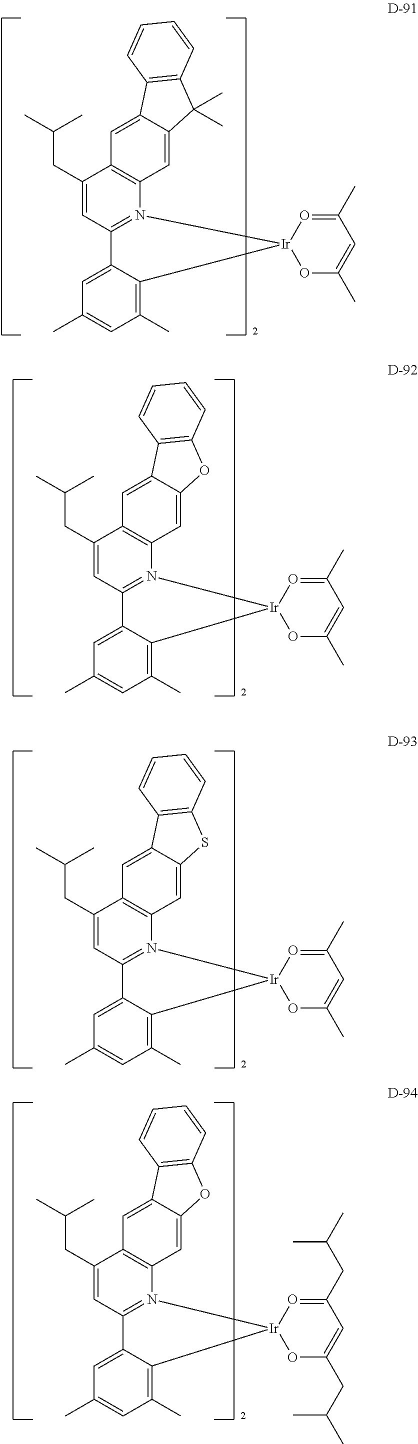

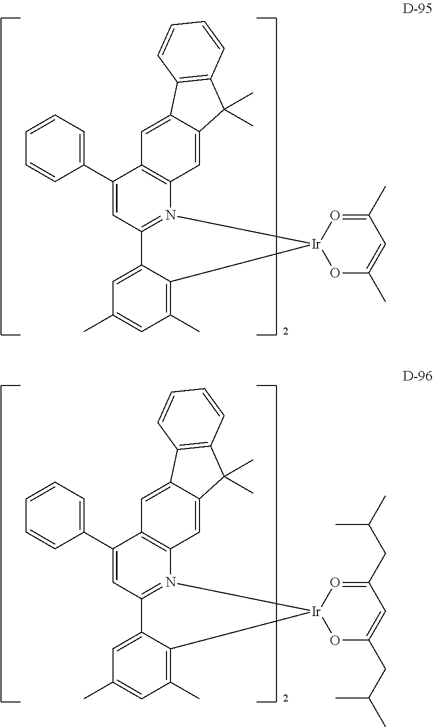

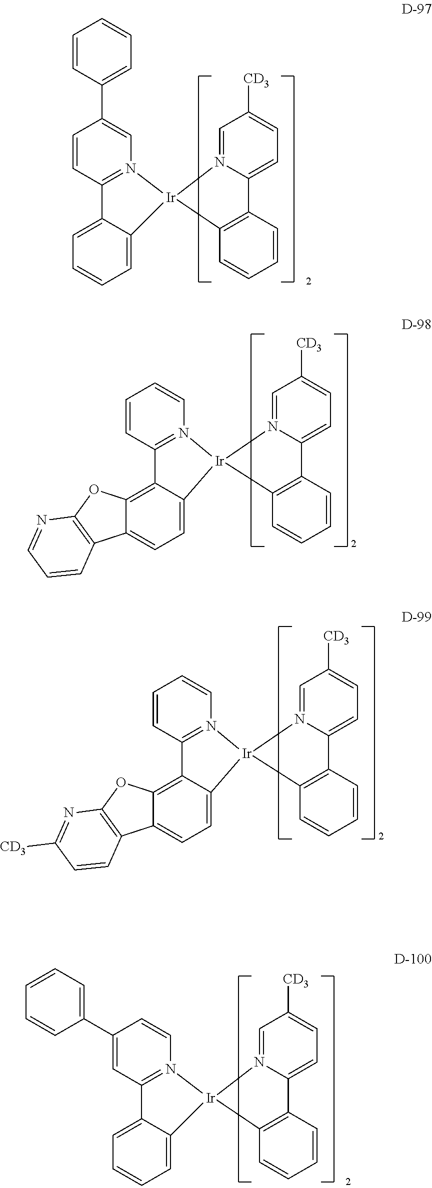

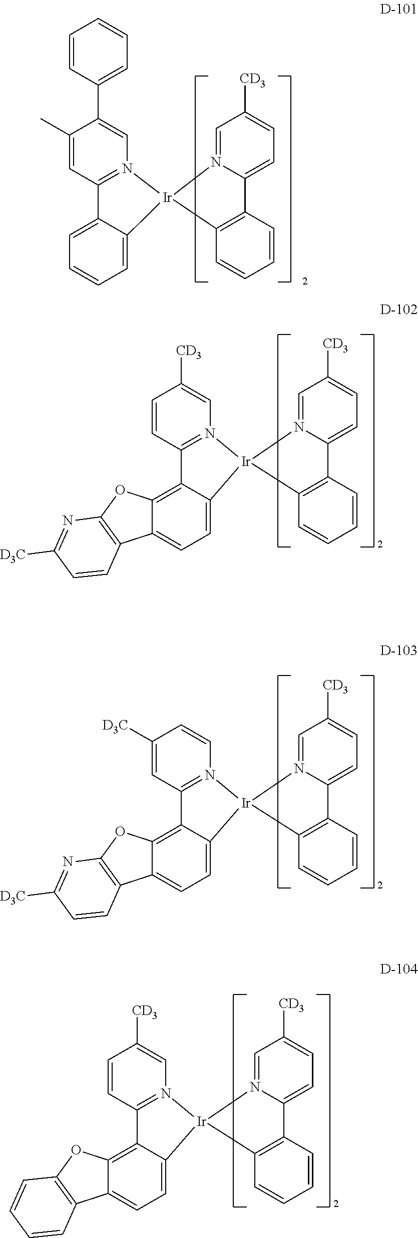

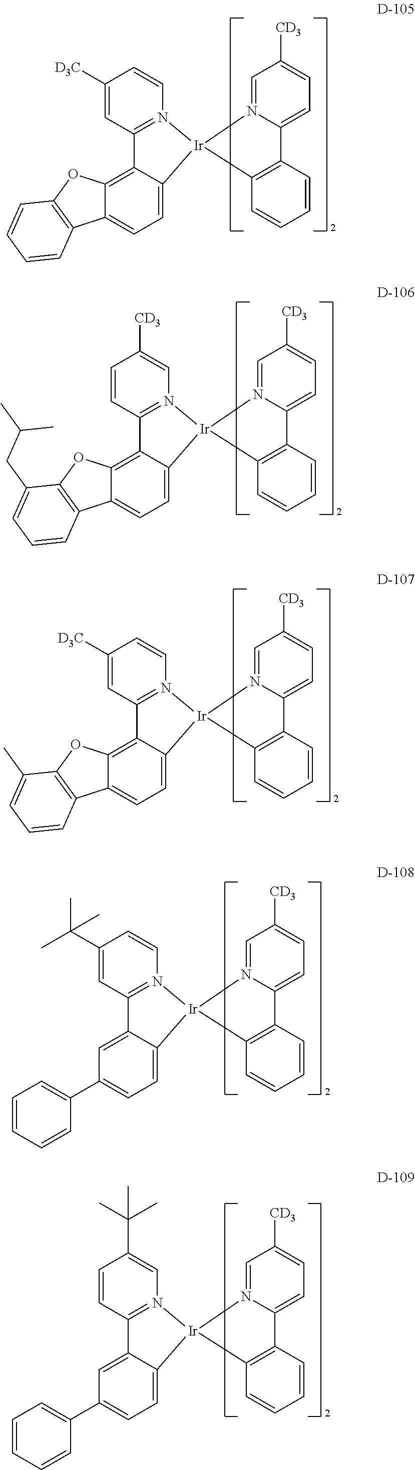

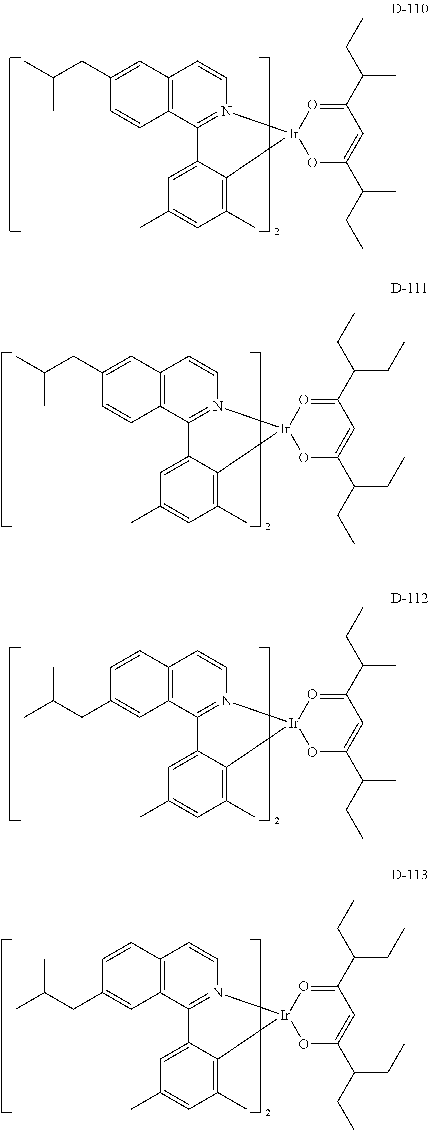

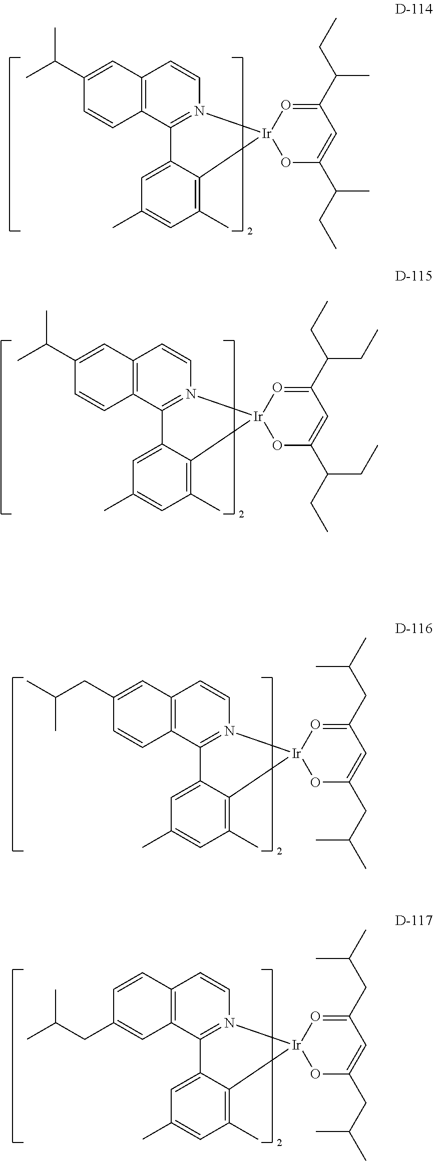

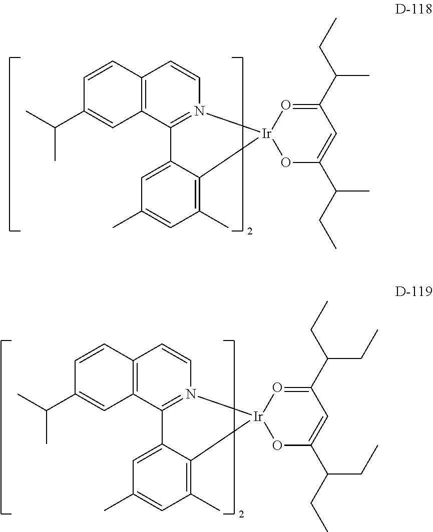

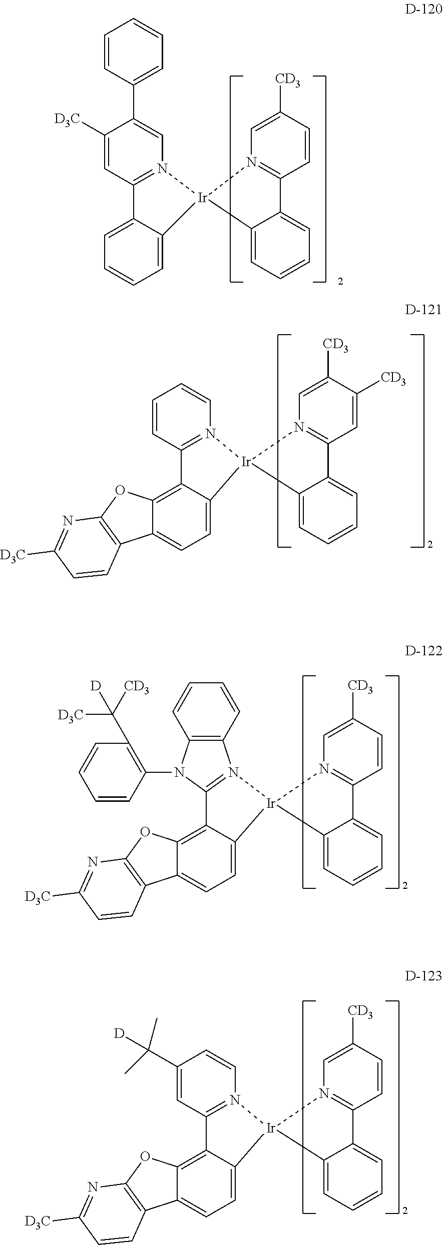

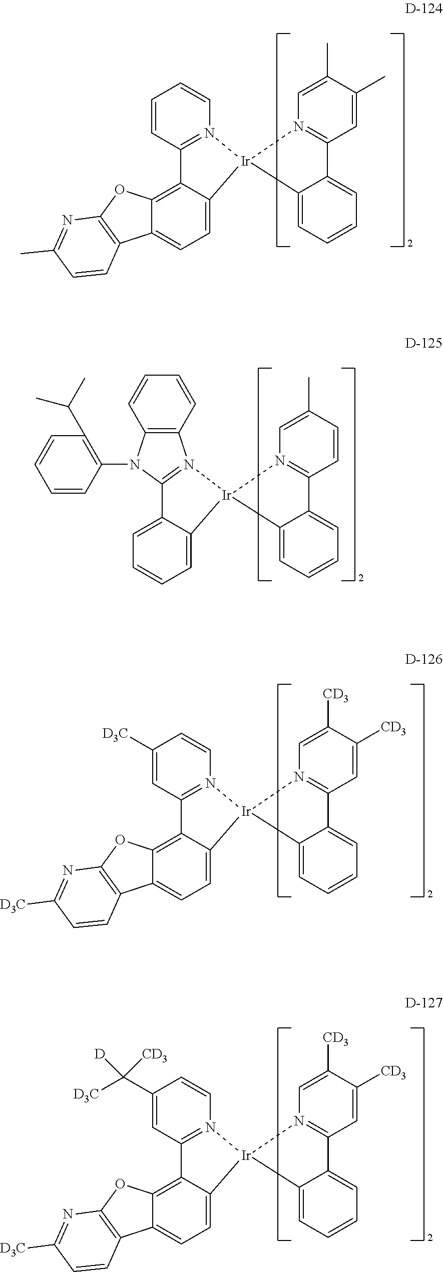

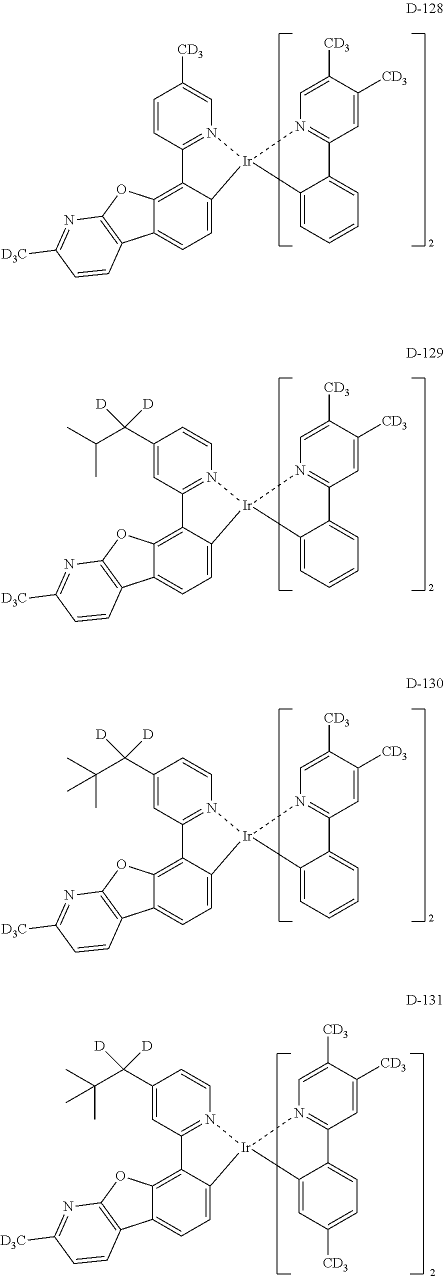

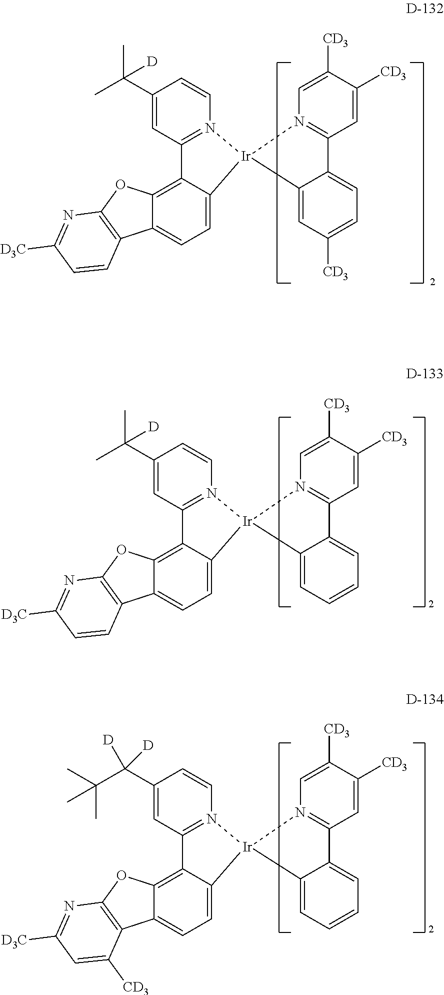

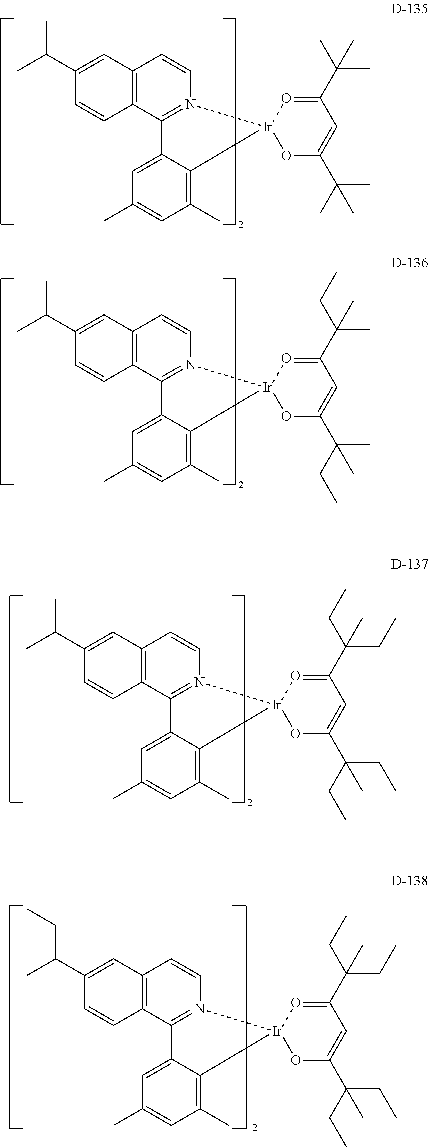

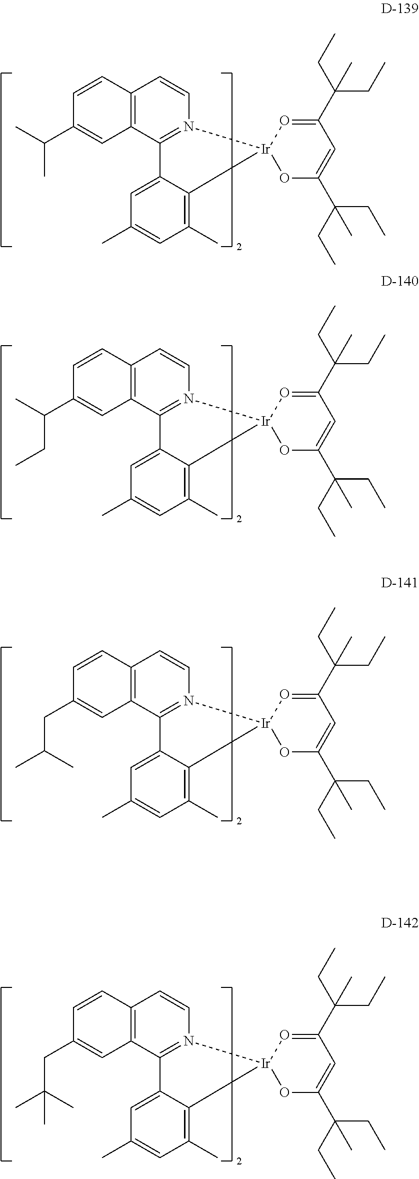

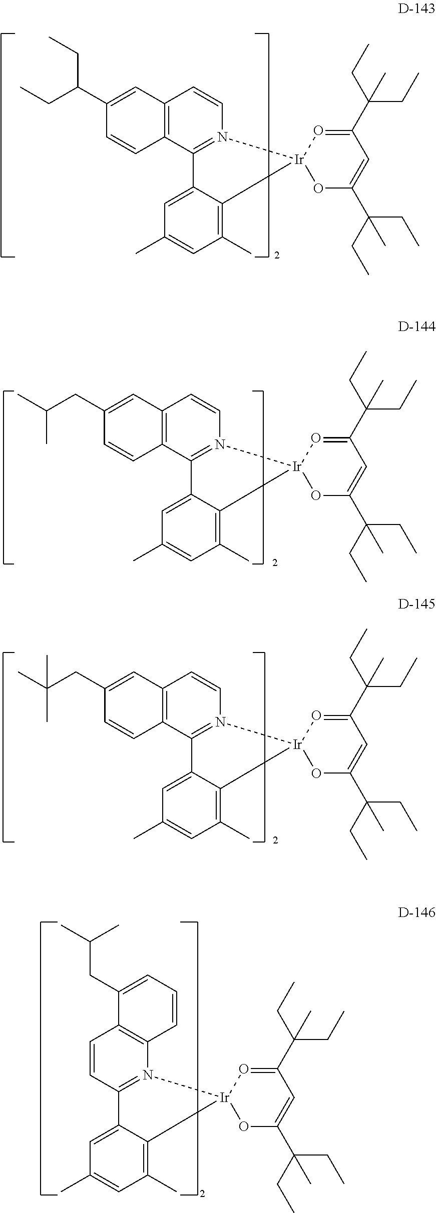

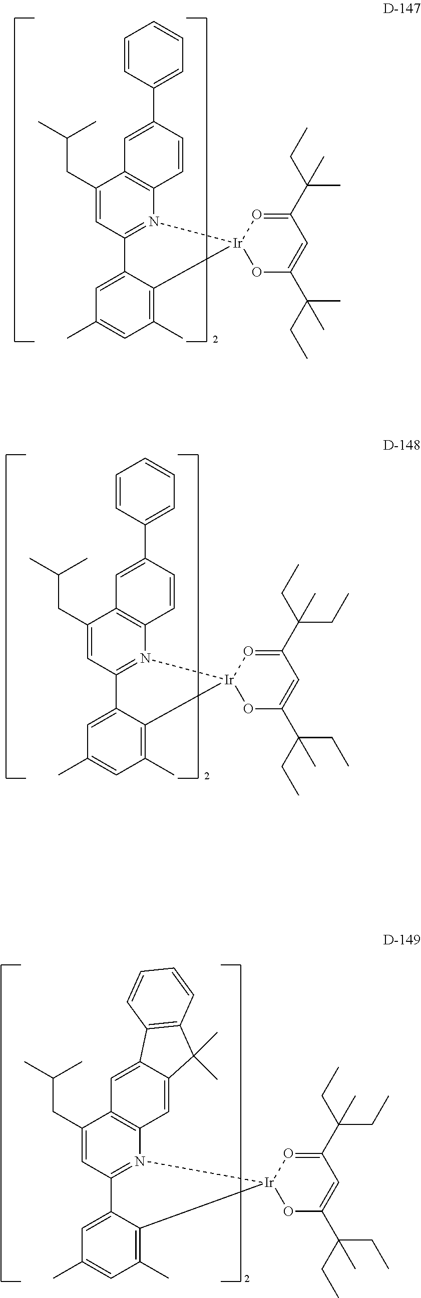

In order to form each layer of the organic electroluminescent device of the present disclosure, dry film-forming methods such as vacuum evaporation, sputtering, plasma, ion plating methods, etc., or wet film-forming methods such as ink jet printing, nozzle printing, slot coating, spin coating, dip coating, flow coating methods, etc., can be used.

When using a wet film-forming method, a thin film can be formed by dissolving or diffusing materials forming each layer into any suitable solvent such as ethanol, chloroform, tetrahydrofuran, dioxane, etc. The solvent can be any one where the materials forming each layer can be dissolved or diffused, and where there are no problems in film-formation capability.

In addition, the first and the second host compounds of the present disclosure may be film-formed by the above-listed methods, commonly by a co-evaporation process or a mixture-evaporation process. The co-evaporation is a mixed deposition method in which two or more materials are placed in a respective individual crucible source and a current is applied to both cells at the same time to evaporate the materials. The mixture-evaporation is a mixed deposition method in which two or more materials are mixed in one crucible source before depositing them, and a current is applied to one cell to evaporate the materials. In addition, if the first and the second host compounds are present in the same layer or different layers in an organic electroluminescent device, the two host compounds may individually form films. For example, the second host compound may be deposited after depositing the first host compound.

The present disclosure may provide a display device by using the plurality of host materials including the compound represented by formula 1 and the compound represented by formula 2. That is, it is possible to produce a display system or a lighting system by using the plurality of host materials of the present disclosure. Specifically, a display system, for example, a display system for white organic light emitting devices, smart phones, tablets, notebooks. PCs, TVs, or cars; or a lighting system, for example, an outdoor or indoor lighting system, can be produced by using the plurality of host materials of the present disclosure.

Hereinafter, the preparation method of the compounds according to the present disclosure and the properties thereof, and the properties of the organic electroluminescent device (OLED) comprising the plurality of host materials of the present disclosure will be explained in detail with reference to the representative compounds of the present disclosure.

However, the following examples are only to describe the characteristics of the OLED comprising the compound according to the present disclosure and the plurality of host materials according to the present disclosure for a detailed understanding of the present disclosure, but the present disclosure is not limited to the following examples.

### Example 1: Preparation of Compound H-47

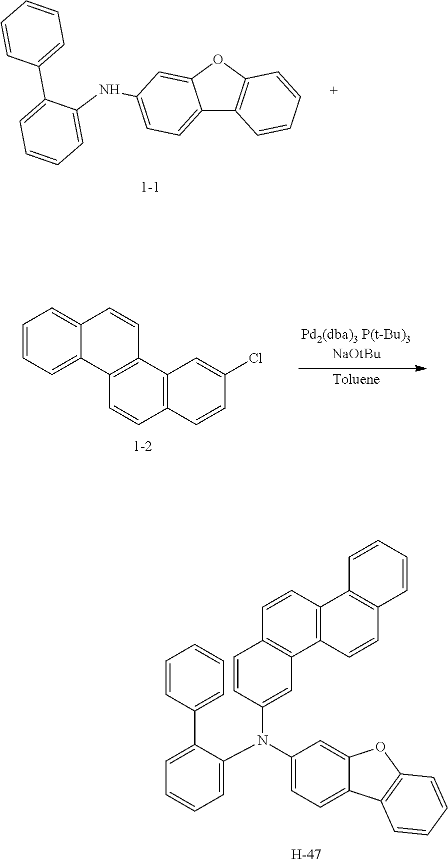

Compound 1-1 (5.0 g, 14.91 mmol), compound 1-2 (4.3 g, 16.40 mmol), Pd₂(dba)₃ (0.7 g, 0.75 mmol), P(t-Bu)₃ (0.3 g/0.5 mL, 1.49 mmol, 0.82 density), and NaOtBu (2.1 g, 22.37 mmol) were dissolved in 74 mL of toluene and stirred under reflux for 3 hours. After completion of the reaction, the mixture was cooled to room temperature, layer-separated with MC/H₂O, and magnesium sulfate was added. The resulting product was separated through a celite filter and recrystallized with toluene to obtain compound H-47 (5.4 g, yield: Compound MW M. P.

| Compound | MW | M.P. |
|---|---|---|
| H-47 | 561.68 | 244° C. |

### Example 2: Preparation of Compound H-152

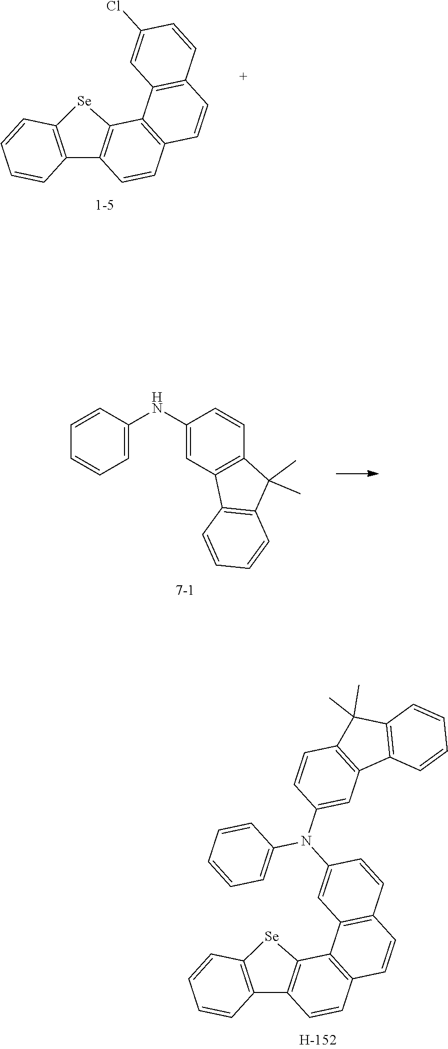 [SMILES: Clc1ccc2ccc3ccc4c5ccccc5[se]c4c3c2c1 ; CC1(C)c2ccccc2-c2cc(Nc3ccccc3)ccc21 ; CC1(C)c2ccccc2-c2cc(N(c3ccccc3)c3ccc4ccc5ccc6c7ccccc7[se]c6c5c4c3)ccc21]

In a flask, compound 1-5 (5 g, 13.67 mmol), compound 7-1 (3.9 g, 13.81 mmol), tris(dibenzylideneacetone)dipalladium (0.62 g, 0.68 mmol), 2-dichlorohexylphosphine-2′,6′-dimethoxybiphenyl (0.56 g, 1.36 mmol), sodium tert-butoxide (3.3 g, 34.1 mmol), and 70 mL of toluene were added, dissolved, and stirred under reflux for 4 hours. After completion of the reaction, the mixture was cooled to room temperature, filtered with silica, dried, and separated by column chromatography to obtain compound H-152 (8.2 g, yield: 97%).

### Example 3: Preparation of Compound C-140

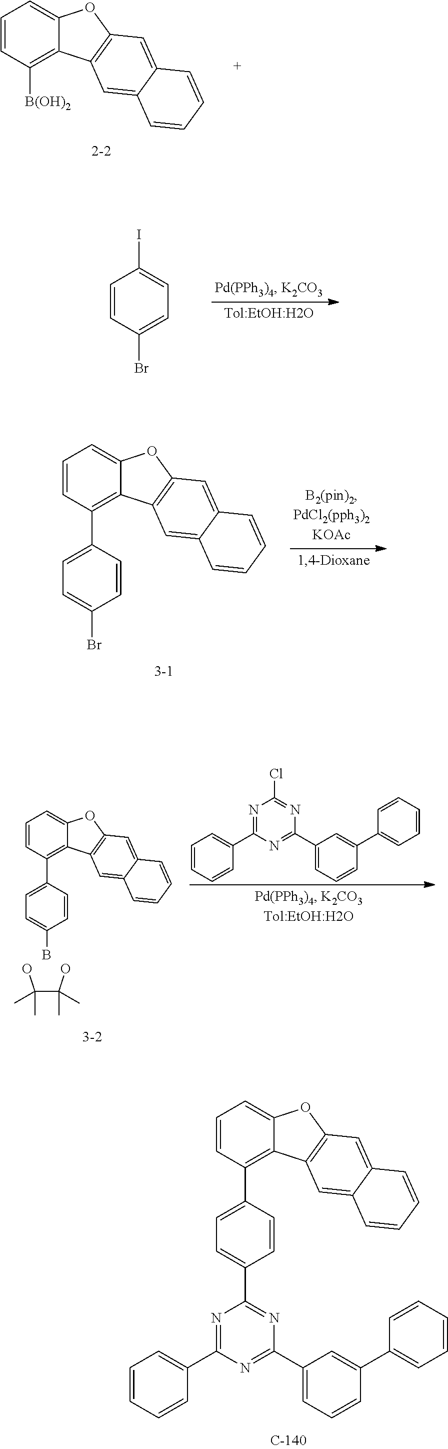

Synthesis of Compound 3-1

In a flask, compound 2-2 (30 g, 114.4 mmol), 1-bromo-4-iodobenzene (39 g, 137.3 mmol), Pd(PPh)₄ (6.6 g, 5.723 mmol), K₂CO₃ (47.4 g, 343.4) mmol), 680 mL of toluene, 170 mL of ethanol, and 170 mL of distilled water were added, and stirred under reflux for 3 hours. After completion of the reaction, the mixture was cooled to room temperature, and extracted with distilled water and ethyl acetate. After the organic layer was distilled under reduced pressure, the solid was filtered to obtain compound 3-1 (33.5 g, yield: 78%).

Synthesis of Compound 3-2

In a flask, compound 3-1 (33.5 g, 89.76 mmol), B₂(pin)₂ (30 g, 116.6 mmol), PdCl₂(pph₃)₂ (6.3 g, 8.976 mmol), KOAc (17.6 g, 179.5 mmol), and 450 mL of 1,4-dioxane were added, and stirred under reflux for 4 hours. After completion of the reaction, the mixture was separated through a celite filter, and separated by column chromatography using MC/Hex to obtain compound 3-2 (31 g, yield: 82%).

Synthesis of Compound C-140

In a flask, compound 3-2 (5 g, 11.89 mmol), 2-([1,1′-biphenyl]-3-yl)-4-chloro-6-phenyl-1,3,5-triazine (4.1 g, 11.89 mmol), Pd(PPh₃)₄ (0.68 g, 0.594 mmol), K₂CO₃ (4.9 g, 35.68 mmol), 80 mL of toluene, 20 mL of ethanol, and 20 mL of distilled water were added, and stirred under reflux for 2 hours. After completion of the reaction, the mixture was cooled to room temperature, and extracted with distilled water and ethyl acetate. The organic layer was distilled under reduced pressure and filtered through silica to obtain compound C-140 (1.9 g, yield: 26%).

### Example 4: Preparation of Compound C-199

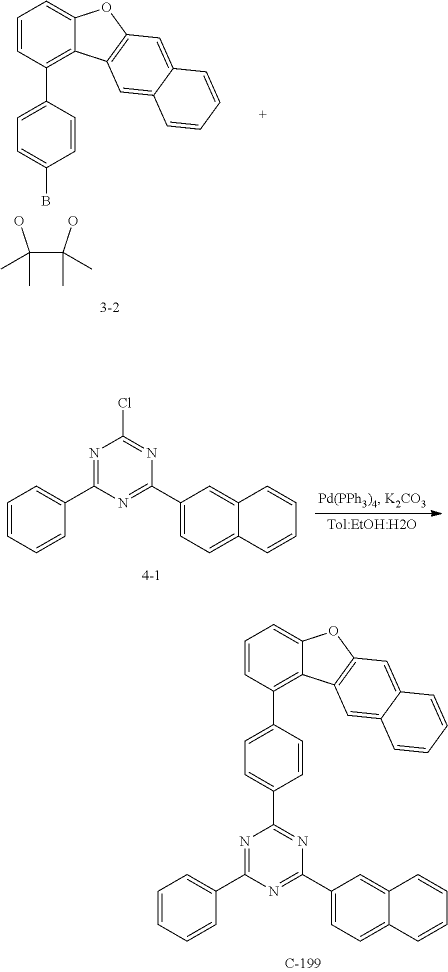

In a flask, compound 3-2 (5 g, 11.89 mmol), compound 4-1 (3.8 g, 11.89 mmol), Pd(PPh₃)₄ (0.68 g, 0.594 mmol), K₂CO₃ (4.9 g, 35.68 mmol), 80 mL of toluene, 20 mL of ethanol, and 20 mL of distilled water were added, and stirred under reflux for 2 hours. After completion of the reaction, the mixture was cooled to room temperature, and extracted with distilled water and ethyl acetate. The organic layer was distilled under reduced pressure and filtered through silica to obtain compound C-199 (3.1 g, yield: 41%).

| Compound | MW | M.P. |
|---|---|---|
| C-199 | 575.6 | 284.2° C. |

### Example 5: Preparation of Compound C-137

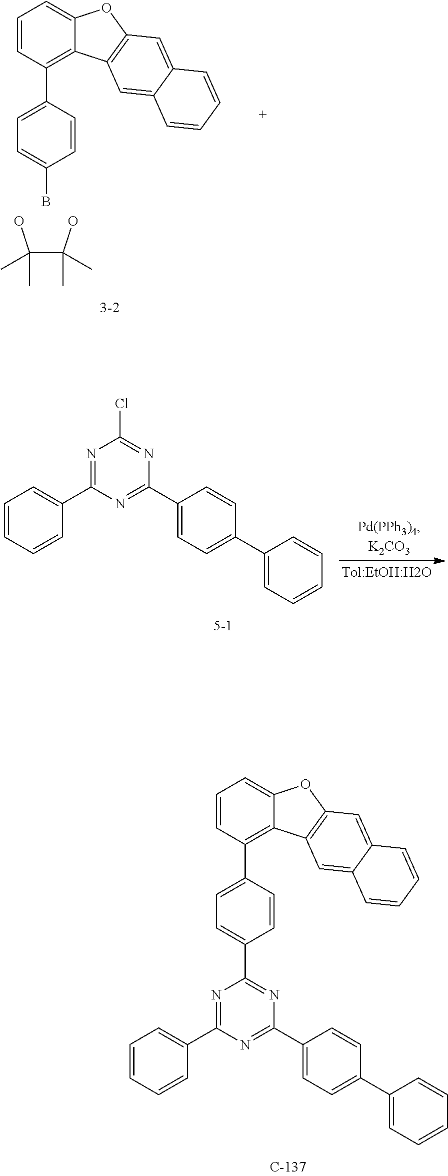

In a flask, compound 3-2 (5 g, 11.89 mmol), compound 5-1 (4.1 g, 11.89 mmol), Pd(PPh₃)₄ (0.68 g, 0.594 mmol), K₂CO₃ (4.9 g, 35.68 mmol), 80 mL toluene, 20 mL of ethanol, and 20 mL of distilled water were added, and stirred under reflux for 2 hours. After completion of the reaction, the mixture was cooled to room temperature, and extracted with distilled water and ethyl acetate. The organic layer was distilled under reduced pressure and filtered through silica to obtain compound C-137 (4 g, yield: 55%).

| Compound | MW | M.P. |
|---|---|---|
| C-137 | 601.6 | 243.7° C. |

### Example 6: Preparation of Compound C-200

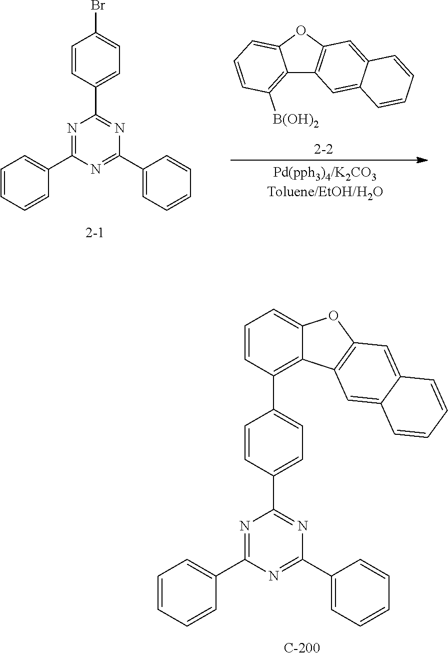 [SMILES: Brc1ccc(-c2nc(-c3ccccc3)nc(-c3ccccc3)n2)cc1 ; OB(O)c1cccc2oc3cc4ccccc4cc3c12 ; c1ccc(-c2nc(-c3ccccc3)nc(-c3ccc(-c4cccc5oc6cc7ccccc7cc6c45)cc3)n2)cc1]

In a flask, compound 2-1 (15 g, 38.6 mmol), compound 2-2 (11.3 g, 42.5 mmol), Pd(pph₃)₄ (2.2 g, 1.93 mmol), K₂CO₃ (10.6 g, 77.2 mmol), 200 mL of toluene, 40 mL of ethanol, and 40 mL of distilled water were added, and stirred at 160° C. After completion of the reaction, methanol and water were added thereto, and the mixture was stirred, and the solvent was removed by filtration under reduced pressure. After separation by column chromatography, methanol was added thereto, and the resulting solid was filtered under reduced pressure to obtain compound C-200 as a white solid (14.7 g, yield: 72.7%).

| Compound | MW | M.P. |
|---|---|---|
| C-200 | 525.61 | 255.3° C. |

### Device Examples 1 and 2: Producing OLEDs According to the Present Disclosure

OLEDs according to the present disclosure were produced. A transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was then mounted on a substrate holder of a vacuum vapor deposition apparatus. Compound HI-1 as shown in Table 2 was introduced into a cell of the vacuum vapor deposition apparatus as a first hole injection compound, and compound HT-1 was introduced into another cell of the vacuum vapor deposition apparatus as a second hole injection compound. The two materials were evaporated at different rates, and compound HI-1 was deposited in a doping amount of 3 wt % based on the total amount of compound HI-1 and compound HT-1 to form a hole injection layer having a thickness of 10 nm. Next, compound HT-1 was deposited on the hole injection layer to form a first hole transport layer having a thickness of 80 nm. Next, compound HT-2 was then introduced into another cell of the vacuum vapor deposition apparatus and was evaporated by applying an electric current to the cell, thereby forming a second hole transport layer having a thickness of 60 nm on the first hole transport layer. After forming the hole injection layer and the hole transport layers, a light-emitting layer was formed thereon as follows: each of the first host compound and the second host compound shown in Table 1 below were introduced into two cells of the vacuum vapor deposition apparatus as hosts, and compound D-39 was introduced into another cell as a dopant. The two host materials were evaporated at a ratio of 1:1 and the dopant material was simultaneously evaporated at a different rate, and the dopant was deposited in a doping amount of 3 wt % based on the total amount of the hosts and the dopant to form a light-emitting layer having a thickness of 40 nm on the second hole transport layer. Subsequently, compound ETL-1 and compound EIL-1 were evaporated in a weight ratio of 50:50 to form an electron transport layer having a thickness of 35 nm on the light-emitting layer. After depositing compound EIL-1 as an electron injection layer having a thickness of 2 nm on the electron transport layer, an Al cathode having a thickness of 80 nm was deposited on the electron injection layer by another vacuum vapor deposition apparatus. Thus, OLEDs were produced. All the materials used for producing the OLEDs were purified by vacuum sublimation at 10⁻⁶ torr.

### Comparative Example 1: Producing an OLED Comprising a Comparative Compound as a Host

An OLED was produced in the same manner as in Device Example 1, except that the second host compound shown in Table 1 below was used solely as a host of the light-emitting layer.

### Comparative Example 2: Producing an OLED Comprising a Combination of Hosts not According to the Present Disclosure

An OLED was produced in the same manner as in Device Example 1, except that the host compounds shown in Table 1 below were used as hosts of the light-emitting layer.

The driving voltage, luminous efficiency, and light-emitting color at a luminance of 1,000 nit, and the time taken for luminance to decrease from 100% to 95% at a luminance of 10,000 nit (lifetime; T95) of the OLEDs produced in Device Examples 1 and 2 and Comparative Examples 1 and 2 are provided in Table 1 below.

TABLE 1

| | | | Driving | Luminous | Light- | |
|---|---|---|---|---|---|---|
| | First | Second | Voltage | Efficiency | Emitting | Lifetime |
| | Host | Host | (V) | (cd/A) | Color | (T95, hr) |
| Comparative | — | C-200 | 3.6 | 31.1 | Red | 15 |
| Example 1 | | | | | | |
| Comparative | T-1 | C-200 | 3.2 | 33.2 | Red | 61 |
| Example 2 | | | | | | |
| Device | H-47 | C-200 | 3.5 | 35.1 | Red | 154 |
| Example 1 | | | | | | |
| Device | H-145 | C-200 | 2.9 | 36.7 | Red | 367 |
| Example 2 | | | | | | |

From Table 1 above, it can be seen that the OLEDs comprising a plurality of compounds according to the present disclosure as host materials exhibit high luminous efficiency, and in particular, significantly improved lifetime property, compared to the OLED using a single host material (Comparative Example 1) and the OLED not comprising the specific combination of host materials according to the present disclosure (Comparative Example 2).

TABLE 2

| | |
|---|---|
| Hole Injection Layer/ Hole Transport Layer | 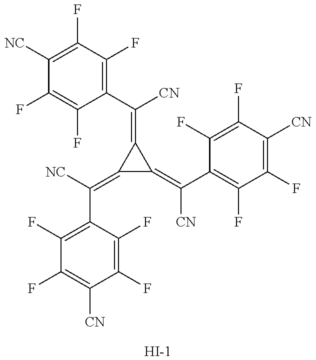 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; [I-]] |
| | 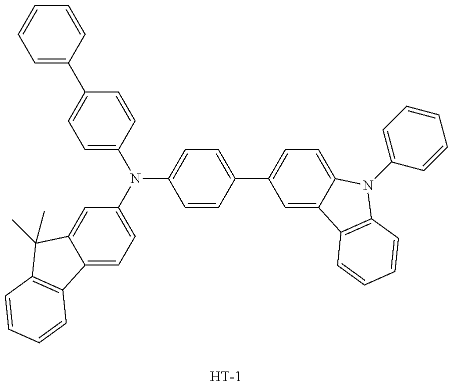 |
| | 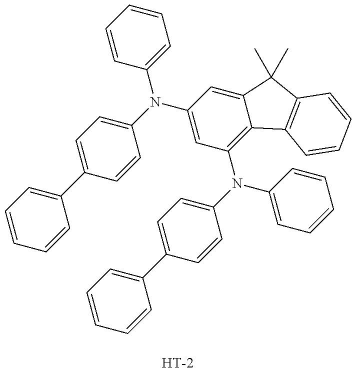 |
| Light- Emitting Layer | 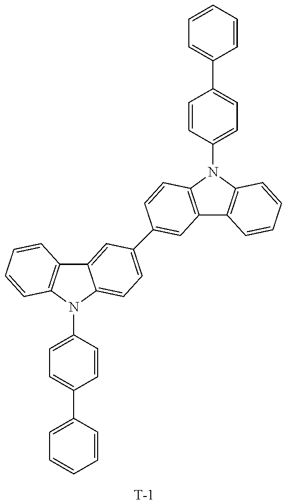 |
| | 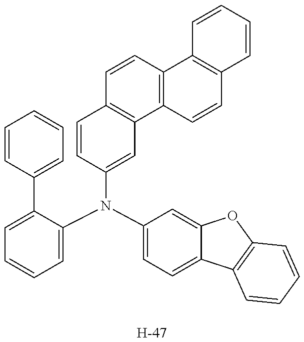 |
| | 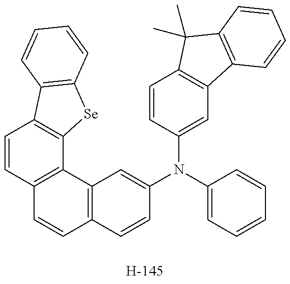 [SMILES: CC1(C)c2ccccc2-c2cc(N(c3ccccc3)c3ccc4ccc5ccc6c7ccccc7[se]c6c5c4c3)ccc21] |
| | 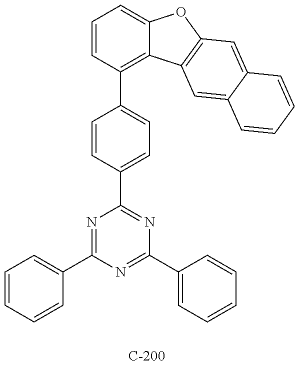 [SMILES: c1ccc(-c2nc(-c3ccccc3)nc(-c3ccc(-c4cccc5oc6cc7ccccc7cc6c45)cc3)n2)cc1] |
| | 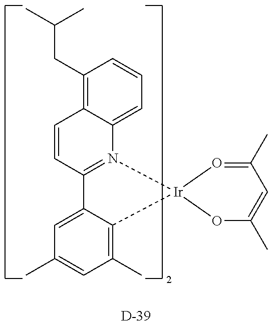 |
| Electron Transport Layer/ Electron Injection Layer | 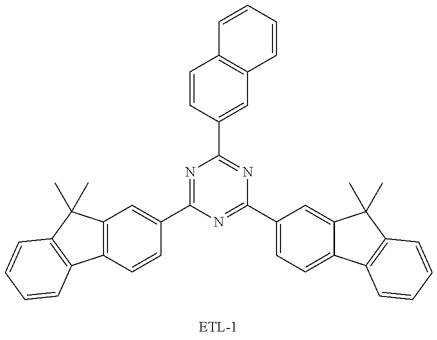 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | 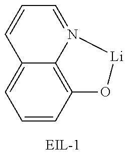 |

1. A plurality of host materials comprising at least one first host compound and at least one second host compound, wherein the first host compound is represented by the following formula 1, and the second host compound is represented by the following formula 2: 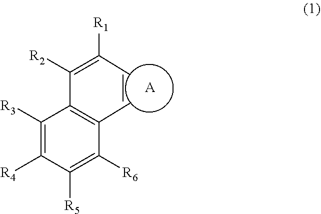
in formula 1,
ring A represents 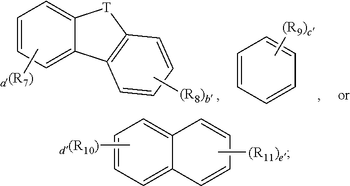
R₁ to R₁₁, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₁-N—(Ar₁)(Ar₂); with the proviso that at least one of R₁ to R₁₁ represents -L₁-N—(Ar₁)(Ar₂);
T represents O, S, or Se;
L₁, each independently, represents a single bond, a substituted or unsubstituted (C6-C30)arylene, or a substituted or unsubstituted (3- to 30-membered)heteroarylene;
Ar₁ and Ar₂, each independently, represent a substituted or unsubstituted (C1-C30)alkyl, a substituted or unsubstituted (C2-C30)alkenyl, a substituted or unsubstituted (C6-C30)aryl, or a substituted or unsubstituted (3- to 30-membered)heteroaryl; and
a′ to e′, each independently, represent an integer of 1 to 4, where if a′ to e′ are each an integer of 2 or more, each of R₇ to each of R₁₁ may be the same as or different from each other; 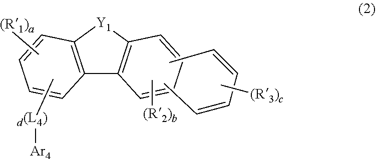
in formula 2,
Y₁ represents O or S;
R′₁ to R′₃, each independently, represent hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
L₄, each independently, represents a single bond, a substituted or unsubstituted (C6-C30)arylene, or a substituted or unsubstituted (3- to 30-membered)heteroarylene;
Ar₄ represents a substituted or unsubstituted (3- to 30-membered)heteroaryl containing a nitrogen(s);
a and d, each independently, represent an integer of 1 to 3, b represents an integer of 1 or 2, and c represents an integer of 1 to 4; where if a to d are each an integer of 2 or more, each of R′₁, each of R′₂, each of R′₃, and each of L₄ may be the same as or different from each other;
with the proviso that when the first host compound comprises a compound in which ring A represents 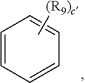
and the second host compound comprises a compound represented by the following formula 2′, at least one of R₃ to R₆, and R₉ is -L₁-N—(Ar₁)(Ar₂); and 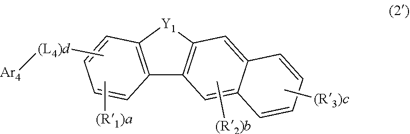
in formula 2′, Y₁, R′₁ to R′₃, L₄, Ar₄, and a to d are as defined in claim 2.

2. The plurality of host materials according to claim 1, wherein the substituent(s) of the substituted alkyl, the substituted aryl, the substituted arylene, the substituted heteroaryl, the substituted heteroarylene, the substituted cycloalkyl, the substituted heterocycloalkyl, the substituted fused ring group of an aliphatic ring(s) and a aromatic ring(s), the substituted trialkylsilyl, the substituted dialkylarylsilyl, the substituted alkyldiarylsilyl, the substituted triarylsilyl, the substituted mono- or di-alkylamino, the substituted mono- or di-arylamino, and the substituted alkylarylamino, each independently, are at least one selected from the group consisting of deuterium; a halogen; a cyano; a carboxyl; a nitro; a hydroxyl; a (C1-C30)alkyl; a halo(C1-C30)alkyl; a (C2-C30)alkenyl; (C2-C30)alkynyl; a (C1-C30)alkoxy; a (C1-C30)alkylthio; a (C3-C30)cycloalkyl; a (C3-C30)cycloalkenyl; a (3- to 7-membered)heterocycloalkyl; a (C6-C30)aryloxy; a (C6-C30)arylthio; a (3- to 30-membered)heteroaryl unsubstituted or substituted with a (C6-C30)aryl(s); a (C6-C30)aryl unsubstituted or substituted with at least one of deuterium, a halogen(s), a cyano(s), a (C1-C30)alkyl(s), a (C6-C30)aryl(s), a (3- to 30-membered)heteroaryl(s) and a tri(C6-C30)arylsilyl(s); a tri(C1-C30)alkylsilyl; a tri(C6-C30)arylsilyl; a di(C1-C30)alkyl(C6-C30)arylsilyl; a (C1-C30)alkyldi(C6-C30)arylsilyl; a fused ring group of a (C3-C30) aliphatic ring(s) and a (C6-C30) aromatic ring(s); an amino; a mono- or di-(C1-C30)alkylamino; a mono- or di-(C2-C30)alkenylamino; a mono- or di-(C6-C30)arylamino; a (C1-C30)alkyl(C6-C30)arylamino; a mono- or di-(3- to 30-membered)heteroarylamino; a (C1-C30)alkyl(3- to 30-membered)heteroarylamino; a (C2-C30)alkenyl(C6-C30)arylamino; a (C2-C30)alkenyl(3- to 30-membered)heteroarylamino; a (C6-C30)aryl(3- to 30-membered)heteroarylamino; a (C1-C30)alkylcarbonyl; a (C1-C30)alkoxycarbonyl; a (C6-C30)arylcarbonyl; a di(C6-C30)arylboronyl; a di(C1-C30)alkylboronyl; a (C1-C30)alkyl(C6-C30)arylboronyl; a (C6-C30)aryl(C1-C30)alkyl; and a (C1-C30)alkyl(C6-C30)aryl.

3. The plurality of host materials according to claim 1, wherein formula 1 is represented by at least one of the following formulas 1-1 to 1-6: 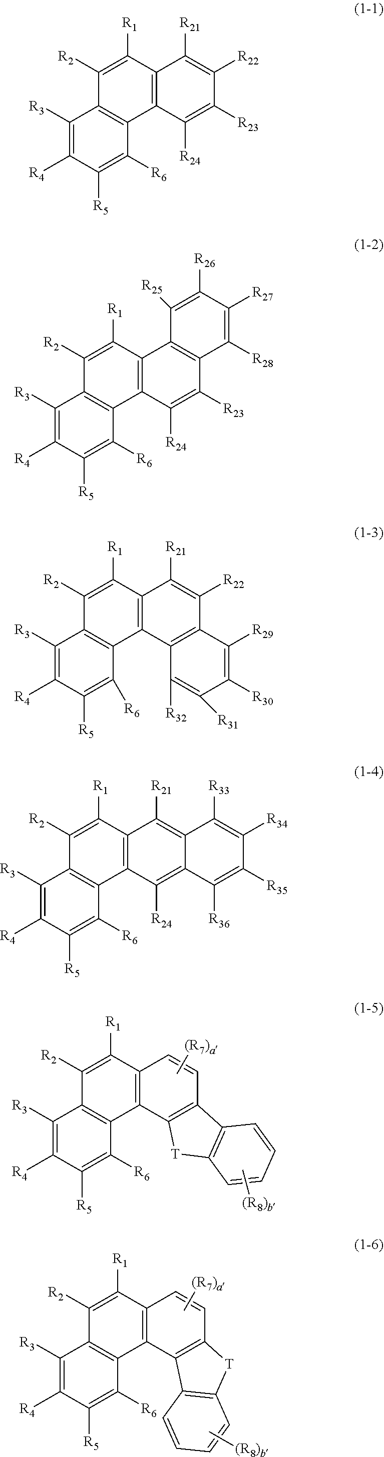
in formulas 1-1 to 1-6,
R₂₁ to R₃₆, each independently, represent hydrogen, deuterium, a halogen, a cyano, a substituted or unsubstituted (C1-C30)alkyl, a substituted or unsubstituted (C3-C30)cycloalkyl, a substituted or unsubstituted (3- to 7-membered)heterocycloalkyl, a substituted or unsubstituted fused ring group of a (C3-C30) aliphatic ring(s) and a (C6-C30) aromatic ring(s), a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, or -L₁-N—(Ar₁)(Ar₂); and
R₁ to R₈, L₁, Ar₁, Ar₂, T, a′ and b′ are as defined in claim 1.

4. The plurality of host materials according to claim 1, wherein formula 2 is represented by at least one of the following formulas 2-1 to 2-12: 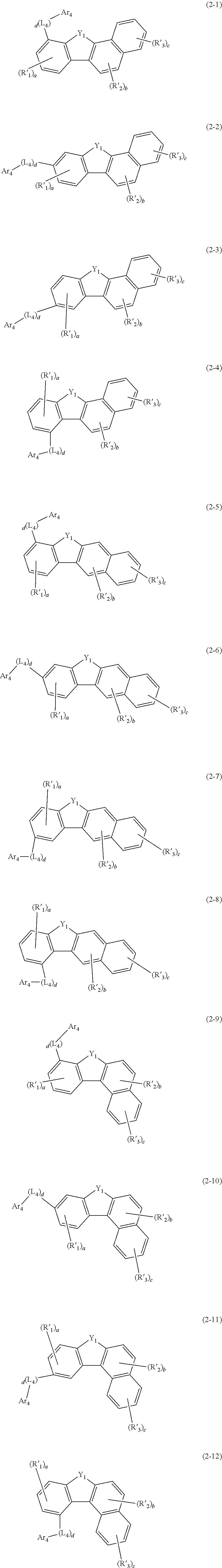
in formulas 2-1 to 2-12,
Y₁, R′₁ to R′, L₄, Ar₄, and a to d are as defined in claim 1.

5. The plurality of host materials according to claim 1, wherein Ar₄ in formula 2 is represented by any one of the following formulas 2′-1 to 2′-5: 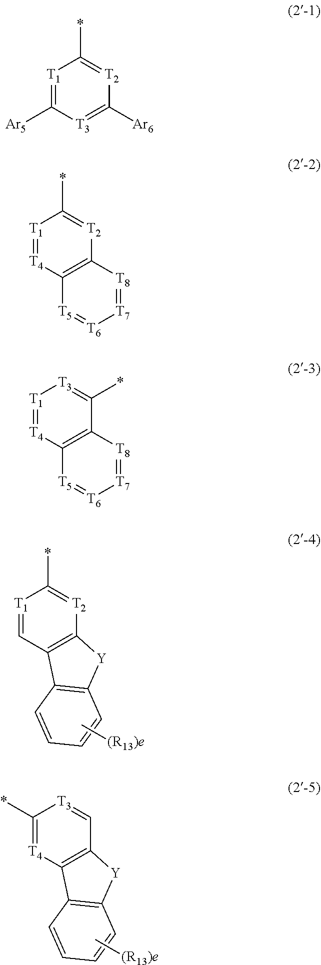
in formulas 2′-1 to 2′-5,
T₁ to T₈, each independently, represent CR₁₂ or N, with the proviso that at least one of T₁ to T₃ in formula 2′-1 represents N, at least one of T₁, T₂, and T₄ to T₈ in formula 2′-2 represents N, at least one of T₁, and T₃ to T₈ in formula 2′-3 represents N, at least one of T₁ and T₂ in formula 2-4 represents N, and at least one of T₃ and T₄ in formula 2′-5 represents N;
Y represents O or S;
R₁₂, Ar₅ and Ar₆, each independently, represent hydrogen, deuterium, a substituted or unsubstituted (C1-C30)alkyl, a substituted or unsubstituted (C6-C30)aryl, or a substituted or unsubstituted (3- to 30-membered)heteroaryl; or R₁₂ may be linked to an adjacent substituent(s) to form a ring(s);
R₁₃, each independently, represents hydrogen, deuterium, a halogen, a cyano, a substituted or unsubstituted (C1-C30)alkyl, a substituted or unsubstituted (C6-C30)aryl, a substituted or unsubstituted (3- to 30-membered)heteroaryl, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C6-C30)arylamino, or a substituted or unsubstituted (C1-C30)alkyl(C6-C30)arylamino;
* represents a bonding position to L₄; and
e represents an integer of 1 to 4, where if e is an integer of 2 or more, each of R₁₃ may be the same as or different from each other.

6. The plurality of host materials according to claim 1, wherein the compound represented by formula 1 is selected from the following compounds.

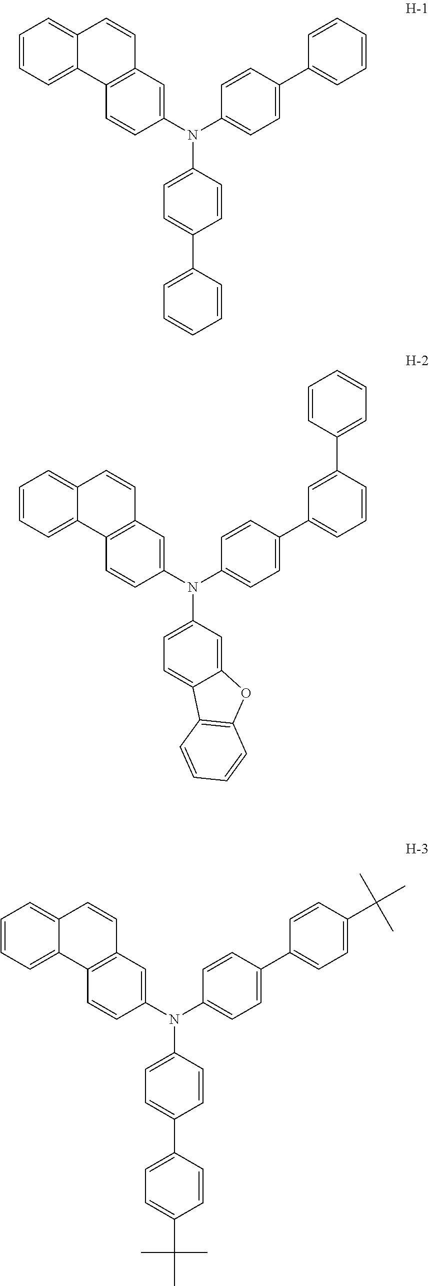 [SMILES: c1ccc(-c2ccc(N(c3ccc(-c4ccccc4)cc3)c3ccc4c(ccc5ccccc54)c3)cc2)cc1 ; c1ccc(-c2cccc(-c3ccc(N(c4ccc5c(ccc6ccccc65)c4)c4ccc5c(c4)oc4ccccc45)cc3)c2)cc1 ; CC(C)(C)c1ccc(-c2ccc(N(c3ccc(-c4ccc(C(C)(C)C)cc4)cc3)c3ccc4c(ccc5ccccc54)c3)cc2)cc1]

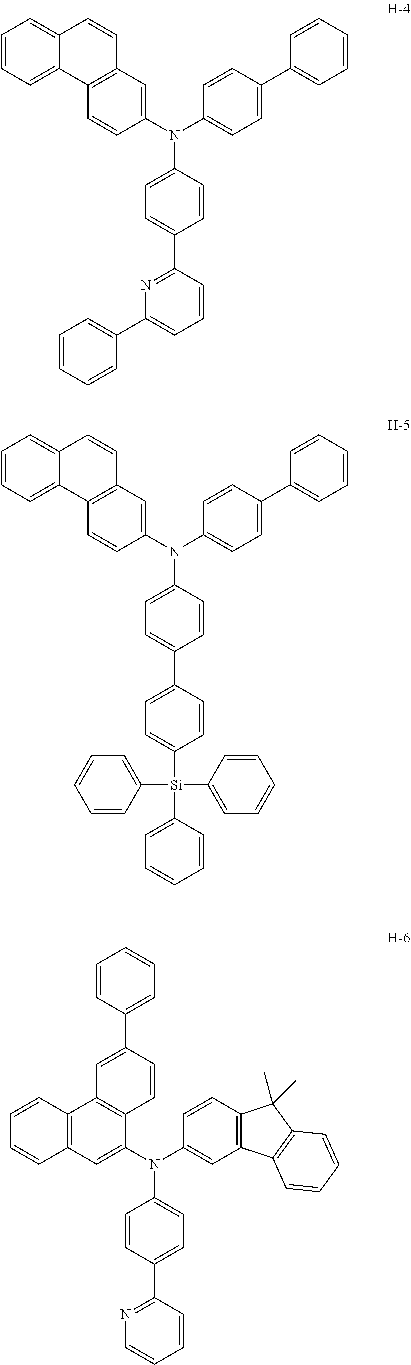 [SMILES: c1ccc(-c2ccc(N(c3ccc(-c4cccc(-c5ccccc5)n4)cc3)c3ccc4c(ccc5ccccc54)c3)cc2)cc1 ; c1ccc(-c2ccc(N(c3ccc(-c4ccc([Si](c5ccccc5)(c5ccccc5)c5ccccc5)cc4)cc3)c3ccc4c(ccc5ccccc54)c3)cc2)cc1 ; CC1(C)c2ccccc2-c2cc(N(c3ccc(-c4ccccn4)cc3)c3cc4ccccc4c4cc(-c5ccccc5)ccc34)ccc21]

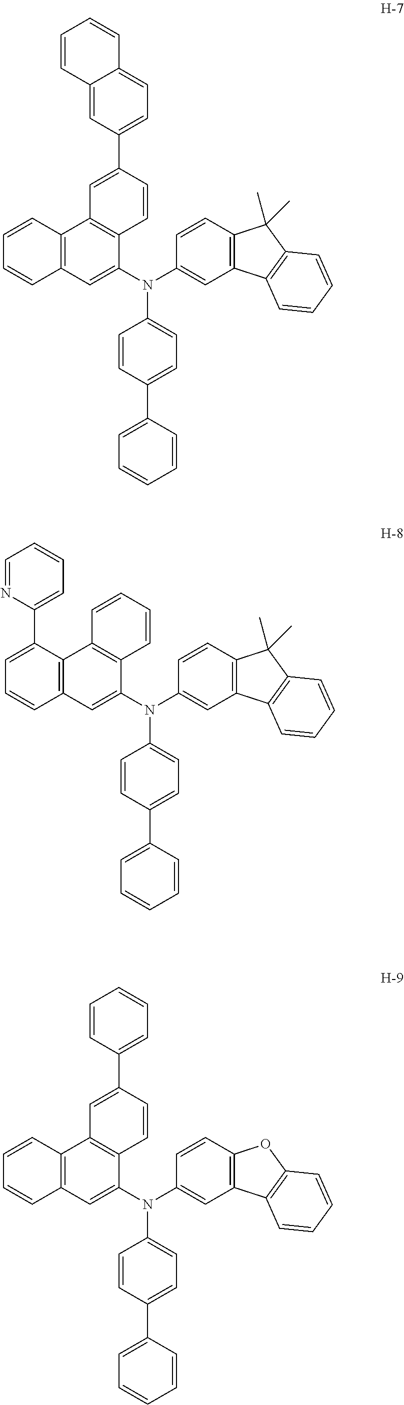 [SMILES: CC1(C)c2ccccc2-c2cc(N(c3ccc(-c4ccccc4)cc3)c3cc4ccccc4c4cc(-c5ccc6ccccc6c5)ccc34)ccc21 ; CC1(C)c2ccccc2-c2cc(N(c3ccc(-c4ccccc4)cc3)c3cc4cccc(-c5ccccn5)c4c4ccccc34)ccc21 ; c1ccc(-c2ccc(N(c3ccc4oc5ccccc5c4c3)c3cc4ccccc4c4cc(-c5ccccc5)ccc34)cc2)cc1]

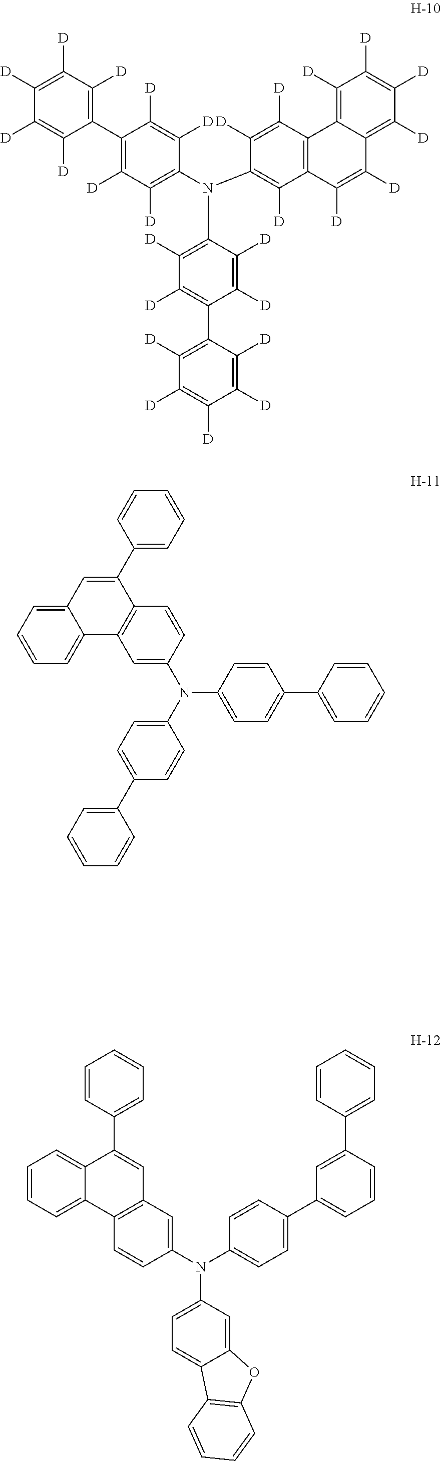 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c([2H])c([2H])c([2H])c([2H])c34)c([2H])c2[2H])c([2H])c1[2H] ; c1ccc(-c2ccc(N(c3ccc(-c4ccccc4)cc3)c3ccc4c(-c5ccccc5)cc5ccccc5c4c3)cc2)cc1 ; c1ccc(-c2cccc(-c3ccc(N(c4ccc5c(c4)cc(-c4ccccc4)c4ccccc45)c4ccc5c(c4)oc4ccccc45)cc3)c2)cc1]

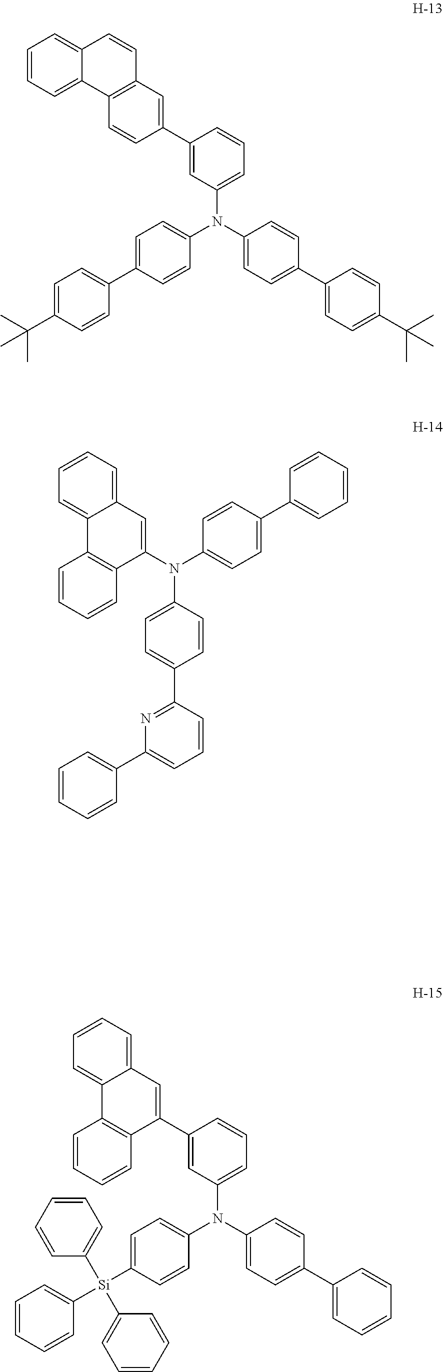 [SMILES: CC(C)(C)c1ccc(-c2ccc(N(c3ccc(-c4ccc(C(C)(C)C)cc4)cc3)c3cccc(-c4ccc5c(ccc6ccccc65)c4)c3)cc2)cc1 ; c1ccc(-c2ccc(N(c3ccc(-c4cccc(-c5ccccc5)n4)cc3)c3cc4ccccc4c4ccccc34)cc2)cc1 ; c1ccc(-c2ccc(N(c3ccc([Si](c4ccccc4)(c4ccccc4)c4ccccc4)cc3)c3cccc(-c4cc5ccccc5c5ccccc45)c3)cc2)cc1]

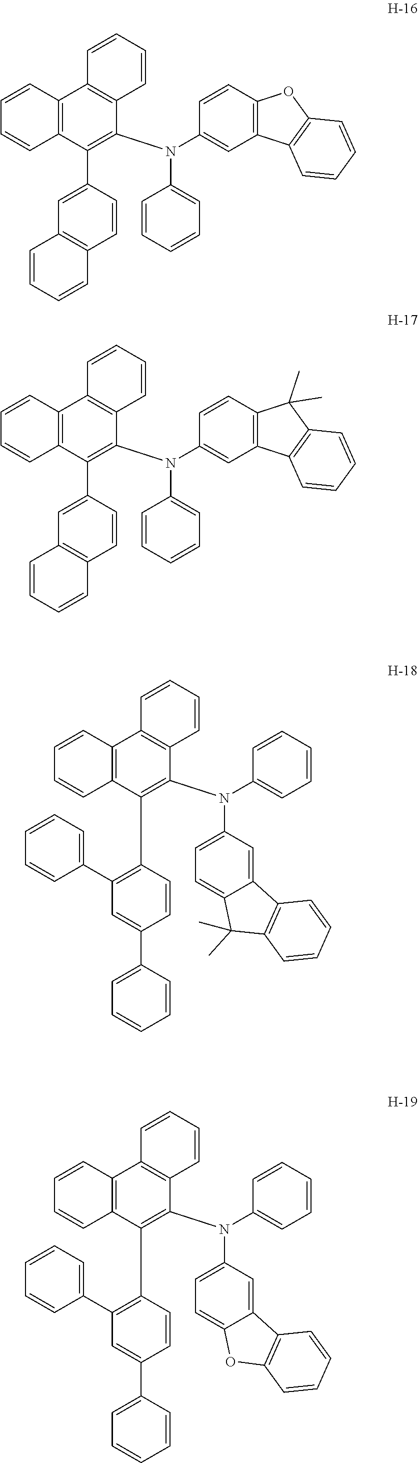 [SMILES: c1ccc(N(c2ccc3oc4ccccc4c3c2)c2c(-c3ccc4ccccc4c3)c3ccccc3c3ccccc23)cc1 ; CC1(C)c2ccccc2-c2cc(N(c3ccccc3)c3c(-c4ccc5ccccc5c4)c4ccccc4c4ccccc34)ccc21 ; CC1(C)c2ccccc2-c2cc(N(c3ccccc3)c3c(-c4ccc(-c5ccccc5)cc4-c4ccccc4)c4ccccc4c4ccccc34)ccc21 ; c1ccc(-c2ccc(-c3c(N(c4ccccc4)c4ccc5oc6ccccc6c5c4)c4ccccc4c4ccccc34)c(-c3ccccc3)c2)cc1]

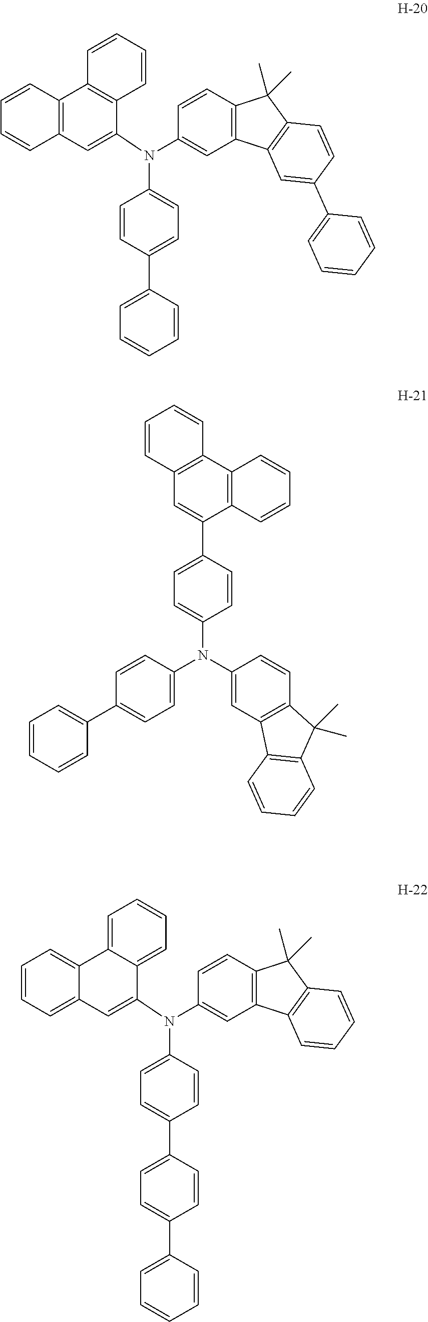 [SMILES: CC1(C)c2ccc(-c3ccccc3)cc2-c2cc(N(c3ccc(-c4ccccc4)cc3)c3cc4ccccc4c4ccccc34)ccc21 ; CC1(C)c2ccccc2-c2cc(N(c3ccc(-c4ccccc4)cc3)c3ccc(-c4cc5ccccc5c5ccccc45)cc3)ccc21 ; CC1(C)c2ccccc2-c2cc(N(c3ccc(-c4ccc(-c5ccccc5)cc4)cc3)c3cc4ccccc4c4ccccc34)ccc21]

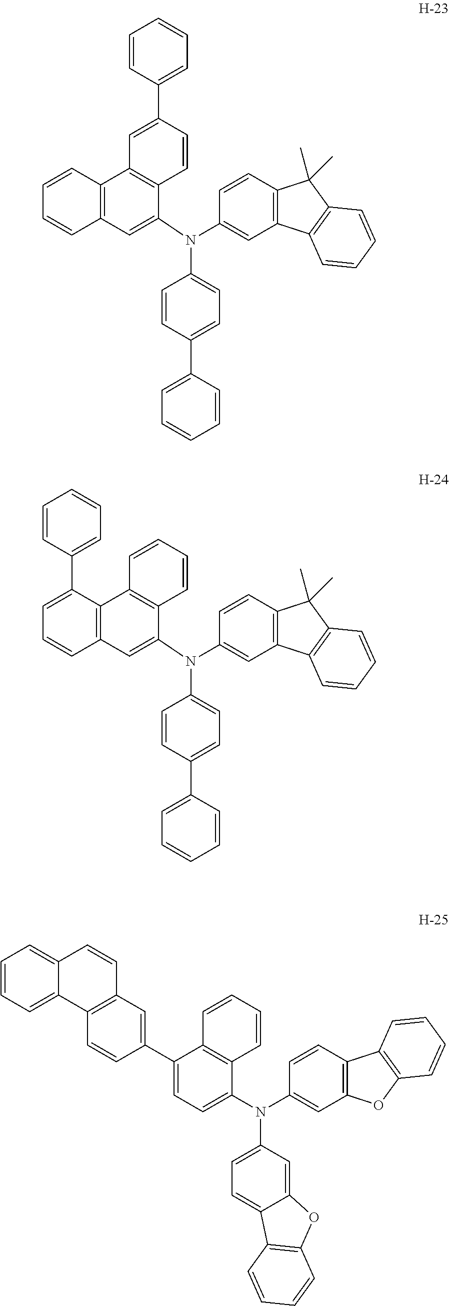 [SMILES: CC1(C)c2ccccc2-c2cc(N(c3ccc(-c4ccccc4)cc3)c3cc4ccccc4c4cc(-c5ccccc5)ccc34)ccc21 ; CC1(C)c2ccccc2-c2cc(N(c3ccc(-c4ccccc4)cc3)c3cc4cccc(-c5ccccc5)c4c4ccccc34)ccc21 ; c1ccc2c(c1)ccc1cc(-c3ccc(N(c4ccc5c(c4)oc4ccccc45)c4ccc5c(c4)oc4ccccc45)c4ccccc34)ccc12]

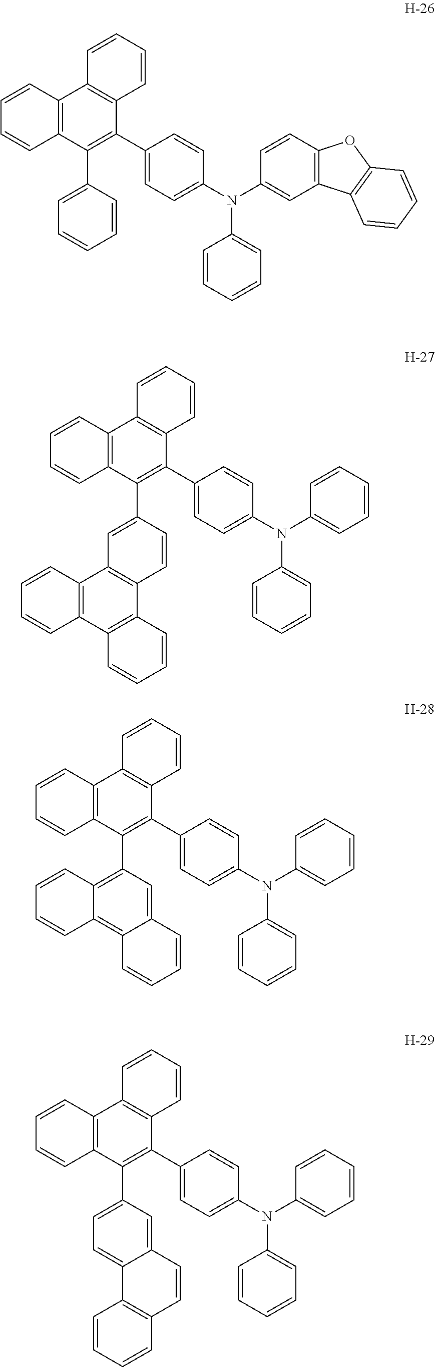

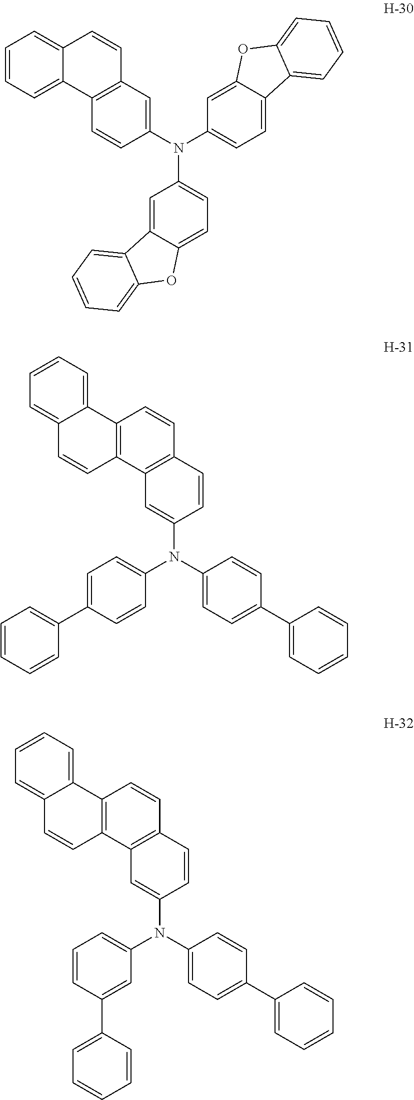 [SMILES: c1ccc2c(c1)ccc1cc(N(c3ccc4c(c3)oc3ccccc34)c3ccc4oc5ccccc5c4c3)ccc12 ; c1ccc(-c2ccc(N(c3ccc(-c4ccccc4)cc3)c3ccc4ccc5c6ccccc6ccc5c4c3)cc2)cc1 ; c1ccc(-c2ccc(N(c3cccc(-c4ccccc4)c3)c3ccc4ccc5c6ccccc6ccc5c4c3)cc2)cc1]

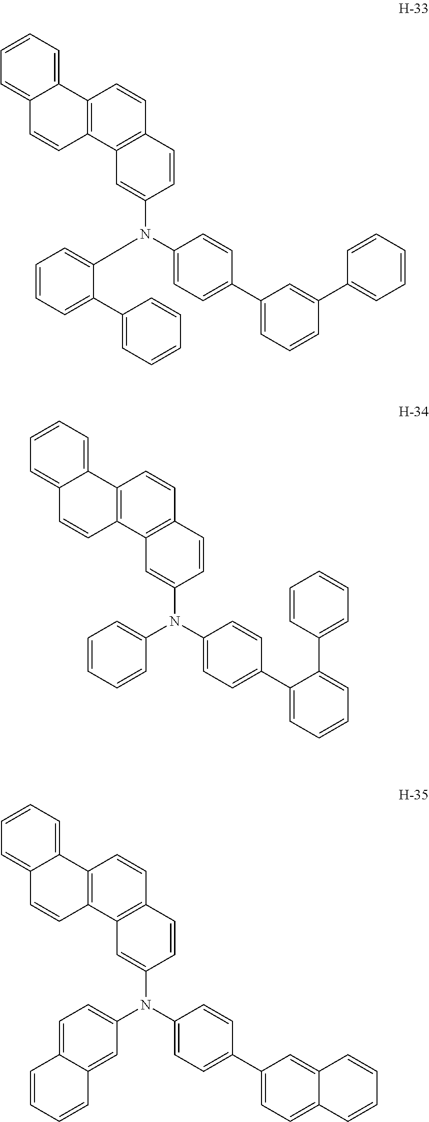 [SMILES: c1ccc(-c2cccc(-c3ccc(N(c4ccc5ccc6c7ccccc7ccc6c5c4)c4ccccc4-c4ccccc4)cc3)c2)cc1 ; c1ccc(-c2ccccc2-c2ccc(N(c3ccccc3)c3ccc4ccc5c6ccccc6ccc5c4c3)cc2)cc1 ; c1ccc2cc(-c3ccc(N(c4ccc5ccccc5c4)c4ccc5ccc6c7ccccc7ccc6c5c4)cc3)ccc2c1]

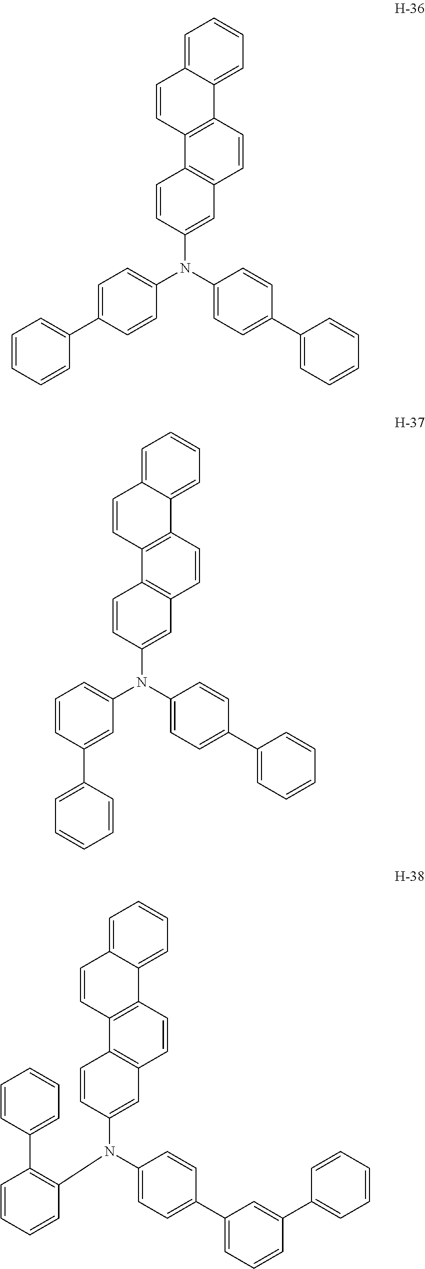 [SMILES: c1ccc(-c2ccc(N(c3ccc(-c4ccccc4)cc3)c3ccc4c(ccc5c6ccccc6ccc45)c3)cc2)cc1 ; c1ccc(-c2ccc(N(c3cccc(-c4ccccc4)c3)c3ccc4c(ccc5c6ccccc6ccc45)c3)cc2)cc1 ; c1ccc(-c2cccc(-c3ccc(N(c4ccc5c(ccc6c7ccccc7ccc56)c4)c4ccccc4-c4ccccc4)cc3)c2)cc1]

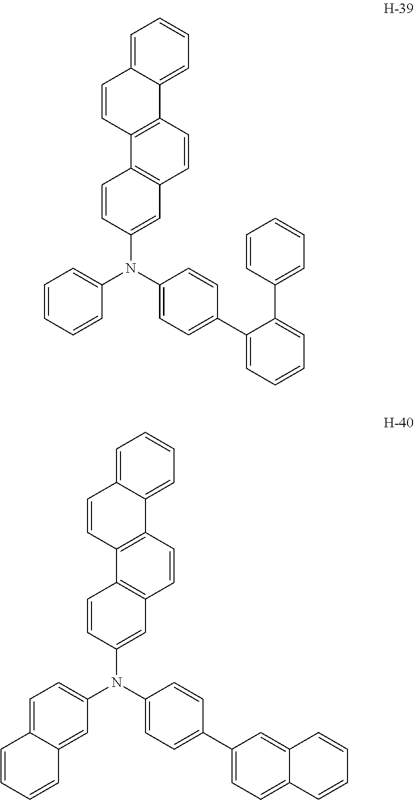 [SMILES: c1ccc(-c2ccccc2-c2ccc(N(c3ccccc3)c3ccc4c(ccc5c6ccccc6ccc45)c3)cc2)cc1 ; c1ccc2cc(-c3ccc(N(c4ccc5ccccc5c4)c4ccc5c(ccc6c7ccccc7ccc56)c4)cc3)ccc2c1]

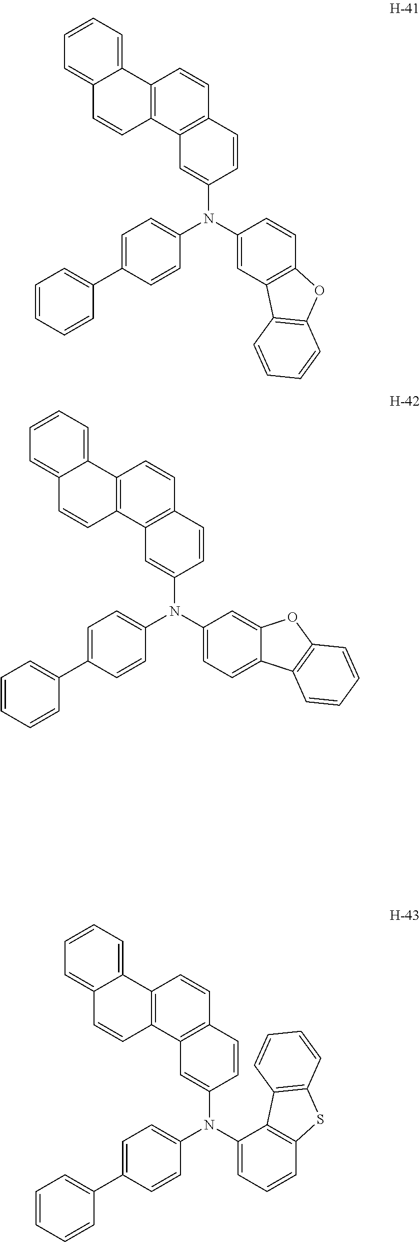 [SMILES: c1ccc(-c2ccc(N(c3ccc4ccc5c6ccccc6ccc5c4c3)c3ccc4oc5ccccc5c4c3)cc2)cc1 ; c1ccc(-c2ccc(N(c3ccc4c(c3)oc3ccccc34)c3ccc4ccc5c6ccccc6ccc5c4c3)cc2)cc1 ; c1ccc(-c2ccc(N(c3ccc4ccc5c6ccccc6ccc5c4c3)c3cccc4sc5ccccc5c34)cc2)cc1]

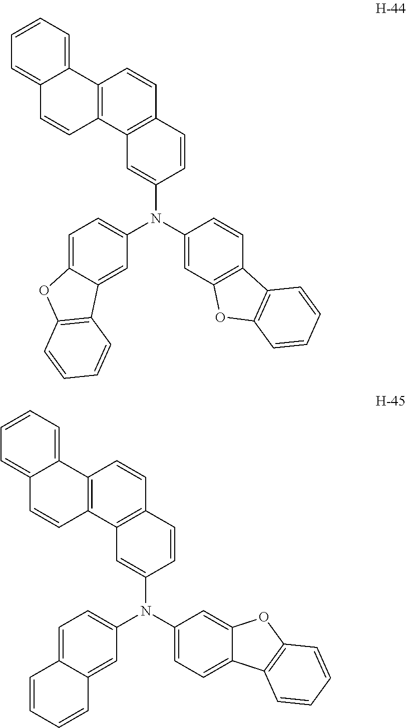 [SMILES: c1ccc2c(c1)ccc1c3cc(N(c4ccc5c(c4)oc4ccccc45)c4ccc5oc6ccccc6c5c4)ccc3ccc21 ; c1ccc2cc(N(c3ccc4c(c3)oc3ccccc34)c3ccc4ccc5c6ccccc6ccc5c4c3)ccc2c1]

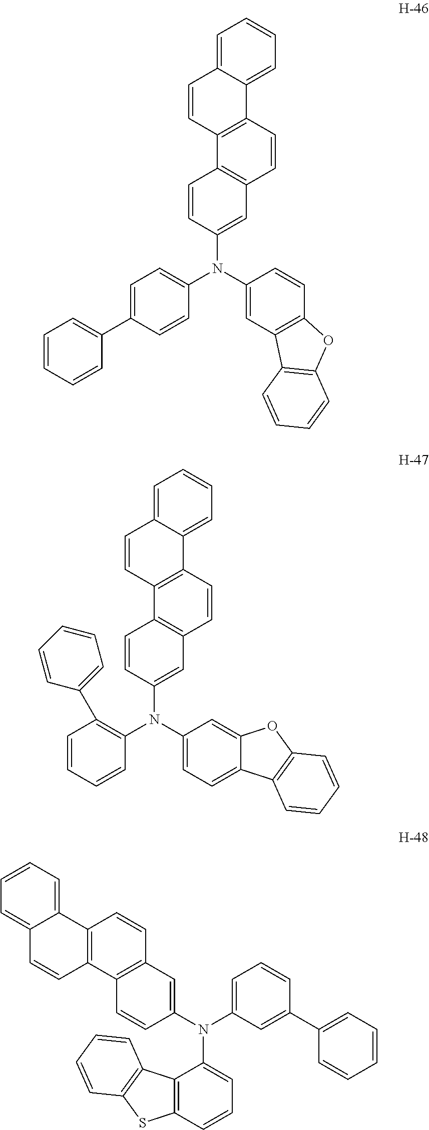 [SMILES: c1ccc(-c2ccc(N(c3ccc4c(ccc5c6ccccc6ccc45)c3)c3ccc4oc5ccccc5c4c3)cc2)cc1 ; c1ccc(-c2ccccc2N(c2ccc3c(ccc4c5ccccc5ccc34)c2)c2ccc3c(c2)oc2ccccc23)cc1 ; c1ccc(-c2cccc(N(c3ccc4c(ccc5c6ccccc6ccc45)c3)c3cccc4sc5ccccc5c34)c2)cc1]

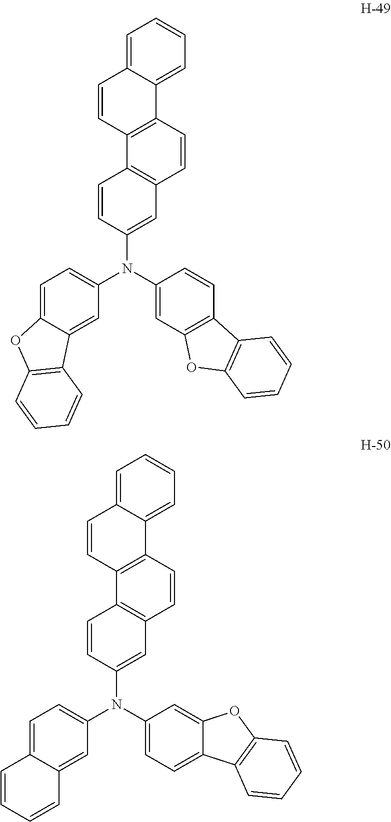 [SMILES: c1ccc2c(c1)ccc1c3ccc(N(c4ccc5c(c4)oc4ccccc45)c4ccc5oc6ccccc6c5c4)cc3ccc21 ; c1ccc2cc(N(c3ccc4c(ccc5c6ccccc6ccc45)c3)c3ccc4c(c3)oc3ccccc34)ccc2c1]

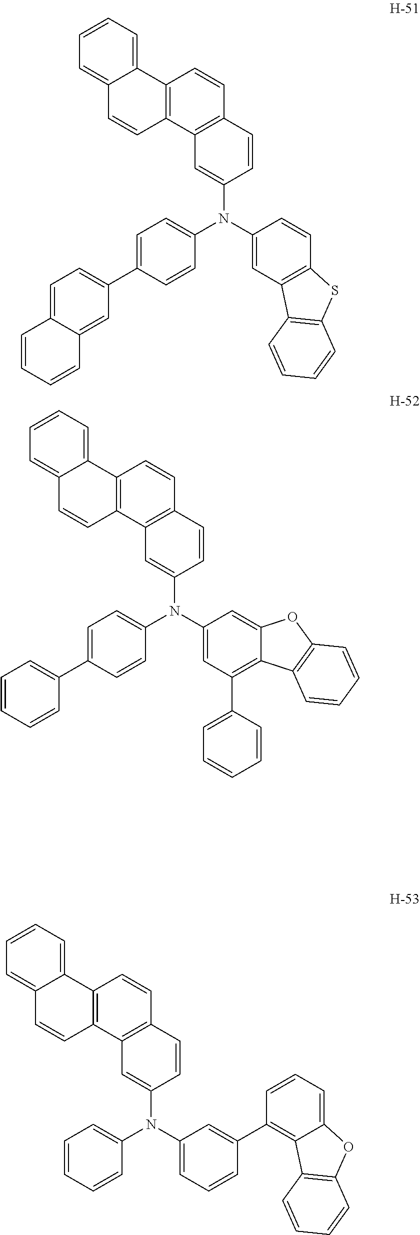

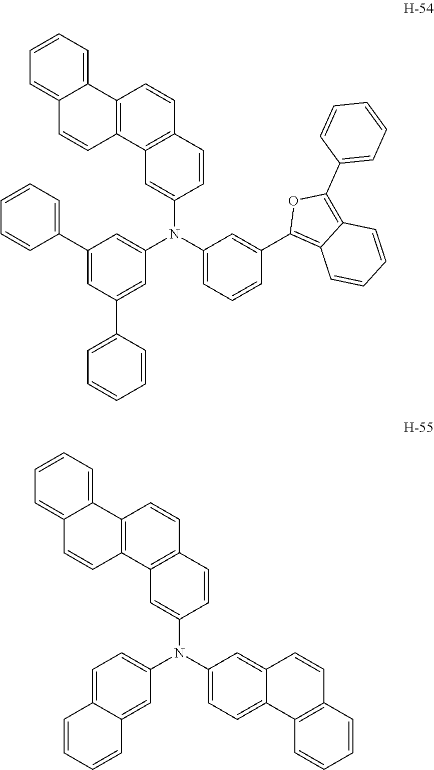 [SMILES: c1ccc(-c2cc(-c3ccccc3)cc(N(c3cccc(-c4oc(-c5ccccc5)c5ccccc45)c3)c3ccc4ccc5c6ccccc6ccc5c4c3)c2)cc1 ; c1ccc2cc(N(c3ccc4c(ccc5ccccc54)c3)c3ccc4ccc5c6ccccc6ccc5c4c3)ccc2c1]

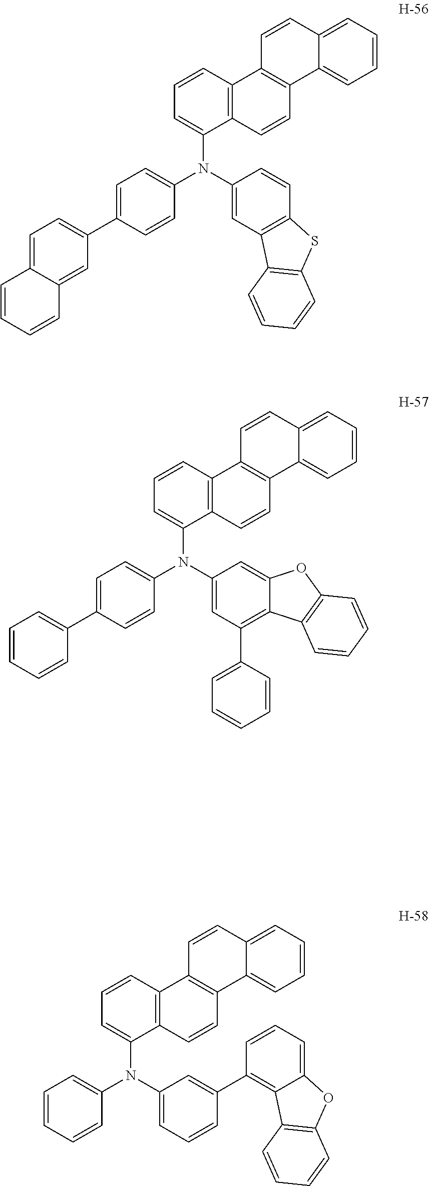 [SMILES: c1ccc2cc(-c3ccc(N(c4ccc5sc6ccccc6c5c4)c4cccc5c4ccc4c6ccccc6ccc54)cc3)ccc2c1 ; c1ccc(-c2ccc(N(c3cc(-c4ccccc4)c4c(c3)oc3ccccc34)c3cccc4c3ccc3c5ccccc5ccc43)cc2)cc1 ; c1ccc(N(c2cccc(-c3cccc4oc5ccccc5c34)c2)c2cccc3c2ccc2c4ccccc4ccc32)cc1]

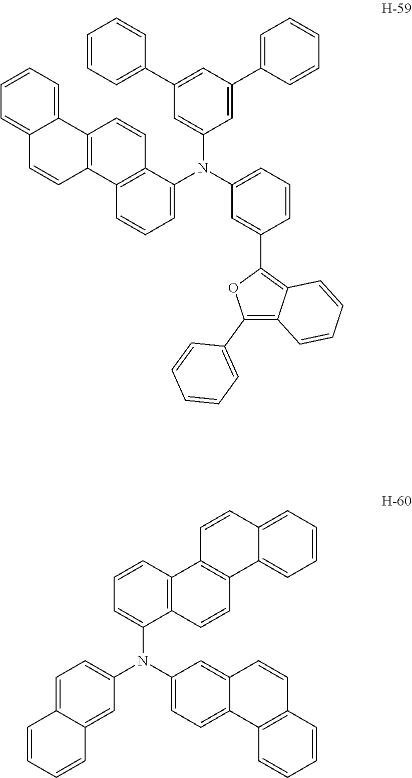 [SMILES: c1ccc(-c2cc(-c3ccccc3)cc(N(c3cccc(-c4oc(-c5ccccc5)c5ccccc45)c3)c3cccc4c3ccc3c5ccccc5ccc43)c2)cc1 ; c1ccc2cc(N(c3ccc4c(ccc5ccccc54)c3)c3cccc4c3ccc3c5ccccc5ccc43)ccc2c1]

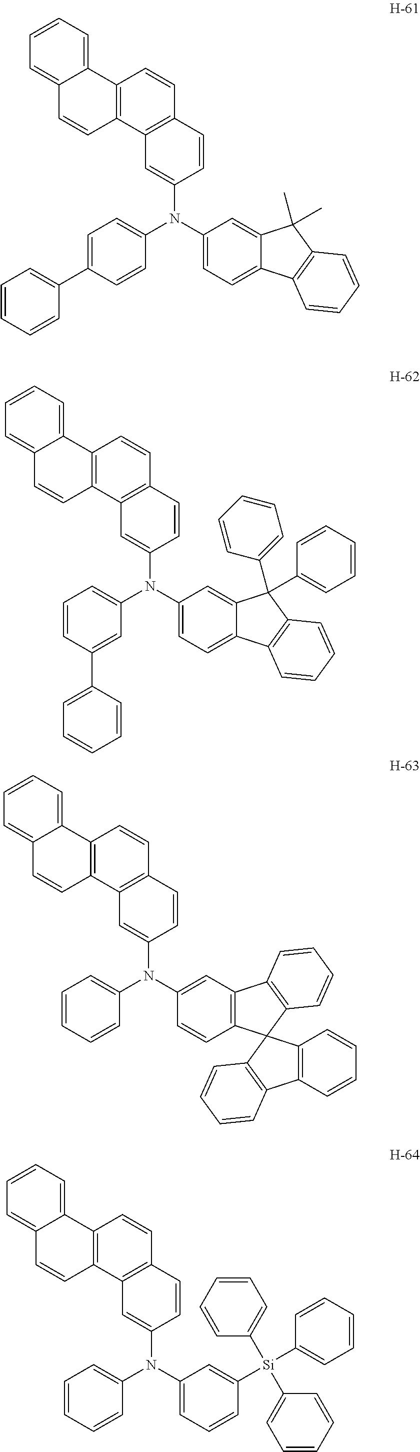 [SMILES: CC1(C)c2ccccc2-c2ccc(N(c3ccc(-c4ccccc4)cc3)c3ccc4ccc5c6ccccc6ccc5c4c3)cc21 ; c1ccc(-c2cccc(N(c3ccc4c(c3)C(c3ccccc3)(c3ccccc3)c3ccccc3-4)c3ccc4ccc5c6ccccc6ccc5c4c3)c2)cc1 ; c1ccc(N(c2ccc3c(c2)-c2ccccc2C32c3ccccc3-c3ccccc32)c2ccc3ccc4c5ccccc5ccc4c3c2)cc1 ; c1ccc(N(c2cccc([Si](c3ccccc3)(c3ccccc3)c3ccccc3)c2)c2ccc3ccc4c5ccccc5ccc4c3c2)cc1]

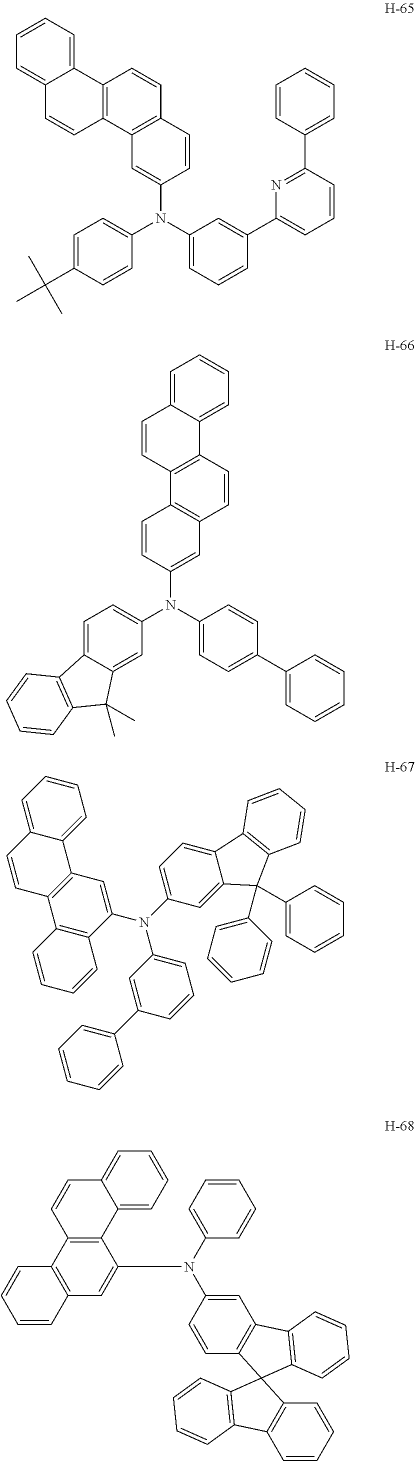

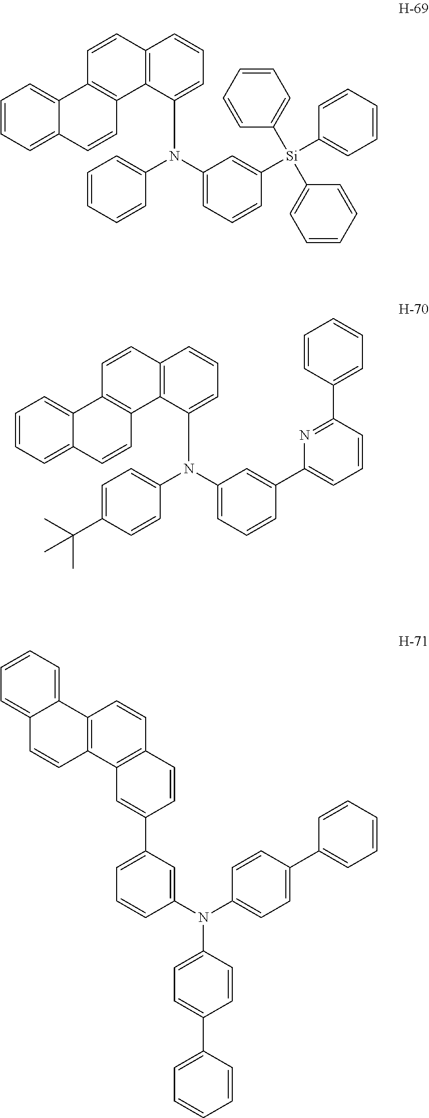

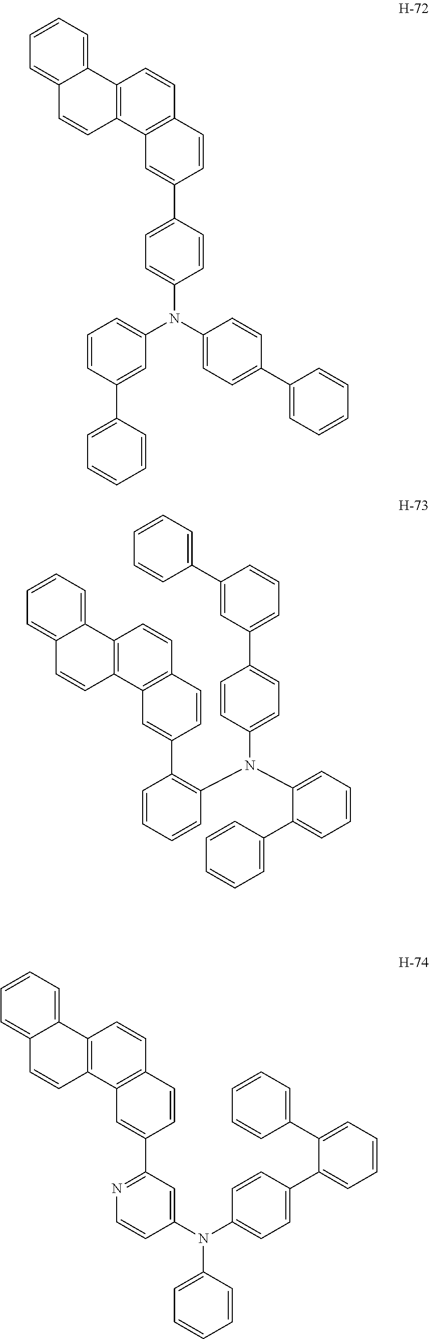 [SMILES: c1ccc(-c2ccc(N(c3ccc(-c4ccc5ccc6c7ccccc7ccc6c5c4)cc3)c3cccc(-c4ccccc4)c3)cc2)cc1 ; c1ccc(-c2cccc(-c3ccc(N(c4ccccc4-c4ccccc4)c4ccccc4-c4ccc5ccc6c7ccccc7ccc6c5c4)cc3)c2)cc1 ; c1ccc(-c2ccccc2-c2ccc(N(c3ccccc3)c3ccnc(-c4ccc5ccc6c7ccccc7ccc6c5c4)c3)cc2)cc1]

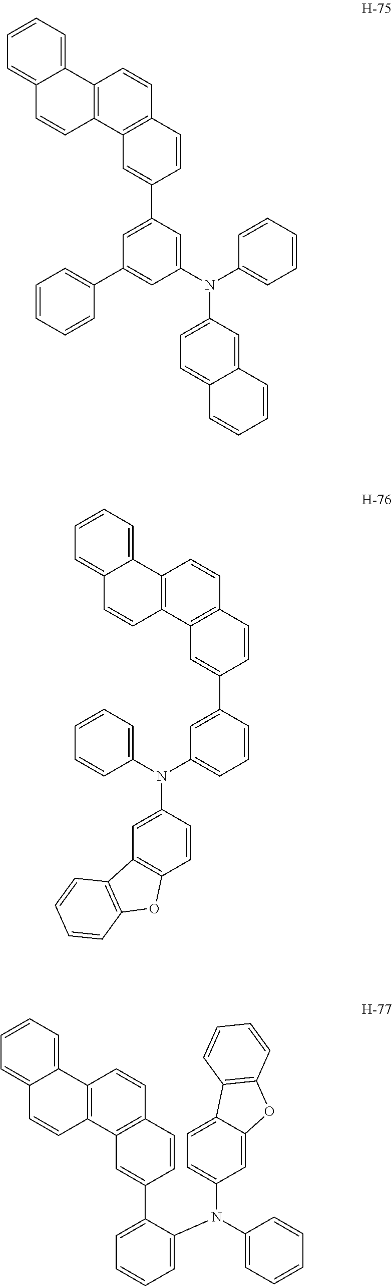 [SMILES: c1ccc(-c2cc(-c3ccc4ccc5c6ccccc6ccc5c4c3)cc(N(c3ccccc3)c3ccc4ccccc4c3)c2)cc1 ; c1ccc(N(c2cccc(-c3ccc4ccc5c6ccccc6ccc5c4c3)c2)c2ccc3oc4ccccc4c3c2)cc1 ; c1ccc(N(c2ccc3c(c2)oc2ccccc23)c2ccccc2-c2ccc3ccc4c5ccccc5ccc4c3c2)cc1]

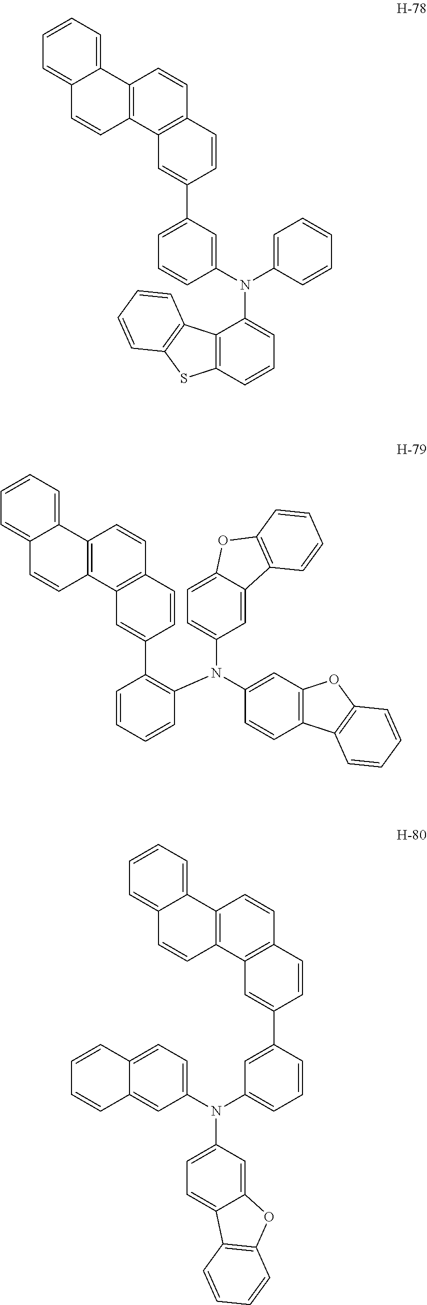 [SMILES: c1ccc(N(c2cccc(-c3ccc4ccc5c6ccccc6ccc5c4c3)c2)c2cccc3sc4ccccc4c23)cc1 ; c1ccc(N(c2ccc3c(c2)oc2ccccc23)c2ccc3oc4ccccc4c3c2)c(-c2ccc3ccc4c5ccccc5ccc4c3c2)c1 ; c1cc(-c2ccc3ccc4c5ccccc5ccc4c3c2)cc(N(c2ccc3ccccc3c2)c2ccc3c(c2)oc2ccccc23)c1]

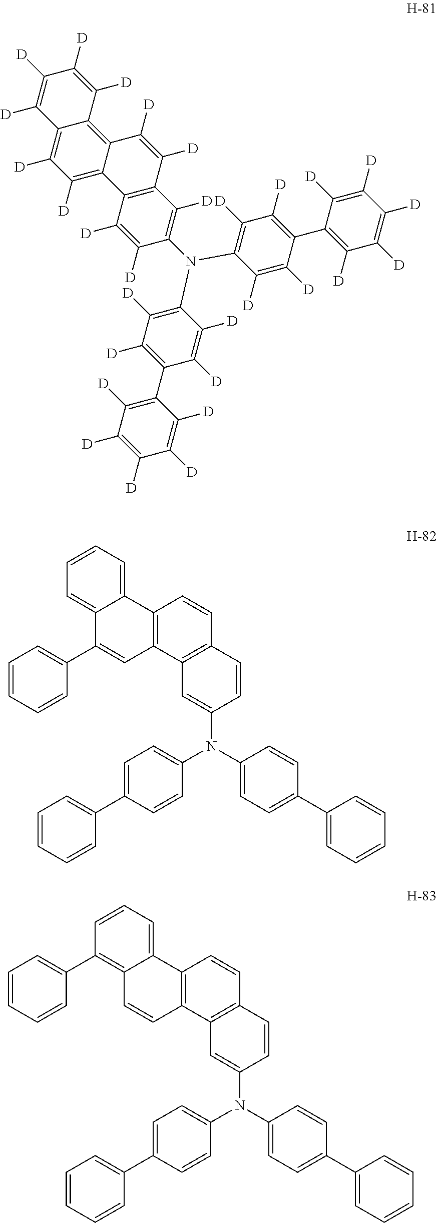 [SMILES: [2H]c1c([2H])c([2H])c(-c2c([2H])c([2H])c(N(c3c([2H])c([2H])c(-c4c([2H])c([2H])c([2H])c([2H])c4[2H])c([2H])c3[2H])c3c([2H])c([2H])c4c(c3[2H])c([2H])c([2H])c3c5c([2H])c([2H])c([2H])c([2H])c5c([2H])c([2H])c43)c([2H])c2[2H])c([2H])c1[2H] ; c1ccc(-c2ccc(N(c3ccc(-c4ccccc4)cc3)c3ccc4ccc5c6ccccc6c(-c6ccccc6)cc5c4c3)cc2)cc1 ; c1ccc(-c2ccc(N(c3ccc(-c4ccccc4)cc3)c3ccc4ccc5c6cccc(-c7ccccc7)c6ccc5c4c3)cc2)cc1]

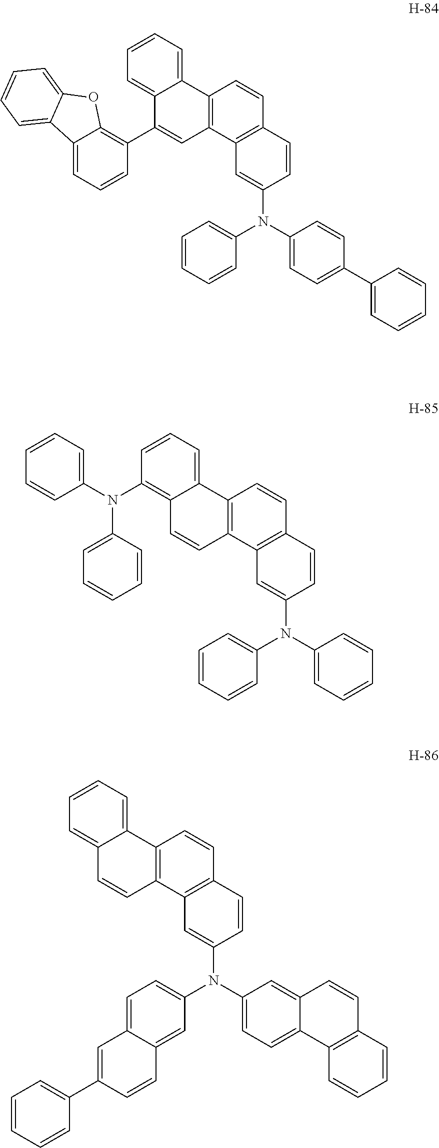 [SMILES: c1ccc(-c2ccc(N(c3ccccc3)c3ccc4ccc5c6ccccc6c(-c6cccc7c6oc6ccccc67)cc5c4c3)cc2)cc1 ; c1ccc(N(c2ccccc2)c2ccc3ccc4c5cccc(N(c6ccccc6)c6ccccc6)c5ccc4c3c2)cc1 ; c1ccc(-c2ccc3cc(N(c4ccc5c(ccc6ccccc65)c4)c4ccc5ccc6c7ccccc7ccc6c5c4)ccc3c2)cc1]

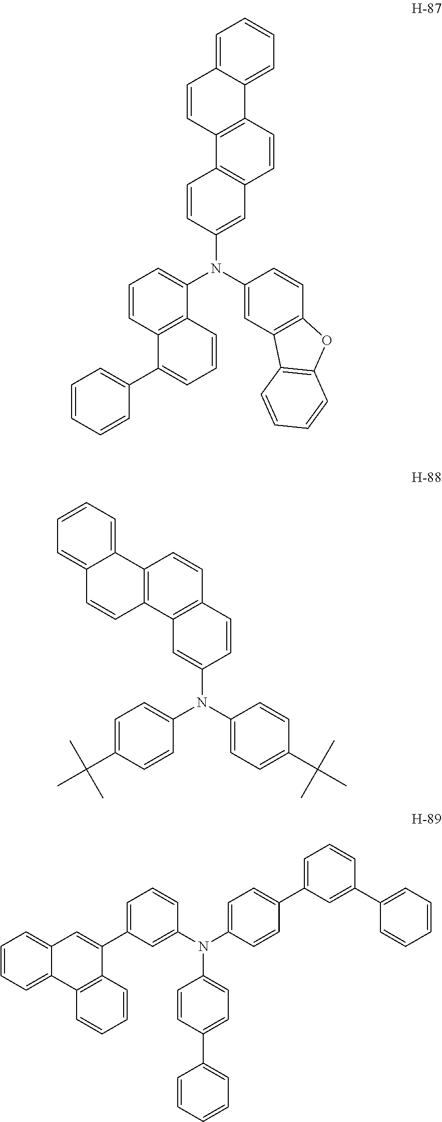 [SMILES: c1ccc(-c2cccc3c(N(c4ccc5c(ccc6c7ccccc7ccc56)c4)c4ccc5oc6ccccc6c5c4)cccc23)cc1 ; CC(C)(C)c1ccc(N(c2ccc(C(C)(C)C)cc2)c2ccc3ccc4c5ccccc5ccc4c3c2)cc1 ; c1ccc(-c2ccc(N(c3ccc(-c4cccc(-c5ccccc5)c4)cc3)c3cccc(-c4cc5ccccc5c5ccccc45)c3)cc2)cc1]

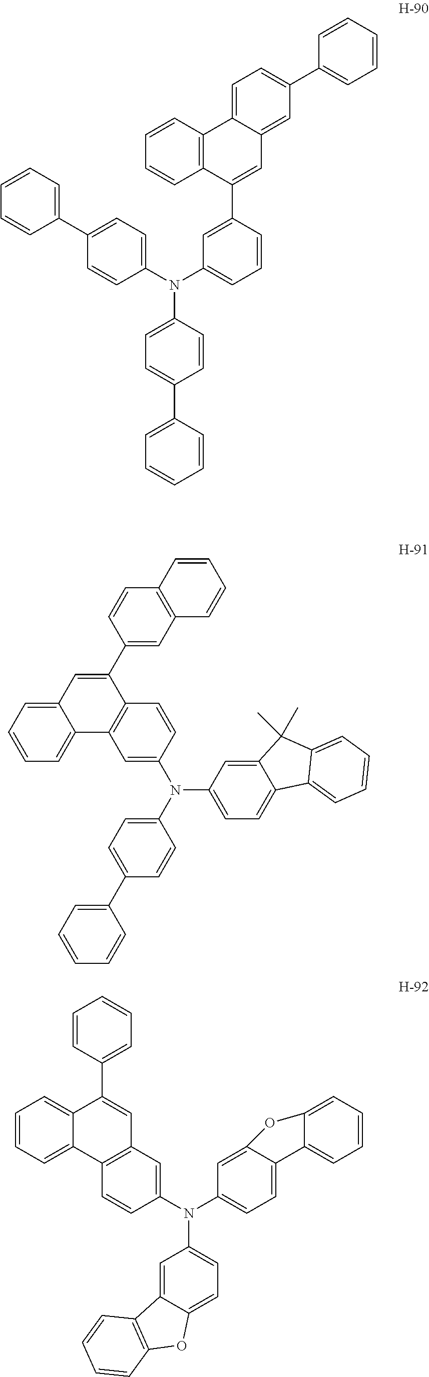

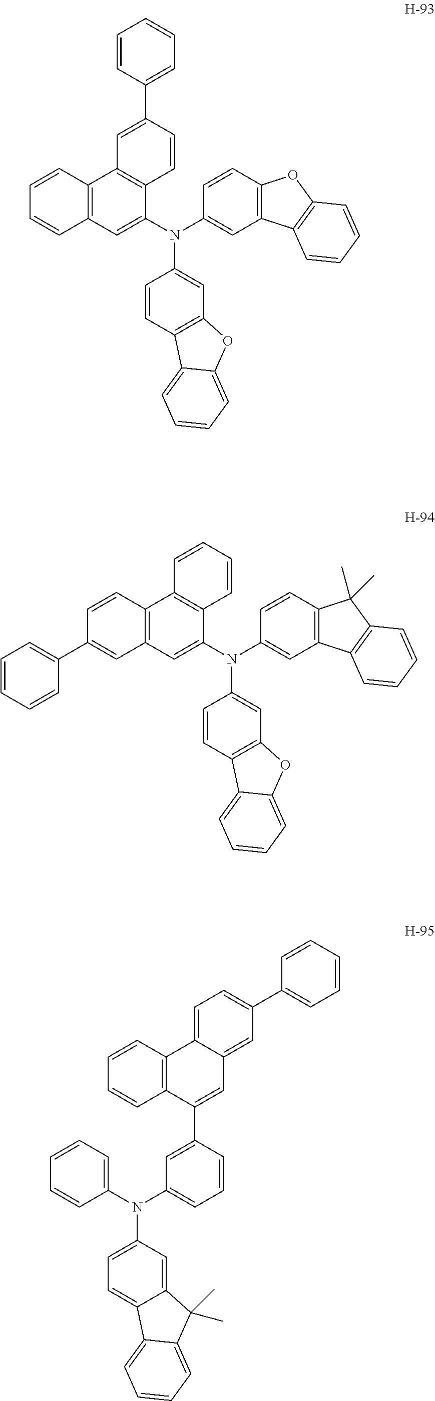 [SMILES: c1ccc(-c2ccc3c(N(c4ccc5c(c4)oc4ccccc45)c4ccc5oc6ccccc6c5c4)cc4ccccc4c3c2)cc1 ; CC1(C)c2ccccc2-c2cc(N(c3ccc4c(c3)oc3ccccc34)c3cc4cc(-c5ccccc5)ccc4c4ccccc34)ccc21 ; CC1(C)c2ccccc2-c2ccc(N(c3ccccc3)c3cccc(-c4cc5cc(-c6ccccc6)ccc5c5ccccc45)c3)cc21]

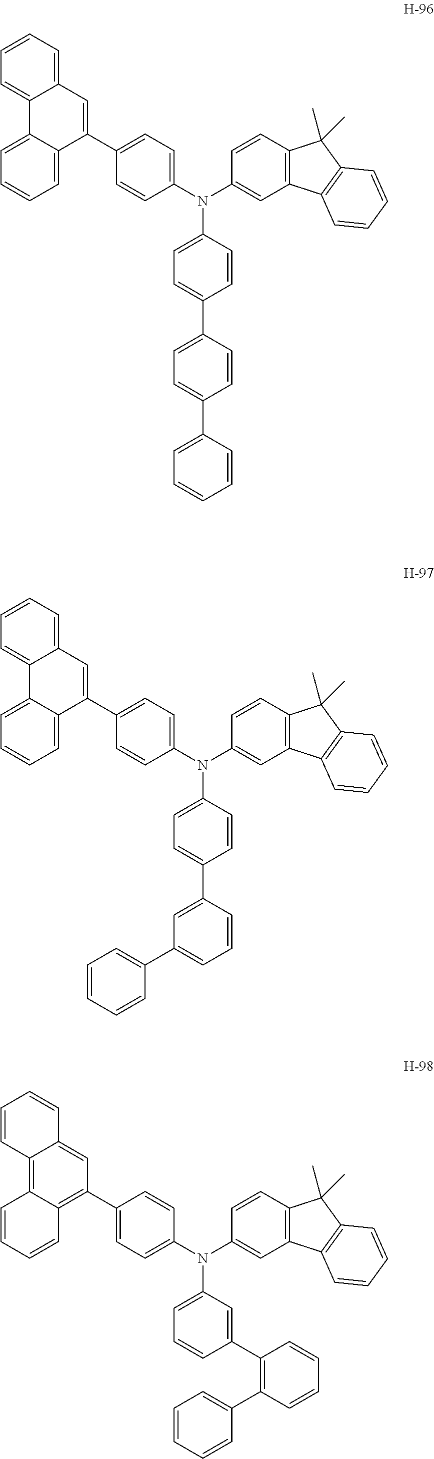 [SMILES: CC1(C)c2ccccc2-c2cc(N(c3ccc(-c4ccc(-c5ccccc5)cc4)cc3)c3ccc(-c4cc5ccccc5c5ccccc45)cc3)ccc21 ; CC1(C)c2ccccc2-c2cc(N(c3ccc(-c4cccc(-c5ccccc5)c4)cc3)c3ccc(-c4cc5ccccc5c5ccccc45)cc3)ccc21 ; CC1(C)c2ccccc2-c2cc(N(c3ccc(-c4cc5ccccc5c5ccccc45)cc3)c3cccc(-c4ccccc4-c4ccccc4)c3)ccc21]

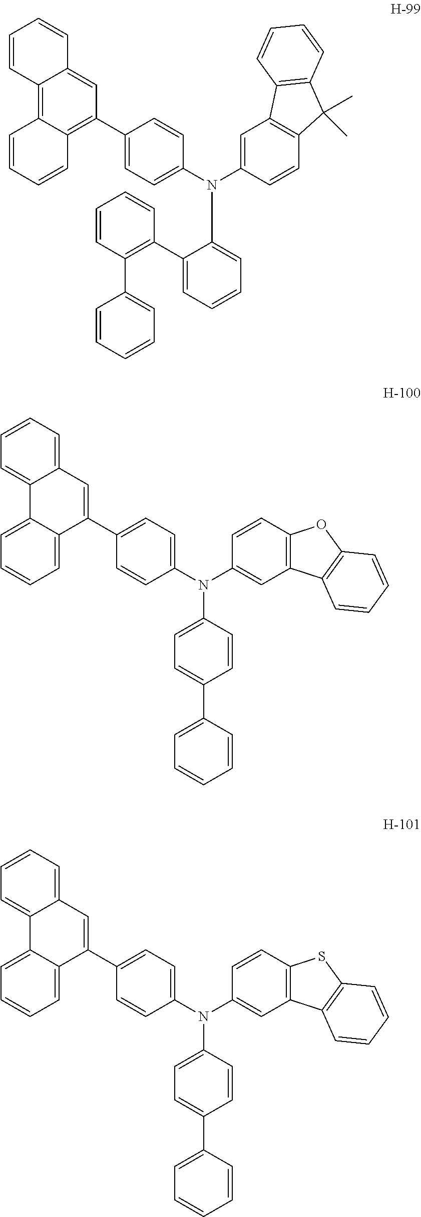 [SMILES: CC1(C)c2ccccc2-c2cc(N(c3ccc(-c4cc5ccccc5c5ccccc45)cc3)c3ccccc3-c3ccccc3-c3ccccc3)ccc21 ; c1ccc(-c2ccc(N(c3ccc(-c4cc5ccccc5c5ccccc45)cc3)c3ccc4oc5ccccc5c4c3)cc2)cc1 ; c1ccc(-c2ccc(N(c3ccc(-c4cc5ccccc5c5ccccc45)cc3)c3ccc4sc5ccccc5c4c3)cc2)cc1]

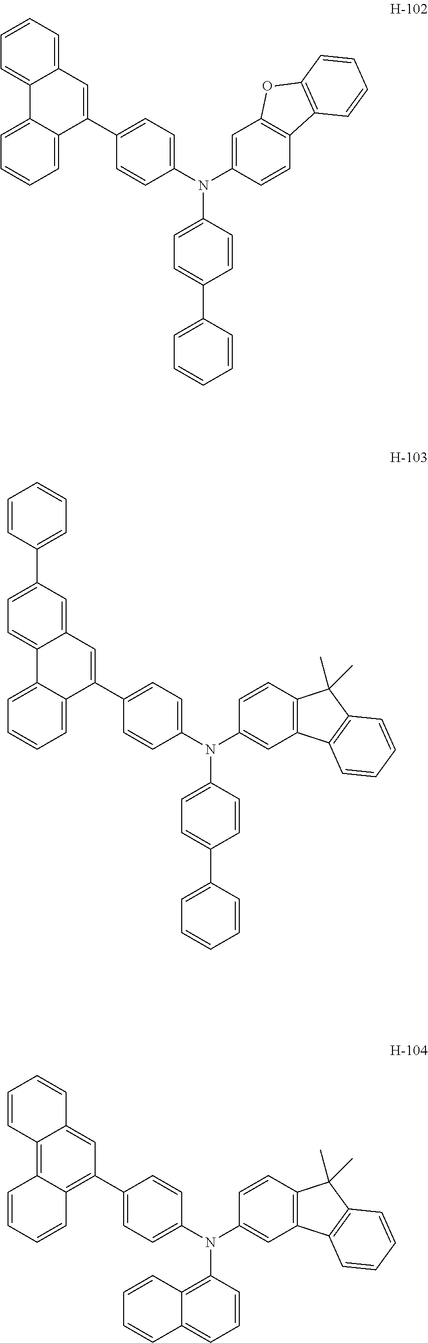 [SMILES: c1ccc(-c2ccc(N(c3ccc(-c4cc5ccccc5c5ccccc45)cc3)c3ccc4c(c3)oc3ccccc34)cc2)cc1 ; CC1(C)c2ccccc2-c2cc(N(c3ccc(-c4ccccc4)cc3)c3ccc(-c4cc5cc(-c6ccccc6)ccc5c5ccccc45)cc3)ccc21 ; CC1(C)c2ccccc2-c2cc(N(c3ccc(-c4cc5ccccc5c5ccccc45)cc3)c3cccc4ccccc34)ccc21]

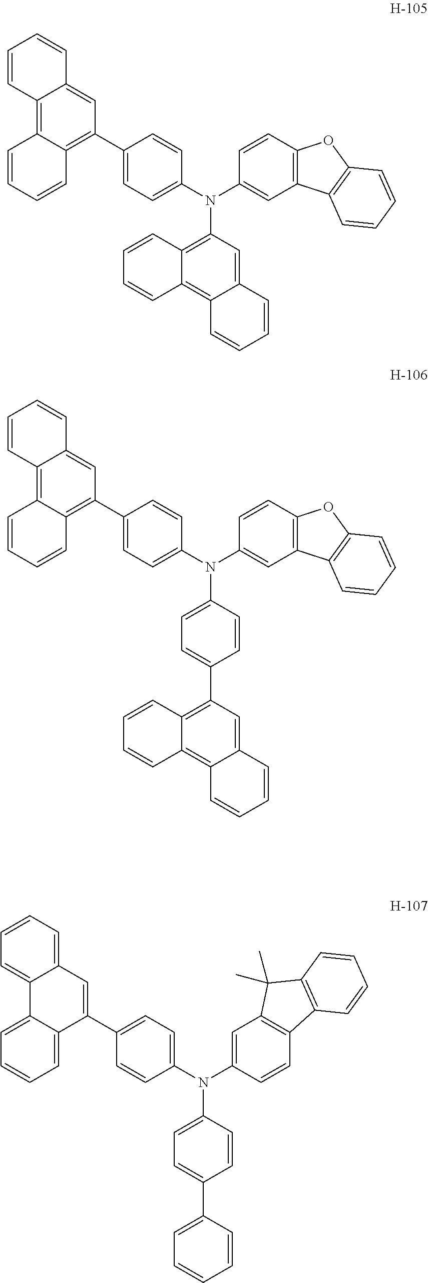 [SMILES: c1ccc2c(c1)cc(-c1ccc(N(c3ccc4oc5ccccc5c4c3)c3cc4ccccc4c4ccccc34)cc1)c1ccccc12 ; c1ccc2c(c1)cc(-c1ccc(N(c3ccc(-c4cc5ccccc5c5ccccc45)cc3)c3ccc4oc5ccccc5c4c3)cc1)c1ccccc12 ; CC1(C)c2ccccc2-c2ccc(N(c3ccc(-c4ccccc4)cc3)c3ccc(-c4cc5ccccc5c5ccccc45)cc3)cc21]

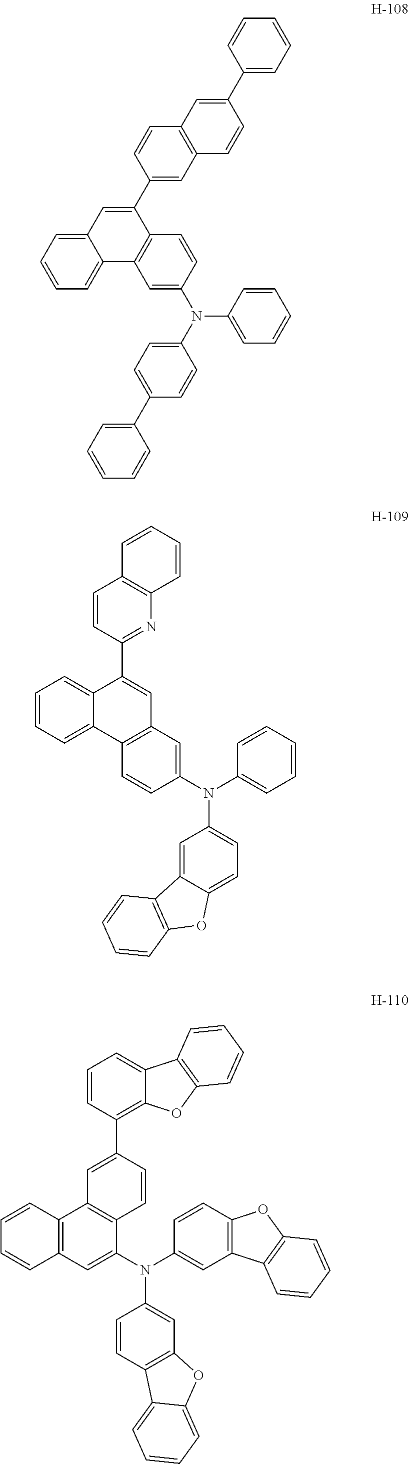

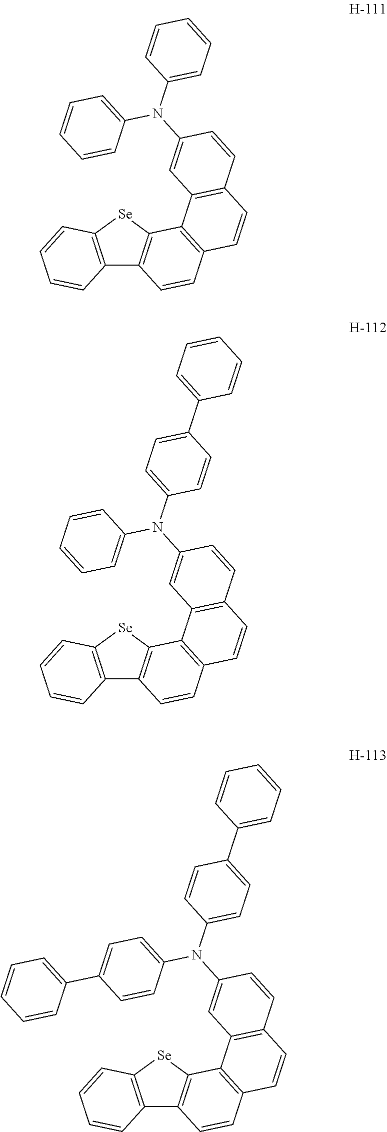 [SMILES: c1ccc(N(c2ccccc2)c2ccc3ccc4ccc5c6ccccc6[se]c5c4c3c2)cc1 ; c1ccc(-c2ccc(N(c3ccccc3)c3ccc4ccc5ccc6c7ccccc7[se]c6c5c4c3)cc2)cc1 ; c1ccc(-c2ccc(N(c3ccc(-c4ccccc4)cc3)c3ccc4ccc5ccc6c7ccccc7[se]c6c5c4c3)cc2)cc1]

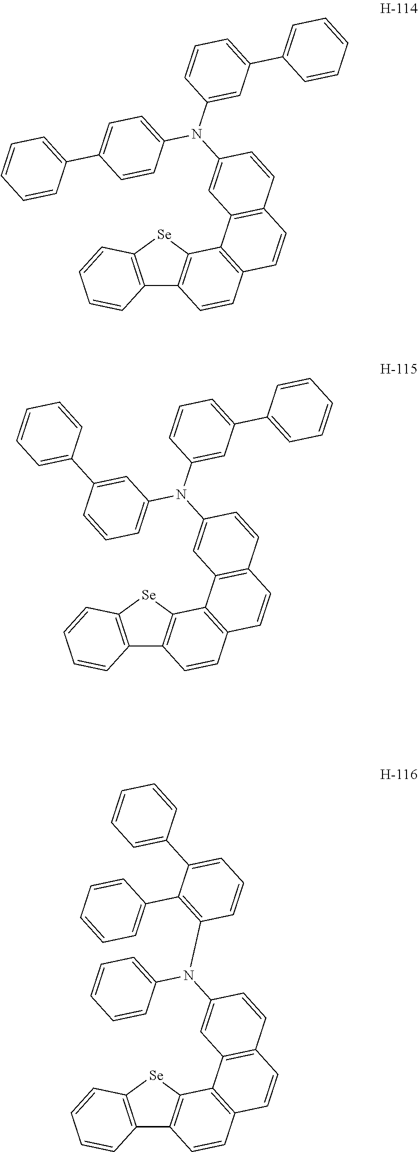 [SMILES: c1ccc(-c2ccc(N(c3cccc(-c4ccccc4)c3)c3ccc4ccc5ccc6c7ccccc7[se]c6c5c4c3)cc2)cc1 ; c1ccc(-c2cccc(N(c3cccc(-c4ccccc4)c3)c3ccc4ccc5ccc6c7ccccc7[se]c6c5c4c3)c2)cc1 ; c1ccc(-c2cccc(N(c3ccccc3)c3ccc4ccc5ccc6c7ccccc7[se]c6c5c4c3)c2-c2ccccc2)cc1]

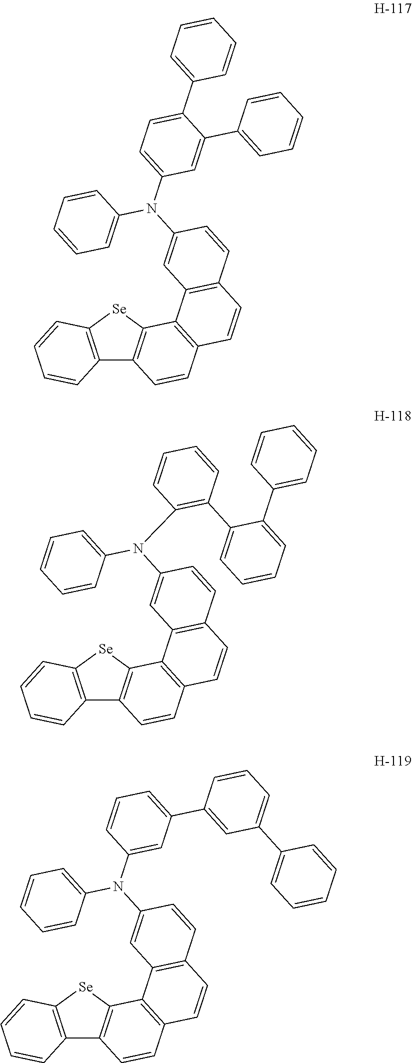 [SMILES: c1ccc(-c2ccc(N(c3ccccc3)c3ccc4ccc5ccc6c7ccccc7[se]c6c5c4c3)cc2-c2ccccc2)cc1 ; c1ccc(-c2ccccc2-c2ccccc2N(c2ccccc2)c2ccc3ccc4ccc5c6ccccc6[se]c5c4c3c2)cc1 ; c1ccc(-c2cccc(-c3cccc(N(c4ccccc4)c4ccc5ccc6ccc7c8ccccc8[se]c7c6c5c4)c3)c2)cc1]

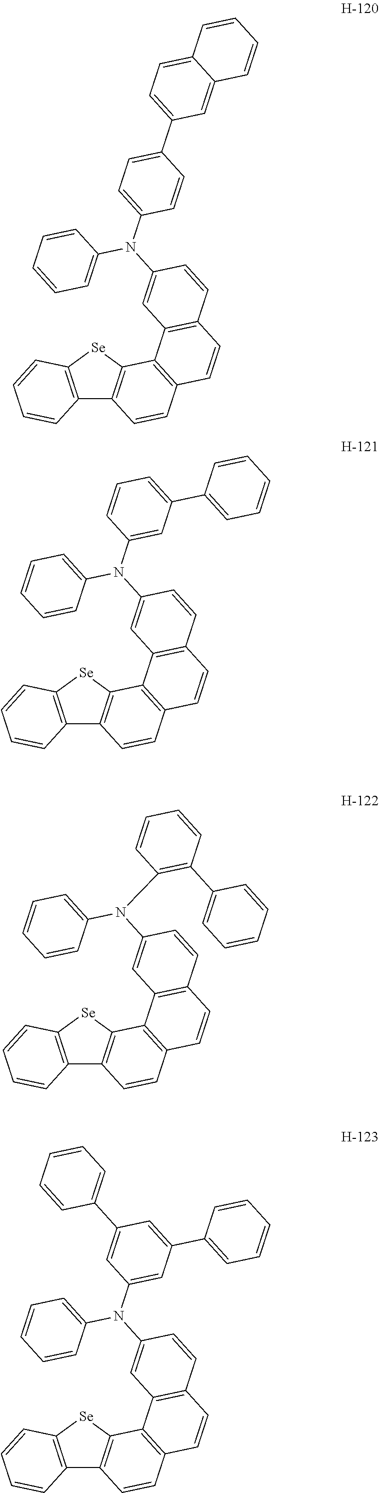 [SMILES: c1ccc(N(c2ccc(-c3ccc4ccccc4c3)cc2)c2ccc3ccc4ccc5c6ccccc6[se]c5c4c3c2)cc1 ; c1ccc(-c2cccc(N(c3ccccc3)c3ccc4ccc5ccc6c7ccccc7[se]c6c5c4c3)c2)cc1 ; c1ccc(-c2ccccc2N(c2ccccc2)c2ccc3ccc4ccc5c6ccccc6[se]c5c4c3c2)cc1 ; c1ccc(-c2cc(-c3ccccc3)cc(N(c3ccccc3)c3ccc4ccc5ccc6c7ccccc7[se]c6c5c4c3)c2)cc1]

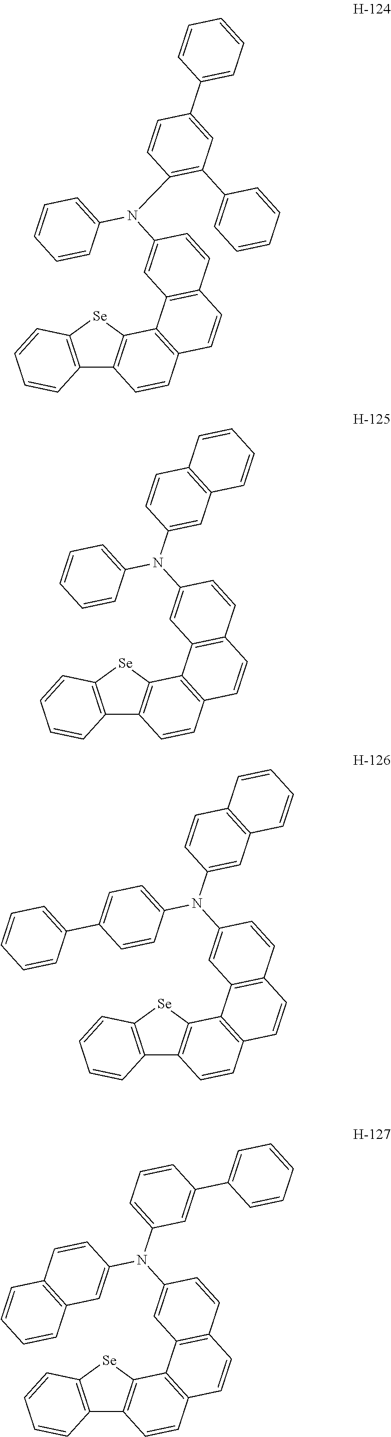 [SMILES: c1ccc(-c2ccc(N(c3ccccc3)c3ccc4ccc5ccc6c7ccccc7[se]c6c5c4c3)c(-c3ccccc3)c2)cc1 ; c1ccc(N(c2ccc3ccccc3c2)c2ccc3ccc4ccc5c6ccccc6[se]c5c4c3c2)cc1 ; c1ccc(-c2ccc(N(c3ccc4ccccc4c3)c3ccc4ccc5ccc6c7ccccc7[se]c6c5c4c3)cc2)cc1 ; c1ccc(-c2cccc(N(c3ccc4ccccc4c3)c3ccc4ccc5ccc6c7ccccc7[se]c6c5c4c3)c2)cc1]

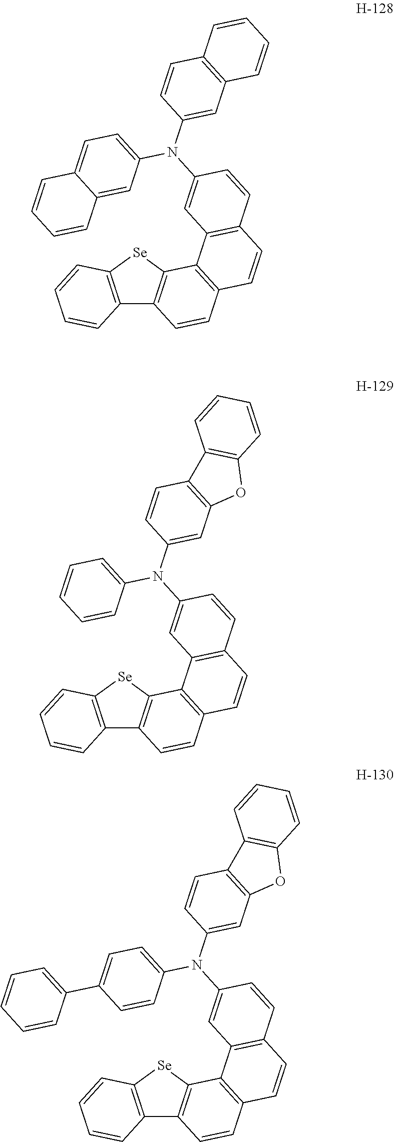 [SMILES: c1ccc2cc(N(c3ccc4ccccc4c3)c3ccc4ccc5ccc6c7ccccc7[se]c6c5c4c3)ccc2c1 ; c1ccc(N(c2ccc3c(c2)oc2ccccc23)c2ccc3ccc4ccc5c6ccccc6[se]c5c4c3c2)cc1 ; c1ccc(-c2ccc(N(c3ccc4c(c3)oc3ccccc34)c3ccc4ccc5ccc6c7ccccc7[se]c6c5c4c3)cc2)cc1]

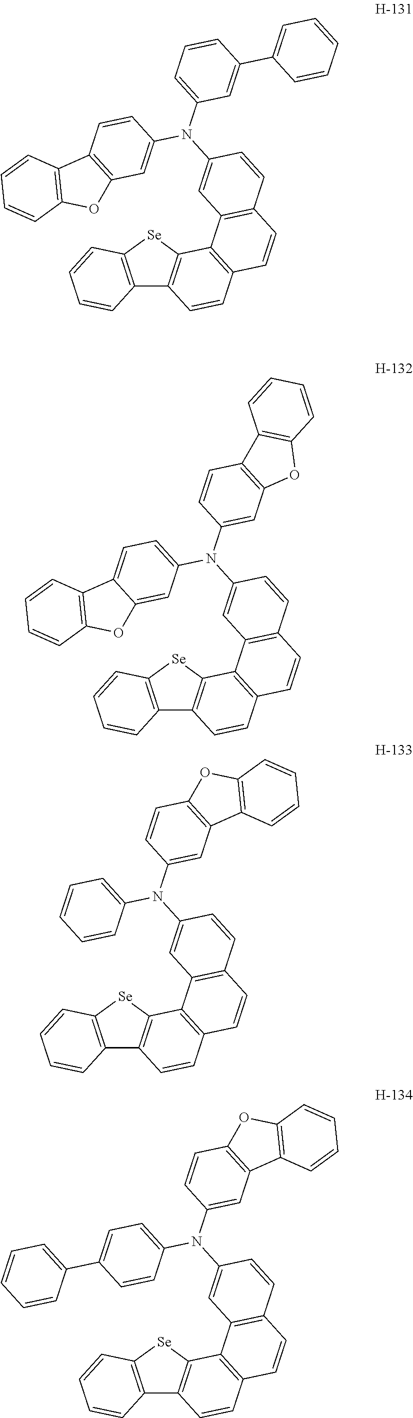

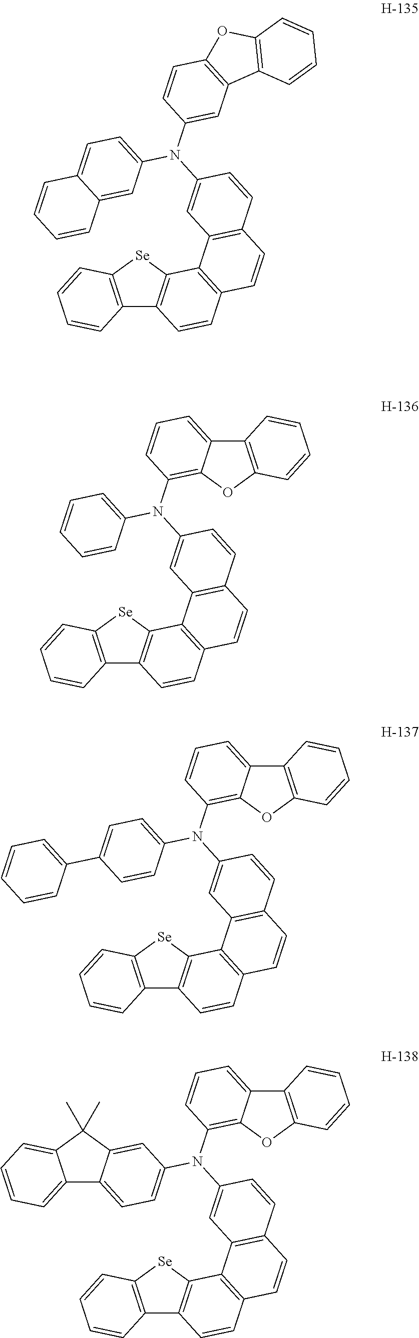 [SMILES: c1ccc2cc(N(c3ccc4oc5ccccc5c4c3)c3ccc4ccc5ccc6c7ccccc7[se]c6c5c4c3)ccc2c1 ; c1ccc(N(c2ccc3ccc4ccc5c6ccccc6[se]c5c4c3c2)c2cccc3c2oc2ccccc23)cc1 ; c1ccc(-c2ccc(N(c3ccc4ccc5ccc6c7ccccc7[se]c6c5c4c3)c3cccc4c3oc3ccccc34)cc2)cc1 ; CC1(C)c2ccccc2-c2ccc(N(c3ccc4ccc5ccc6c7ccccc7[se]c6c5c4c3)c3cccc4c3oc3ccccc34)cc21]

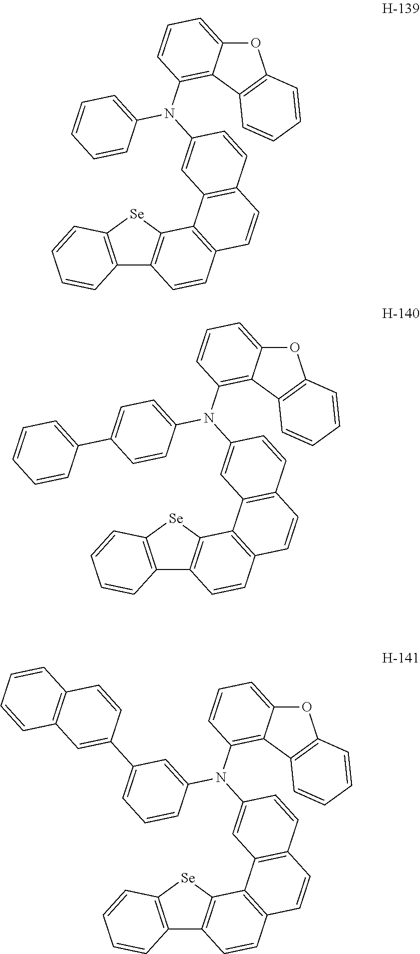 [SMILES: c1ccc(N(c2ccc3ccc4ccc5c6ccccc6[se]c5c4c3c2)c2cccc3oc4ccccc4c23)cc1 ; c1ccc(-c2ccc(N(c3ccc4ccc5ccc6c7ccccc7[se]c6c5c4c3)c3cccc4oc5ccccc5c34)cc2)cc1 ; c1cc(-c2ccc3ccccc3c2)cc(N(c2ccc3ccc4ccc5c6ccccc6[se]c5c4c3c2)c2cccc3oc4ccccc4c23)c1]

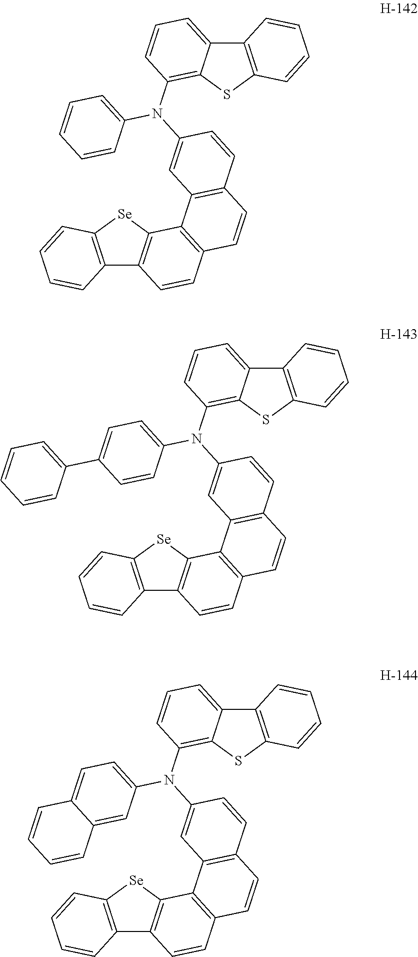 [SMILES: c1ccc(N(c2ccc3ccc4ccc5c6ccccc6[se]c5c4c3c2)c2cccc3c2sc2ccccc23)cc1 ; c1ccc(-c2ccc(N(c3ccc4ccc5ccc6c7ccccc7[se]c6c5c4c3)c3cccc4c3sc3ccccc34)cc2)cc1 ; c1ccc2cc(N(c3ccc4ccc5ccc6c7ccccc7[se]c6c5c4c3)c3cccc4c3sc3ccccc34)ccc2c1]

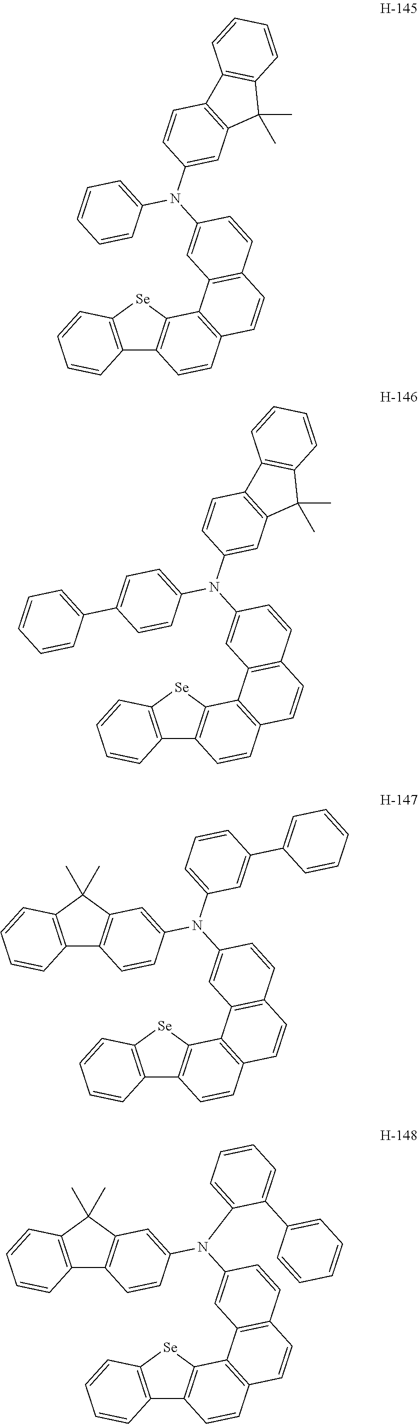 [SMILES: CC1(C)c2ccccc2-c2ccc(N(c3ccccc3)c3ccc4ccc5ccc6c7ccccc7[se]c6c5c4c3)cc21 ; CC1(C)c2ccccc2-c2ccc(N(c3ccc(-c4ccccc4)cc3)c3ccc4ccc5ccc6c7ccccc7[se]c6c5c4c3)cc21 ; CC1(C)c2ccccc2-c2ccc(N(c3cccc(-c4ccccc4)c3)c3ccc4ccc5ccc6c7ccccc7[se]c6c5c4c3)cc21 ; CC1(C)c2ccccc2-c2ccc(N(c3ccc4ccc5ccc6c7ccccc7[se]c6c5c4c3)c3ccccc3-c3ccccc3)cc21]

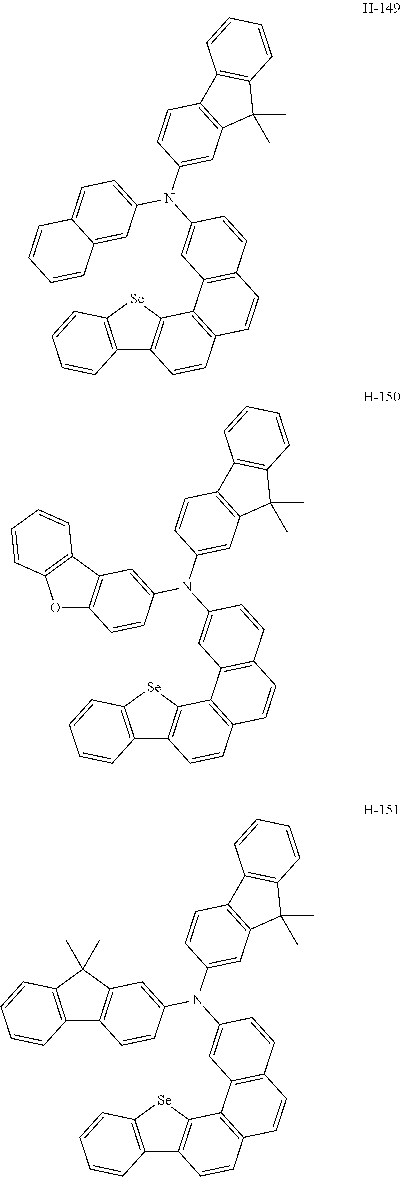 [SMILES: CC1(C)c2ccccc2-c2ccc(N(c3ccc4ccccc4c3)c3ccc4ccc5ccc6c7ccccc7[se]c6c5c4c3)cc21 ; CC1(C)c2ccccc2-c2ccc(N(c3ccc4oc5ccccc5c4c3)c3ccc4ccc5ccc6c7ccccc7[se]c6c5c4c3)cc21 ; CC1(C)c2ccccc2-c2ccc(N(c3ccc4c(c3)C(C)(C)c3ccccc3-4)c3ccc4ccc5ccc6c7ccccc7[se]c6c5c4c3)cc21]

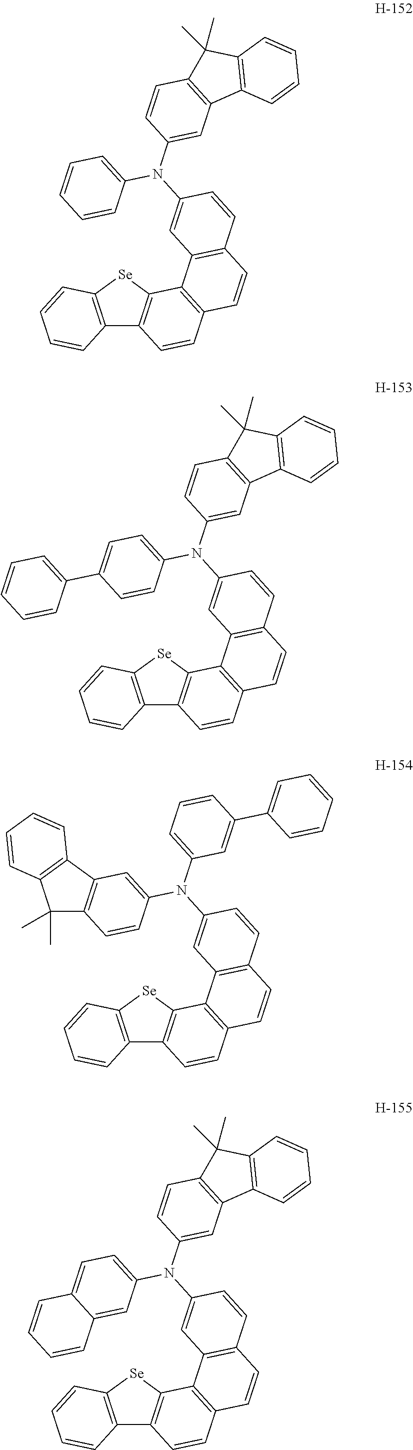 [SMILES: CC1(C)c2ccccc2-c2cc(N(c3ccccc3)c3ccc4ccc5ccc6c7ccccc7[se]c6c5c4c3)ccc21 ; CC1(C)c2ccccc2-c2cc(N(c3ccc(-c4ccccc4)cc3)c3ccc4ccc5ccc6c7ccccc7[se]c6c5c4c3)ccc21 ; CC1(C)c2ccccc2-c2cc(N(c3cccc(-c4ccccc4)c3)c3ccc4ccc5ccc6c7ccccc7[se]c6c5c4c3)ccc21 ; CC1(C)c2ccccc2-c2cc(N(c3ccc4ccccc4c3)c3ccc4ccc5ccc6c7ccccc7[se]c6c5c4c3)ccc21]

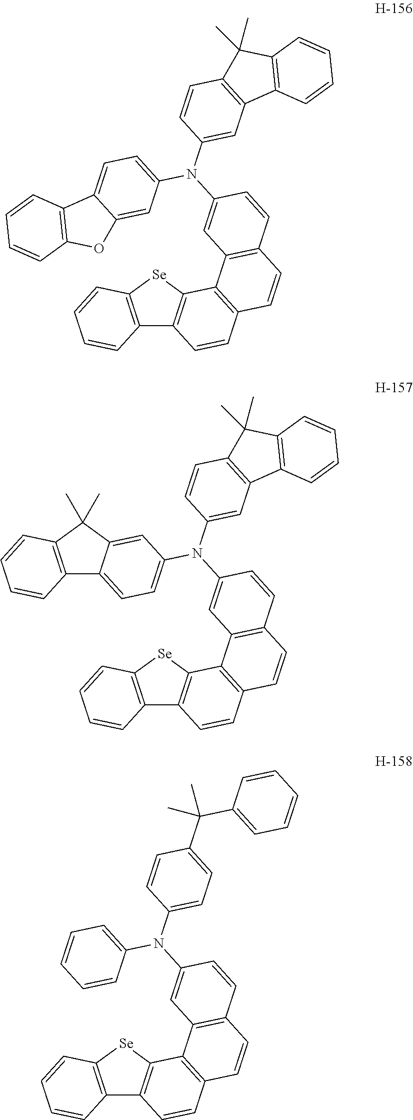 [SMILES: CC1(C)c2ccccc2-c2cc(N(c3ccc4c(c3)oc3ccccc34)c3ccc4ccc5ccc6c7ccccc7[se]c6c5c4c3)ccc21 ; CC1(C)c2ccccc2-c2cc(N(c3ccc4c(c3)C(C)(C)c3ccccc3-4)c3ccc4ccc5ccc6c7ccccc7[se]c6c5c4c3)ccc21 ; CC(C)(c1ccccc1)c1ccc(N(c2ccccc2)c2ccc3ccc4ccc5c6ccccc6[se]c5c4c3c2)cc1]

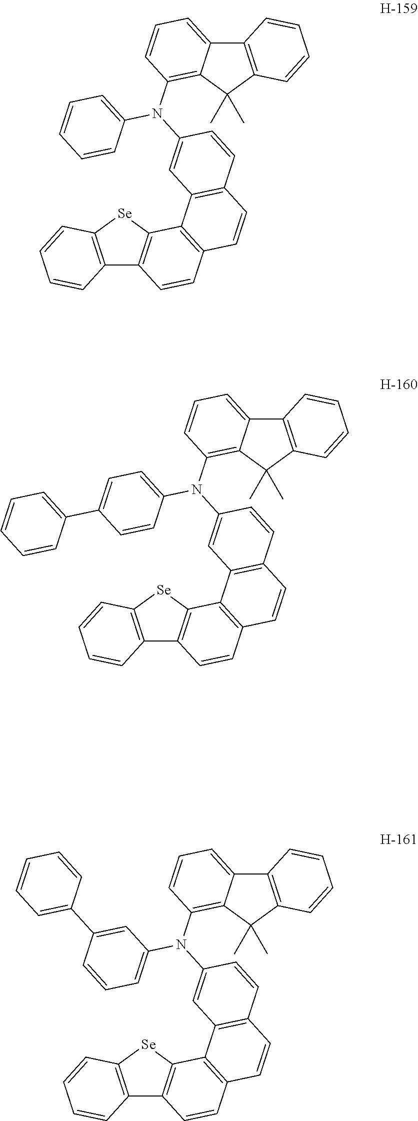 [SMILES: CC1(C)c2ccccc2-c2cccc(N(c3ccccc3)c3ccc4ccc5ccc6c7ccccc7[se]c6c5c4c3)c21 ; CC1(C)c2ccccc2-c2cccc(N(c3ccc(-c4ccccc4)cc3)c3ccc4ccc5ccc6c7ccccc7[se]c6c5c4c3)c21 ; CC1(C)c2ccccc2-c2cccc(N(c3cccc(-c4ccccc4)c3)c3ccc4ccc5ccc6c7ccccc7[se]c6c5c4c3)c21]

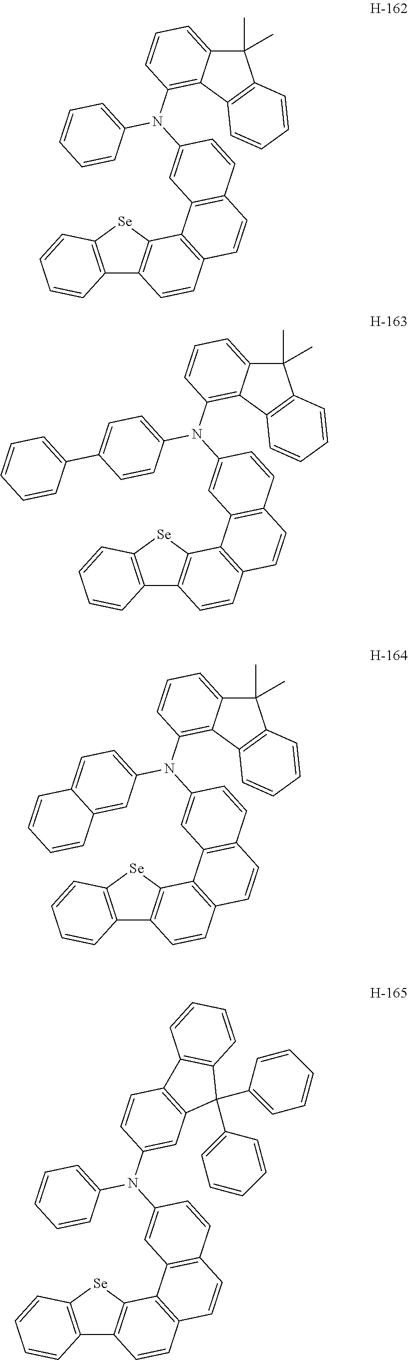 [SMILES: CC1(C)c2ccccc2-c2c(N(c3ccccc3)c3ccc4ccc5ccc6c7ccccc7[se]c6c5c4c3)cccc21 ; CC1(C)c2ccccc2-c2c(N(c3ccc(-c4ccccc4)cc3)c3ccc4ccc5ccc6c7ccccc7[se]c6c5c4c3)cccc21 ; CC1(C)c2ccccc2-c2c(N(c3ccc4ccccc4c3)c3ccc4ccc5ccc6c7ccccc7[se]c6c5c4c3)cccc21 ; c1ccc(N(c2ccc3c(c2)C(c2ccccc2)(c2ccccc2)c2ccccc2-3)c2ccc3ccc4ccc5c6ccccc6[se]c5c4c3c2)cc1]

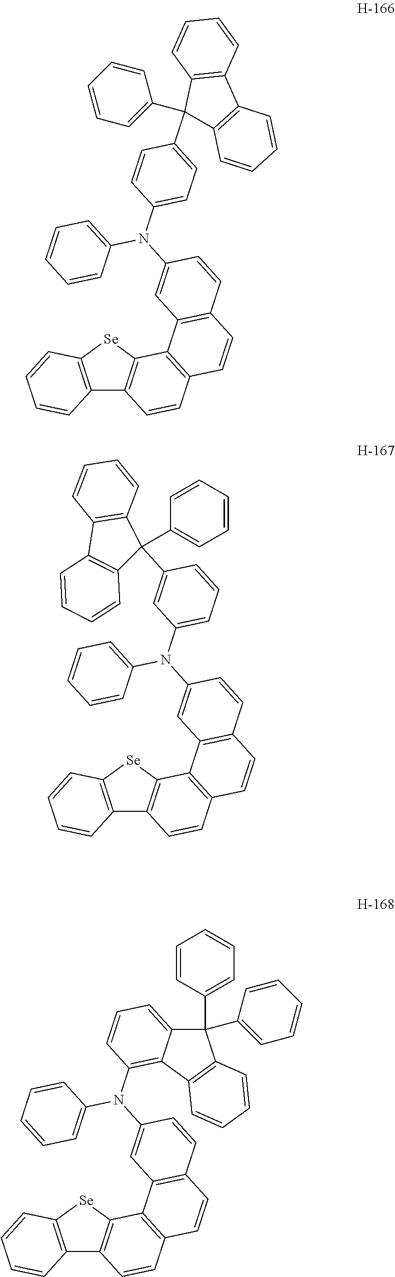 [SMILES: c1ccc(N(c2ccc(C3(c4ccccc4)c4ccccc4-c4ccccc43)cc2)c2ccc3ccc4ccc5c6ccccc6[se]c5c4c3c2)cc1 ; c1ccc(N(c2cccc(C3(c4ccccc4)c4ccccc4-c4ccccc43)c2)c2ccc3ccc4ccc5c6ccccc6[se]c5c4c3c2)cc1 ; c1ccc(N(c2ccc3ccc4ccc5c6ccccc6[se]c5c4c3c2)c2cccc3c2-c2ccccc2C3(c2ccccc2)c2ccccc2)cc1]

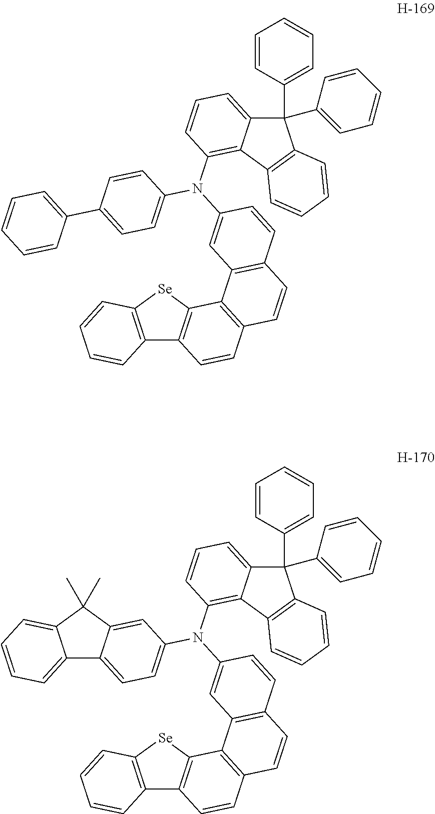 [SMILES: c1ccc(-c2ccc(N(c3ccc4ccc5ccc6c7ccccc7[se]c6c5c4c3)c3cccc4c3-c3ccccc3C4(c3ccccc3)c3ccccc3)cc2)cc1 ; CC1(C)c2ccccc2-c2ccc(N(c3ccc4ccc5ccc6c7ccccc7[se]c6c5c4c3)c3cccc4c3-c3ccccc3C4(c3ccccc3)c3ccccc3)cc21]

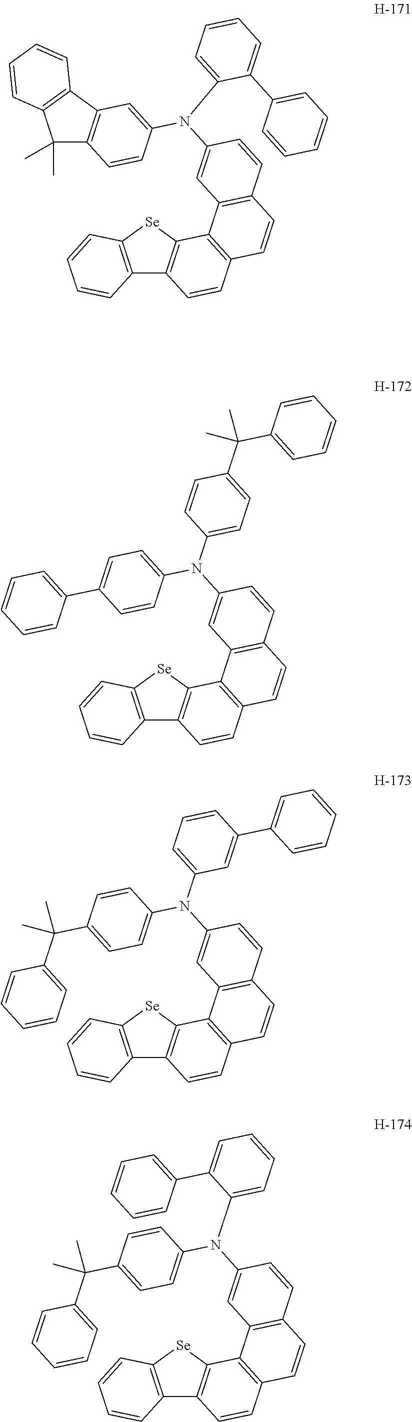 [SMILES: CC1(C)c2ccccc2-c2cc(N(c3ccc4ccc5ccc6c7ccccc7[se]c6c5c4c3)c3ccccc3-c3ccccc3)ccc21 ; CC(C)(c1ccccc1)c1ccc(N(c2ccc(-c3ccccc3)cc2)c2ccc3ccc4ccc5c6ccccc6[se]c5c4c3c2)cc1 ; CC(C)(c1ccccc1)c1ccc(N(c2cccc(-c3ccccc3)c2)c2ccc3ccc4ccc5c6ccccc6[se]c5c4c3c2)cc1 ; CC(C)(c1ccccc1)c1ccc(N(c2ccc3ccc4ccc5c6ccccc6[se]c5c4c3c2)c2ccccc2-c2ccccc2)cc1]

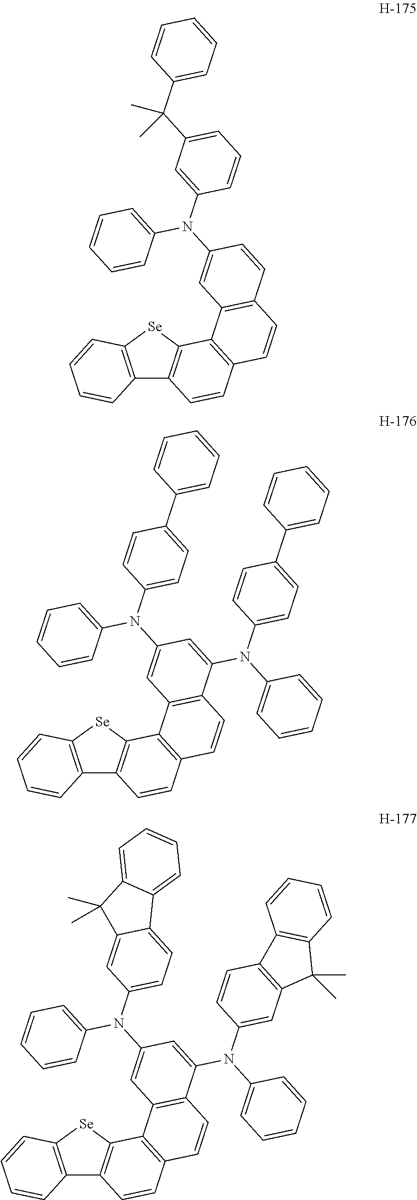

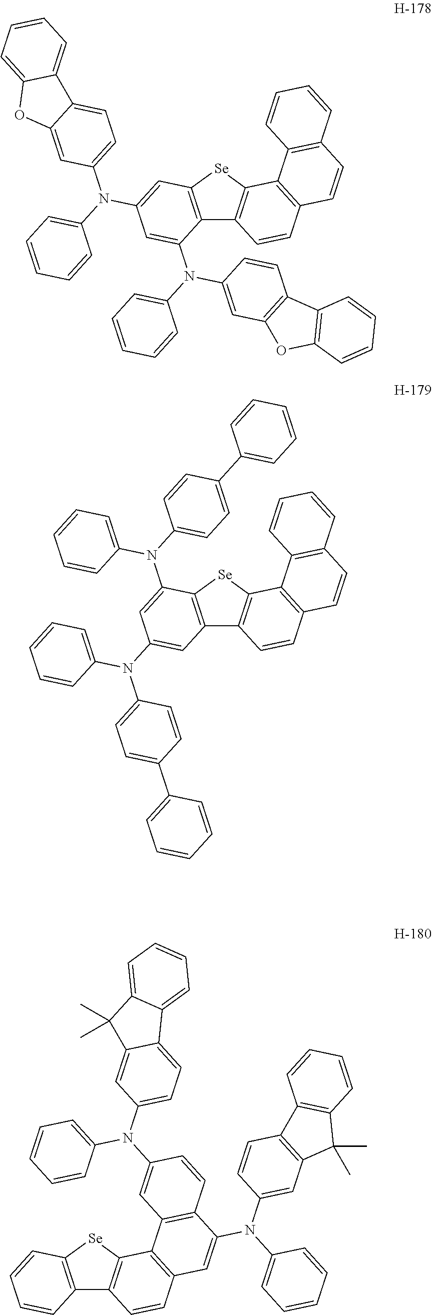 [SMILES: c1ccc(N(c2ccc3c(c2)oc2ccccc23)c2cc(N(c3ccccc3)c3ccc4c(c3)oc3ccccc34)c3c(c2)[se]c2c3ccc3ccc4ccccc4c32)cc1 ; c1ccc(-c2ccc(N(c3ccccc3)c3cc(N(c4ccccc4)c4ccc(-c5ccccc5)cc4)c4[se]c5c(ccc6ccc7ccccc7c65)c4c3)cc2)cc1 ; CC1(C)c2ccccc2-c2ccc(N(c3ccccc3)c3ccc4c(N(c5ccccc5)c5ccc6c(c5)C(C)(C)c5ccccc5-6)cc5ccc6c7ccccc7[se]c6c5c4c3)cc21]

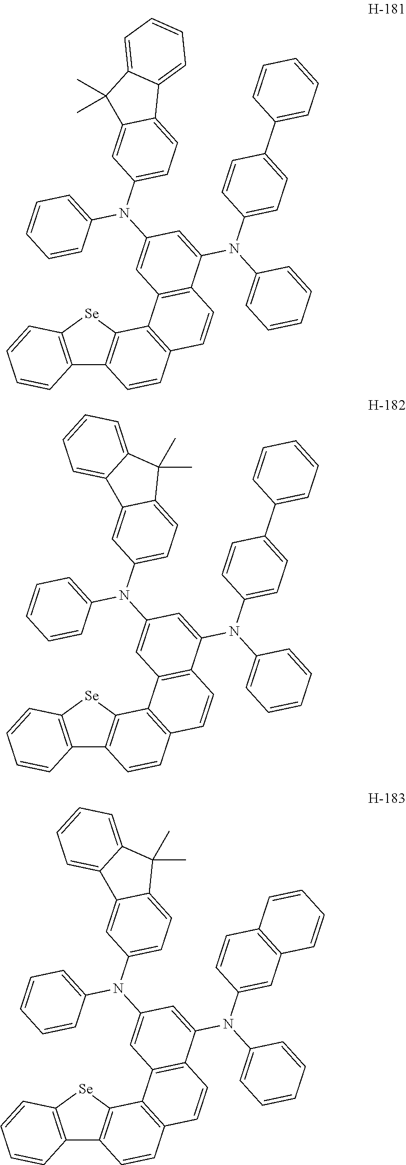

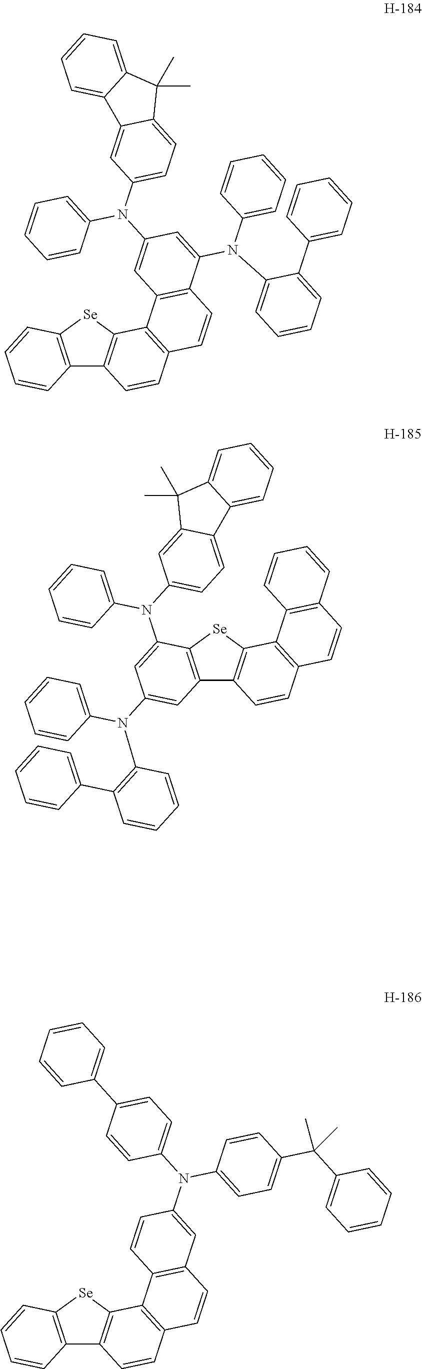 [SMILES: CC1(C)c2ccccc2-c2cc(N(c3ccccc3)c3cc(N(c4ccccc4)c4ccccc4-c4ccccc4)c4ccc5ccc6c7ccccc7[se]c6c5c4c3)ccc21 ; CC1(C)c2ccccc2-c2ccc(N(c3ccccc3)c3cc(N(c4ccccc4)c4ccccc4-c4ccccc4)cc4c3[se]c3c4ccc4ccc5ccccc5c43)cc21 ; CC(C)(c1ccccc1)c1ccc(N(c2ccc(-c3ccccc3)cc2)c2ccc3c(ccc4ccc5c6ccccc6[se]c5c43)c2)cc1]

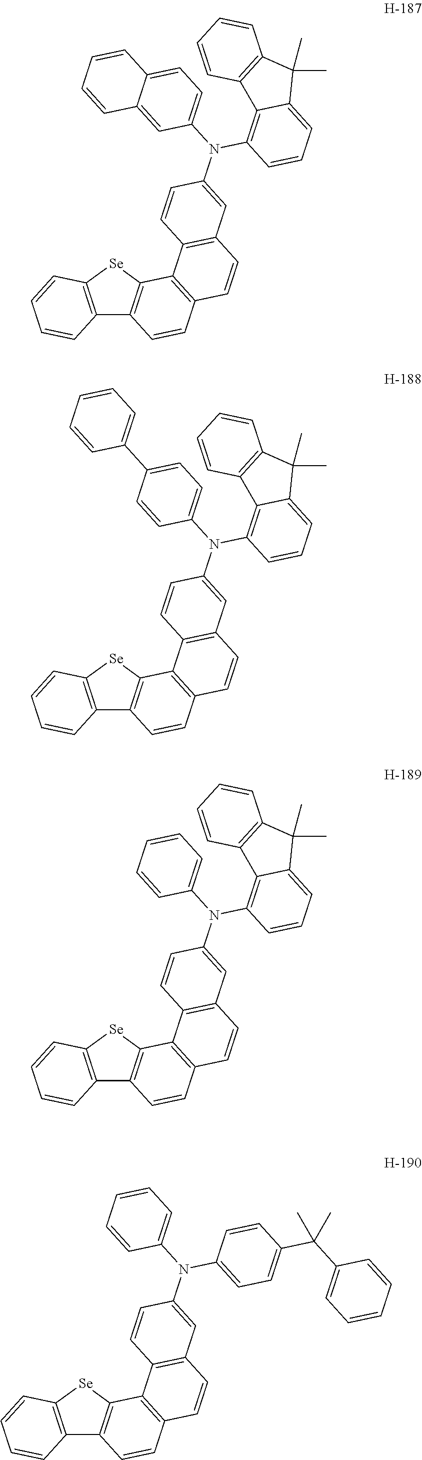 [SMILES: CC1(C)c2ccccc2-c2c(N(c3ccc4ccccc4c3)c3ccc4c(ccc5ccc6c7ccccc7[se]c6c54)c3)cccc21 ; CC1(C)c2ccccc2-c2c(N(c3ccc(-c4ccccc4)cc3)c3ccc4c(ccc5ccc6c7ccccc7[se]c6c54)c3)cccc21 ; CC1(C)c2ccccc2-c2c(N(c3ccccc3)c3ccc4c(ccc5ccc6c7ccccc7[se]c6c54)c3)cccc21 ; CC(C)(c1ccccc1)c1ccc(N(c2ccccc2)c2ccc3c(ccc4ccc5c6ccccc6[se]c5c43)c2)cc1]

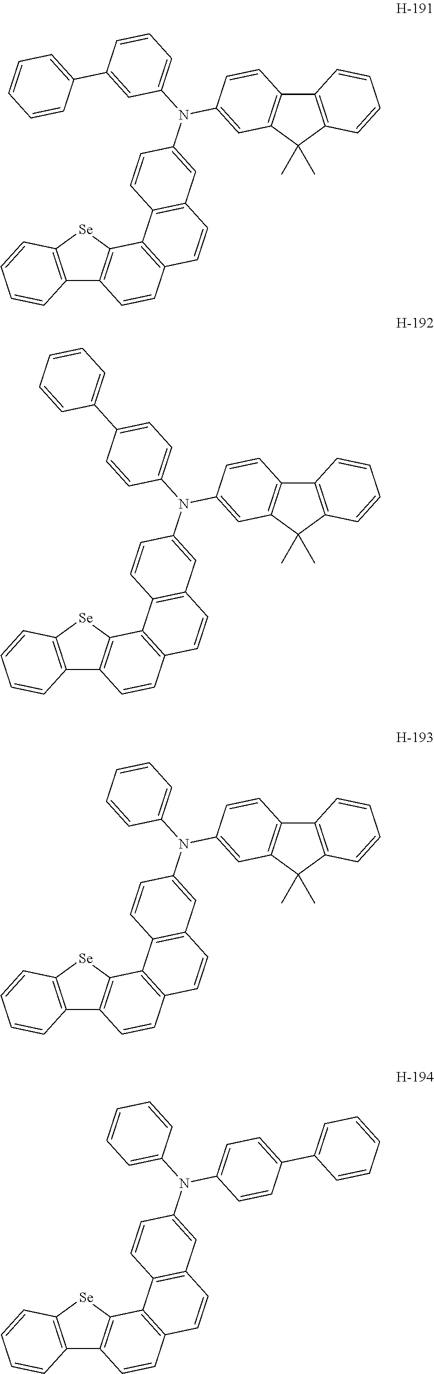 [SMILES: CC1(C)c2ccccc2-c2ccc(N(c3cccc(-c4ccccc4)c3)c3ccc4c(ccc5ccc6c7ccccc7[se]c6c54)c3)cc21 ; CC1(C)c2ccccc2-c2ccc(N(c3ccc(-c4ccccc4)cc3)c3ccc4c(ccc5ccc6c7ccccc7[se]c6c54)c3)cc21 ; CC1(C)c2ccccc2-c2ccc(N(c3ccccc3)c3ccc4c(ccc5ccc6c7ccccc7[se]c6c54)c3)cc21 ; c1ccc(-c2ccc(N(c3ccccc3)c3ccc4c(ccc5ccc6c7ccccc7[se]c6c54)c3)cc2)cc1]

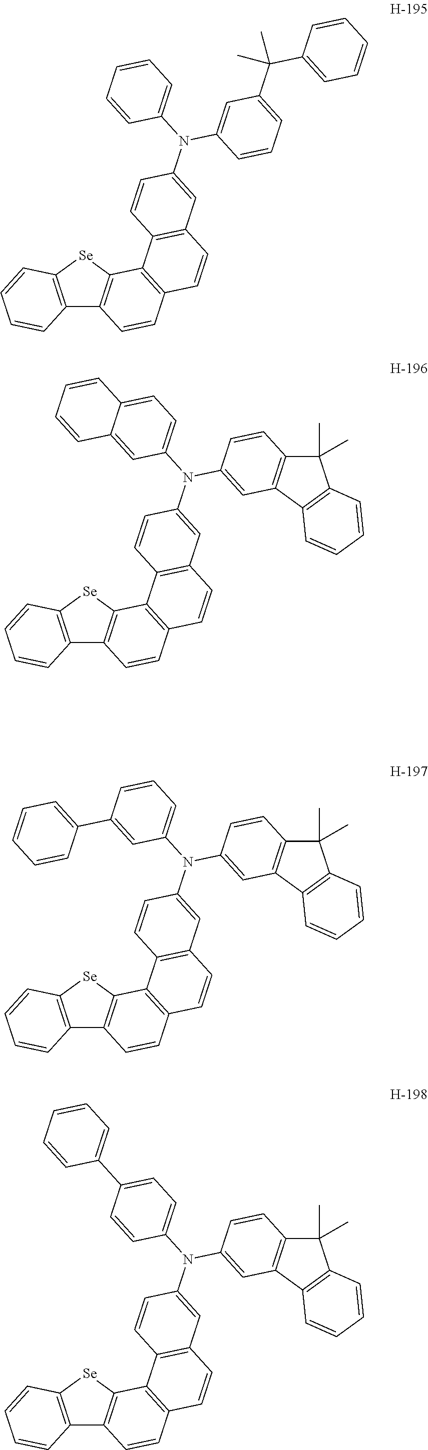 [SMILES: CC(C)(c1ccccc1)c1cccc(N(c2ccccc2)c2ccc3c(ccc4ccc5c6ccccc6[se]c5c43)c2)c1 ; CC1(C)c2ccccc2-c2cc(N(c3ccc4ccccc4c3)c3ccc4c(ccc5ccc6c7ccccc7[se]c6c54)c3)ccc21 ; CC1(C)c2ccccc2-c2cc(N(c3cccc(-c4ccccc4)c3)c3ccc4c(ccc5ccc6c7ccccc7[se]c6c54)c3)ccc21 ; CC1(C)c2ccccc2-c2cc(N(c3ccc(-c4ccccc4)cc3)c3ccc4c(ccc5ccc6c7ccccc7[se]c6c54)c3)ccc21]

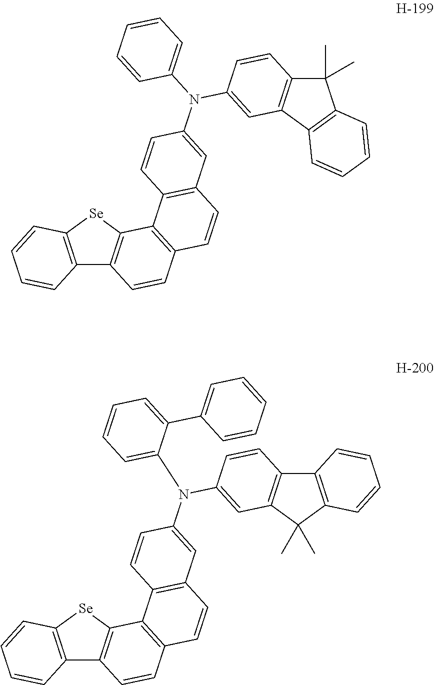 [SMILES: CC1(C)c2ccccc2-c2cc(N(c3ccccc3)c3ccc4c(ccc5ccc6c7ccccc7[se]c6c54)c3)ccc21 ; CC1(C)c2ccccc2-c2ccc(N(c3ccc4c(ccc5ccc6c7ccccc7[se]c6c54)c3)c3ccccc3-c3ccccc3)cc21]

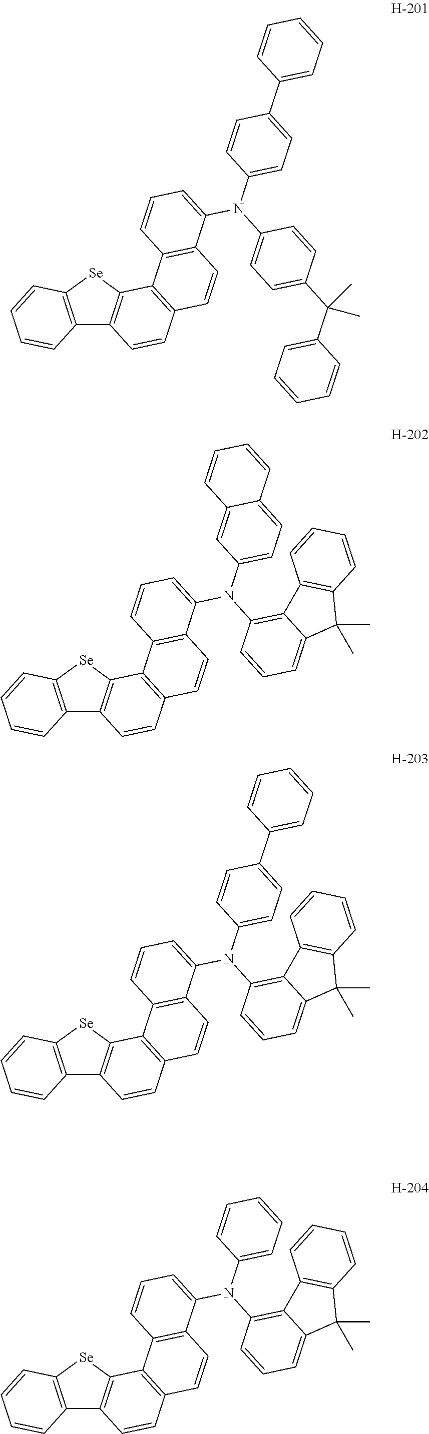 [SMILES: CC(C)(c1ccccc1)c1ccc(N(c2ccc(-c3ccccc3)cc2)c2cccc3c2ccc2ccc4c5ccccc5[se]c4c23)cc1 ; CC1(C)c2ccccc2-c2c(N(c3ccc4ccccc4c3)c3cccc4c3ccc3ccc5c6ccccc6[se]c5c34)cccc21 ; CC1(C)c2ccccc2-c2c(N(c3ccc(-c4ccccc4)cc3)c3cccc4c3ccc3ccc5c6ccccc6[se]c5c34)cccc21 ; CC1(C)c2ccccc2-c2c(N(c3ccccc3)c3cccc4c3ccc3ccc5c6ccccc6[se]c5c34)cccc21]

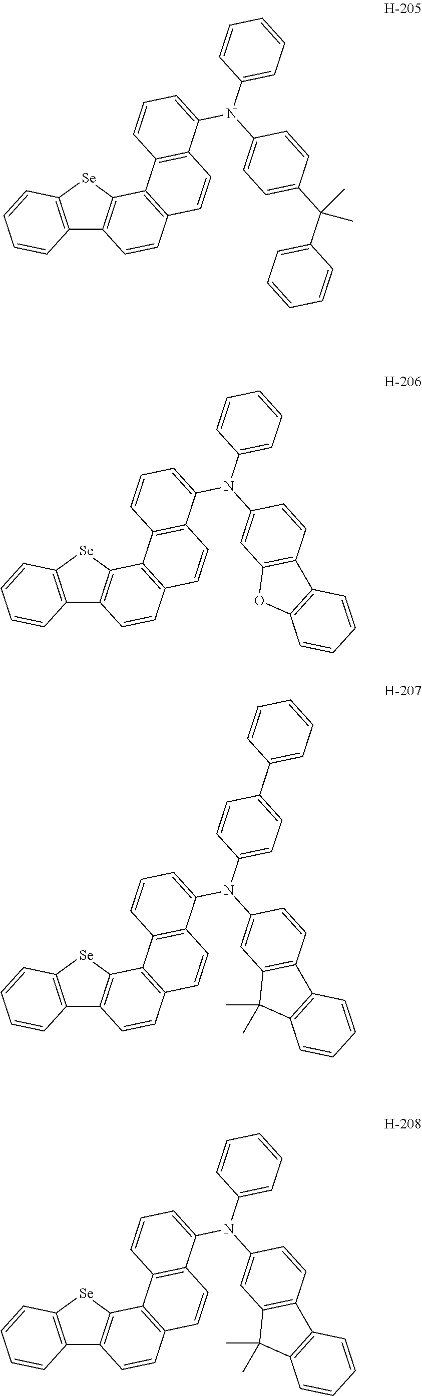 [SMILES: CC(C)(c1ccccc1)c1ccc(N(c2ccccc2)c2cccc3c2ccc2ccc4c5ccccc5[se]c4c23)cc1 ; c1ccc(N(c2ccc3c(c2)oc2ccccc23)c2cccc3c2ccc2ccc4c5ccccc5[se]c4c23)cc1 ; CC1(C)c2ccccc2-c2ccc(N(c3ccc(-c4ccccc4)cc3)c3cccc4c3ccc3ccc5c6ccccc6[se]c5c34)cc21 ; CC1(C)c2ccccc2-c2ccc(N(c3ccccc3)c3cccc4c3ccc3ccc5c6ccccc6[se]c5c34)cc21]

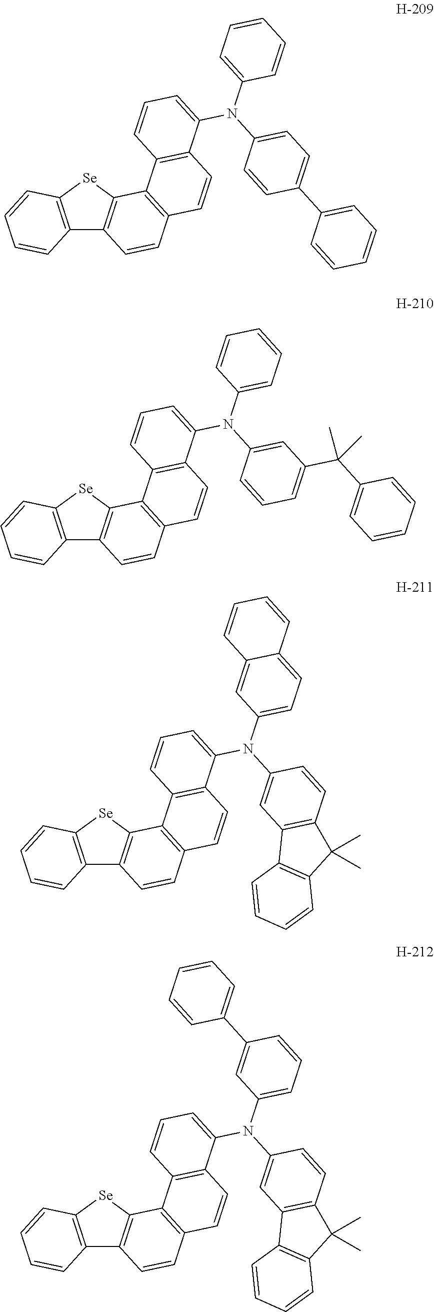 [SMILES: c1ccc(-c2ccc(N(c3ccccc3)c3cccc4c3ccc3ccc5c6ccccc6[se]c5c34)cc2)cc1 ; CC(C)(c1ccccc1)c1cccc(N(c2ccccc2)c2cccc3c2ccc2ccc4c5ccccc5[se]c4c23)c1 ; CC1(C)c2ccccc2-c2cc(N(c3ccc4ccccc4c3)c3cccc4c3ccc3ccc5c6ccccc6[se]c5c34)ccc21 ; CC1(C)c2ccccc2-c2cc(N(c3cccc(-c4ccccc4)c3)c3cccc4c3ccc3ccc5c6ccccc6[se]c5c34)ccc21]

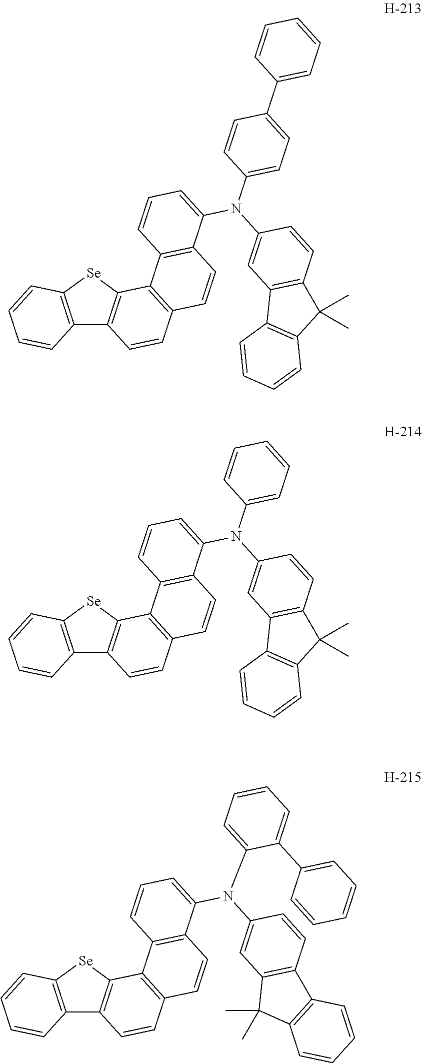 [SMILES: CC1(C)c2ccccc2-c2cc(N(c3ccc(-c4ccccc4)cc3)c3cccc4c3ccc3ccc5c6ccccc6[se]c5c34)ccc21 ; CC1(C)c2ccccc2-c2cc(N(c3ccccc3)c3cccc4c3ccc3ccc5c6ccccc6[se]c5c34)ccc21 ; CC1(C)c2ccccc2-c2ccc(N(c3ccccc3-c3ccccc3)c3cccc4c3ccc3ccc5c6ccccc6[se]c5c34)cc21]

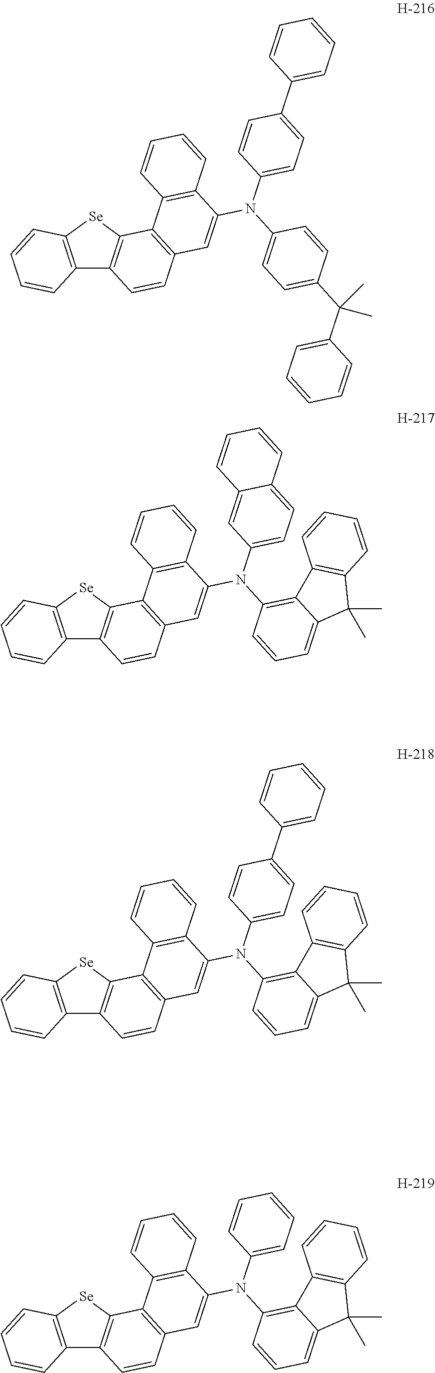 [SMILES: CC(C)(c1ccccc1)c1ccc(N(c2ccc(-c3ccccc3)cc2)c2cc3ccc4c5ccccc5[se]c4c3c3ccccc23)cc1 ; CC1(C)c2ccccc2-c2c(N(c3ccc4ccccc4c3)c3cc4ccc5c6ccccc6[se]c5c4c4ccccc34)cccc21 ; CC1(C)c2ccccc2-c2c(N(c3ccc(-c4ccccc4)cc3)c3cc4ccc5c6ccccc6[se]c5c4c4ccccc34)cccc21 ; CC1(C)c2ccccc2-c2c(N(c3ccccc3)c3cc4ccc5c6ccccc6[se]c5c4c4ccccc34)cccc21]

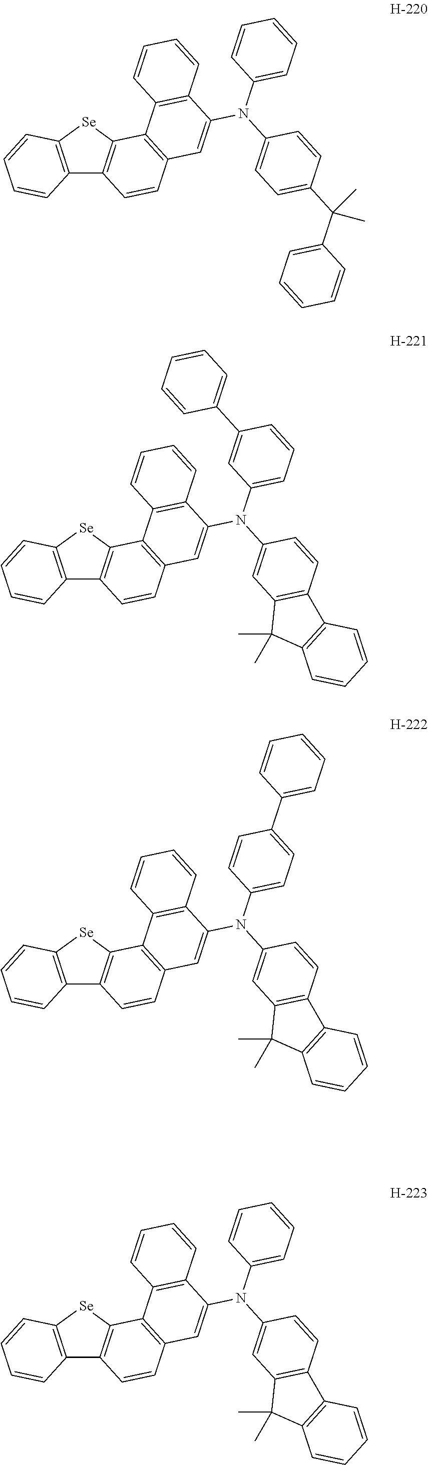 [SMILES: CC(C)(c1ccccc1)c1ccc(N(c2ccccc2)c2cc3ccc4c5ccccc5[se]c4c3c3ccccc23)cc1 ; CC1(C)c2ccccc2-c2ccc(N(c3cccc(-c4ccccc4)c3)c3cc4ccc5c6ccccc6[se]c5c4c4ccccc34)cc21 ; CC1(C)c2ccccc2-c2ccc(N(c3ccc(-c4ccccc4)cc3)c3cc4ccc5c6ccccc6[se]c5c4c4ccccc34)cc21 ; CC1(C)c2ccccc2-c2ccc(N(c3ccccc3)c3cc4ccc5c6ccccc6[se]c5c4c4ccccc34)cc21]

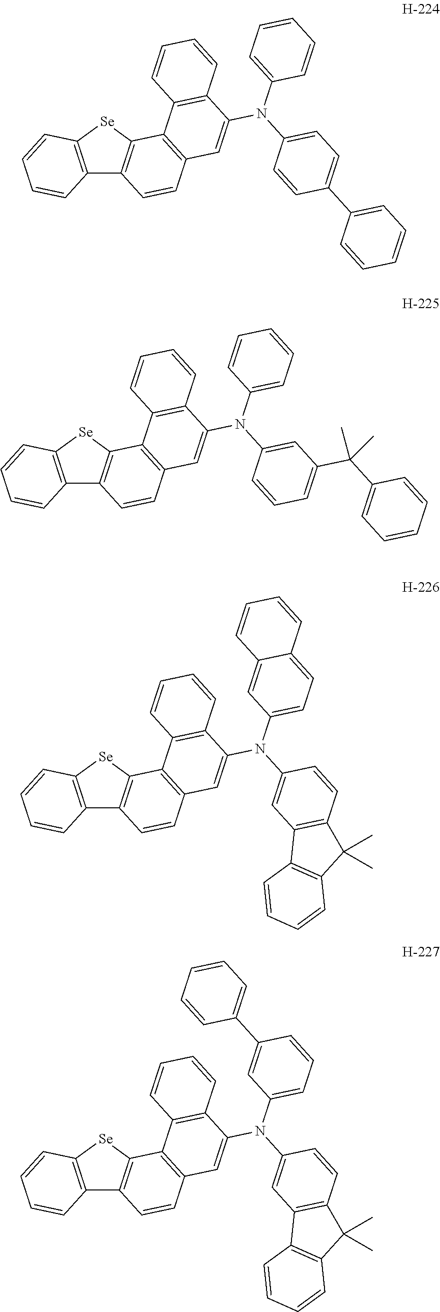 [SMILES: c1ccc(-c2ccc(N(c3ccccc3)c3cc4ccc5c6ccccc6[se]c5c4c4ccccc34)cc2)cc1 ; CC(C)(c1ccccc1)c1cccc(N(c2ccccc2)c2cc3ccc4c5ccccc5[se]c4c3c3ccccc23)c1 ; CC1(C)c2ccccc2-c2cc(N(c3ccc4ccccc4c3)c3cc4ccc5c6ccccc6[se]c5c4c4ccccc34)ccc21 ; CC1(C)c2ccccc2-c2cc(N(c3cccc(-c4ccccc4)c3)c3cc4ccc5c6ccccc6[se]c5c4c4ccccc34)ccc21]

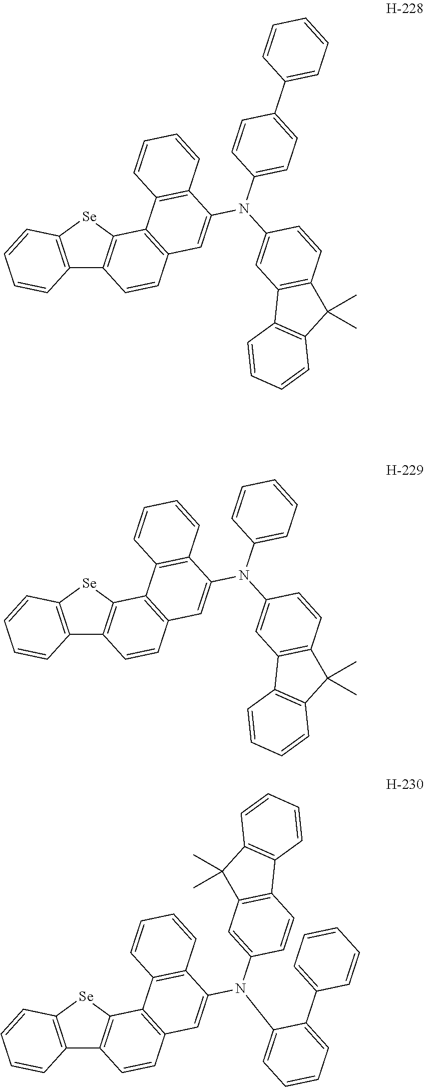

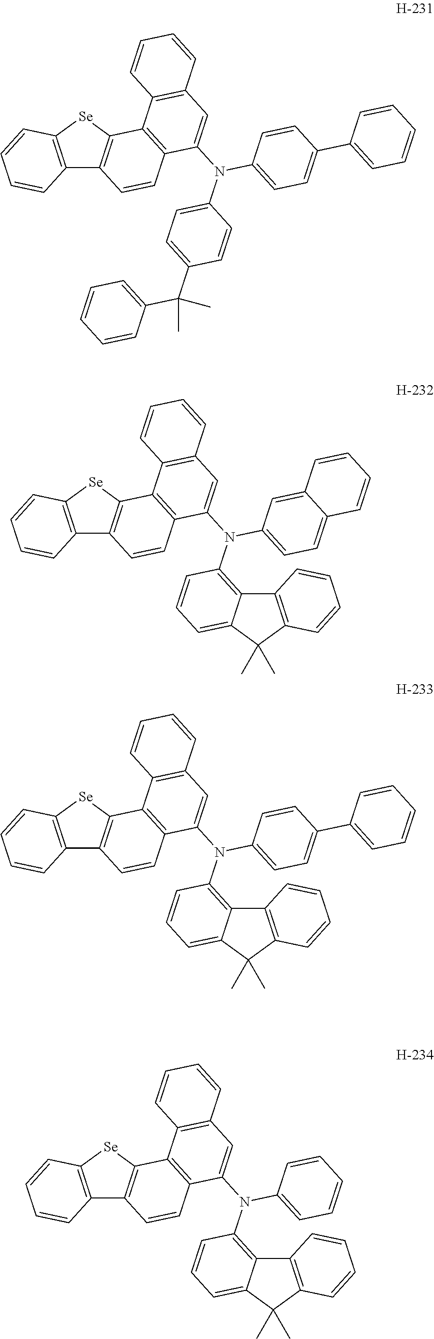 [SMILES: CC(C)(c1ccccc1)c1ccc(N(c2ccc(-c3ccccc3)cc2)c2cc3ccccc3c3c2ccc2c4ccccc4[se]c23)cc1 ; CC1(C)c2ccccc2-c2c(N(c3ccc4ccccc4c3)c3cc4ccccc4c4c3ccc3c5ccccc5[se]c34)cccc21 ; CC1(C)c2ccccc2-c2c(N(c3ccc(-c4ccccc4)cc3)c3cc4ccccc4c4c3ccc3c5ccccc5[se]c34)cccc21 ; CC1(C)c2ccccc2-c2c(N(c3ccccc3)c3cc4ccccc4c4c3ccc3c5ccccc5[se]c34)cccc21]

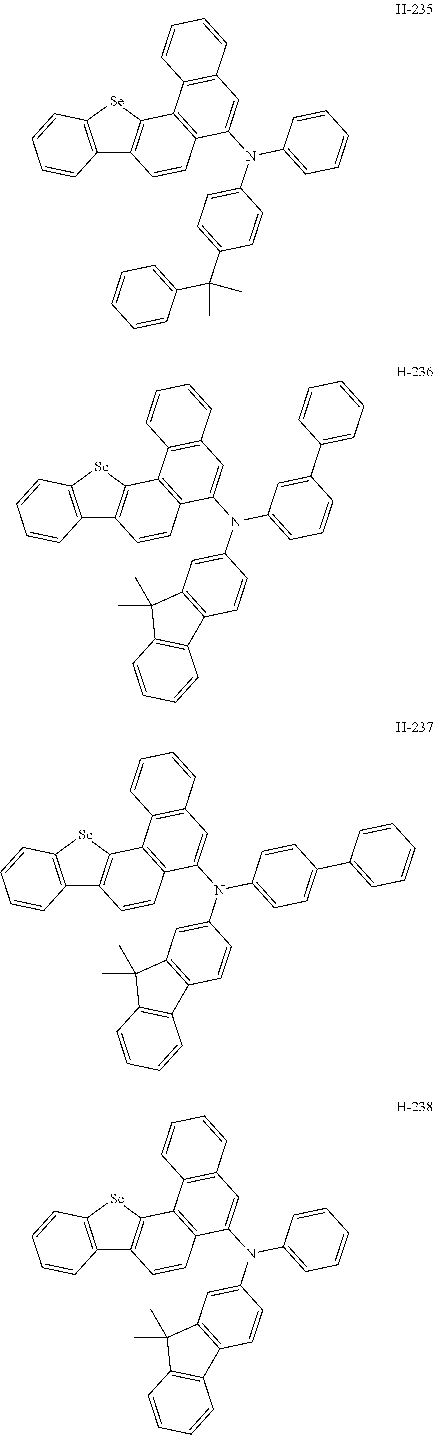 [SMILES: CC(C)(c1ccccc1)c1ccc(N(c2ccccc2)c2cc3ccccc3c3c2ccc2c4ccccc4[se]c23)cc1 ; CC1(C)c2ccccc2-c2ccc(N(c3cccc(-c4ccccc4)c3)c3cc4ccccc4c4c3ccc3c5ccccc5[se]c34)cc21 ; CC1(C)c2ccccc2-c2ccc(N(c3ccc(-c4ccccc4)cc3)c3cc4ccccc4c4c3ccc3c5ccccc5[se]c34)cc21 ; CC1(C)c2ccccc2-c2ccc(N(c3ccccc3)c3cc4ccccc4c4c3ccc3c5ccccc5[se]c34)cc21]

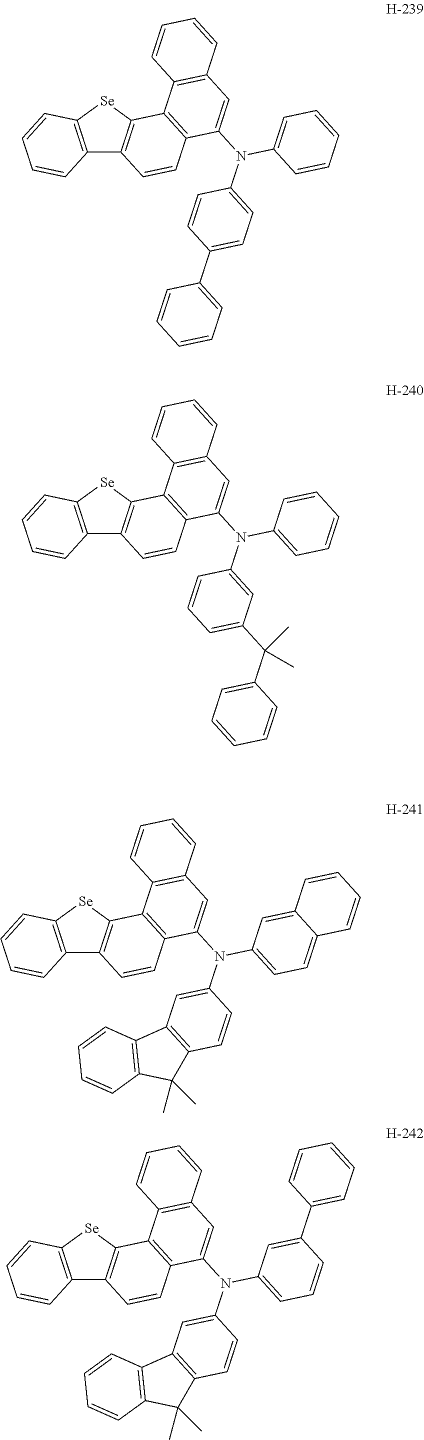

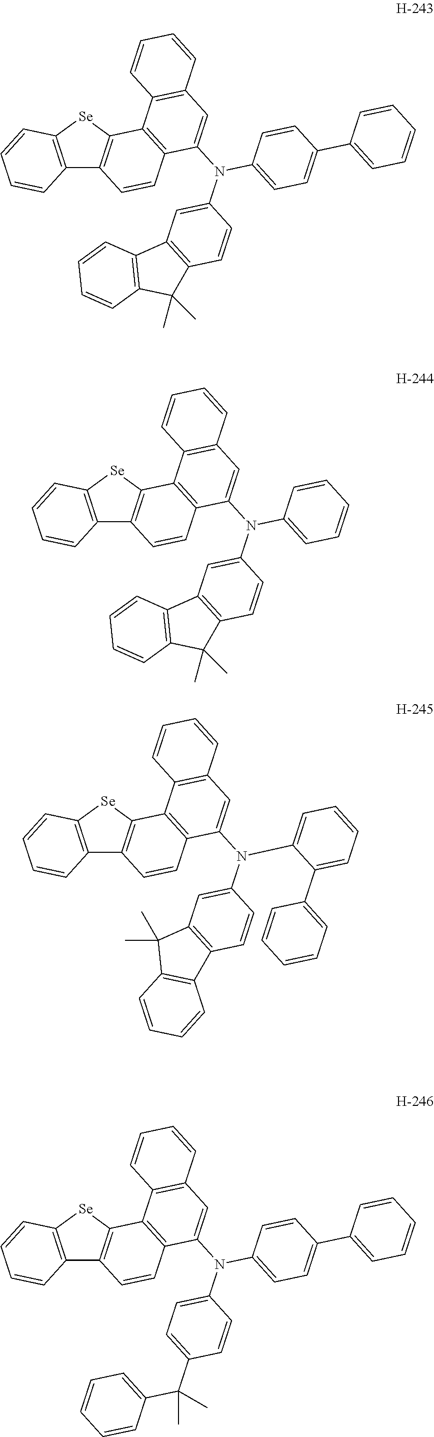

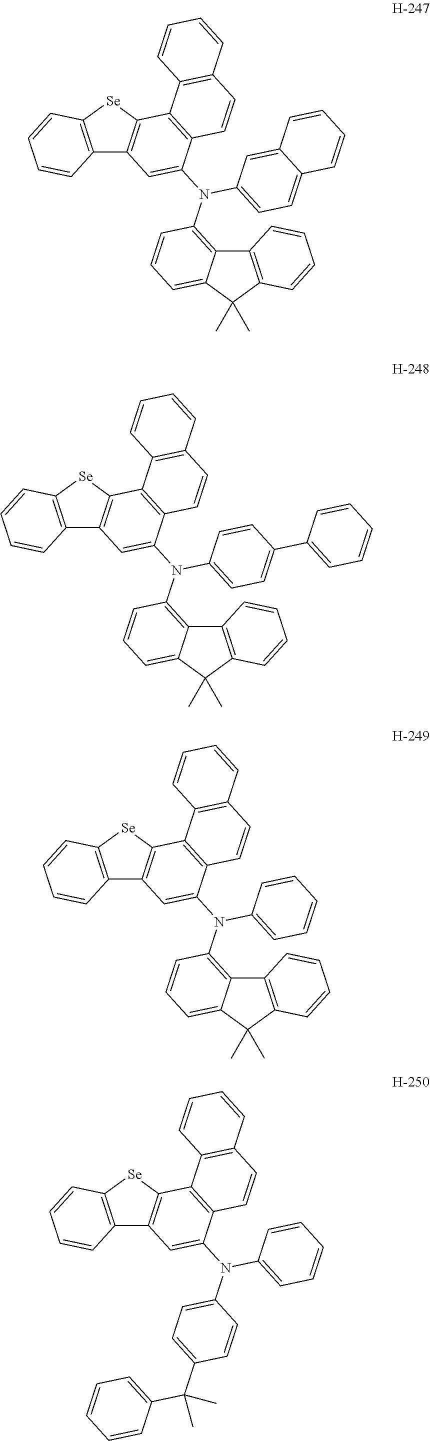

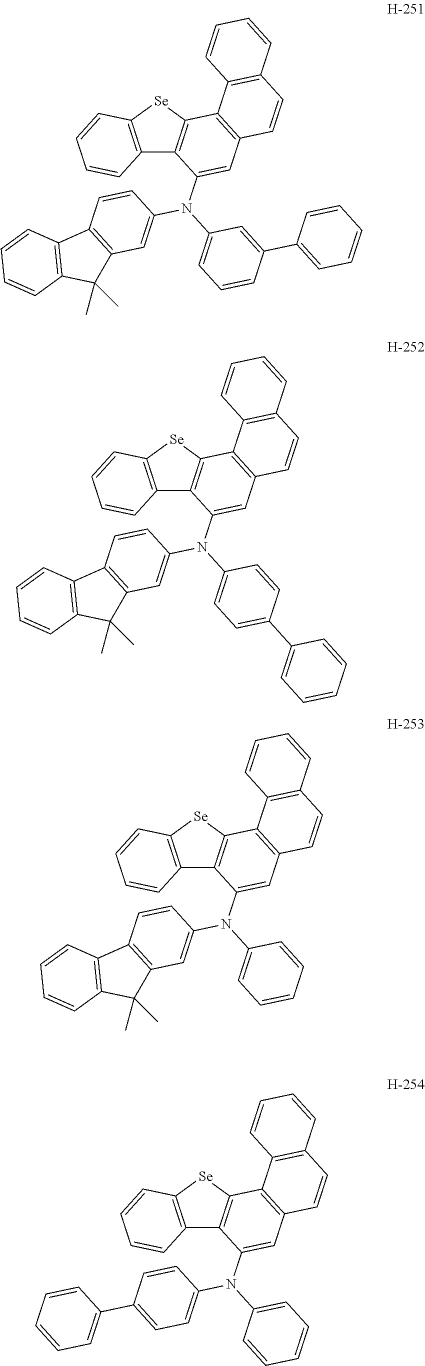

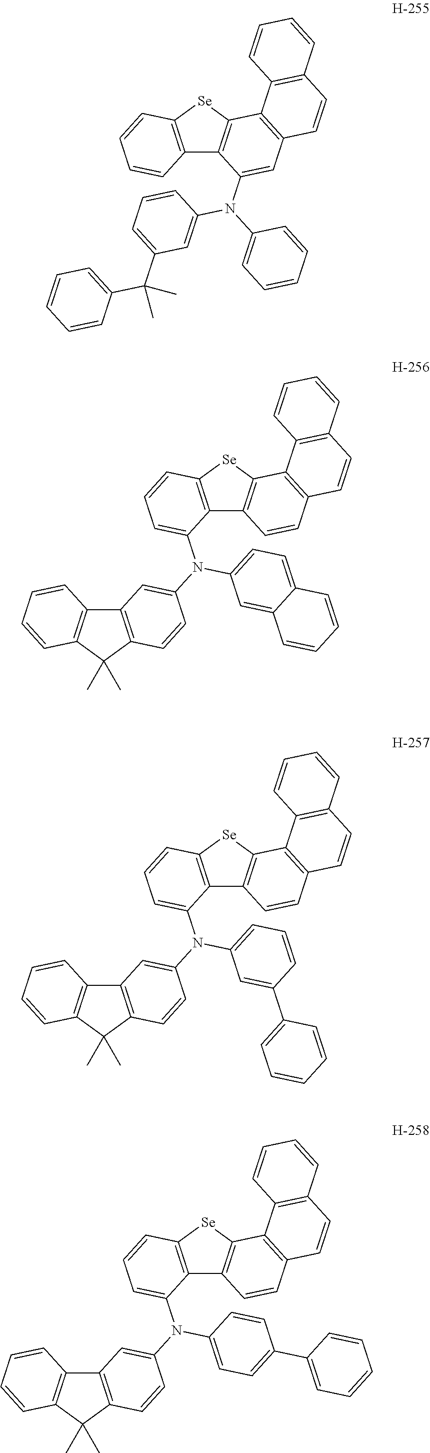 [SMILES: CC(C)(c1ccccc1)c1cccc(N(c2ccccc2)c2cc3ccc4ccccc4c3c3[se]c4ccccc4c23)c1 ; CC1(C)c2ccccc2-c2cc(N(c3ccc4ccccc4c3)c3cccc4[se]c5c(ccc6ccc7ccccc7c65)c34)ccc21 ; CC1(C)c2ccccc2-c2cc(N(c3cccc(-c4ccccc4)c3)c3cccc4[se]c5c(ccc6ccc7ccccc7c65)c34)ccc21 ; CC1(C)c2ccccc2-c2cc(N(c3ccc(-c4ccccc4)cc3)c3cccc4[se]c5c(ccc6ccc7ccccc7c65)c34)ccc21]

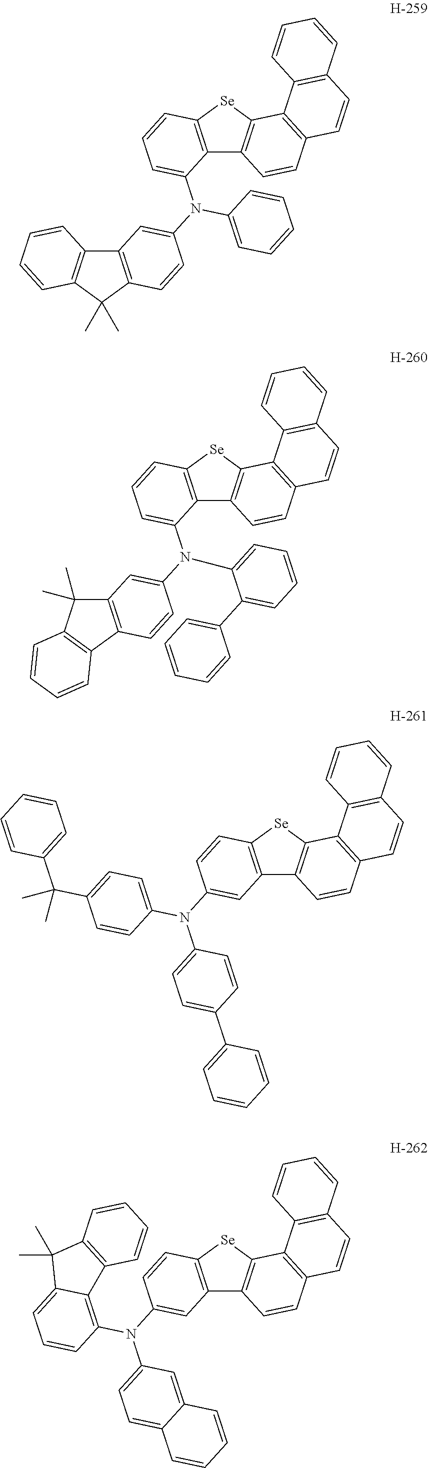 [SMILES: CC1(C)c2ccccc2-c2cc(N(c3ccccc3)c3cccc4[se]c5c(ccc6ccc7ccccc7c65)c34)ccc21 ; CC1(C)c2ccccc2-c2ccc(N(c3ccccc3-c3ccccc3)c3cccc4[se]c5c(ccc6ccc7ccccc7c65)c34)cc21 ; CC(C)(c1ccccc1)c1ccc(N(c2ccc(-c3ccccc3)cc2)c2ccc3[se]c4c(ccc5ccc6ccccc6c54)c3c2)cc1 ; CC1(C)c2ccccc2-c2c(N(c3ccc4ccccc4c3)c3ccc4[se]c5c(ccc6ccc7ccccc7c65)c4c3)cccc21]

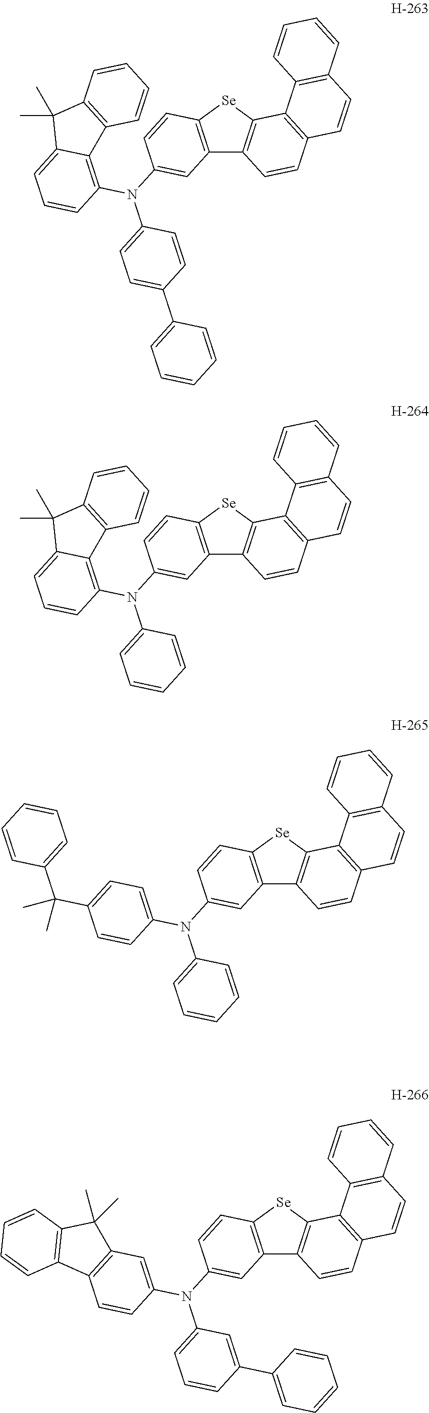 [SMILES: CC1(C)c2ccccc2-c2c(N(c3ccc(-c4ccccc4)cc3)c3ccc4[se]c5c(ccc6ccc7ccccc7c65)c4c3)cccc21 ; CC1(C)c2ccccc2-c2c(N(c3ccccc3)c3ccc4[se]c5c(ccc6ccc7ccccc7c65)c4c3)cccc21 ; CC(C)(c1ccccc1)c1ccc(N(c2ccccc2)c2ccc3[se]c4c(ccc5ccc6ccccc6c54)c3c2)cc1 ; CC1(C)c2ccccc2-c2ccc(N(c3cccc(-c4ccccc4)c3)c3ccc4[se]c5c(ccc6ccc7ccccc7c65)c4c3)cc21]

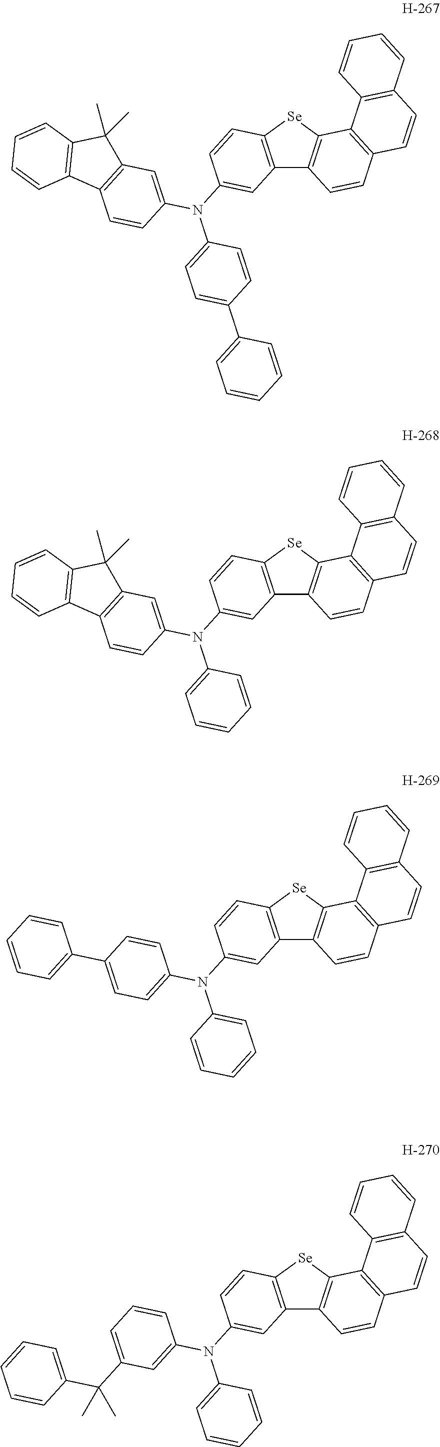 [SMILES: CC1(C)c2ccccc2-c2ccc(N(c3ccc(-c4ccccc4)cc3)c3ccc4[se]c5c(ccc6ccc7ccccc7c65)c4c3)cc21 ; CC1(C)c2ccccc2-c2ccc(N(c3ccccc3)c3ccc4[se]c5c(ccc6ccc7ccccc7c65)c4c3)cc21 ; c1ccc(-c2ccc(N(c3ccccc3)c3ccc4[se]c5c(ccc6ccc7ccccc7c65)c4c3)cc2)cc1 ; CC(C)(c1ccccc1)c1cccc(N(c2ccccc2)c2ccc3[se]c4c(ccc5ccc6ccccc6c54)c3c2)c1]

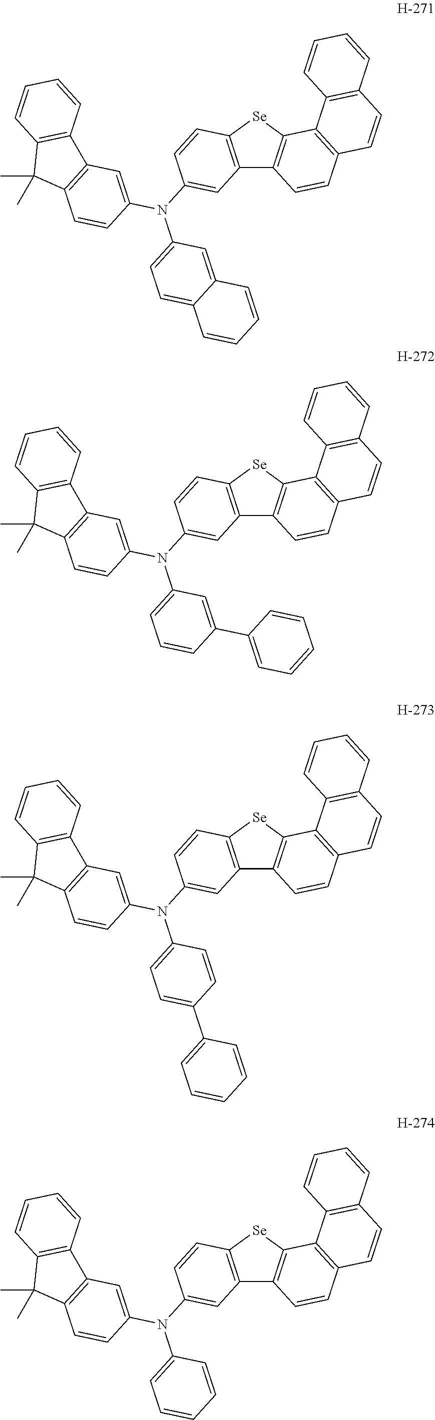 [SMILES: CC1(C)c2ccccc2-c2cc(N(c3ccc4ccccc4c3)c3ccc4[se]c5c(ccc6ccc7ccccc7c65)c4c3)ccc21 ; CC1(C)c2ccccc2-c2cc(N(c3cccc(-c4ccccc4)c3)c3ccc4[se]c5c(ccc6ccc7ccccc7c65)c4c3)ccc21 ; CC1(C)c2ccccc2-c2cc(N(c3ccc(-c4ccccc4)cc3)c3ccc4[se]c5c(ccc6ccc7ccccc7c65)c4c3)ccc21 ; CC1(C)c2ccccc2-c2cc(N(c3ccccc3)c3ccc4[se]c5c(ccc6ccc7ccccc7c65)c4c3)ccc21]

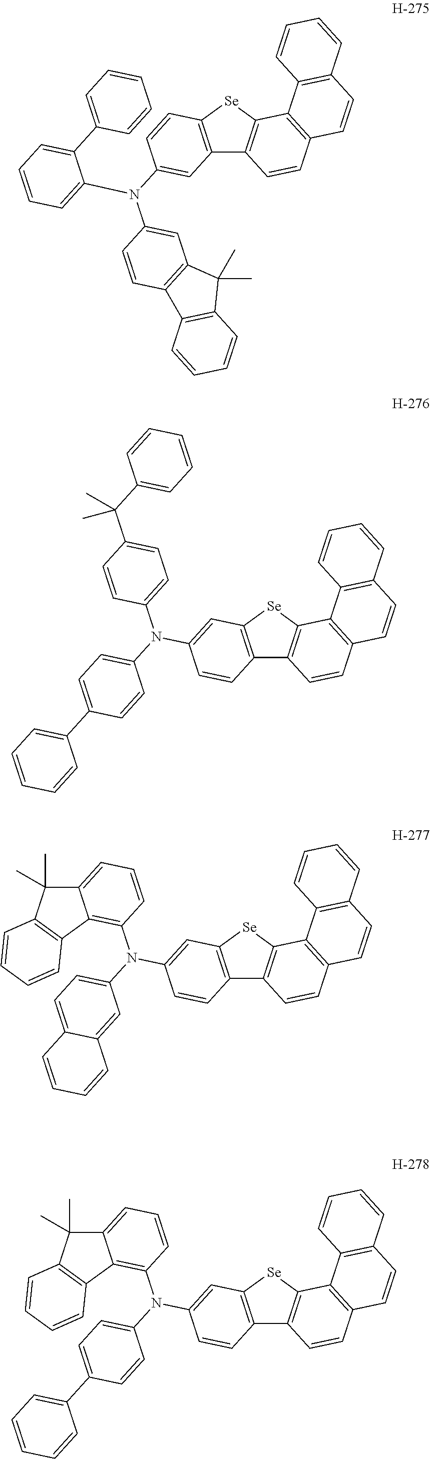 [SMILES: CC1(C)c2ccccc2-c2ccc(N(c3ccc4[se]c5c(ccc6ccc7ccccc7c65)c4c3)c3ccccc3-c3ccccc3)cc21 ; CC(C)(c1ccccc1)c1ccc(N(c2ccc(-c3ccccc3)cc2)c2ccc3c(c2)[se]c2c3ccc3ccc4ccccc4c32)cc1 ; CC1(C)c2ccccc2-c2c(N(c3ccc4ccccc4c3)c3ccc4c(c3)[se]c3c4ccc4ccc5ccccc5c43)cccc21 ; CC1(C)c2ccccc2-c2c(N(c3ccc(-c4ccccc4)cc3)c3ccc4c(c3)[se]c3c4ccc4ccc5ccccc5c43)cccc21]

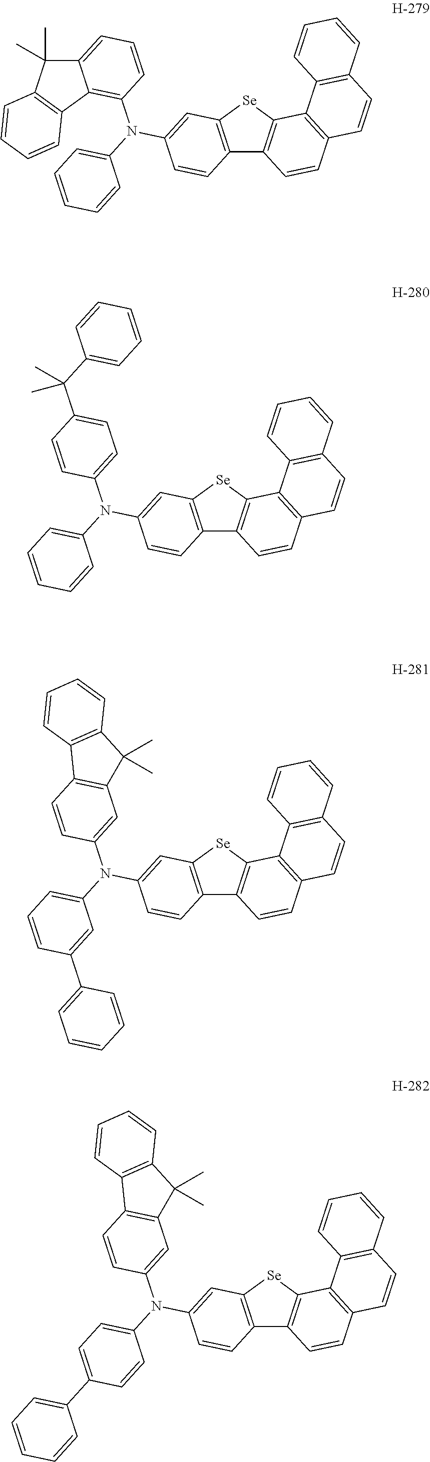 [SMILES: CC1(C)c2ccccc2-c2c(N(c3ccccc3)c3ccc4c(c3)[se]c3c4ccc4ccc5ccccc5c43)cccc21 ; CC(C)(c1ccccc1)c1ccc(N(c2ccccc2)c2ccc3c(c2)[se]c2c3ccc3ccc4ccccc4c32)cc1 ; CC1(C)c2ccccc2-c2ccc(N(c3cccc(-c4ccccc4)c3)c3ccc4c(c3)[se]c3c4ccc4ccc5ccccc5c43)cc21 ; CC1(C)c2ccccc2-c2ccc(N(c3ccc(-c4ccccc4)cc3)c3ccc4c(c3)[se]c3c4ccc4ccc5ccccc5c43)cc21]

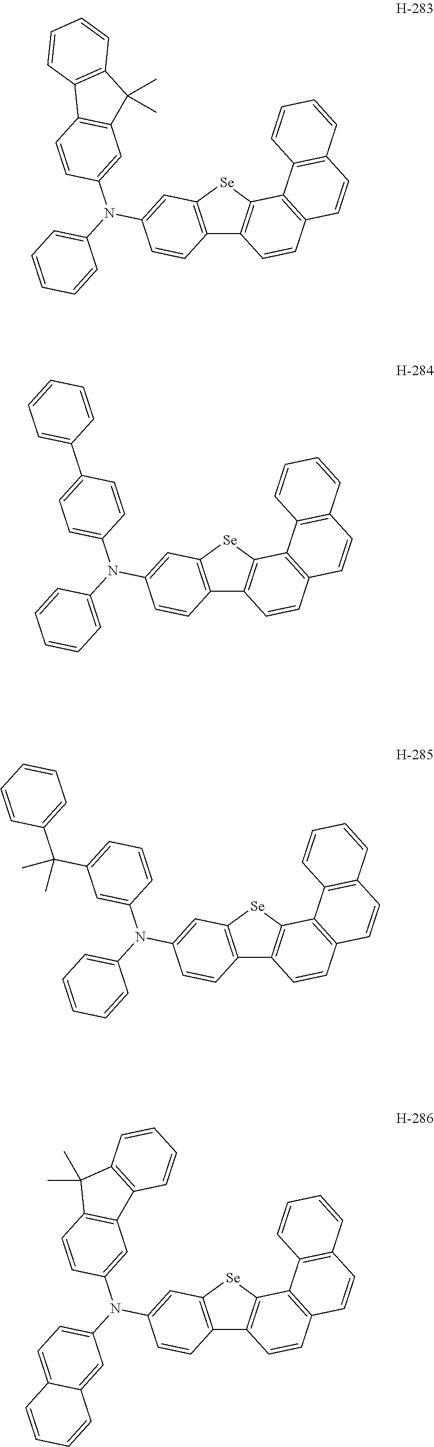 [SMILES: CC1(C)c2ccccc2-c2ccc(N(c3ccccc3)c3ccc4c(c3)[se]c3c4ccc4ccc5ccccc5c43)cc21 ; c1ccc(-c2ccc(N(c3ccccc3)c3ccc4c(c3)[se]c3c4ccc4ccc5ccccc5c43)cc2)cc1 ; CC(C)(c1ccccc1)c1cccc(N(c2ccccc2)c2ccc3c(c2)[se]c2c3ccc3ccc4ccccc4c32)c1 ; CC1(C)c2ccccc2-c2cc(N(c3ccc4ccccc4c3)c3ccc4c(c3)[se]c3c4ccc4ccc5ccccc5c43)ccc21]

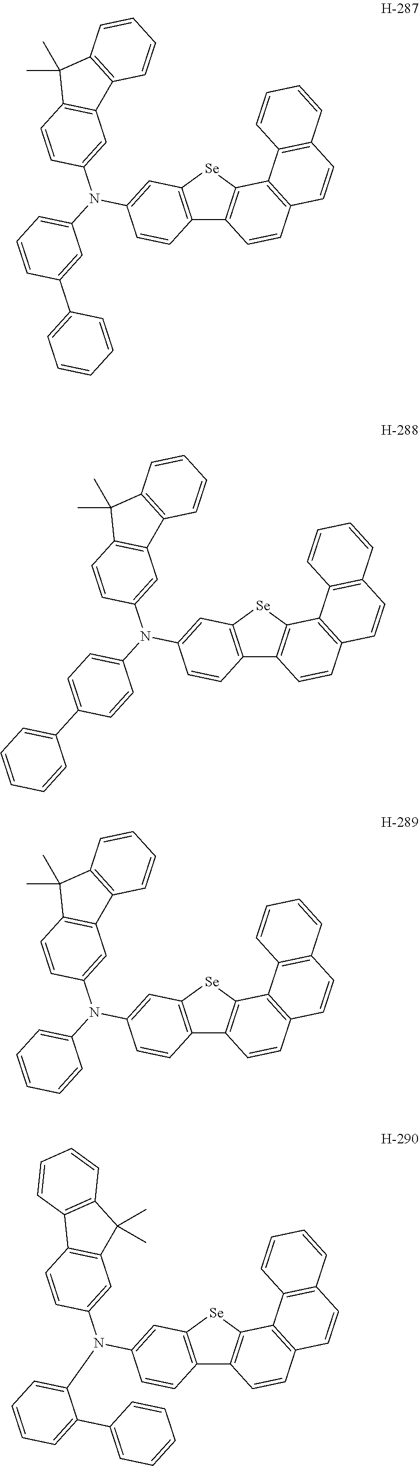 [SMILES: CC1(C)c2ccccc2-c2cc(N(c3cccc(-c4ccccc4)c3)c3ccc4c(c3)[se]c3c4ccc4ccc5ccccc5c43)ccc21 ; CC1(C)c2ccccc2-c2cc(N(c3ccc(-c4ccccc4)cc3)c3ccc4c(c3)[se]c3c4ccc4ccc5ccccc5c43)ccc21 ; CC1(C)c2ccccc2-c2cc(N(c3ccccc3)c3ccc4c(c3)[se]c3c4ccc4ccc5ccccc5c43)ccc21 ; CC1(C)c2ccccc2-c2ccc(N(c3ccc4c(c3)[se]c3c4ccc4ccc5ccccc5c43)c3ccccc3-c3ccccc3)cc21]

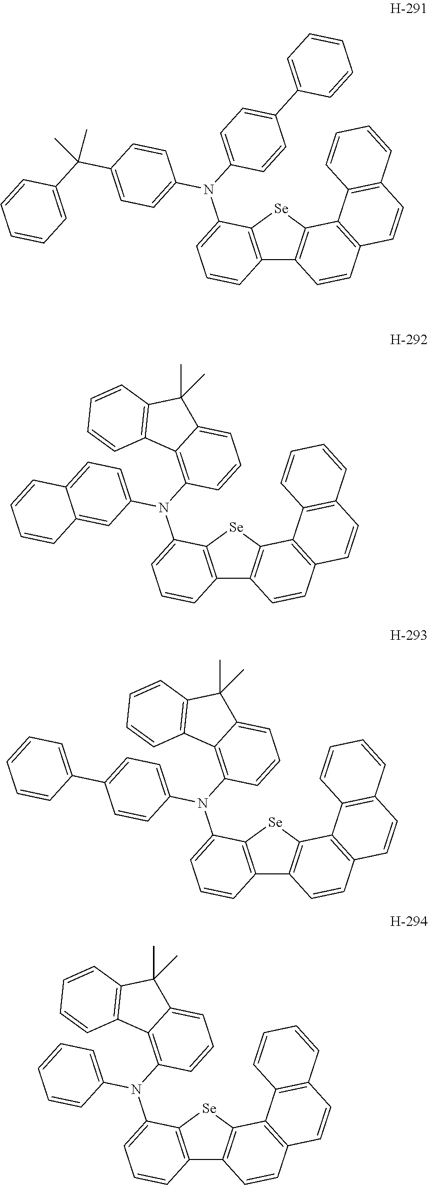 [SMILES: CC(C)(c1ccccc1)c1ccc(N(c2ccc(-c3ccccc3)cc2)c2cccc3c2[se]c2c3ccc3ccc4ccccc4c32)cc1 ; CC1(C)c2ccccc2-c2c(N(c3ccc4ccccc4c3)c3cccc4c3[se]c3c4ccc4ccc5ccccc5c43)cccc21 ; CC1(C)c2ccccc2-c2c(N(c3ccc(-c4ccccc4)cc3)c3cccc4c3[se]c3c4ccc4ccc5ccccc5c43)cccc21 ; CC1(C)c2ccccc2-c2c(N(c3ccccc3)c3cccc4c3[se]c3c4ccc4ccc5ccccc5c43)cccc21]

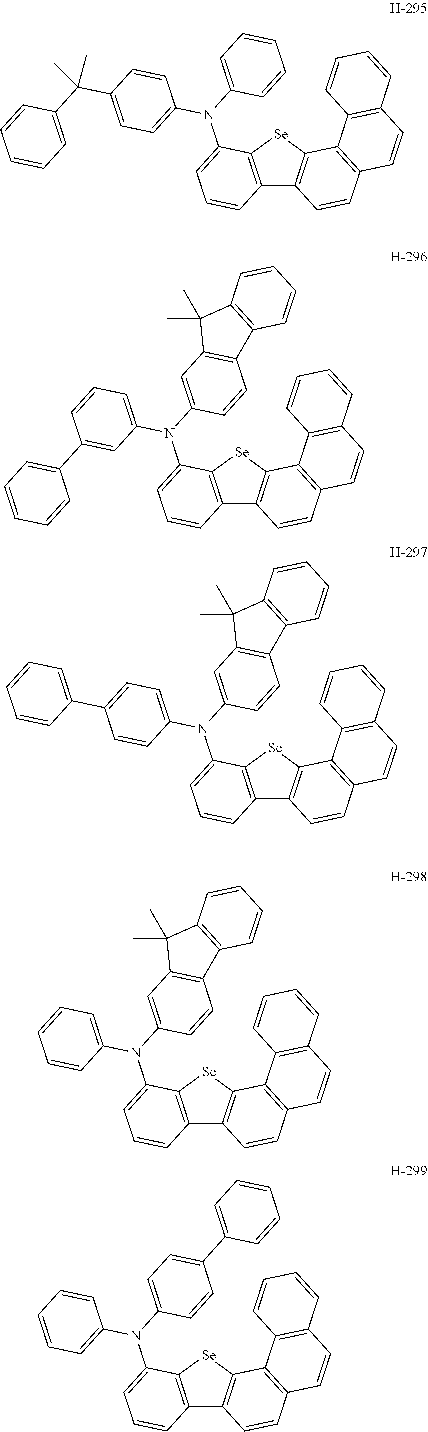

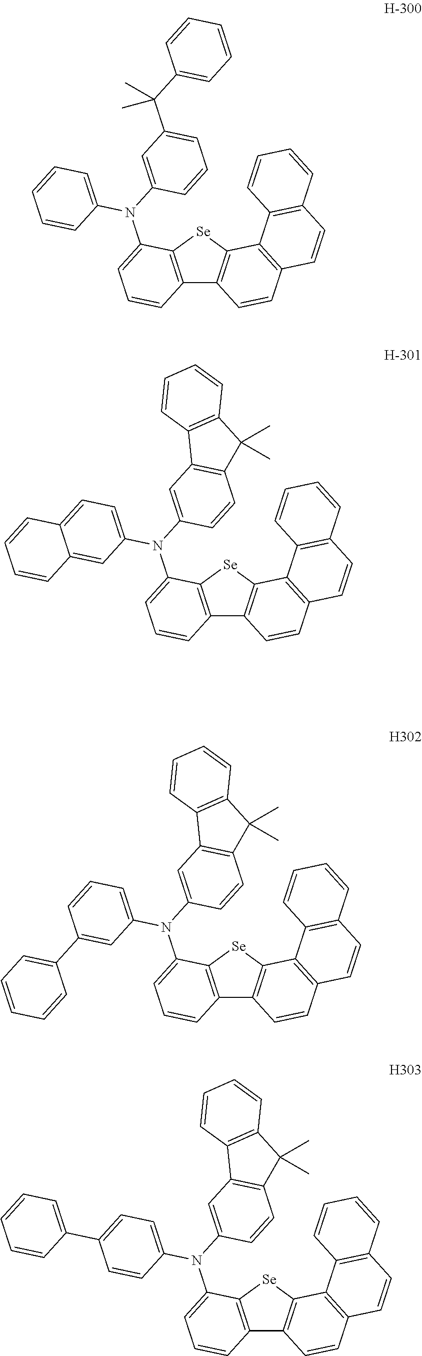 [SMILES: CC(C)(c1ccccc1)c1cccc(N(c2ccccc2)c2cccc3c2[se]c2c3ccc3ccc4ccccc4c32)c1 ; CC1(C)c2ccccc2-c2cc(N(c3ccc4ccccc4c3)c3cccc4c3[se]c3c4ccc4ccc5ccccc5c43)ccc21 ; CC1(C)c2ccccc2-c2cc(N(c3cccc(-c4ccccc4)c3)c3cccc4c3[se]c3c4ccc4ccc5ccccc5c43)ccc21 ; CC1(C)c2ccccc2-c2cc(N(c3ccc(-c4ccccc4)cc3)c3cccc4c3[se]c3c4ccc4ccc5ccccc5c43)ccc21]

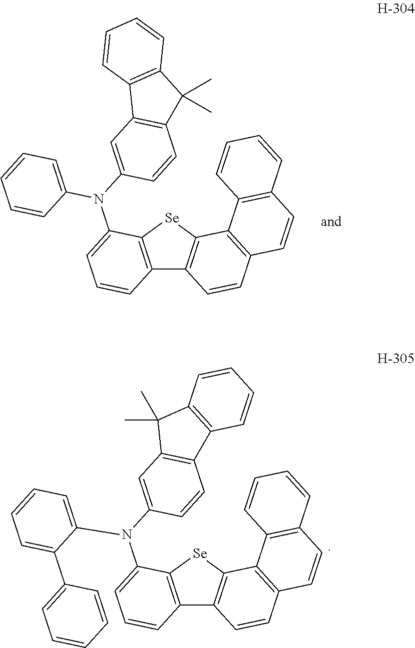 [SMILES: CC1(C)c2ccccc2-c2cc(N(c3ccccc3)c3cccc4c3[se]c3c4ccc4ccc5ccccc5c43)ccc21 ; CC1(C)c2ccccc2-c2ccc(N(c3ccccc3-c3ccccc3)c3cccc4c3[se]c3c4ccc4ccc5ccccc5c43)cc21]

7. The plurality of host materials according to claim 1, wherein the compound represented by formula 2 is selected from the following compounds:

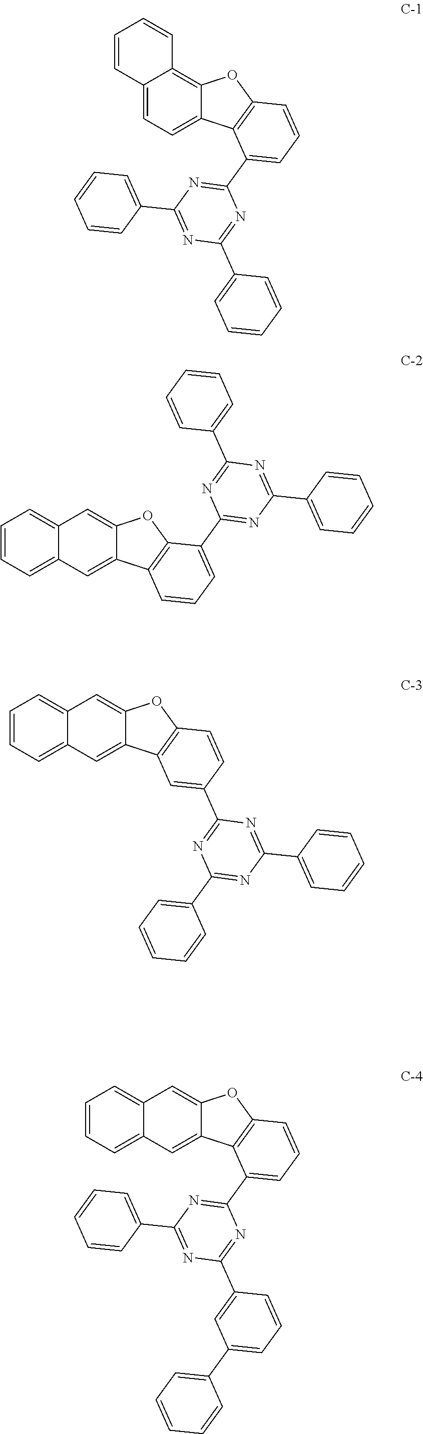

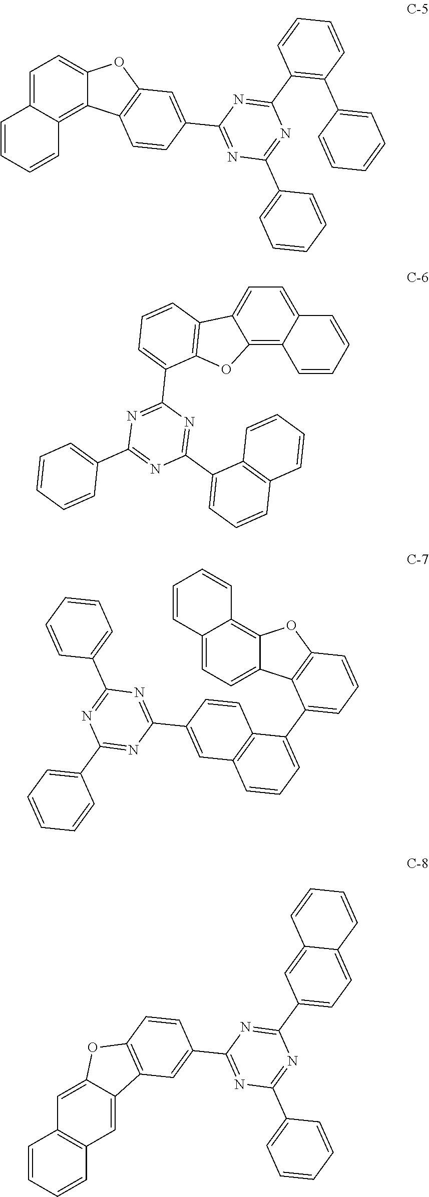

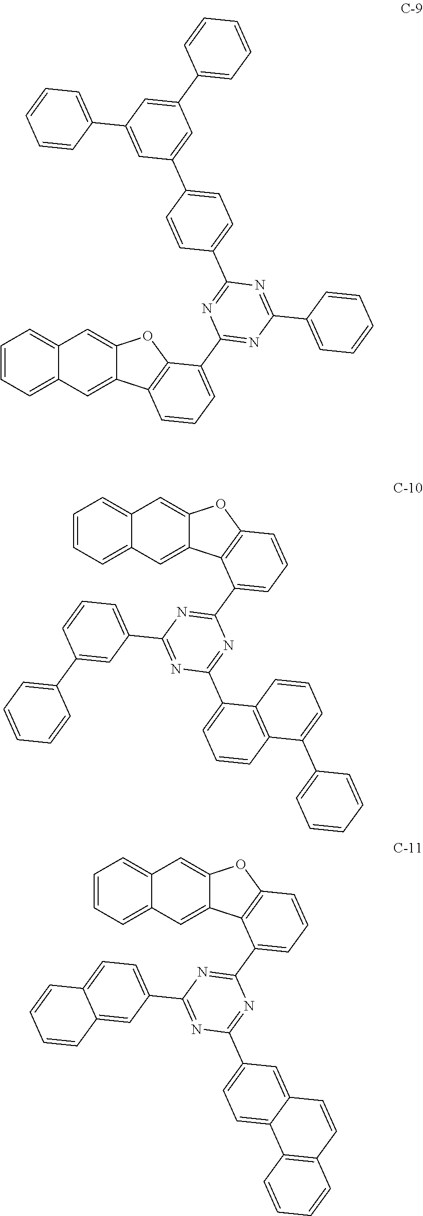 [SMILES: c1ccc(-c2cc(-c3ccccc3)cc(-c3ccc(-c4nc(-c5ccccc5)nc(-c5cccc6c5oc5cc7ccccc7cc56)n4)cc3)c2)cc1 ; c1ccc(-c2cccc(-c3nc(-c4cccc5c(-c6ccccc6)cccc45)nc(-c4cccc5oc6cc7ccccc7cc6c45)n3)c2)cc1 ; c1ccc2cc(-c3nc(-c4ccc5c(ccc6ccccc65)c4)nc(-c4cccc5oc6cc7ccccc7cc6c45)n3)ccc2c1]

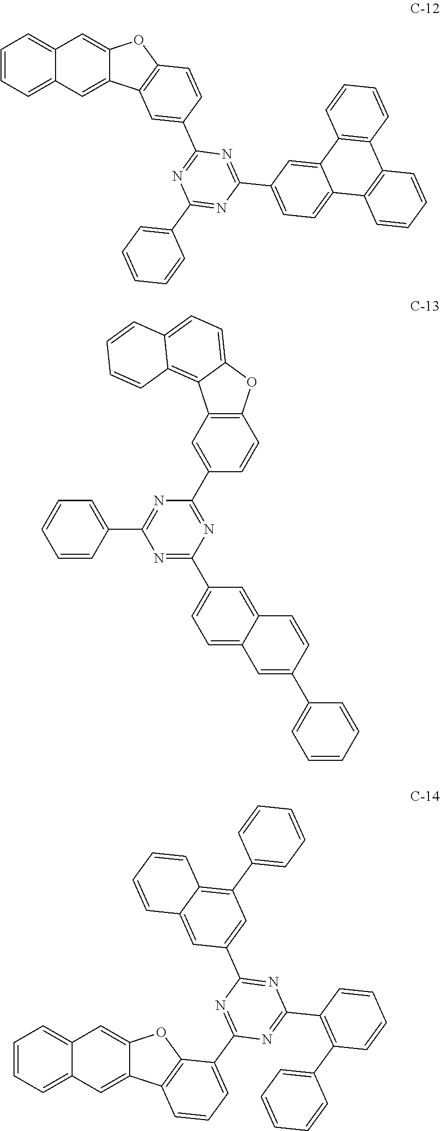

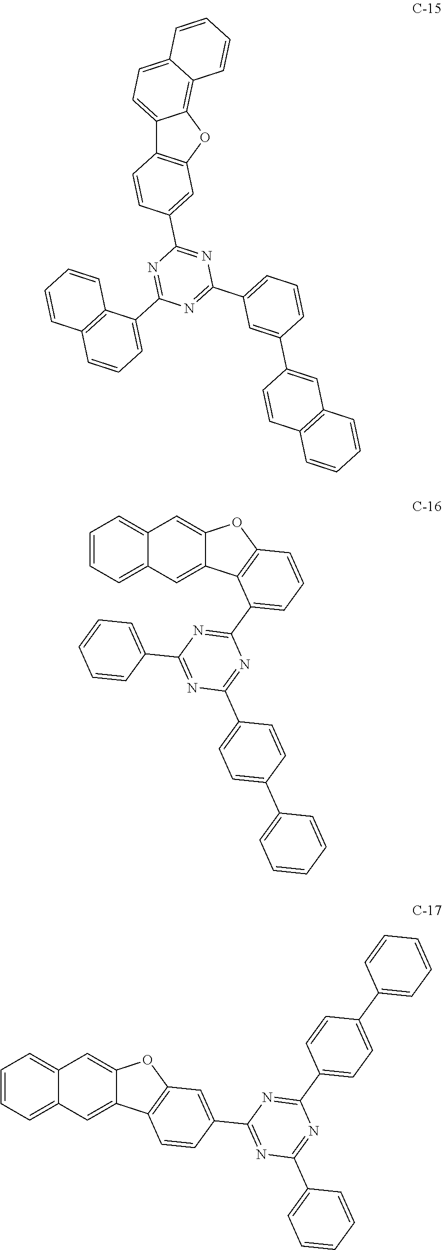 [SMILES: c1cc(-c2ccc3ccccc3c2)cc(-c2nc(-c3ccc4c(c3)oc3c5ccccc5ccc43)nc(-c3cccc4ccccc34)n2)c1 ; c1ccc(-c2ccc(-c3nc(-c4ccccc4)nc(-c4cccc5oc6cc7ccccc7cc6c45)n3)cc2)cc1 ; c1ccc(-c2ccc(-c3nc(-c4ccccc4)nc(-c4ccc5c(c4)oc4cc6ccccc6cc45)n3)cc2)cc1]

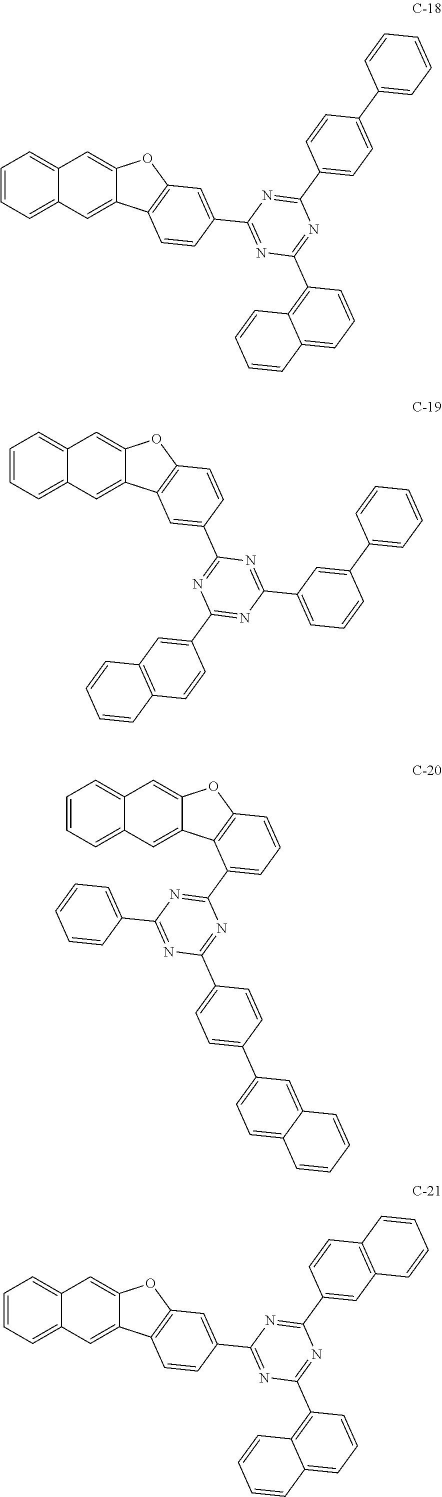 [SMILES: c1ccc(-c2ccc(-c3nc(-c4ccc5c(c4)oc4cc6ccccc6cc45)nc(-c4cccc5ccccc45)n3)cc2)cc1 ; c1ccc(-c2cccc(-c3nc(-c4ccc5ccccc5c4)nc(-c4ccc5oc6cc7ccccc7cc6c5c4)n3)c2)cc1 ; c1ccc(-c2nc(-c3ccc(-c4ccc5ccccc5c4)cc3)nc(-c3cccc4oc5cc6ccccc6cc5c34)n2)cc1 ; c1ccc2cc(-c3nc(-c4ccc5c(c4)oc4cc6ccccc6cc45)nc(-c4cccc5ccccc45)n3)ccc2c1]

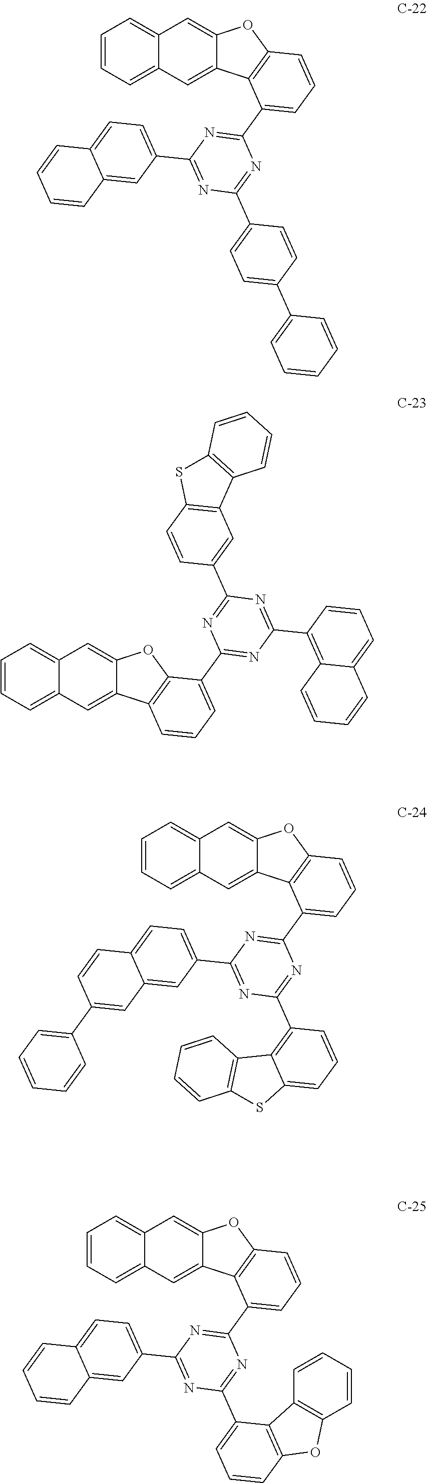 [SMILES: c1ccc(-c2ccc(-c3nc(-c4ccc5ccccc5c4)nc(-c4cccc5oc6cc7ccccc7cc6c45)n3)cc2)cc1 ; c1ccc2cc3c(cc2c1)oc1c(-c2nc(-c4ccc5sc6ccccc6c5c4)nc(-c4cccc5ccccc45)n2)cccc13 ; c1ccc(-c2ccc3ccc(-c4nc(-c5cccc6oc7cc8ccccc8cc7c56)nc(-c5cccc6sc7ccccc7c56)n4)cc3c2)cc1 ; c1ccc2cc(-c3nc(-c4cccc5oc6ccccc6c45)nc(-c4cccc5oc6cc7ccccc7cc6c45)n3)ccc2c1]

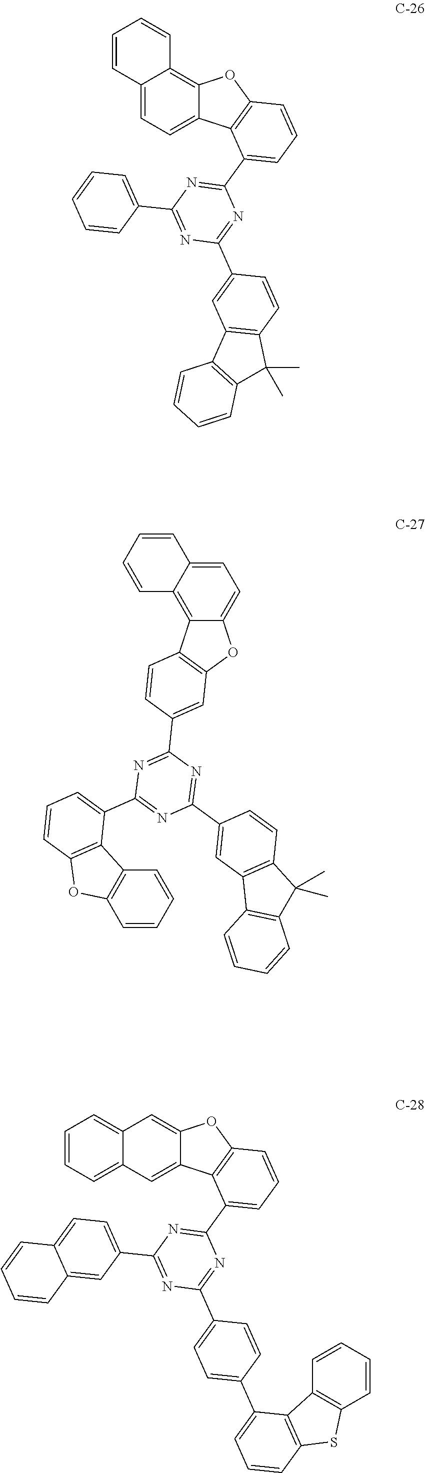 [SMILES: CC1(C)c2ccccc2-c2cc(-c3nc(-c4ccccc4)nc(-c4cccc5oc6c7ccccc7ccc6c45)n3)ccc21 ; CC1(C)c2ccccc2-c2cc(-c3nc(-c4ccc5c(c4)oc4ccc6ccccc6c45)nc(-c4cccc5oc6ccccc6c45)n3)ccc21 ; c1ccc2cc(-c3nc(-c4ccc(-c5cccc6sc7ccccc7c56)cc4)nc(-c4cccc5oc6cc7ccccc7cc6c45)n3)ccc2c1]

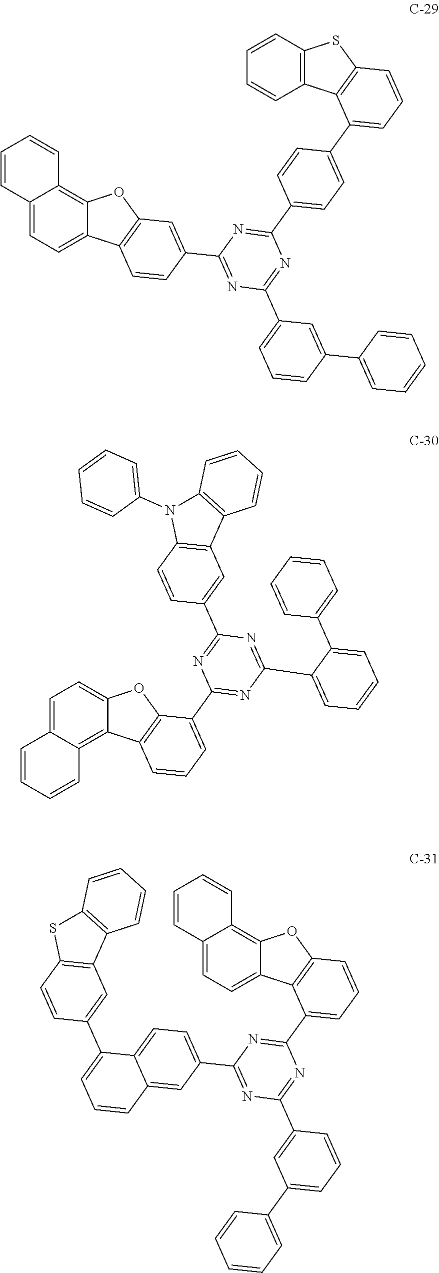 [SMILES: c1ccc(-c2cccc(-c3nc(-c4ccc(-c5cccc6sc7ccccc7c56)cc4)nc(-c4ccc5c(c4)oc4c6ccccc6ccc54)n3)c2)cc1 ; c1ccc(-c2ccccc2-c2nc(-c3ccc4c(c3)c3ccccc3n4-c3ccccc3)nc(-c3cccc4c3oc3ccc5ccccc5c34)n2)cc1 ; c1ccc(-c2cccc(-c3nc(-c4ccc5c(-c6ccc7sc8ccccc8c7c6)cccc5c4)nc(-c4cccc5oc6c7ccccc7ccc6c45)n3)c2)cc1]

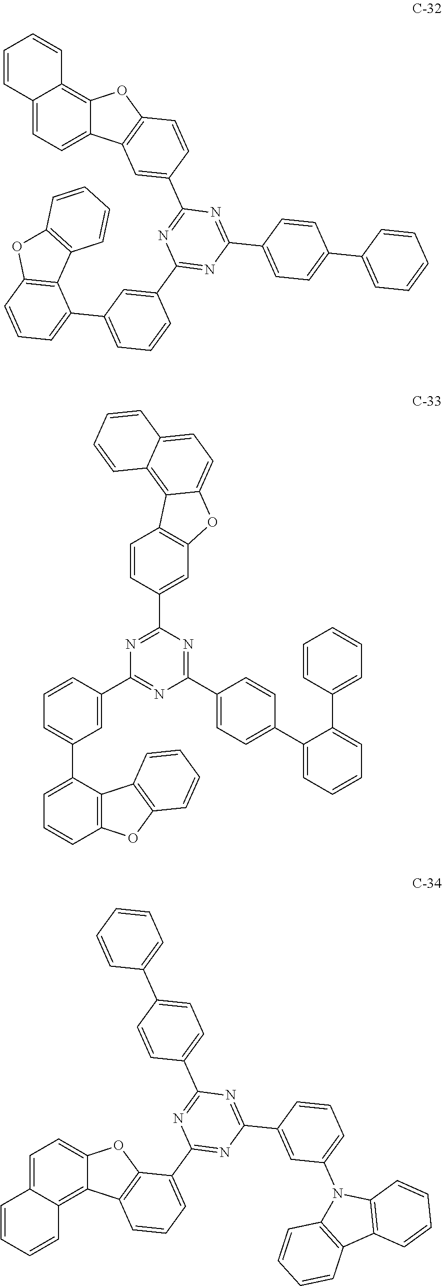 [SMILES: c1ccc(-c2ccc(-c3nc(-c4cccc(-c5cccc6oc7ccccc7c56)c4)nc(-c4ccc5oc6c7ccccc7ccc6c5c4)n3)cc2)cc1 ; c1ccc(-c2ccccc2-c2ccc(-c3nc(-c4cccc(-c5cccc6oc7ccccc7c56)c4)nc(-c4ccc5c(c4)oc4ccc6ccccc6c45)n3)cc2)cc1 ; c1ccc(-c2ccc(-c3nc(-c4cccc(-n5c6ccccc6c6ccccc65)c4)nc(-c4cccc5c4oc4ccc6ccccc6c45)n3)cc2)cc1]

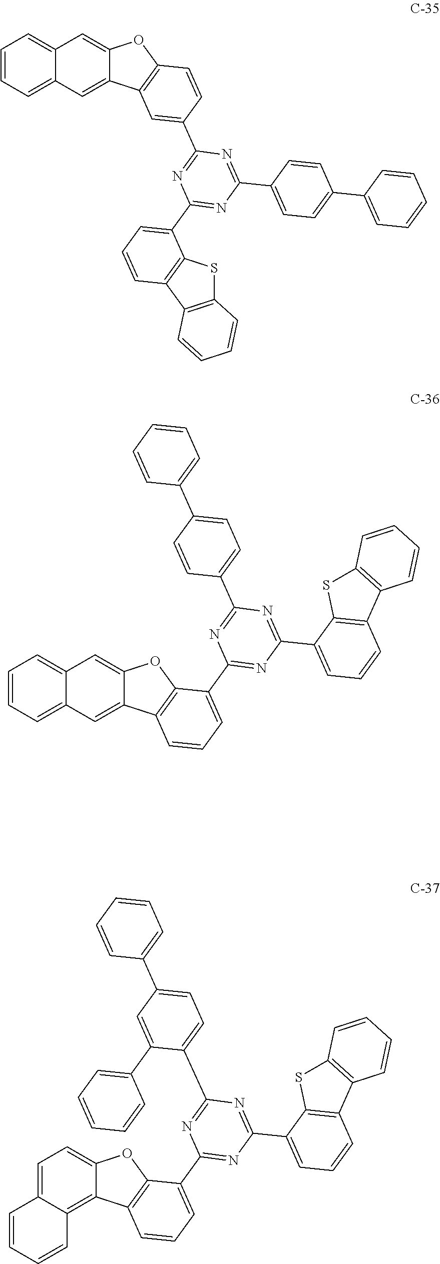 [SMILES: c1ccc(-c2ccc(-c3nc(-c4ccc5oc6cc7ccccc7cc6c5c4)nc(-c4cccc5c4sc4ccccc45)n3)cc2)cc1 ; c1ccc(-c2ccc(-c3nc(-c4cccc5c4oc4cc6ccccc6cc45)nc(-c4cccc5c4sc4ccccc45)n3)cc2)cc1 ; c1ccc(-c2ccc(-c3nc(-c4cccc5c4oc4ccc6ccccc6c45)nc(-c4cccc5c4sc4ccccc45)n3)c(-c3ccccc3)c2)cc1]

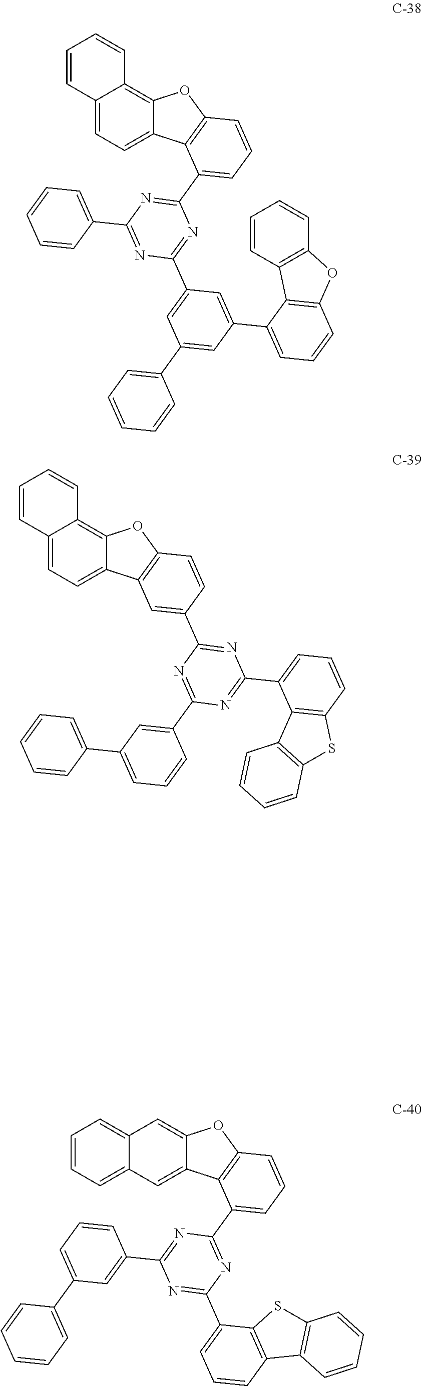 [SMILES: c1ccc(-c2cc(-c3nc(-c4ccccc4)nc(-c4cccc5oc6c7ccccc7ccc6c45)n3)cc(-c3cccc4oc5ccccc5c34)c2)cc1 ; c1ccc(-c2cccc(-c3nc(-c4ccc5oc6c7ccccc7ccc6c5c4)nc(-c4cccc5sc6ccccc6c45)n3)c2)cc1 ; c1ccc(-c2cccc(-c3nc(-c4cccc5c4sc4ccccc45)nc(-c4cccc5oc6cc7ccccc7cc6c45)n3)c2)cc1]

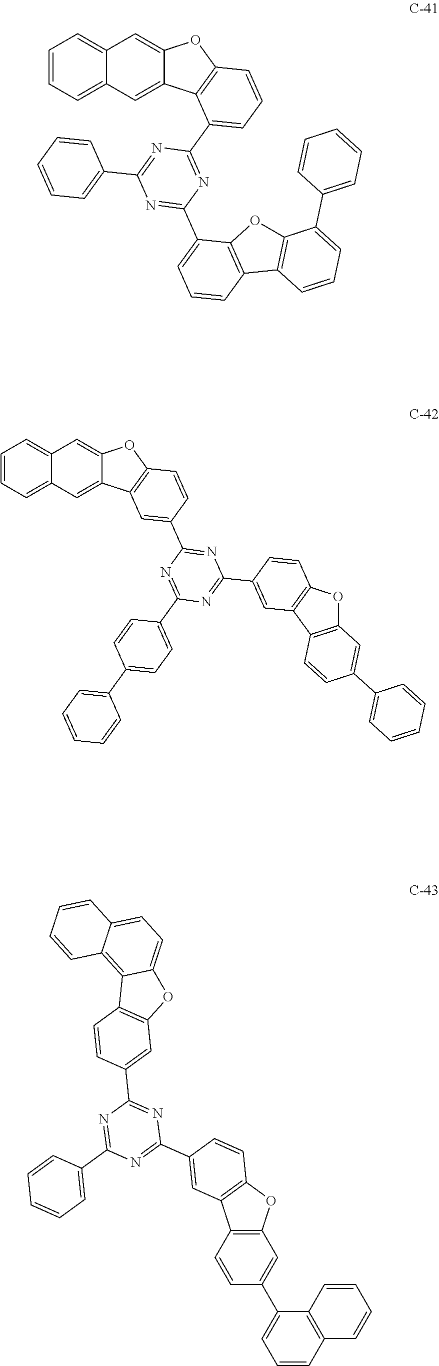 [SMILES: c1ccc(-c2nc(-c3cccc4c3oc3c(-c5ccccc5)cccc34)nc(-c3cccc4oc5cc6ccccc6cc5c34)n2)cc1 ; c1ccc(-c2ccc(-c3nc(-c4ccc5oc6cc(-c7ccccc7)ccc6c5c4)nc(-c4ccc5oc6cc7ccccc7cc6c5c4)n3)cc2)cc1 ; c1ccc(-c2nc(-c3ccc4c(c3)oc3ccc5ccccc5c34)nc(-c3ccc4oc5cc(-c6cccc7ccccc67)ccc5c4c3)n2)cc1]

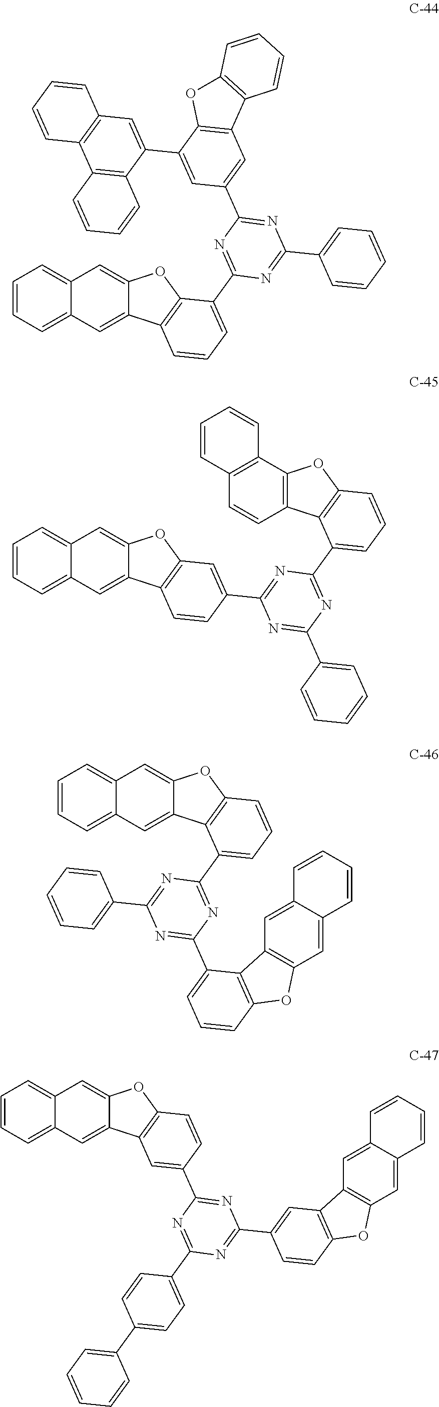 [SMILES: c1ccc(-c2nc(-c3cc(-c4cc5ccccc5c5ccccc45)c4oc5ccccc5c4c3)nc(-c3cccc4c3oc3cc5ccccc5cc34)n2)cc1 ; c1ccc(-c2nc(-c3ccc4c(c3)oc3cc5ccccc5cc34)nc(-c3cccc4oc5c6ccccc6ccc5c34)n2)cc1 ; c1ccc(-c2nc(-c3cccc4oc5cc6ccccc6cc5c34)nc(-c3cccc4oc5cc6ccccc6cc5c34)n2)cc1 ; c1ccc(-c2ccc(-c3nc(-c4ccc5oc6cc7ccccc7cc6c5c4)nc(-c4ccc5oc6cc7ccccc7cc6c5c4)n3)cc2)cc1]

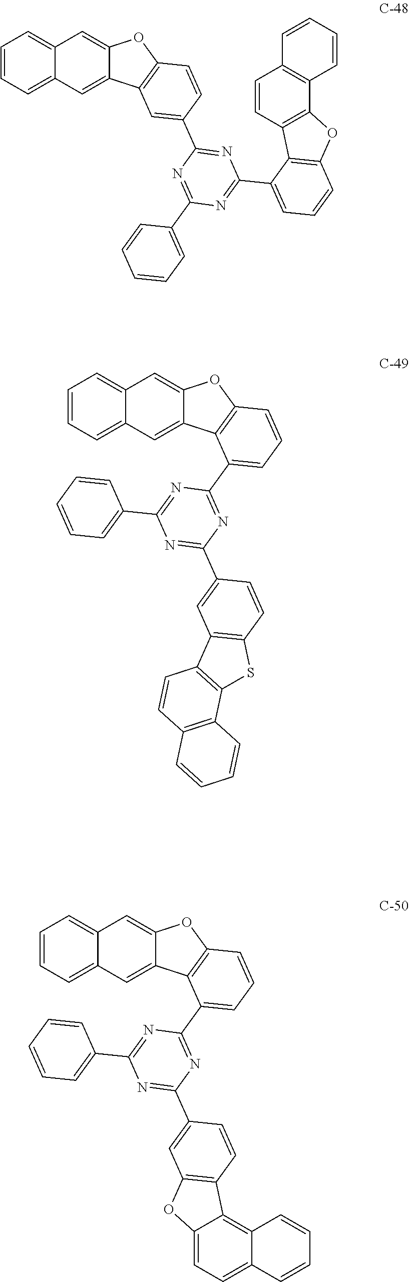 [SMILES: c1ccc(-c2nc(-c3ccc4oc5cc6ccccc6cc5c4c3)nc(-c3cccc4oc5c6ccccc6ccc5c34)n2)cc1 ; c1ccc(-c2nc(-c3ccc4sc5c6ccccc6ccc5c4c3)nc(-c3cccc4oc5cc6ccccc6cc5c34)n2)cc1 ; c1ccc(-c2nc(-c3ccc4c(c3)oc3ccc5ccccc5c34)nc(-c3cccc4oc5cc6ccccc6cc5c34)n2)cc1]

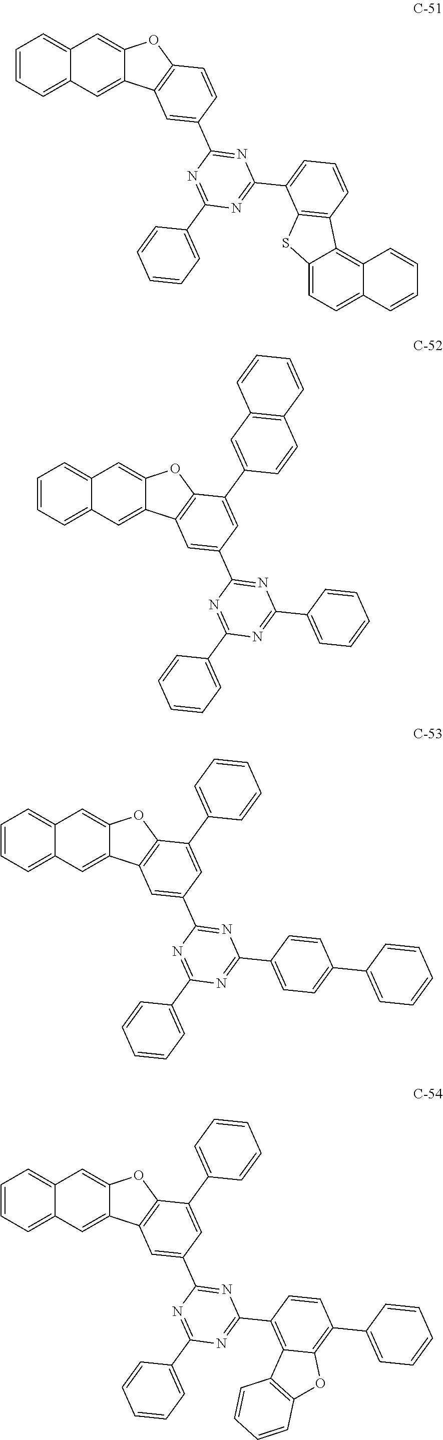 [SMILES: c1ccc(-c2nc(-c3ccc4oc5cc6ccccc6cc5c4c3)nc(-c3cccc4c3sc3ccc5ccccc5c34)n2)cc1 ; c1ccc(-c2nc(-c3ccccc3)nc(-c3cc(-c4ccc5ccccc5c4)c4oc5cc6ccccc6cc5c4c3)n2)cc1 ; c1ccc(-c2ccc(-c3nc(-c4ccccc4)nc(-c4cc(-c5ccccc5)c5oc6cc7ccccc7cc6c5c4)n3)cc2)cc1 ; c1ccc(-c2nc(-c3cc(-c4ccccc4)c4oc5cc6ccccc6cc5c4c3)nc(-c3ccc(-c4ccccc4)c4oc5ccccc5c34)n2)cc1]

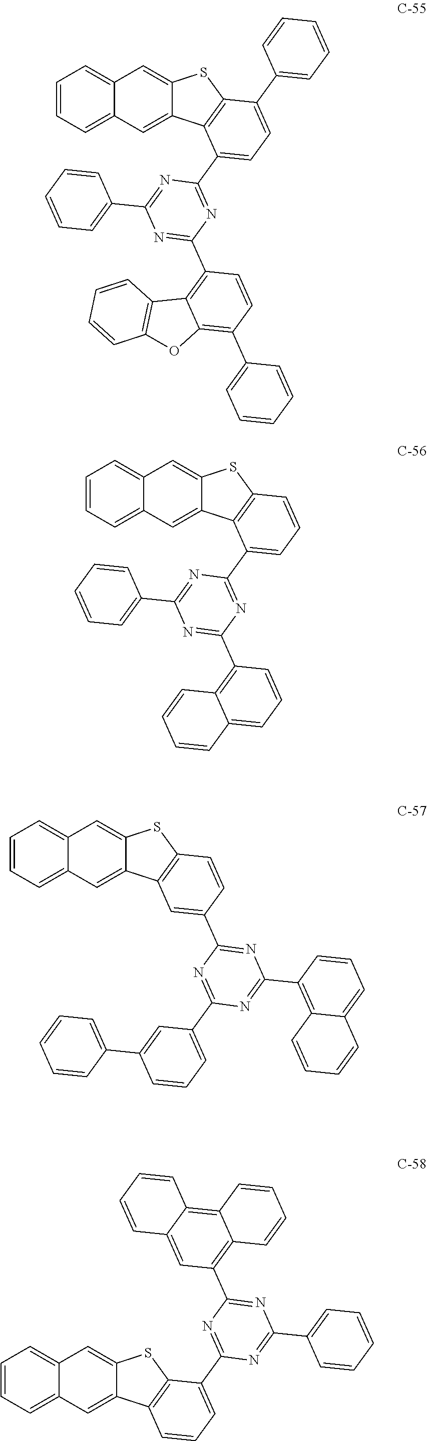 [SMILES: c1ccc(-c2nc(-c3ccc(-c4ccccc4)c4oc5ccccc5c34)nc(-c3ccc(-c4ccccc4)c4sc5cc6ccccc6cc5c34)n2)cc1 ; c1ccc(-c2nc(-c3cccc4ccccc34)nc(-c3cccc4sc5cc6ccccc6cc5c34)n2)cc1 ; c1ccc(-c2cccc(-c3nc(-c4ccc5sc6cc7ccccc7cc6c5c4)nc(-c4cccc5ccccc45)n3)c2)cc1 ; c1ccc(-c2nc(-c3cc4ccccc4c4ccccc34)nc(-c3cccc4c3sc3cc5ccccc5cc34)n2)cc1]

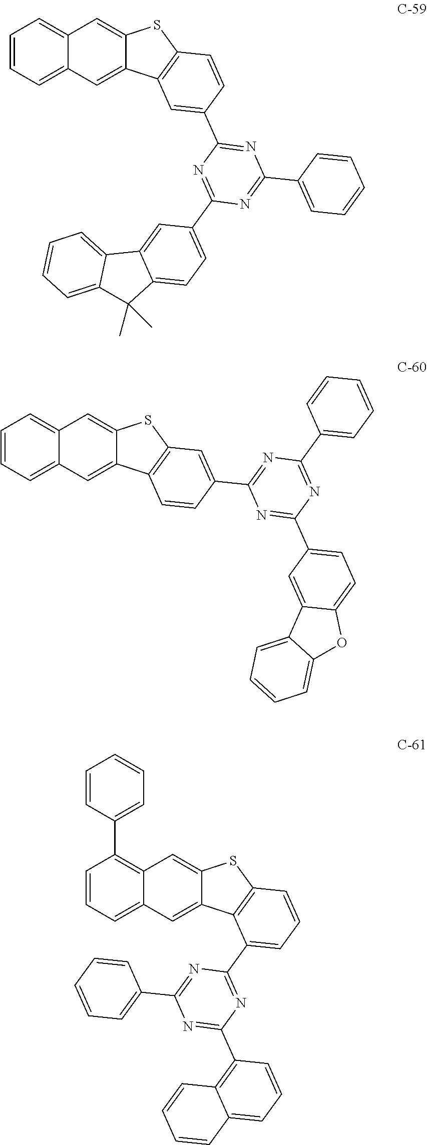 [SMILES: CC1(C)c2ccccc2-c2cc(-c3nc(-c4ccccc4)nc(-c4ccc5sc6cc7ccccc7cc6c5c4)n3)ccc21 ; c1ccc(-c2nc(-c3ccc4c(c3)sc3cc5ccccc5cc34)nc(-c3ccc4oc5ccccc5c4c3)n2)cc1 ; c1ccc(-c2nc(-c3cccc4ccccc34)nc(-c3cccc4sc5cc6c(-c7ccccc7)cccc6cc5c34)n2)cc1]

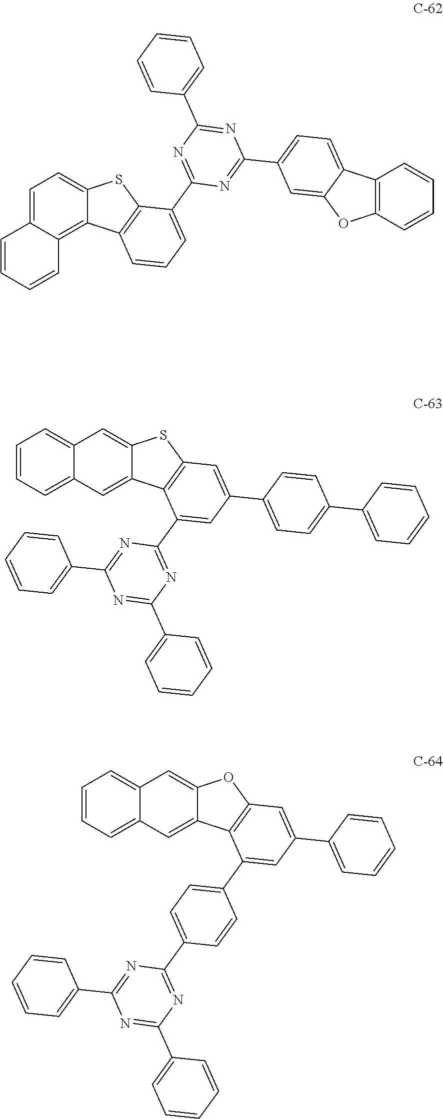 [SMILES: c1ccc(-c2nc(-c3ccc4c(c3)oc3ccccc34)nc(-c3cccc4c3sc3ccc5ccccc5c34)n2)cc1 ; c1ccc(-c2ccc(-c3cc(-c4nc(-c5ccccc5)nc(-c5ccccc5)n4)c4c(c3)sc3cc5ccccc5cc34)cc2)cc1 ; c1ccc(-c2cc(-c3ccc(-c4nc(-c5ccccc5)nc(-c5ccccc5)n4)cc3)c3c(c2)oc2cc4ccccc4cc23)cc1]

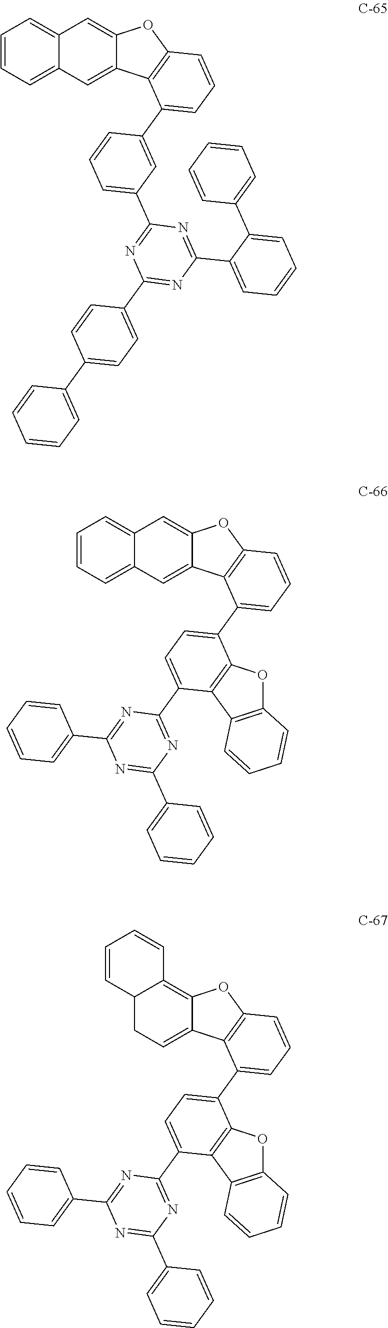 [SMILES: c1ccc(-c2ccc(-c3nc(-c4cccc(-c5cccc6oc7cc8ccccc8cc7c56)c4)nc(-c4ccccc4-c4ccccc4)n3)cc2)cc1 ; c1ccc(-c2nc(-c3ccccc3)nc(-c3ccc(-c4cccc5oc6cc7ccccc7cc6c45)c4oc5ccccc5c34)n2)cc1 ; C1=CC2=c3oc4cccc(-c5ccc(-c6nc(-c7ccccc7)nc(-c7ccccc7)n6)c6c5oc5ccccc56)c4c3=CCC2C=C1]

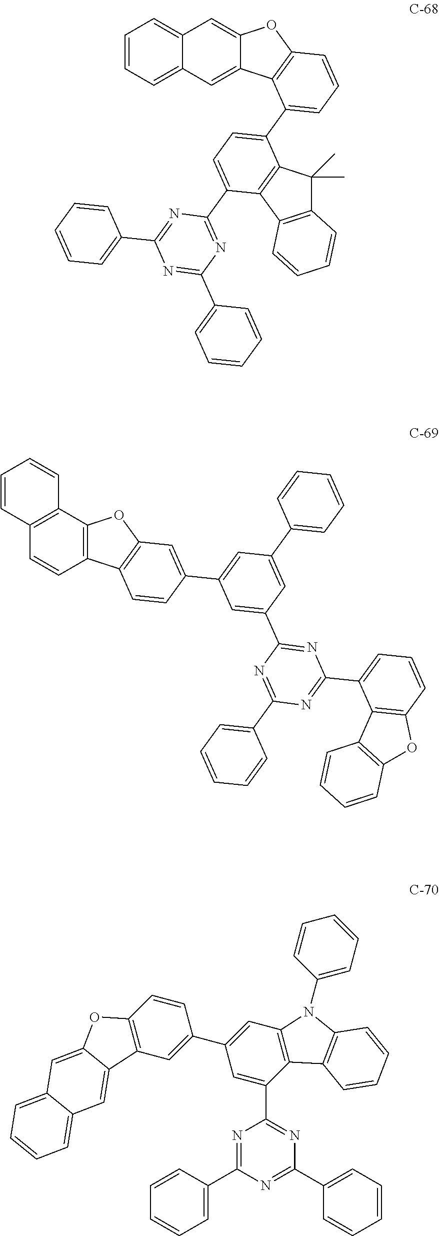 [SMILES: CC1(C)c2ccccc2-c2c(-c3nc(-c4ccccc4)nc(-c4ccccc4)n3)ccc(-c3cccc4oc5cc6ccccc6cc5c34)c21 ; c1ccc(-c2cc(-c3ccc4c(c3)oc3c5ccccc5ccc43)cc(-c3nc(-c4ccccc4)nc(-c4cccc5oc6ccccc6c45)n3)c2)cc1 ; c1ccc(-c2nc(-c3ccccc3)nc(-c3cc(-c4ccc5oc6cc7ccccc7cc6c5c4)cc4c3c3ccccc3n4-c3ccccc3)n2)cc1]

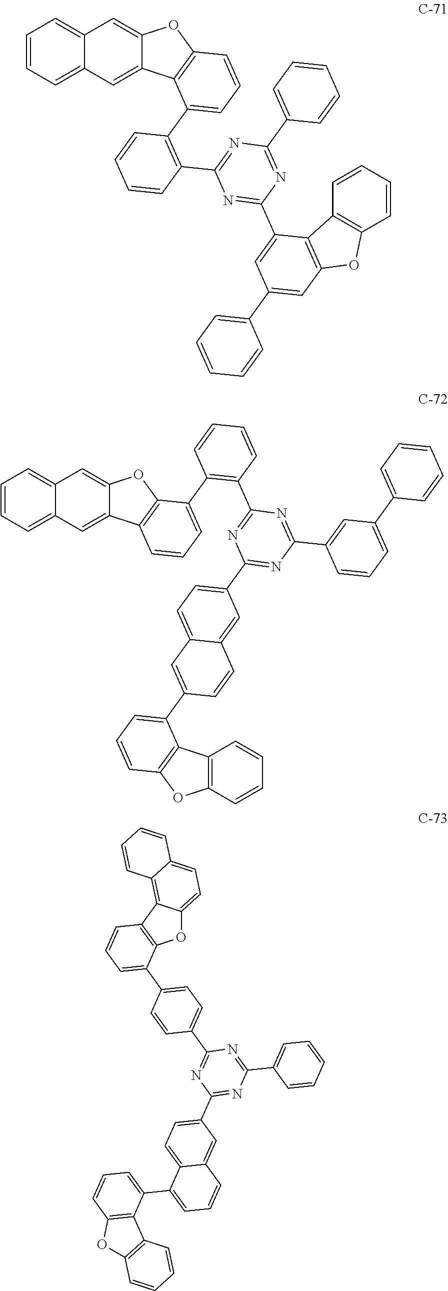

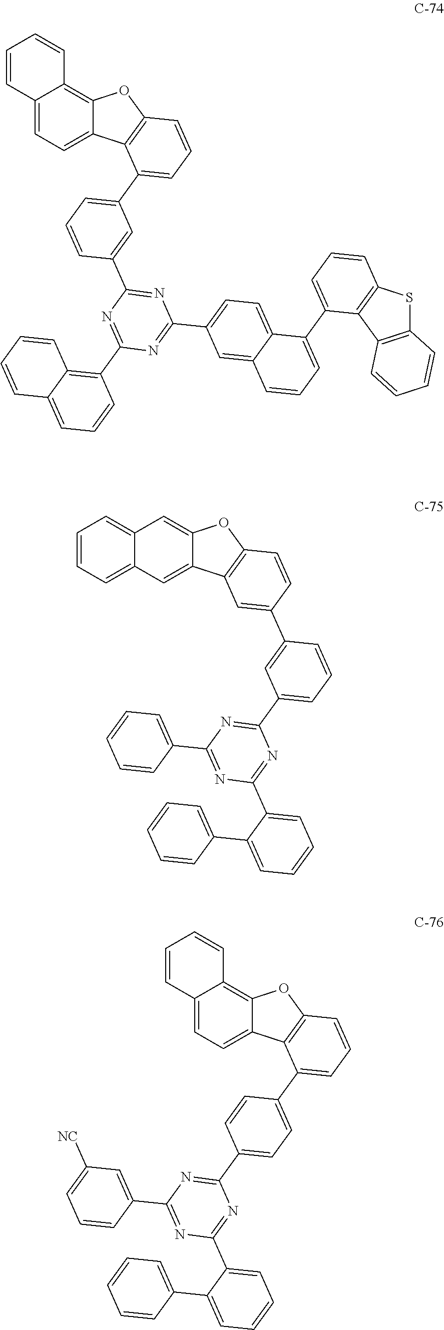 [SMILES: c1cc(-c2nc(-c3ccc4c(-c5cccc6sc7ccccc7c56)cccc4c3)nc(-c3cccc4ccccc34)n2)cc(-c2cccc3oc4c5ccccc5ccc4c23)c1 ; c1ccc(-c2nc(-c3cccc(-c4ccc5oc6cc7ccccc7cc6c5c4)c3)nc(-c3ccccc3-c3ccccc3)n2)cc1 ; N#Cc1cccc(-c2nc(-c3ccc(-c4cccc5oc6c7ccccc7ccc6c45)cc3)nc(-c3ccccc3-c3ccccc3)n2)c1]

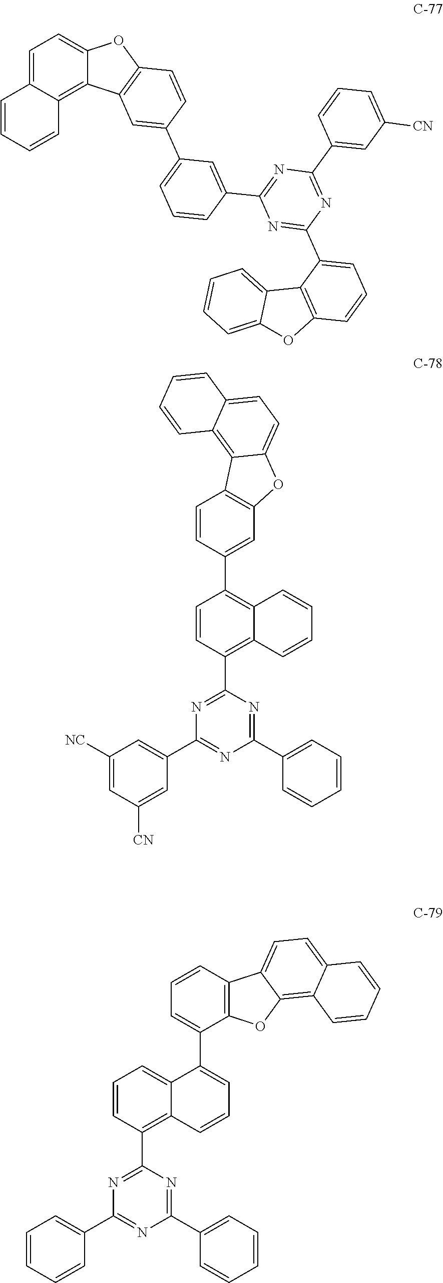

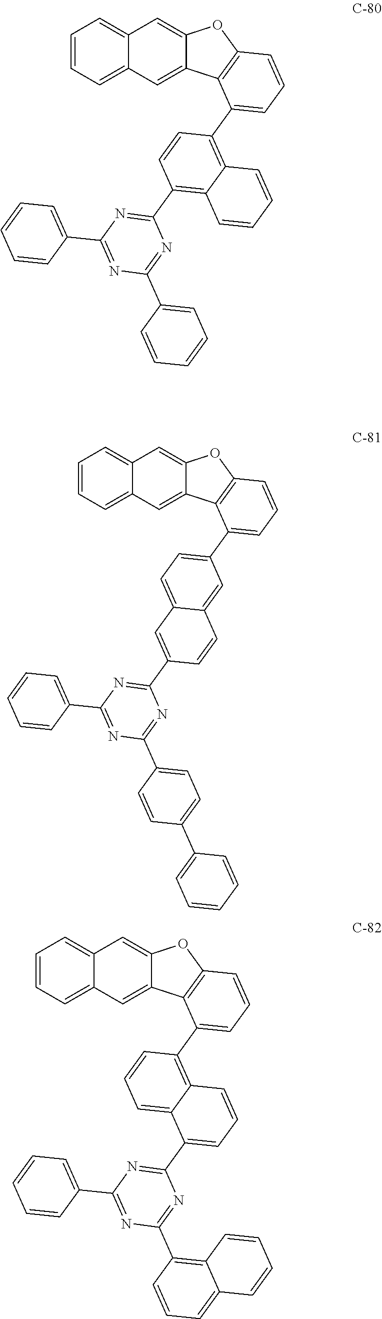

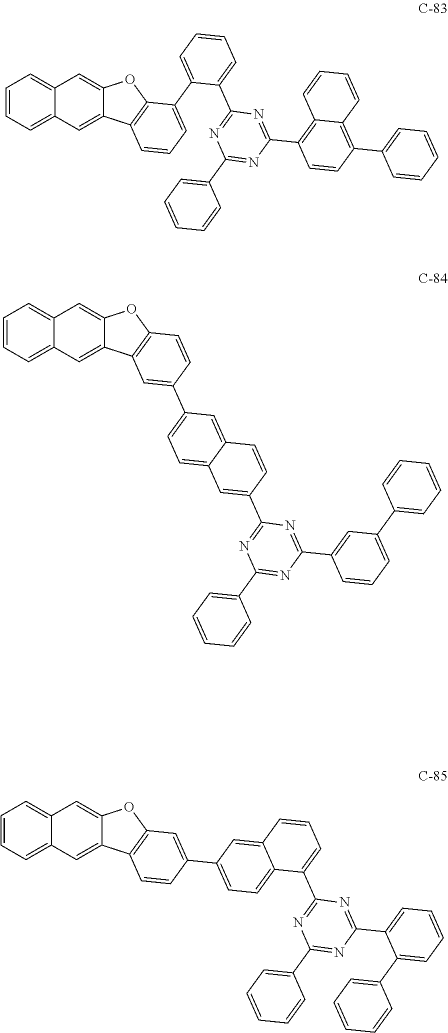 [SMILES: c1ccc(-c2nc(-c3ccccc3-c3cccc4c3oc3cc5ccccc5cc34)nc(-c3ccc(-c4ccccc4)c4ccccc34)n2)cc1 ; c1ccc(-c2cccc(-c3nc(-c4ccccc4)nc(-c4ccc5cc(-c6ccc7oc8cc9ccccc9cc8c7c6)ccc5c4)n3)c2)cc1 ; c1ccc(-c2nc(-c3ccccc3-c3ccccc3)nc(-c3cccc4cc(-c5ccc6c(c5)oc5cc7ccccc7cc56)ccc34)n2)cc1]

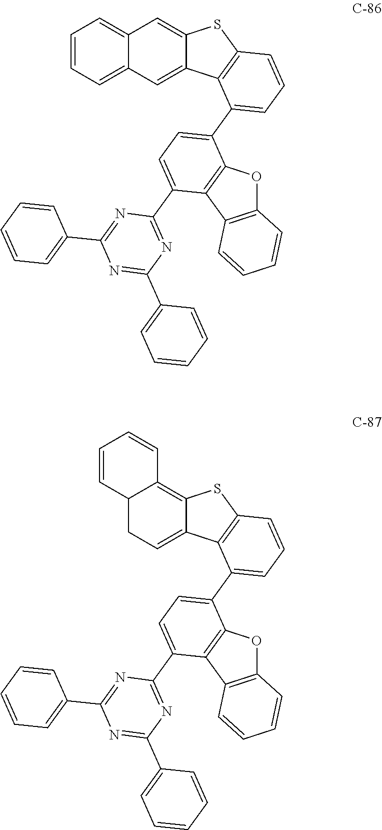 [SMILES: c1ccc(-c2nc(-c3ccccc3)nc(-c3ccc(-c4cccc5sc6cc7ccccc7cc6c45)c4oc5ccccc5c34)n2)cc1 ; C1=CC2=c3sc4cccc(-c5ccc(-c6nc(-c7ccccc7)nc(-c7ccccc7)n6)c6c5oc5ccccc56)c4c3=CCC2C=C1]

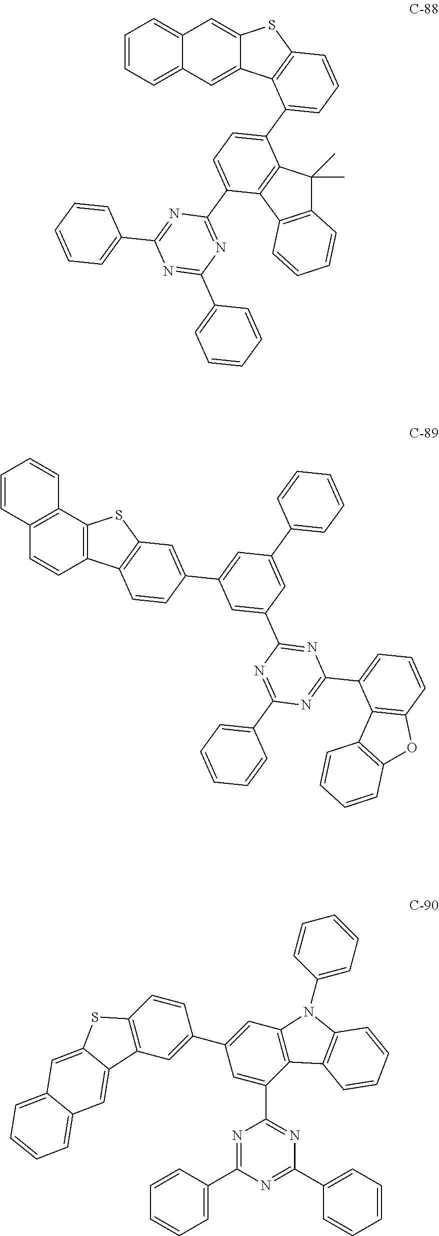 [SMILES: CC1(C)c2ccccc2-c2c(-c3nc(-c4ccccc4)nc(-c4ccccc4)n3)ccc(-c3cccc4sc5cc6ccccc6cc5c34)c21 ; c1ccc(-c2cc(-c3ccc4c(c3)sc3c5ccccc5ccc43)cc(-c3nc(-c4ccccc4)nc(-c4cccc5oc6ccccc6c45)n3)c2)cc1 ; c1ccc(-c2nc(-c3ccccc3)nc(-c3cc(-c4ccc5sc6cc7ccccc7cc6c5c4)cc4c3c3ccccc3n4-c3ccccc3)n2)cc1]

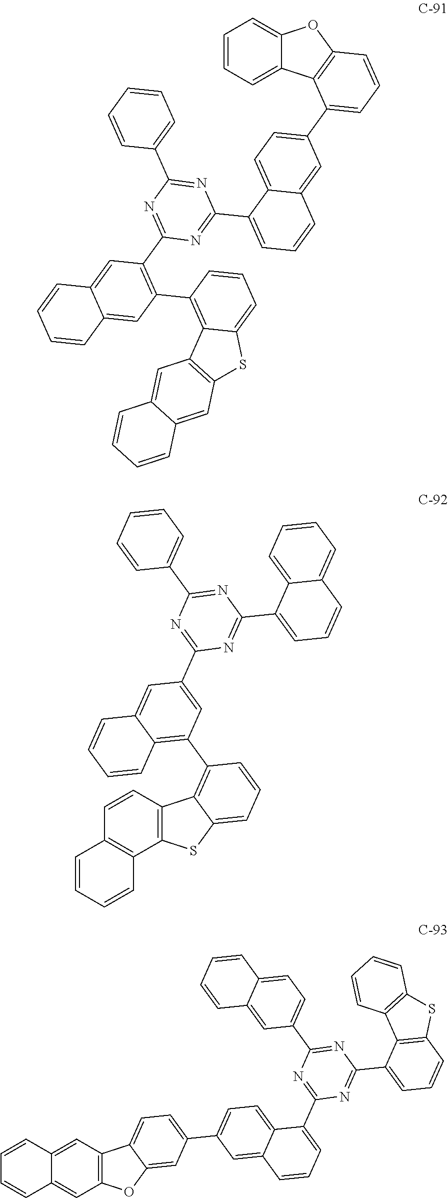

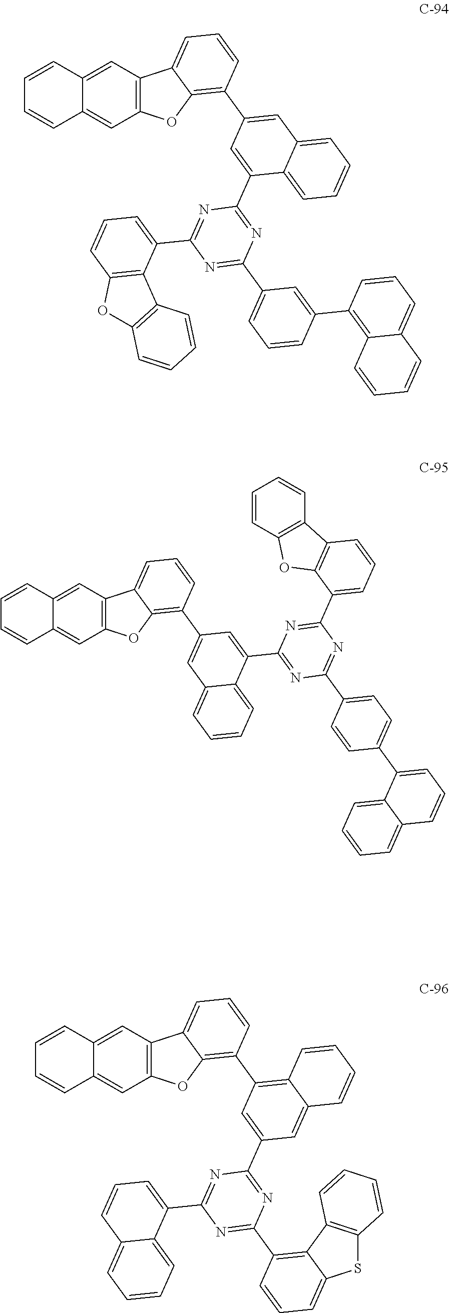 [SMILES: c1cc(-c2nc(-c3cc(-c4cccc5c4oc4cc6ccccc6cc45)cc4ccccc34)nc(-c3cccc4oc5ccccc5c34)n2)cc(-c2cccc3ccccc23)c1 ; c1ccc2cc3c(cc2c1)oc1c(-c2cc(-c4nc(-c5ccc(-c6cccc7ccccc67)cc5)nc(-c5cccc6c5oc5ccccc56)n4)c4ccccc4c2)cccc13 ; c1ccc2cc3c(cc2c1)oc1c(-c2cc(-c4nc(-c5cccc6ccccc56)nc(-c5cccc6sc7ccccc7c56)n4)cc4ccccc24)cccc13]

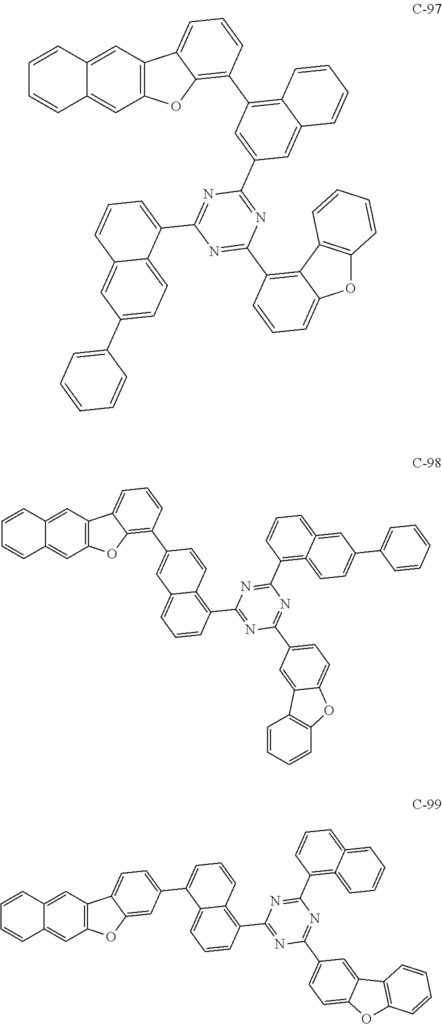

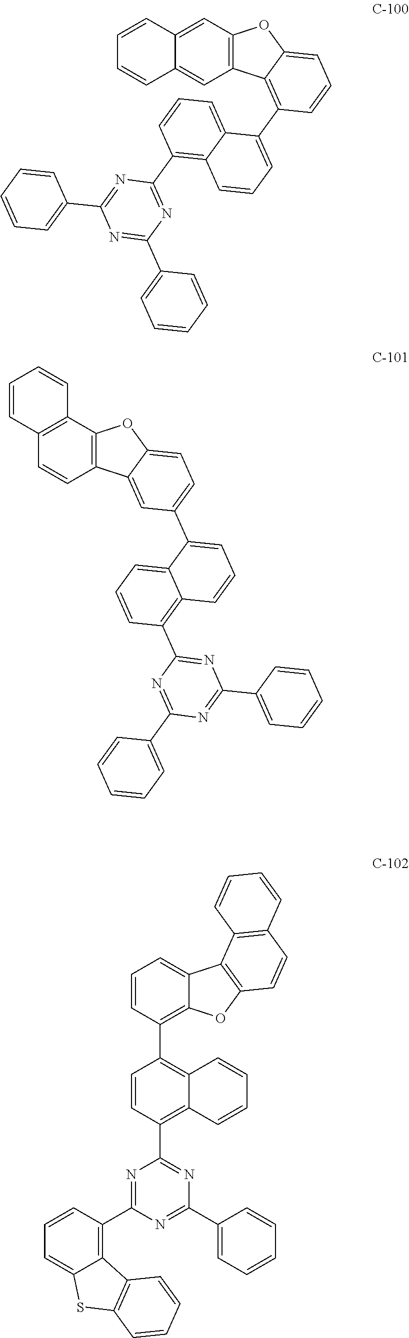 [SMILES: c1ccc(-c2nc(-c3ccccc3)nc(-c3cccc4c(-c5cccc6oc7cc8ccccc8cc7c56)cccc34)n2)cc1 ; c1ccc(-c2nc(-c3ccccc3)nc(-c3cccc4c(-c5ccc6oc7c8ccccc8ccc7c6c5)cccc34)n2)cc1 ; c1ccc(-c2nc(-c3ccc(-c4cccc5c4oc4ccc6ccccc6c45)c4ccccc34)nc(-c3cccc4sc5ccccc5c34)n2)cc1]

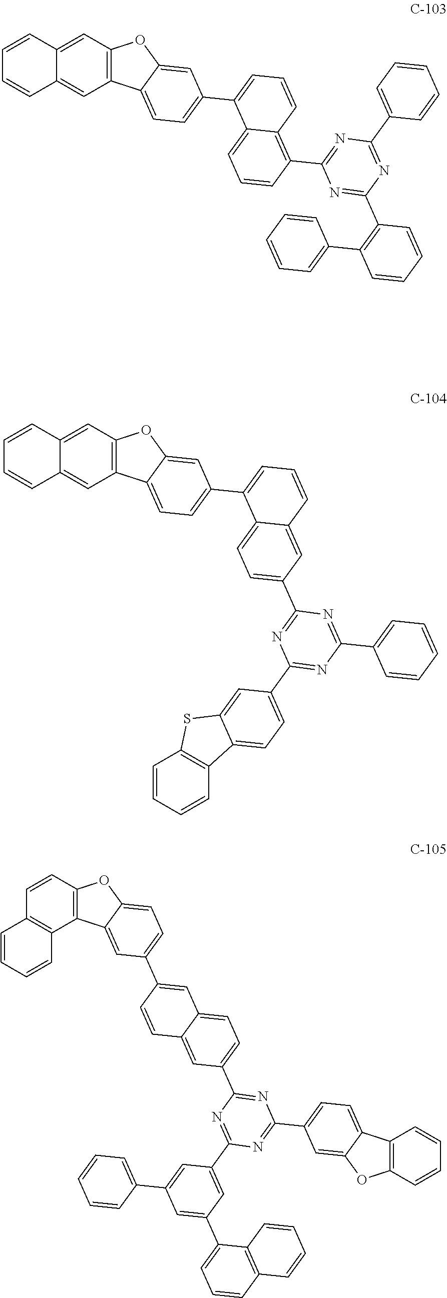 [SMILES: c1ccc(-c2nc(-c3ccccc3-c3ccccc3)nc(-c3cccc4c(-c5ccc6c(c5)oc5cc7ccccc7cc56)cccc34)n2)cc1 ; c1ccc(-c2nc(-c3ccc4c(-c5ccc6c(c5)oc5cc7ccccc7cc56)cccc4c3)nc(-c3ccc4c(c3)sc3ccccc34)n2)cc1 ; c1ccc(-c2cc(-c3nc(-c4ccc5cc(-c6ccc7oc8ccc9ccccc9c8c7c6)ccc5c4)nc(-c4ccc5c(c4)oc4ccccc45)n3)cc(-c3cccc4ccccc34)c2)cc1]

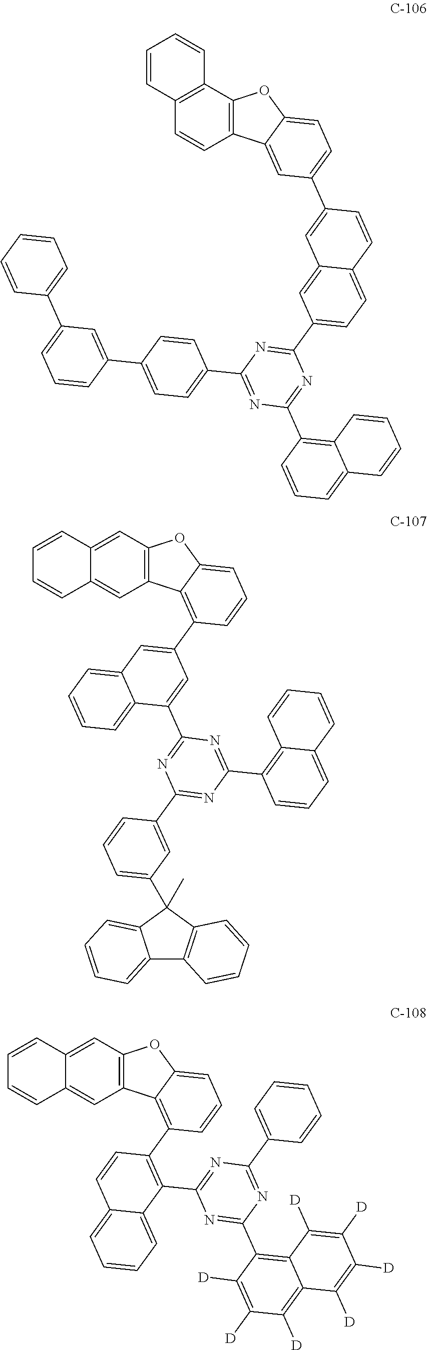 [SMILES: c1ccc(-c2cccc(-c3ccc(-c4nc(-c5ccc6ccc(-c7ccc8oc9c%10ccccc%10ccc9c8c7)cc6c5)nc(-c5cccc6ccccc56)n4)cc3)c2)cc1 ; CC1(c2cccc(-c3nc(-c4cccc5ccccc45)nc(-c4cc(-c5cccc6oc7cc8ccccc8cc7c56)cc5ccccc45)n3)c2)c2ccccc2-c2ccccc21 ; [2H]c1c([2H])c([2H])c2c(-c3nc(-c4ccccc4)nc(-c4c(-c5cccc6oc7cc8ccccc8cc7c56)ccc5ccccc45)n3)c([2H])c([2H])c([2H])c2c1[2H]]

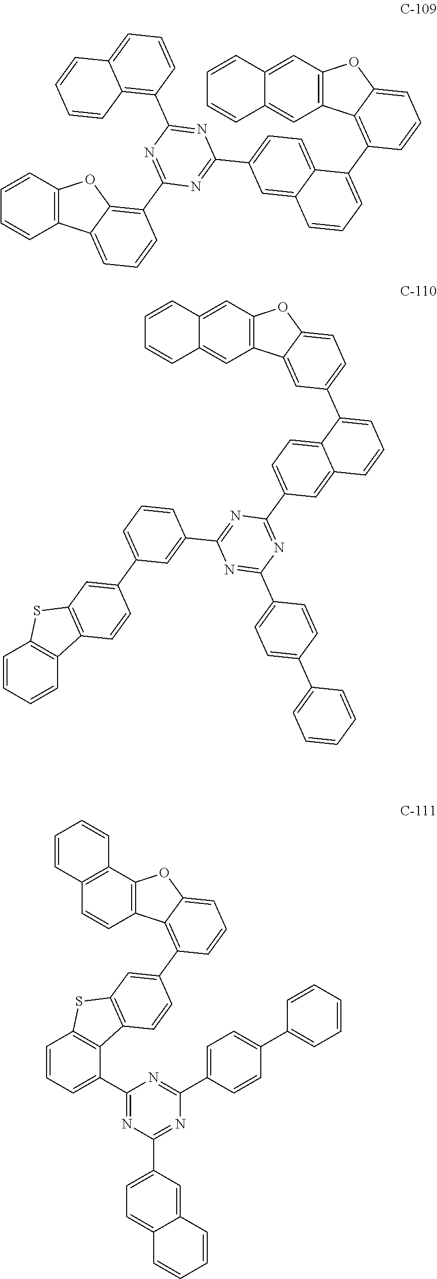 [SMILES: c1ccc2cc3c(cc2c1)oc1cccc(-c2cccc4cc(-c5nc(-c6cccc7ccccc67)nc(-c6cccc7c6oc6ccccc67)n5)ccc24)c13 ; c1ccc(-c2ccc(-c3nc(-c4cccc(-c5ccc6c(c5)sc5ccccc56)c4)nc(-c4ccc5c(-c6ccc7oc8cc9ccccc9cc8c7c6)cccc5c4)n3)cc2)cc1 ; c1ccc(-c2ccc(-c3nc(-c4ccc5ccccc5c4)nc(-c4cccc5sc6cc(-c7cccc8oc9c%10ccccc%10ccc9c78)ccc6c45)n3)cc2)cc1]

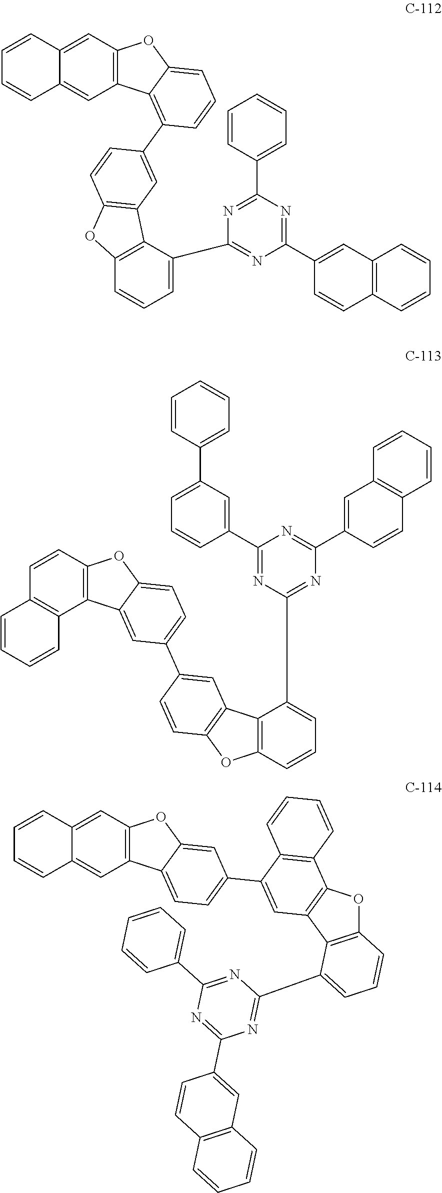

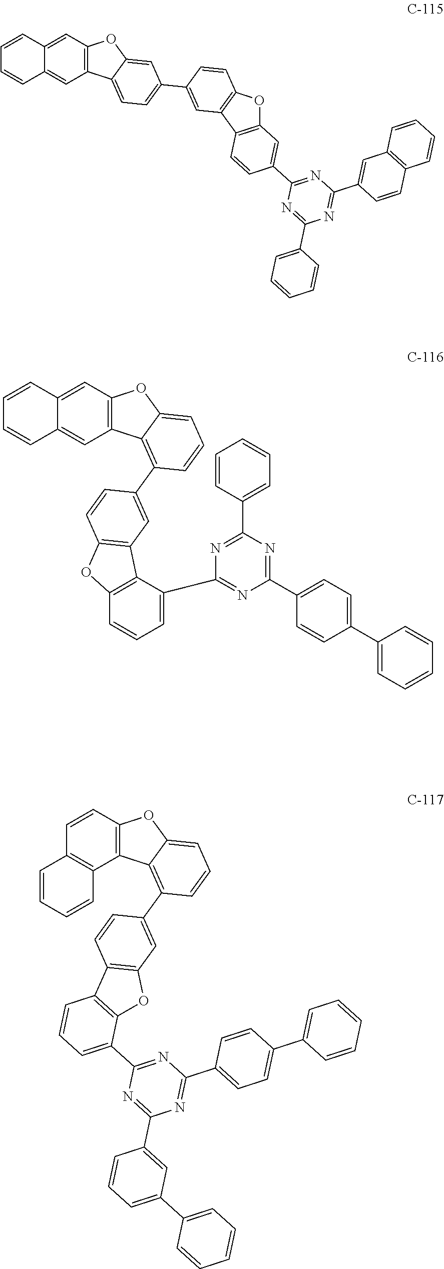 [SMILES: c1ccc(-c2nc(-c3ccc4ccccc4c3)nc(-c3ccc4c(c3)oc3ccc(-c5ccc6c(c5)oc5cc7ccccc7cc56)cc34)n2)cc1 ; c1ccc(-c2ccc(-c3nc(-c4ccccc4)nc(-c4cccc5oc6ccc(-c7cccc8oc9cc%10ccccc%10cc9c78)cc6c45)n3)cc2)cc1 ; c1ccc(-c2ccc(-c3nc(-c4cccc(-c5ccccc5)c4)nc(-c4cccc5c4oc4cc(-c6cccc7oc8ccc9ccccc9c8c67)ccc45)n3)cc2)cc1]

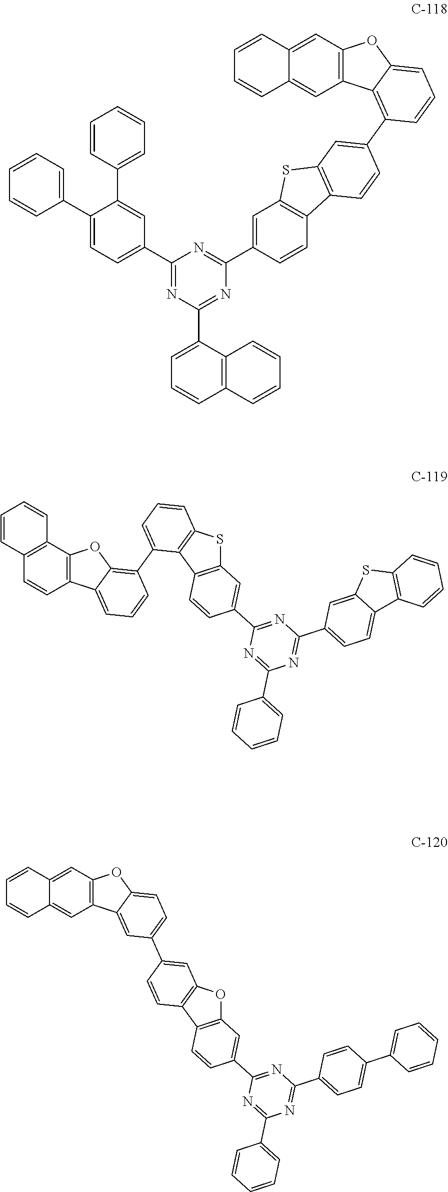 [SMILES: c1ccc(-c2ccc(-c3nc(-c4ccc5c(c4)sc4cc(-c6cccc7oc8cc9ccccc9cc8c67)ccc45)nc(-c4cccc5ccccc45)n3)cc2-c2ccccc2)cc1 ; c1ccc(-c2nc(-c3ccc4c(c3)sc3ccccc34)nc(-c3ccc4c(c3)sc3cccc(-c5cccc6c5oc5c7ccccc7ccc65)c34)n2)cc1 ; c1ccc(-c2ccc(-c3nc(-c4ccccc4)nc(-c4ccc5c(c4)oc4cc(-c6ccc7oc8cc9ccccc9cc8c7c6)ccc45)n3)cc2)cc1]

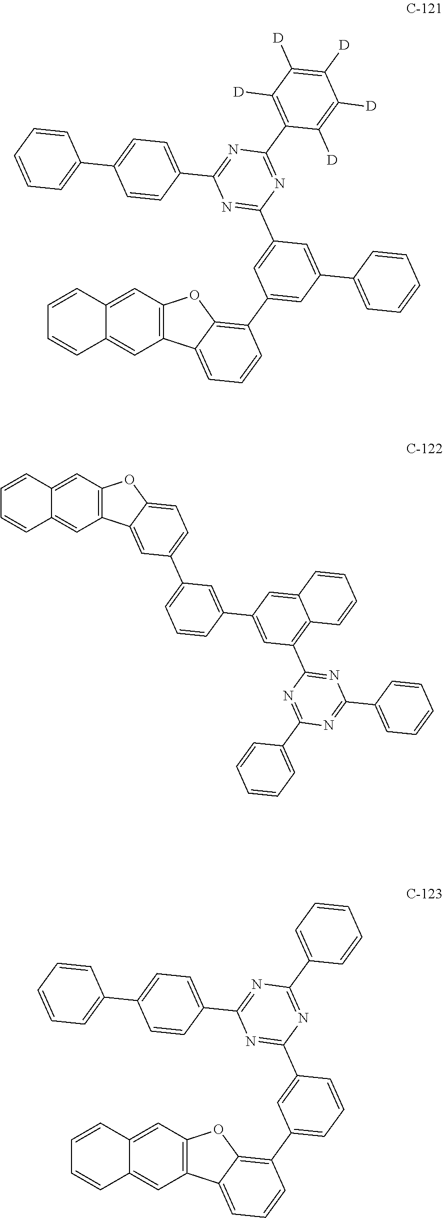 [SMILES: [2H]c1c([2H])c([2H])c(-c2nc(-c3ccc(-c4ccccc4)cc3)nc(-c3cc(-c4ccccc4)cc(-c4cccc5c4oc4cc6ccccc6cc45)c3)n2)c([2H])c1[2H] ; c1ccc(-c2nc(-c3ccccc3)nc(-c3cc(-c4cccc(-c5ccc6oc7cc8ccccc8cc7c6c5)c4)cc4ccccc34)n2)cc1 ; c1ccc(-c2ccc(-c3nc(-c4ccccc4)nc(-c4cccc(-c5cccc6c5oc5cc7ccccc7cc56)c4)n3)cc2)cc1]

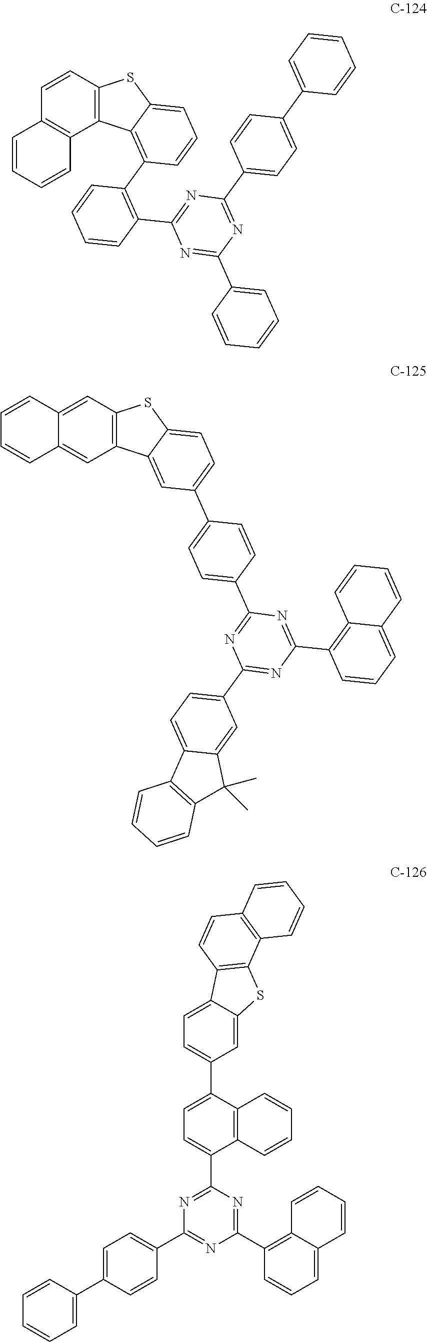 [SMILES: c1ccc(-c2ccc(-c3nc(-c4ccccc4)nc(-c4ccccc4-c4cccc5sc6ccc7ccccc7c6c45)n3)cc2)cc1 ; CC1(C)c2ccccc2-c2ccc(-c3nc(-c4ccc(-c5ccc6sc7cc8ccccc8cc7c6c5)cc4)nc(-c4cccc5ccccc45)n3)cc21 ; c1ccc(-c2ccc(-c3nc(-c4cccc5ccccc45)nc(-c4ccc(-c5ccc6c(c5)sc5c7ccccc7ccc65)c5ccccc45)n3)cc2)cc1]

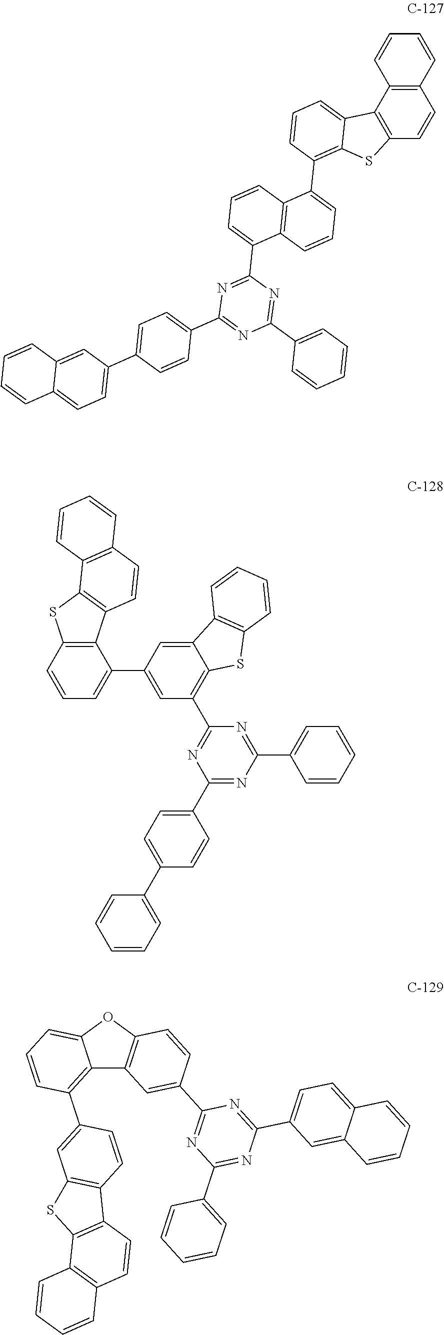 [SMILES: c1ccc(-c2nc(-c3ccc(-c4ccc5ccccc5c4)cc3)nc(-c3cccc4c(-c5cccc6c5sc5ccc7ccccc7c56)cccc34)n2)cc1 ; c1ccc(-c2ccc(-c3nc(-c4ccccc4)nc(-c4cc(-c5cccc6sc7c8ccccc8ccc7c56)cc5c4sc4ccccc45)n3)cc2)cc1 ; c1ccc(-c2nc(-c3ccc4ccccc4c3)nc(-c3ccc4oc5cccc(-c6ccc7c(c6)sc6c8ccccc8ccc76)c5c4c3)n2)cc1]

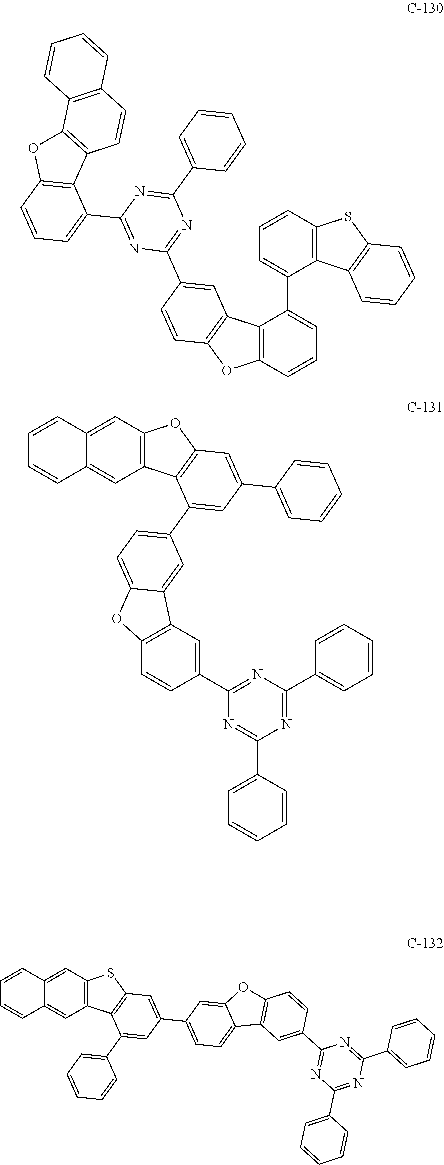 [SMILES: c1ccc(-c2nc(-c3ccc4oc5cccc(-c6cccc7sc8ccccc8c67)c5c4c3)nc(-c3cccc4oc5c6ccccc6ccc5c34)n2)cc1 ; c1ccc(-c2cc(-c3ccc4oc5ccc(-c6nc(-c7ccccc7)nc(-c7ccccc7)n6)cc5c4c3)c3c(c2)oc2cc4ccccc4cc23)cc1 ; c1ccc(-c2nc(-c3ccccc3)nc(-c3ccc4oc5cc(-c6cc(-c7ccccc7)c7c(c6)sc6cc8ccccc8cc67)ccc5c4c3)n2)cc1]

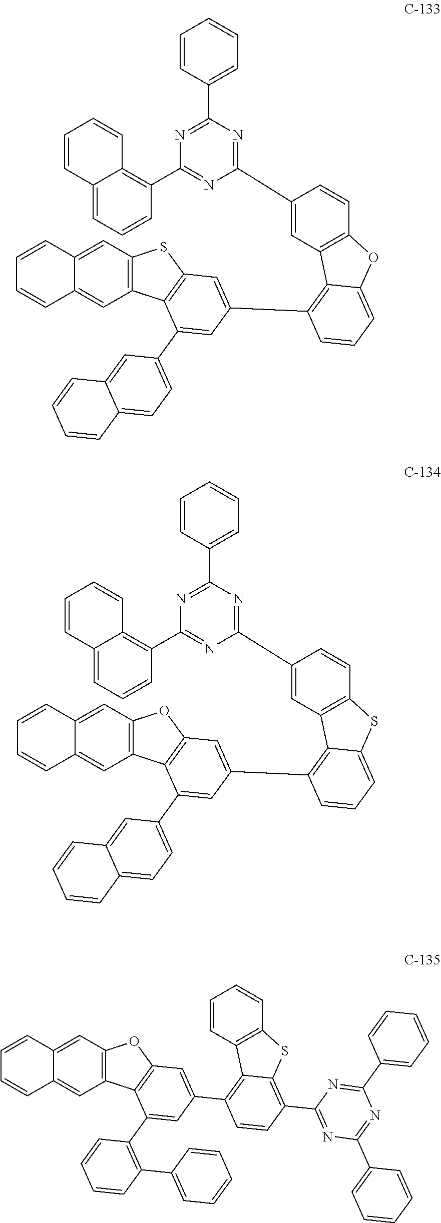 [SMILES: c1ccc(-c2nc(-c3ccc4oc5cccc(-c6cc(-c7ccc8ccccc8c7)c7c(c6)sc6cc8ccccc8cc67)c5c4c3)nc(-c3cccc4ccccc34)n2)cc1 ; c1ccc(-c2nc(-c3ccc4sc5cccc(-c6cc(-c7ccc8ccccc8c7)c7c(c6)oc6cc8ccccc8cc67)c5c4c3)nc(-c3cccc4ccccc34)n2)cc1 ; c1ccc(-c2nc(-c3ccccc3)nc(-c3ccc(-c4cc(-c5ccccc5-c5ccccc5)c5c(c4)oc4cc6ccccc6cc45)c4c3sc3ccccc34)n2)cc1]

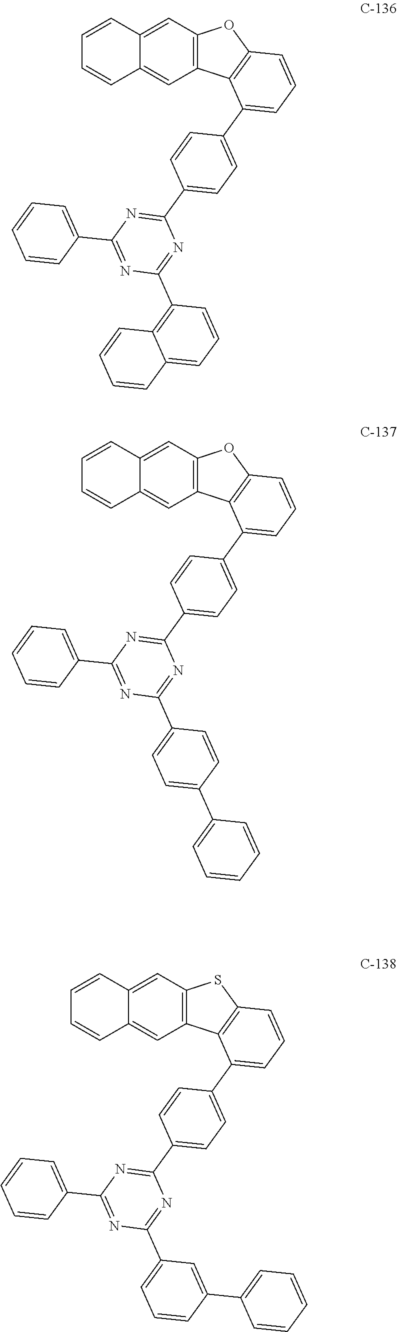 [SMILES: c1ccc(-c2nc(-c3ccc(-c4cccc5oc6cc7ccccc7cc6c45)cc3)nc(-c3cccc4ccccc34)n2)cc1 ; c1ccc(-c2ccc(-c3nc(-c4ccccc4)nc(-c4ccc(-c5cccc6oc7cc8ccccc8cc7c56)cc4)n3)cc2)cc1 ; c1ccc(-c2cccc(-c3nc(-c4ccccc4)nc(-c4ccc(-c5cccc6sc7cc8ccccc8cc7c56)cc4)n3)c2)cc1]

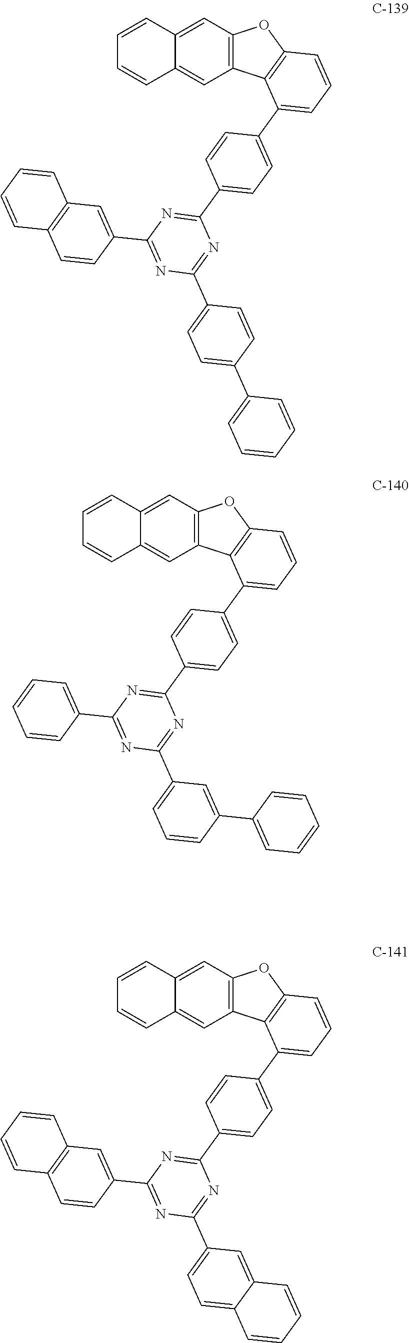 [SMILES: c1ccc(-c2ccc(-c3nc(-c4ccc(-c5cccc6oc7cc8ccccc8cc7c56)cc4)nc(-c4ccc5ccccc5c4)n3)cc2)cc1 ; c1ccc(-c2cccc(-c3nc(-c4ccccc4)nc(-c4ccc(-c5cccc6oc7cc8ccccc8cc7c56)cc4)n3)c2)cc1 ; c1ccc2cc(-c3nc(-c4ccc(-c5cccc6oc7cc8ccccc8cc7c56)cc4)nc(-c4ccc5ccccc5c4)n3)ccc2c1]

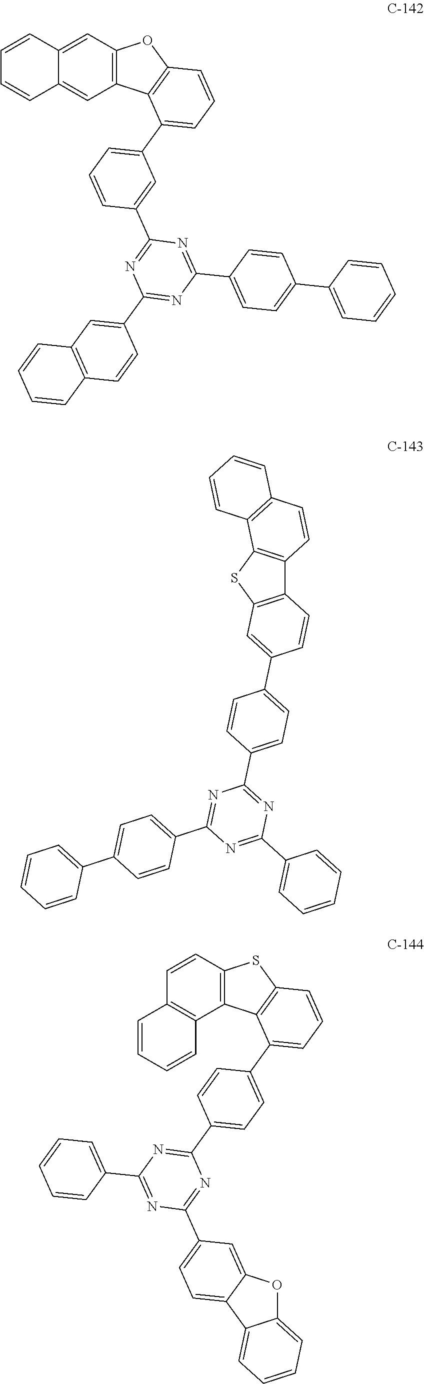 [SMILES: c1ccc(-c2ccc(-c3nc(-c4cccc(-c5cccc6oc7cc8ccccc8cc7c56)c4)nc(-c4ccc5ccccc5c4)n3)cc2)cc1 ; c1ccc(-c2ccc(-c3nc(-c4ccccc4)nc(-c4ccc(-c5ccc6c(c5)sc5c7ccccc7ccc65)cc4)n3)cc2)cc1 ; c1ccc(-c2nc(-c3ccc(-c4cccc5sc6ccc7ccccc7c6c45)cc3)nc(-c3ccc4c(c3)oc3ccccc34)n2)cc1]

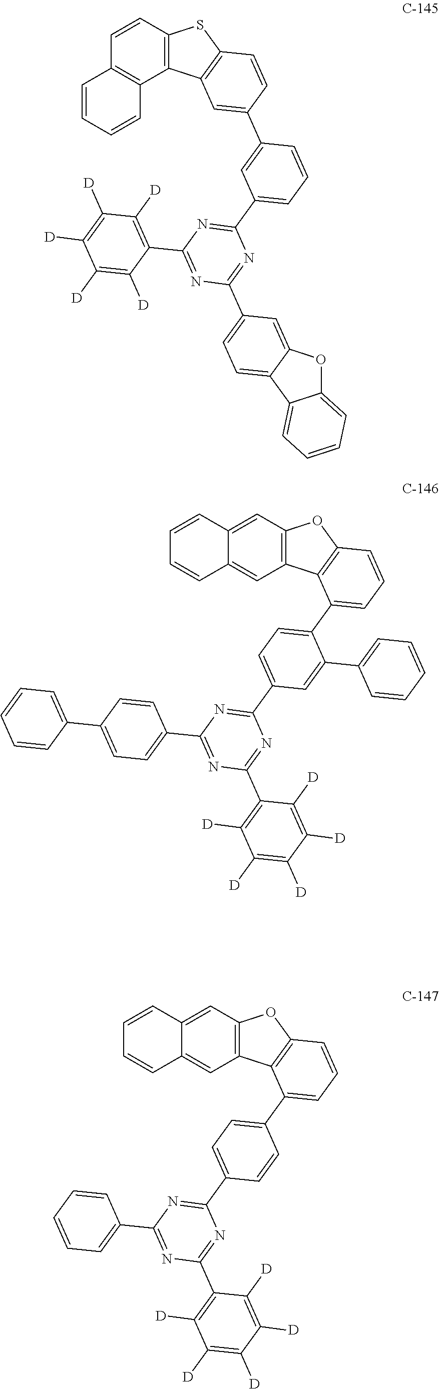 [SMILES: [2H]c1c([2H])c([2H])c(-c2nc(-c3cccc(-c4ccc5sc6ccc7ccccc7c6c5c4)c3)nc(-c3ccc4c(c3)oc3ccccc34)n2)c([2H])c1[2H] ; [2H]c1c([2H])c([2H])c(-c2nc(-c3ccc(-c4ccccc4)cc3)nc(-c3ccc(-c4cccc5oc6cc7ccccc7cc6c45)c(-c4ccccc4)c3)n2)c([2H])c1[2H] ; [2H]c1c([2H])c([2H])c(-c2nc(-c3ccccc3)nc(-c3ccc(-c4cccc5oc6cc7ccccc7cc6c45)cc3)n2)c([2H])c1[2H]]

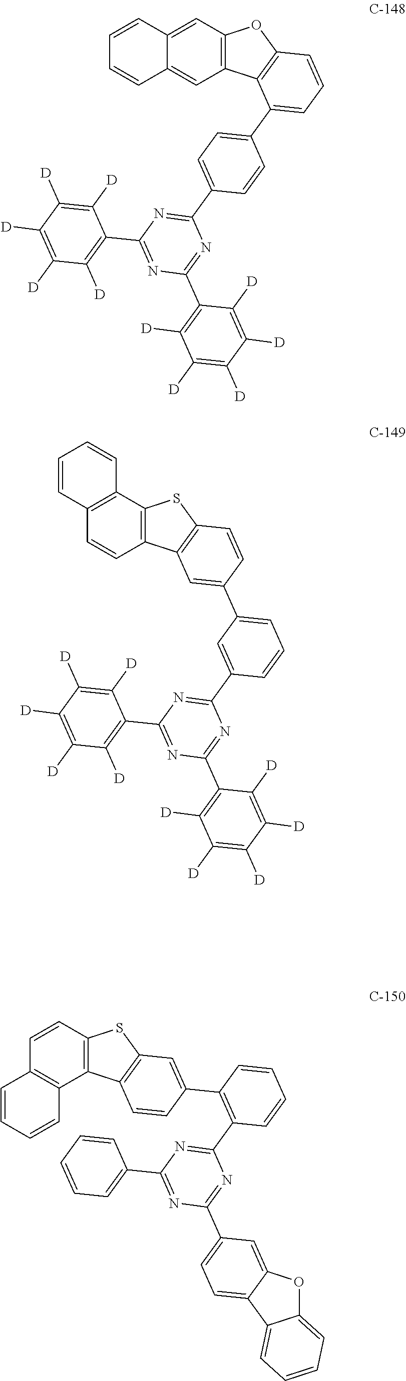 [SMILES: [2H]c1c([2H])c([2H])c(-c2nc(-c3ccc(-c4cccc5oc6cc7ccccc7cc6c45)cc3)nc(-c3c([2H])c([2H])c([2H])c([2H])c3[2H])n2)c([2H])c1[2H] ; [2H]c1c([2H])c([2H])c(-c2nc(-c3cccc(-c4ccc5sc6c7ccccc7ccc6c5c4)c3)nc(-c3c([2H])c([2H])c([2H])c([2H])c3[2H])n2)c([2H])c1[2H] ; c1ccc(-c2nc(-c3ccc4c(c3)oc3ccccc34)nc(-c3ccccc3-c3ccc4c(c3)sc3ccc5ccccc5c34)n2)cc1]

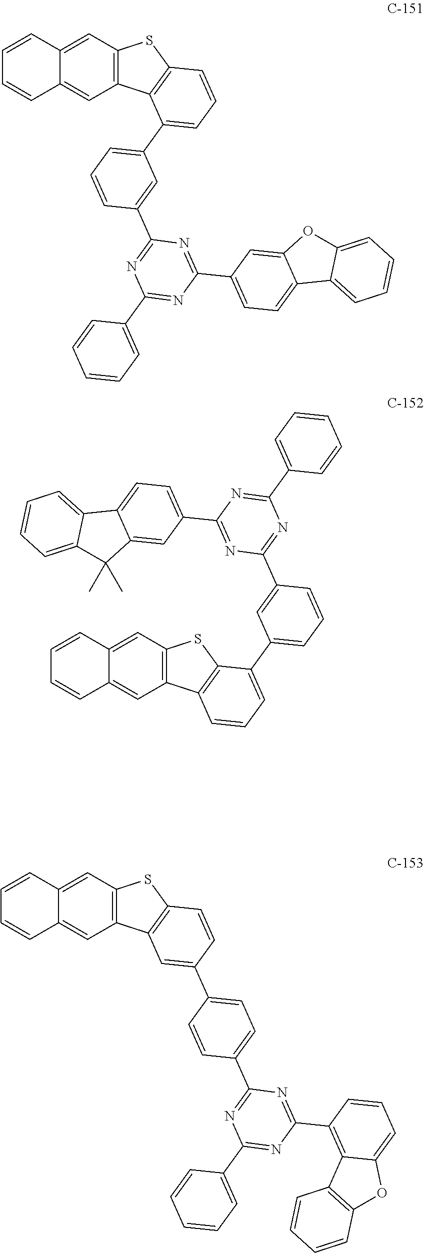 [SMILES: c1ccc(-c2nc(-c3cccc(-c4cccc5sc6cc7ccccc7cc6c45)c3)nc(-c3ccc4c(c3)oc3ccccc34)n2)cc1 ; CC1(C)c2ccccc2-c2ccc(-c3nc(-c4ccccc4)nc(-c4cccc(-c5cccc6c5sc5cc7ccccc7cc56)c4)n3)cc21 ; c1ccc(-c2nc(-c3ccc(-c4ccc5sc6cc7ccccc7cc6c5c4)cc3)nc(-c3cccc4oc5ccccc5c34)n2)cc1]

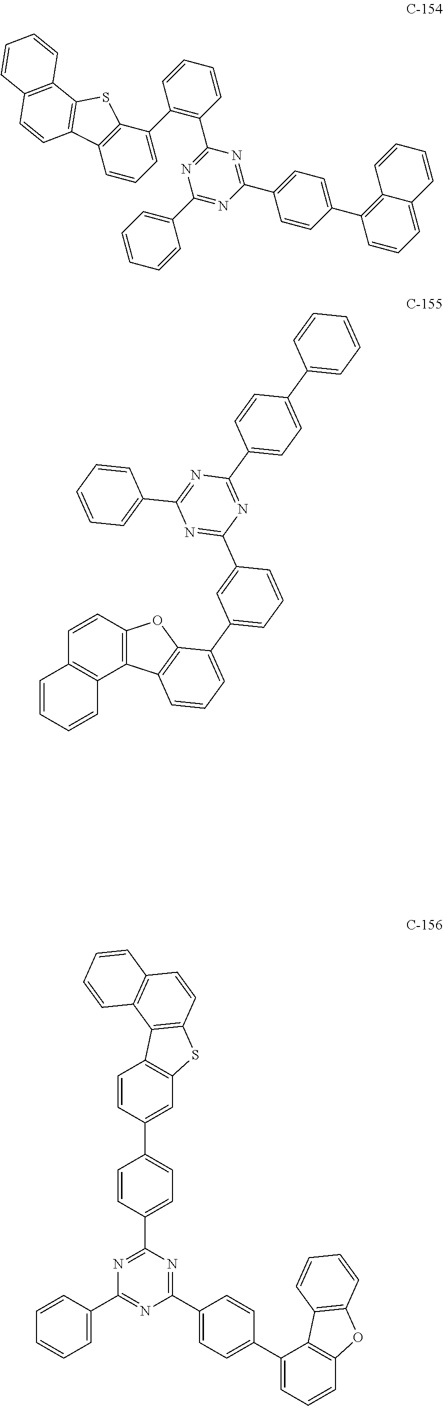 [SMILES: c1ccc(-c2nc(-c3ccc(-c4cccc5ccccc45)cc3)nc(-c3ccccc3-c3cccc4c3sc3c5ccccc5ccc43)n2)cc1 ; c1ccc(-c2ccc(-c3nc(-c4ccccc4)nc(-c4cccc(-c5cccc6c5oc5ccc7ccccc7c56)c4)n3)cc2)cc1 ; c1ccc(-c2nc(-c3ccc(-c4ccc5c(c4)sc4ccc6ccccc6c45)cc3)nc(-c3ccc(-c4cccc5oc6ccccc6c45)cc3)n2)cc1]

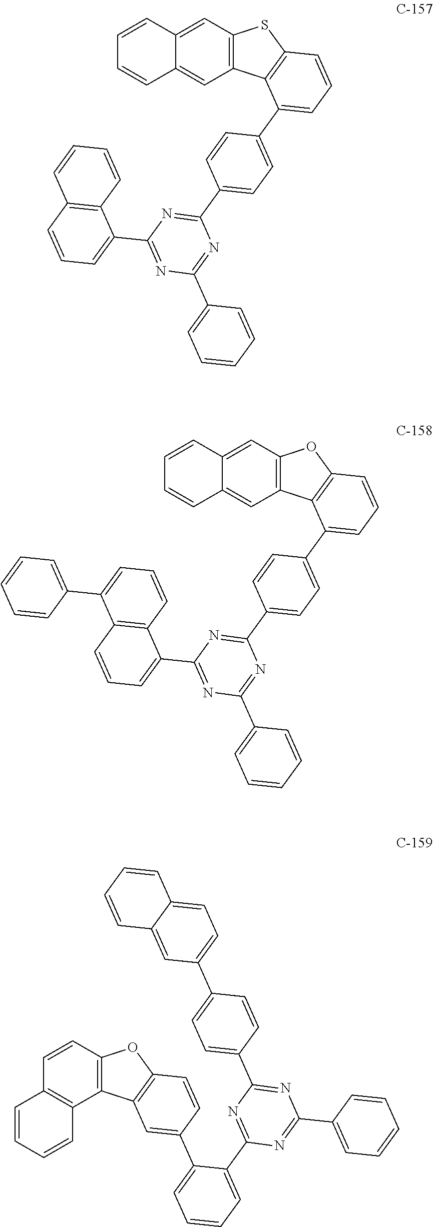

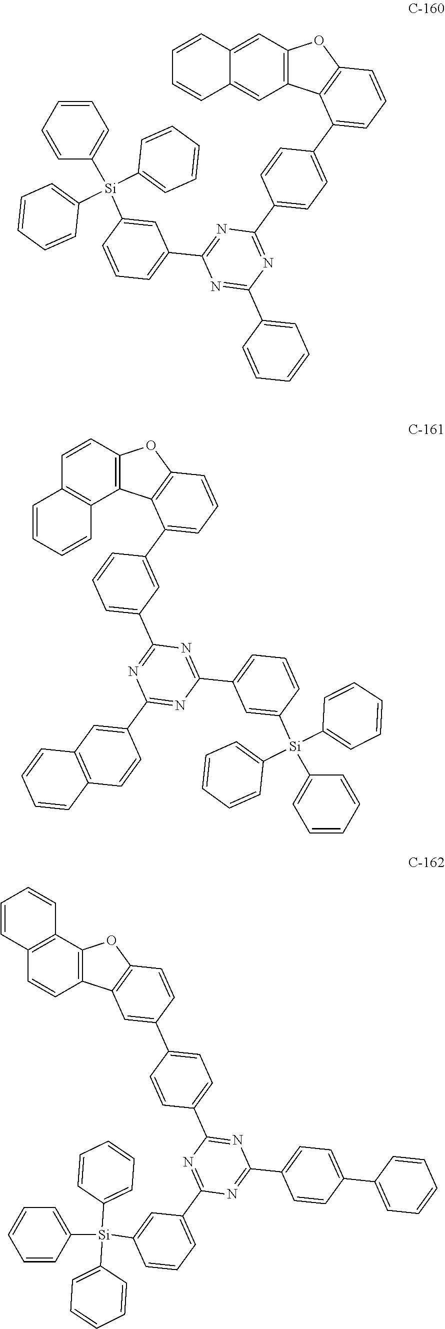 [SMILES: c1ccc(-c2nc(-c3ccc(-c4cccc5oc6cc7ccccc7cc6c45)cc3)nc(-c3cccc([Si](c4ccccc4)(c4ccccc4)c4ccccc4)c3)n2)cc1 ; c1ccc([Si](c2ccccc2)(c2ccccc2)c2cccc(-c3nc(-c4cccc(-c5cccc6oc7ccc8ccccc8c7c56)c4)nc(-c4ccc5ccccc5c4)n3)c2)cc1 ; c1ccc(-c2ccc(-c3nc(-c4ccc(-c5ccc6oc7c8ccccc8ccc7c6c5)cc4)nc(-c4cccc([Si](c5ccccc5)(c5ccccc5)c5ccccc5)c4)n3)cc2)cc1]

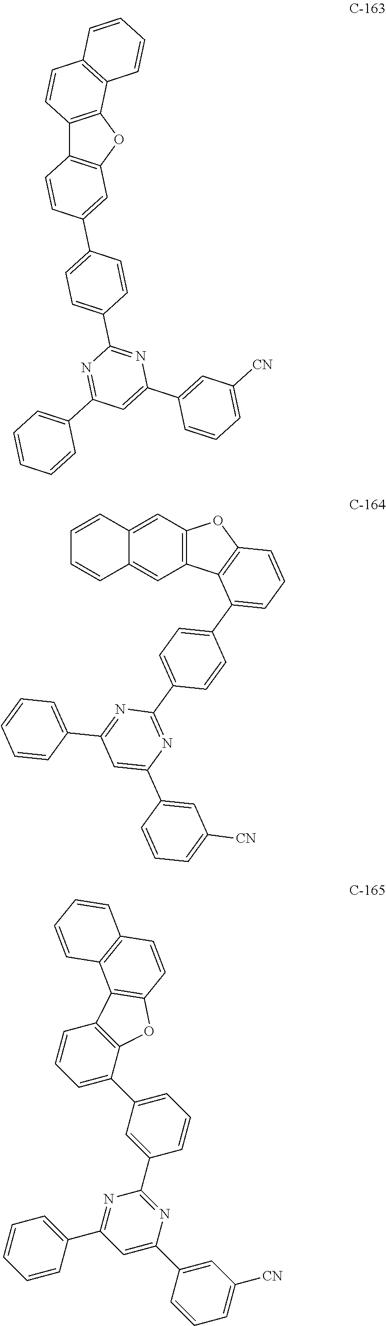 [SMILES: N#Cc1cccc(-c2cc(-c3ccccc3)nc(-c3ccc(-c4ccc5c(c4)oc4c6ccccc6ccc54)cc3)n2)c1 ; N#Cc1cccc(-c2cc(-c3ccccc3)nc(-c3ccc(-c4cccc5oc6cc7ccccc7cc6c45)cc3)n2)c1 ; N#Cc1cccc(-c2cc(-c3ccccc3)nc(-c3cccc(-c4cccc5c4oc4ccc6ccccc6c45)c3)n2)c1]

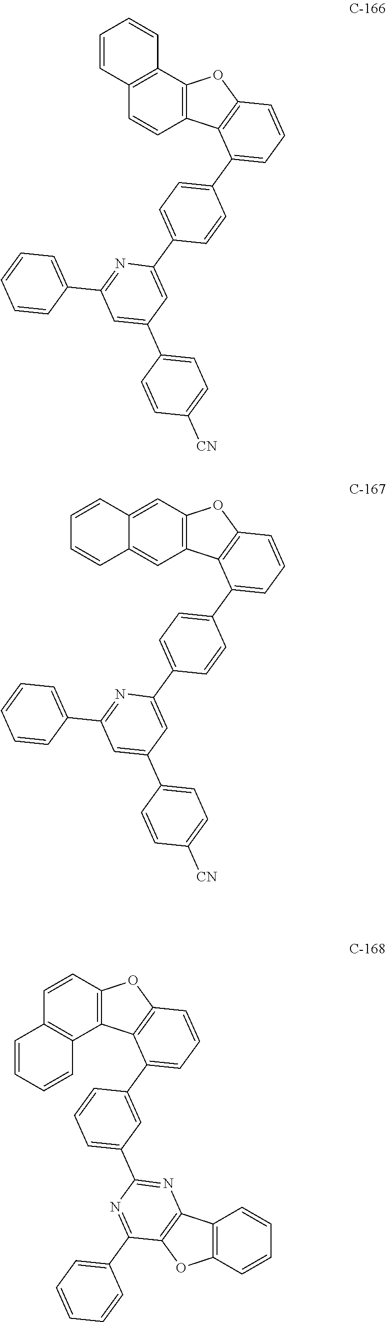 [SMILES: N#Cc1ccc(-c2cc(-c3ccccc3)nc(-c3ccc(-c4cccc5oc6c7ccccc7ccc6c45)cc3)c2)cc1 ; N#Cc1ccc(-c2cc(-c3ccccc3)nc(-c3ccc(-c4cccc5oc6cc7ccccc7cc6c45)cc3)c2)cc1 ; c1ccc(-c2nc(-c3cccc(-c4cccc5oc6ccc7ccccc7c6c45)c3)nc3c2oc2ccccc23)cc1]

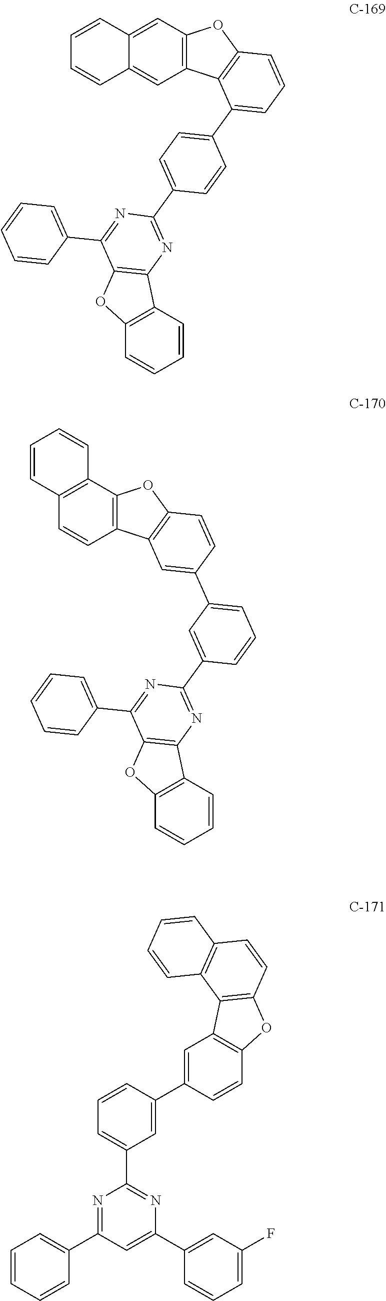 [SMILES: c1ccc(-c2nc(-c3ccc(-c4cccc5oc6cc7ccccc7cc6c45)cc3)nc3c2oc2ccccc23)cc1 ; c1ccc(-c2nc(-c3cccc(-c4ccc5oc6c7ccccc7ccc6c5c4)c3)nc3c2oc2ccccc23)cc1 ; Fc1cccc(-c2cc(-c3ccccc3)nc(-c3cccc(-c4ccc5oc6ccc7ccccc7c6c5c4)c3)n2)c1]

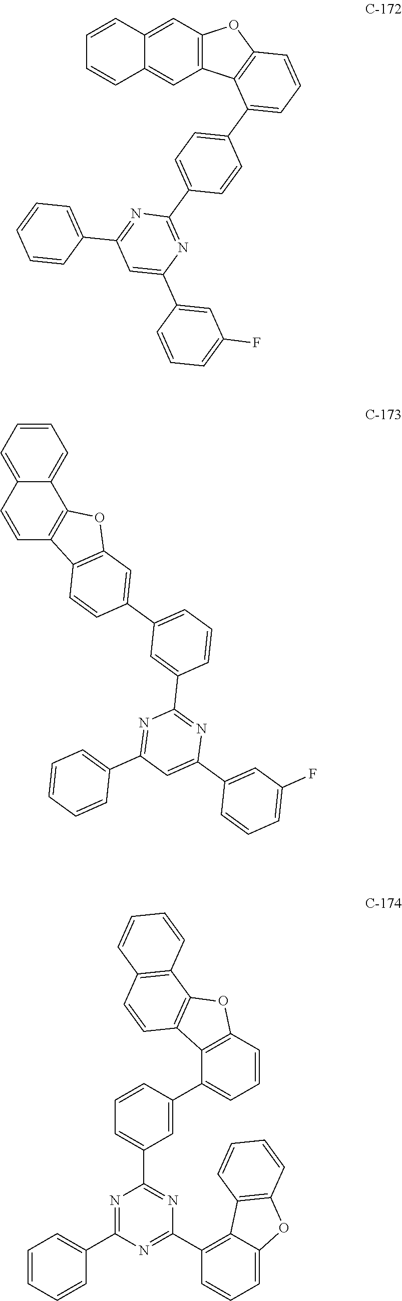 [SMILES: Fc1cccc(-c2cc(-c3ccccc3)nc(-c3ccc(-c4cccc5oc6cc7ccccc7cc6c45)cc3)n2)c1 ; Fc1cccc(-c2cc(-c3ccccc3)nc(-c3cccc(-c4ccc5c(c4)oc4c6ccccc6ccc54)c3)n2)c1 ; c1ccc(-c2nc(-c3cccc(-c4cccc5oc6c7ccccc7ccc6c45)c3)nc(-c3cccc4oc5ccccc5c34)n2)cc1]

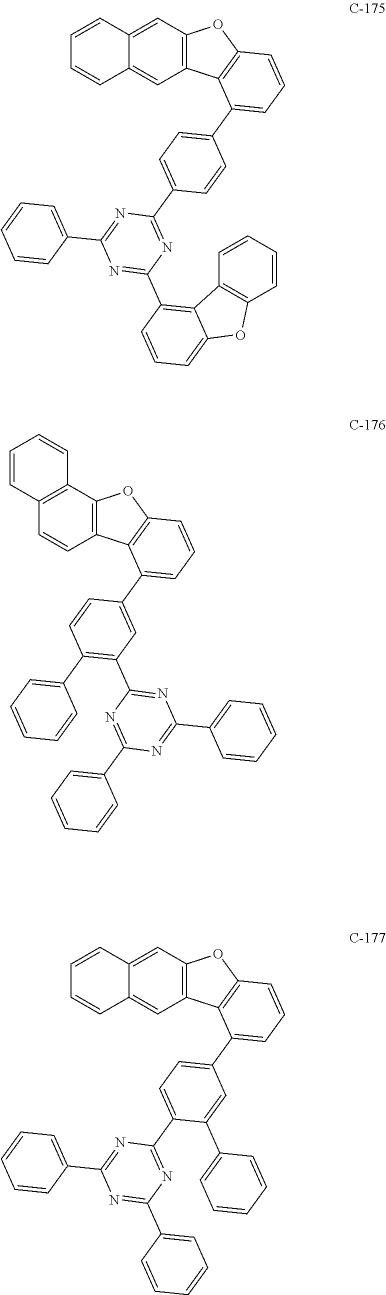 [SMILES: c1ccc(-c2nc(-c3ccc(-c4cccc5oc6cc7ccccc7cc6c45)cc3)nc(-c3cccc4oc5ccccc5c34)n2)cc1 ; c1ccc(-c2nc(-c3ccccc3)nc(-c3cc(-c4cccc5oc6c7ccccc7ccc6c45)ccc3-c3ccccc3)n2)cc1 ; c1ccc(-c2nc(-c3ccccc3)nc(-c3ccc(-c4cccc5oc6cc7ccccc7cc6c45)cc3-c3ccccc3)n2)cc1]

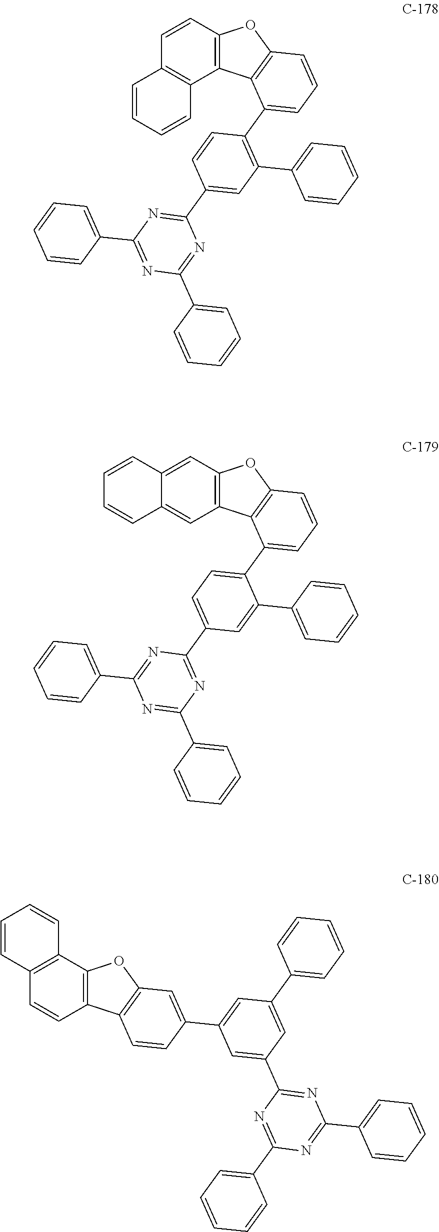 [SMILES: c1ccc(-c2nc(-c3ccccc3)nc(-c3ccc(-c4cccc5oc6ccc7ccccc7c6c45)c(-c4ccccc4)c3)n2)cc1 ; c1ccc(-c2nc(-c3ccccc3)nc(-c3ccc(-c4cccc5oc6cc7ccccc7cc6c45)c(-c4ccccc4)c3)n2)cc1 ; c1ccc(-c2cc(-c3ccc4c(c3)oc3c5ccccc5ccc43)cc(-c3nc(-c4ccccc4)nc(-c4ccccc4)n3)c2)cc1]

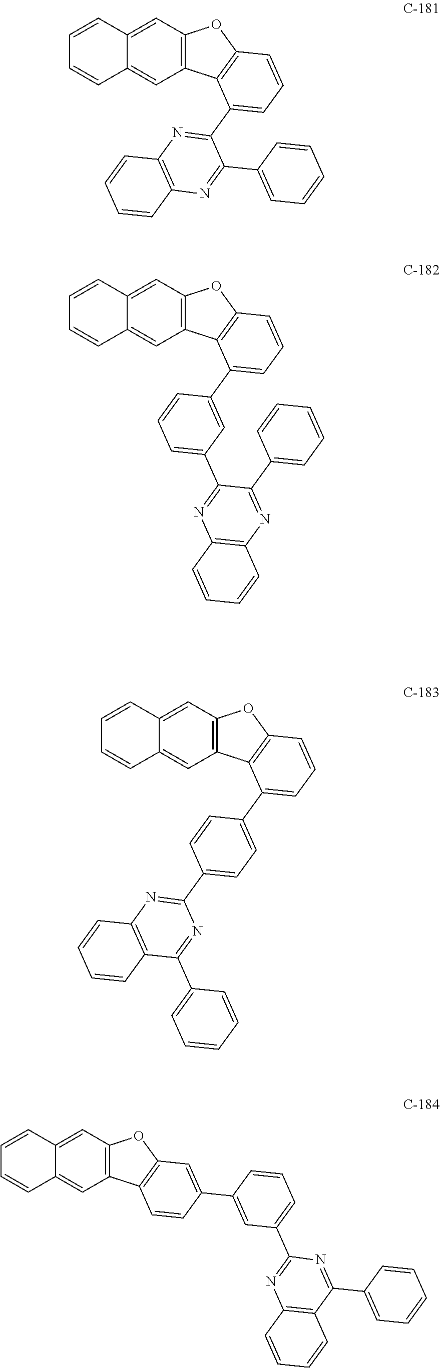 [SMILES: c1ccc(-c2nc3ccccc3nc2-c2cccc3oc4cc5ccccc5cc4c23)cc1 ; c1ccc(-c2nc3ccccc3nc2-c2cccc(-c3cccc4oc5cc6ccccc6cc5c34)c2)cc1 ; c1ccc(-c2nc(-c3ccc(-c4cccc5oc6cc7ccccc7cc6c45)cc3)nc3ccccc23)cc1 ; c1ccc(-c2nc(-c3cccc(-c4ccc5c(c4)oc4cc6ccccc6cc45)c3)nc3ccccc23)cc1]

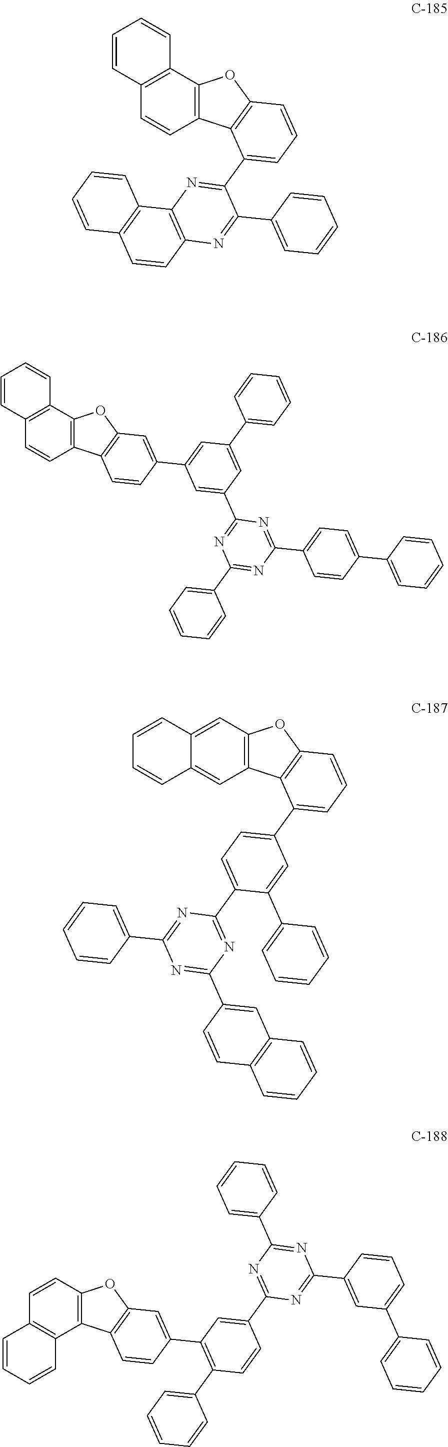 [SMILES: c1ccc(-c2nc3ccc4ccccc4c3nc2-c2cccc3oc4c5ccccc5ccc4c23)cc1 ; c1ccc(-c2ccc(-c3nc(-c4ccccc4)nc(-c4cc(-c5ccccc5)cc(-c5ccc6c(c5)oc5c7ccccc7ccc65)c4)n3)cc2)cc1 ; c1ccc(-c2nc(-c3ccc4ccccc4c3)nc(-c3ccc(-c4cccc5oc6cc7ccccc7cc6c45)cc3-c3ccccc3)n2)cc1 ; c1ccc(-c2cccc(-c3nc(-c4ccccc4)nc(-c4ccc(-c5ccccc5)c(-c5ccc6c(c5)oc5ccc7ccccc7c56)c4)n3)c2)cc1]

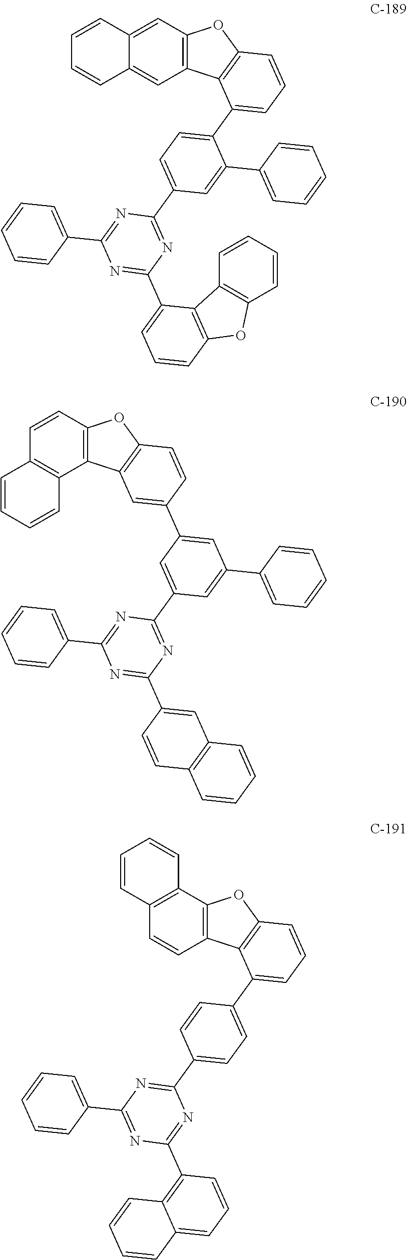 [SMILES: c1ccc(-c2nc(-c3ccc(-c4cccc5oc6cc7ccccc7cc6c45)c(-c4ccccc4)c3)nc(-c3cccc4oc5ccccc5c34)n2)cc1 ; c1ccc(-c2cc(-c3ccc4oc5ccc6ccccc6c5c4c3)cc(-c3nc(-c4ccccc4)nc(-c4ccc5ccccc5c4)n3)c2)cc1 ; c1ccc(-c2nc(-c3ccc(-c4cccc5oc6c7ccccc7ccc6c45)cc3)nc(-c3cccc4ccccc34)n2)cc1]

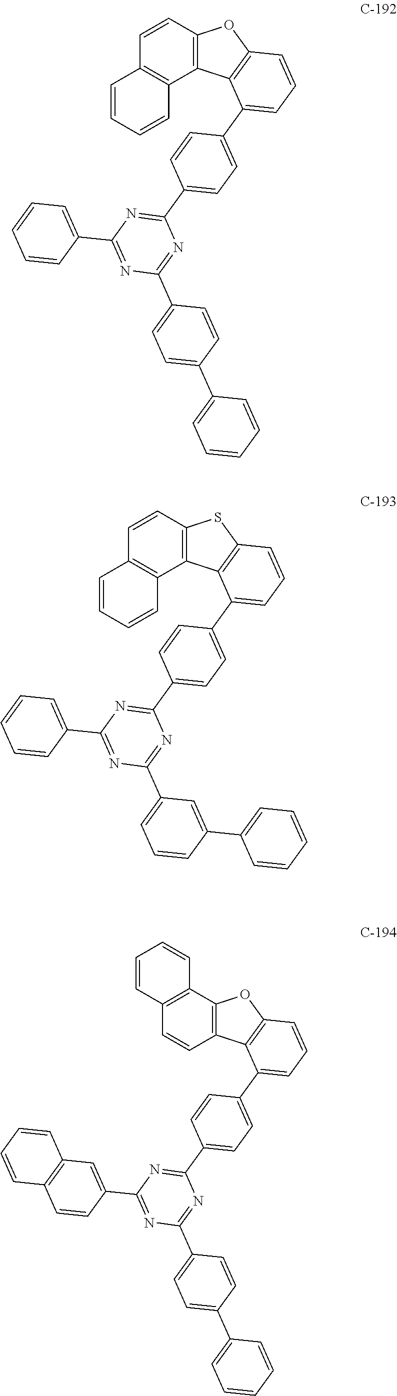 [SMILES: c1ccc(-c2ccc(-c3nc(-c4ccccc4)nc(-c4ccc(-c5cccc6oc7ccc8ccccc8c7c56)cc4)n3)cc2)cc1 ; c1ccc(-c2cccc(-c3nc(-c4ccccc4)nc(-c4ccc(-c5cccc6sc7ccc8ccccc8c7c56)cc4)n3)c2)cc1 ; c1ccc(-c2ccc(-c3nc(-c4ccc(-c5cccc6oc7c8ccccc8ccc7c56)cc4)nc(-c4ccc5ccccc5c4)n3)cc2)cc1]

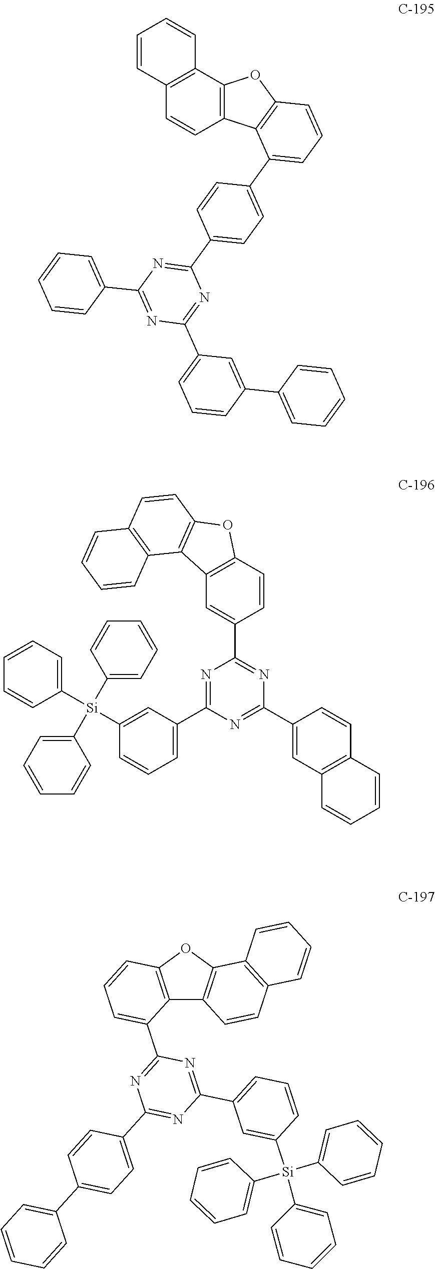 [SMILES: c1ccc(-c2cccc(-c3nc(-c4ccccc4)nc(-c4ccc(-c5cccc6oc7c8ccccc8ccc7c56)cc4)n3)c2)cc1 ; c1ccc([Si](c2ccccc2)(c2ccccc2)c2cccc(-c3nc(-c4ccc5ccccc5c4)nc(-c4ccc5oc6ccc7ccccc7c6c5c4)n3)c2)cc1 ; c1ccc(-c2ccc(-c3nc(-c4cccc([Si](c5ccccc5)(c5ccccc5)c5ccccc5)c4)nc(-c4cccc5oc6c7ccccc7ccc6c45)n3)cc2)cc1]

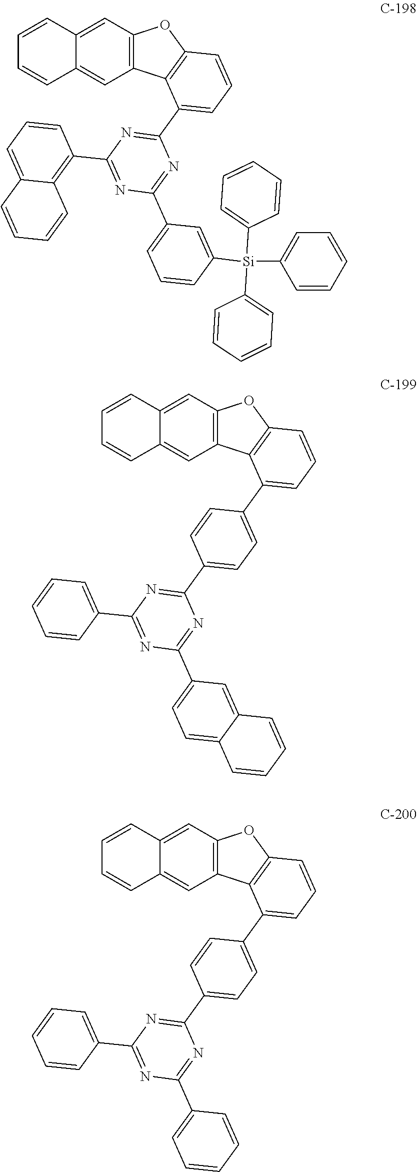

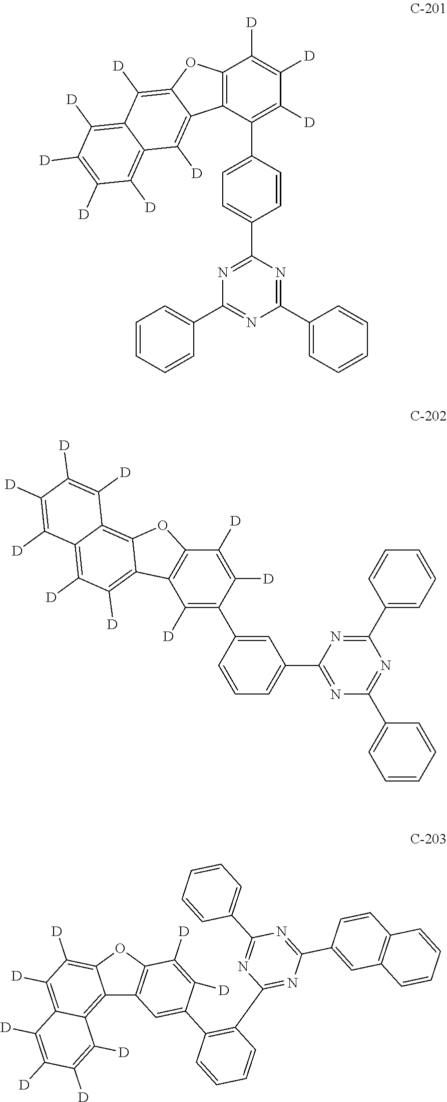 [SMILES: [2H]c1c([2H])c(-c2ccc(-c3nc(-c4ccccc4)nc(-c4ccccc4)n3)cc2)c2c(oc3c([2H])c4c([2H])c([2H])c([2H])c([2H])c4c([2H])c32)c1[2H] ; [2H]c1c(-c2cccc(-c3nc(-c4ccccc4)nc(-c4ccccc4)n3)c2)c([2H])c2c(oc3c4c([2H])c([2H])c([2H])c([2H])c4c([2H])c([2H])c32)c1[2H] ; [2H]c1c(-c2ccccc2-c2nc(-c3ccccc3)nc(-c3ccc4ccccc4c3)n2)cc2c(oc3c([2H])c([2H])c4c([2H])c([2H])c([2H])c([2H])c4c32)c1[2H]]

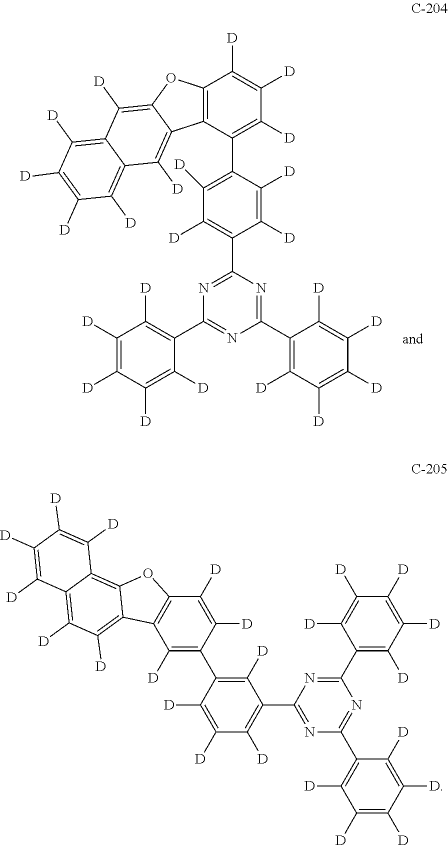

8. An organic electroluminescent device comprising an anode, a cathode, and at least one light-emitting layer between the anode and the cathode, wherein at least one of the light-emitting layers comprises the plurality of host materials according to claim 1.
